SEQUENCE LISTING (10 sequences)

SEQ ID NO: 1 - protein, 11 aa, synthetic construct
  SITE 6: note = Phosphotyrosine
  SITE 7: note = Hydroxyproline
AVYPHYXFWAR

SEQ ID NO: 2 - protein, 7 aa, synthetic construct
AKAKAKP

SEQ ID NO: 3 - protein, 33 aa, synthetic construct
KKKKKKKKKKGIGKFLHSAKKFGKAFVGEIMNS

SEQ ID NO: 4 - protein, 33 aa, synthetic construct; identical to SEQ ID NO: 3

SEQ ID NO: 5 - protein, 32 aa, synthetic construct
  SITE 1: note = Succinimidyl4-[N-maleimidomethyl]cyclohexane-1-carb oxylate modified residue
KKKKKKKKKGIGKFLHSAKKFGKAFVGEIMNS

SEQ ID NO: 6 - protein, 32 aa, synthetic construct; identical to SEQ ID NO: 5
  SITE 1: note = Dibenzocyclooctyne modified residue

SEQ ID NO: 7 - protein, 33 aa, synthetic construct; identical to SEQ ID NO: 3

SEQ ID NO: 8 - protein, 11 aa, synthetic construct
NDCQEFMTYHI

SEQ ID NO: 9 - protein, 9 aa, synthetic construct
  SITE 9: note = Amidated residue
  SITE 1..6: note = Intrachain disulfide bond
CYIQNCPLA

SEQ ID NO: 10 - protein, 11 aa, synthetic construct
  SITE 1: note = D-Amino acid
  SITE 2: note = N-methyl modified residue
  SITE 3: note = N-methyl modified residue
  SITE 4: note = N-methyl modified residue
  SITE 5: note = (3R,4R,6E)-6,7-didehydro-3-hydroxy-N,4-dimethyl-L-2 -aminooctanoic acid
  SITE 6: note = 2-Aminobutyric acid
  SITE 7: note = Sarcosine
  SITE 8: note = N-methyl modified residue
  SITE 10: note = N-methyl modified residue
ALLVTXXLVLA

(19) United States
(12) Patent Application Publication
(10) Pub. No.: US 2026/0056193 A1
(43) Pub. Date: Feb. 26, 2026

(54) FUNCTIONALIZED MACROMOLECULES AND SYSTEMS, METHODS OF SYNTHESIS, AND USES THEREOF

(71) Applicant: Seer, Inc., Redwood City, CA (US)

(72) Inventors: Hongwei XIA, Fremont, CA (US); Evan O’BRIEN, Oakland, CA (US); Jacob FABER-RICO, Belmont, CA (US); Craig STOLARCZYK, San Mateo, CA (US); Asim SIDDIQUI, San Francisco, CA (US); Emir YASUN, Pleasant Hill, CA (US); Niloufar HOSSEINI NASSAB, Sunnyvale, CA (US)

(21) Appl. No.: 19/097,711

(22) Filed: Apr. 1, 2025

Related U.S. Application Data
(63) Continuation of application No. PCT/US2023/075863, filed on Oct. 3, 2023.
(60) Provisional application No. 63/378,225, filed on Oct. 3, 2022.
(60) Provisional application No. 63/382,289, filed on Nov. 3, 2022.

(51) Int. Cl. G01N 33/543; C08F 122/14; C08F 122/22; C08F 122/24; C08F 126/06; G01N 33/68

(52) U.S. Cl. CPC G01N 33/5434; C08F 122/14; C08F 122/22; C08F 122/24; C08F 126/06; G01N 33/6848

(57) ABSTRACT

Disclosed herein are macromolecules, compositions thereof, and methods of making the same. In one aspect, the macromolecule are immobilized to a surface. In one aspect, provided herein are compositions, systems and kits comprising the macromolecules.

### CROSS-REFERENCE

The present application is a continuation of International Application No. PCT/US2023/075863, filed Oct. 3, 2023 which claims the benefit of U.S. Provisional Application No. 63/378,225, filed Oct. 3, 2022, and U.S. Provisional Application No. 63/382,289, filed Nov. 3, 2022, the content of each of which is incorporated by reference herein in its entirety.

### SEQUENCE LISTING

The instant application contains a Sequence Listing which has been submitted electronically in XML format and is hereby incorporated by reference in its entirety. Said XML copy, created on Oct. 7, 2025, is named 53344-766_301_SL.xml and is 15,532 bytes in size.

### BACKGROUND

Analysis of low abundance biomolecules is a significant challenge in proteomics. While some recent progress has improved capture of low-abundance biomolecules from samples, the compositions that aid in detecting low abundance biomolecules may be improved. Accordingly, improved structures and compositions are needed for detecting low abundance biomolecules from samples. The present disclosure provides structures and compositions thereof to address this need.

### SUMMARY

In one aspect, described herein is a macromolecule comprising a recurring unit of Formula (I-A):

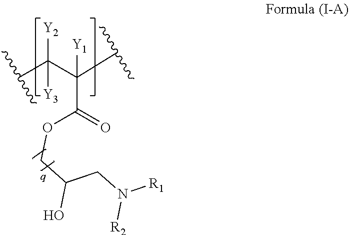

wherein each of Y₁, Y₂, and Y₃ is independently selected from hydrogen or C₁-C₆ alkyl; R₁ is hydrogen, optionally substituted succinate, C₁-C₆ alkyl sulfone, or phthalate,
R₂ is C₁-C₆ hydroxy, optionally substituted aryl, optionally substituted heteroaryl, optionally substituted C₃-C₆ heterocycloalkyl, C₁-C₁₂ amine, optionally substituted C₃-C₆ dicyloalkyl methane, C₁-C₆ alkyl guanidine, C₁-C₆ ether, optionally substituted —C₁-C₆ disulfide, C₁-C₆ thiol, optionally substituted succinate, optionally substituted C₁-C₁₂ alkylamine, C₁-C₆ alkyl acetamide, C₅-C₁₁ optionally substituted cycloalkyl, or C₁-C₆ aminophthalate; or
R₁ and R₂ are taken together with the nitrogen to which they are attached to form an optionally substituted heterocycle; or
R₁ and R₂ are taken together with the nitrogen to which they are attached to form an azide group; and
q is an integer between 1 and 6.

In some embodiments, Y₁ is C₁-C₃ alkyl. In some embodiments, Y₁ is C₁ alkyl. In some embodiments, each of Y₂ and Y₃ is hydrogen. In some embodiments, q is an integer between 1 and 3. In some embodiments, q is 1.

In some embodiments, R₁ is hydrogen and R₂ is selected from optionally substituted C₃-C₆ dicyloalkyl methane, optionally substituted aryl, optionally substituted heteroaryl, optionally substituted C₃-C₆ heterocycloalkyl, C₁-C₆ hydroxy, C₁-C₆ ether, optionally substituted —C₁-C₆ disulfide, optionally substituted succinate, optionally substituted C₁-C₆ alkylamine, C₁-C₆ alkyl acetamide, or C₁-C₆ alkyl guanidine.

In some embodiments, R₂ is optionally substituted dicyclohexyl methane. In some embodiments, R₂ is amino dicyclohexylmethane.

In some embodiments, R₂ is optionally substituted aryl. In some embodiments, R₂ is halogenated toluene. In some embodiments, R₂ is 2-flourotoluene.

In some embodiments, R₂ is C₁-C₆ hydroxy. In some embodiments, R₂ is C₃-C₆ hydroxy. In some embodiments, R₂ is —(CH₂)₆OH.

In some embodiments, R₂ is C₁-C₆ ether. In some embodiments, R₂ is —CH₂OCH₃, —CH₂CH₂OCH₃, —CH₂CH₂OCH₂CH₃, or —CH₂OCH₂CH₃. In some embodiments, R₂ is —CH₂CH₂OCH₃.

In some embodiments, R₂ is C₁-C₆ acetamide. In some embodiments, R₂ is —(CH₂)₂ acetamide.

In some embodiments, R₂ is optionally substituted di-C₁-C₆ alkyl disulfide. In some embodiments, R₂ is —CH₂CH₂—S—S—CH₂CH₂NH₂.

In some embodiments, R₂ is optionally substituted succinate. In some embodiments, R₂ is —(CH₂)₁₋₆NH(C═O)CH₂CH₂COOH. In some embodiments, R₂ is —(CH₂)₆NH(C═O)CH₂CH₂COOH.

In some embodiments, R₂ is optionally substituted heteroaryl. In some embodiments, R₂ is —(CH₂)₁₋₆ imidazole. In some embodiments, R₂ is —(CH₂)₃ imidazole. In some embodiments, R₂ is disubstituted C₂-C₄ imidazole. In some embodiments, R₂ is dipropyl imidazole.

In some embodiments, R₂ is optionally substituted heterocyclalkyl. In some embodiments, R₂ is —(CH₂)₁₋₆ pyrrolidine. In some embodiments, R₂ is —(CH₂)₂ pyrrolidine.

In some embodiments, R₂ is optionally substituted C₁-C₆ alkylamine. In some embodiments, R₂ is —(CH₂)₁₋₃ dimethylamine. In some embodiments, R₂ is —(CH₂)₂ dimethylamine.

In some embodiments, R₂ is C₁-C₆ guanidine. In some embodiments, R₂ is —(CH₂)₂ guanidine.

In some embodiments, each of R₁ and R₂ is nitrogen. In some embodiments, R₁ and R₂ are taken together to form an optionally substituted heterocycle. In some embodiments, the optionally substituted heterocycle is a triazole. In some embodiments, the optionally substituted triazole comprises benzylamide. In some embodiments, the benzylamide is halogenated.

In some embodiments, R₁ is optionally substituted succinate and R₂ is optionally substituted C₃-C₆ dicyloalkyl methane, optionally substituted aryl, C₁-C₆ thiol, or optionally substituted succinate. In some embodiments, R₁ is optionally substituted succinate (e.g., —C(═O)CH₂CH₂COOH).

In some embodiments, R₁ is succinate and R₂ is optionally substituted C₃-C₆ dicyloalkyl methane, optionally substituted aryl, C₁-C₆ thiol, or optionally substituted succinate. In some embodiments, R₂ is dicyclohexylmethane succinate. In some embodiments, R₂ is optionally substituted aryl. In some embodiments, R₂ is 2-flourotoluene. In some embodiments, R₂ is C₁-C₆ thiol. In some embodiments, R₂ is —(CH₂)₂SH. In some embodiments, R₂ is optionally substituted succinate. In some embodiments, R₂ is —(CH₂)₁₋₁₂NH(C═O)CH₂CH₂COOH. In some embodiments, R₂ is —(CH₂)₁₋₃NH(C═O)CH₂CH₂COOH. In some embodiments, R₂ is —(CH₂)₂NH(C═O)CH₂CH₂COOH. In some embodiments, R₂ is —(CH₂)₁₀₋₁₂NH(C═O)CH₂CH₂COOH. In some embodiments, R₂ is —(CH₂)₁₂NH(C═O)CH₂CH₂COOH.

In some embodiments, R₁ is C₂-C₁₂ alkenyl succinate and R₂ is substituted succinate. In some embodiments, R₁ is C₈ alkenyl succinate and R₂ is C₈ alkenyl ethylaminosuccinate.

In some embodiments, R₁ is C₁-C₆ sulfone and R₂ is optionally substituted C₁-C₆ alkylamine. In some embodiments, R₁ is —(CH₂)₃SOOOH and R₂ is —(CH₂)₁₋₆N(CH₃)₂(CH₂CH₂CH₂SOOOH) or —(CH₂)₁₋₆N(CH₂CH₂CH₂SOOOH)₂. In some embodiments, R₂ is —(CH₂)₂N(CH₃)₂(CH₂CH₂CH₂SOOOH). In some embodiments, R₂ is —(CH₂)₂N(CH₂CH₂CH₂SOOOH)₂.

In some embodiments, R₁ is phthalate and R₂ is C₁-C₆ aminophthalate. In some embodiments, R₂ is C₂-C₆ aminophthalate. In some embodiments, R₂ is C₂ aminophthalate. In some embodiments, R₂ is C₆ aminophthalate.

In one aspect, described herein is a macromolecule comprising a recurring unit of Formula (II):

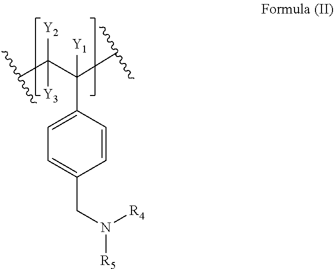

wherein each of Y₁, Y₂, and Y₃ is independently selected from hydrogen or C₁-C₆ alkyl;
R₄ is hydrogen or C₁-C₆ thiol; and
R₅ is succinate, C₁-C₆ thiol, optionally substituted aryl, or optionally substituted —C₁-C₆ disulfide.

In some embodiments, Y₁ is C₁-C₃ alkyl. In some embodiments, Y₁ is C₁ alkyl. In some embodiments, each of Y₂ and Y₃ is hydrogen

In some embodiments, R₄ is hydrogen and R₅ is optionally substituted —C₁-C₆ disulfide. In some embodiments, R₅ is optionally substituted di-C₁-C₆ alkyl disulfide. In some embodiments, R₅ is —CH₂CH₂—S—S—CH₂CH₂NH₂. In some embodiments, R₄ is C₁-C₆ thiol and R₅ is succinate. In some embodiments, R₄ is C₁-C₃ thiol. In some embodiments, R₄ is —(CH₂)₂SH.

In one aspect, described herein is a macromolecule comprising a recurring unit of Formula (III-A):

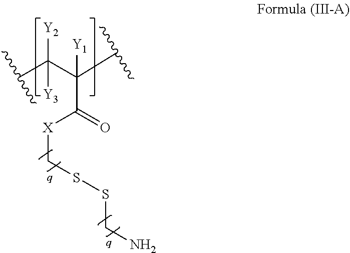

wherein each of Y₁, Y₂, and Y₃ is independently selected from hydrogen or C₁-C₆ alkyl; X is O or NH; and
each q is independently an integer between 1 and 6.

In some embodiments, Y₁ is C₁-C₃ alkyl. In some embodiments, Y₁ is C₁ alkyl. In some embodiments, each of Y₂ and Y₃ is hydrogen. In some embodiments, q is 2 or 3. In some embodiments, each q is 2.

In some embodiments, the recurring unit of Formula (I), (I-A), (I-A′), (II), (II′), (III), (III′), or (III-A) is selected from Table 1.

In some embodiments, the recurring unit of Formula (I), (I-A), (I-A′), (II), (II′), (III), (III′), or (III-A) is selected from

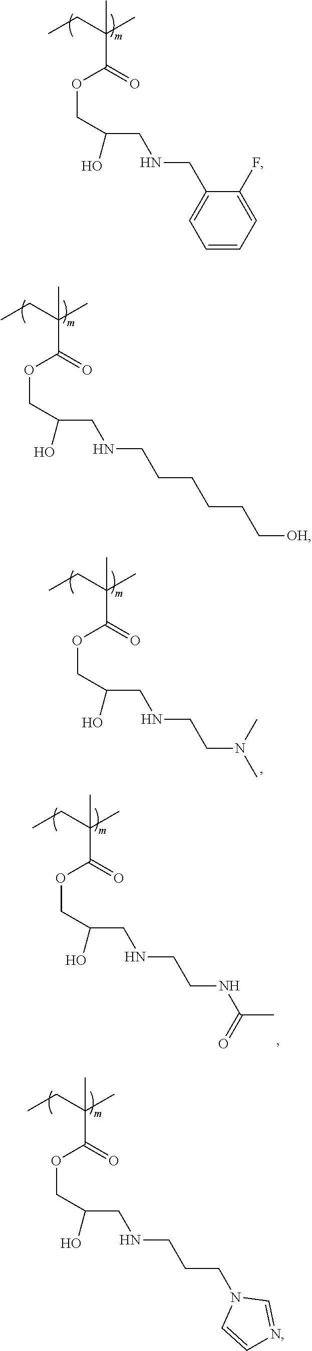

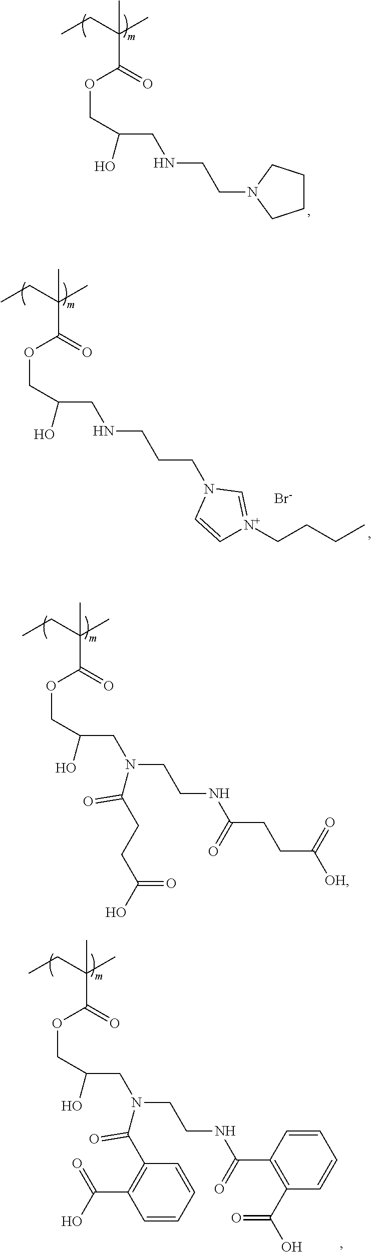

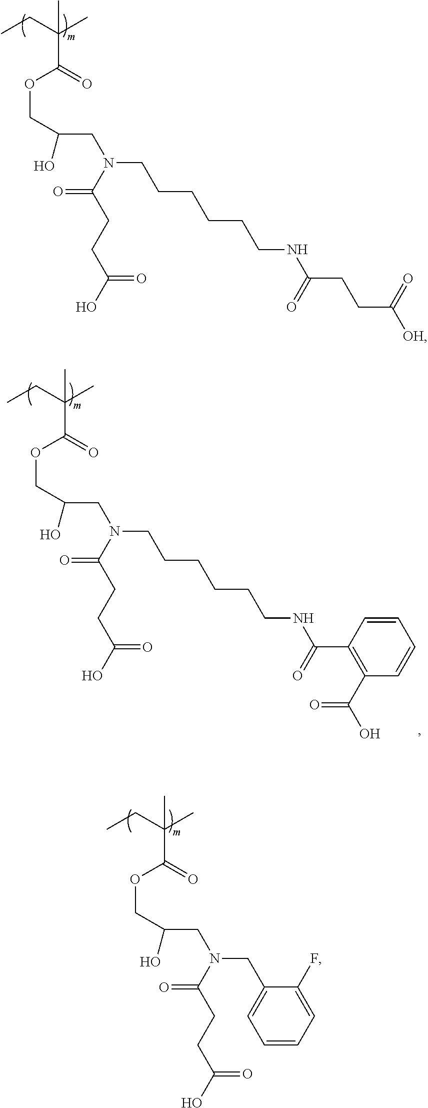

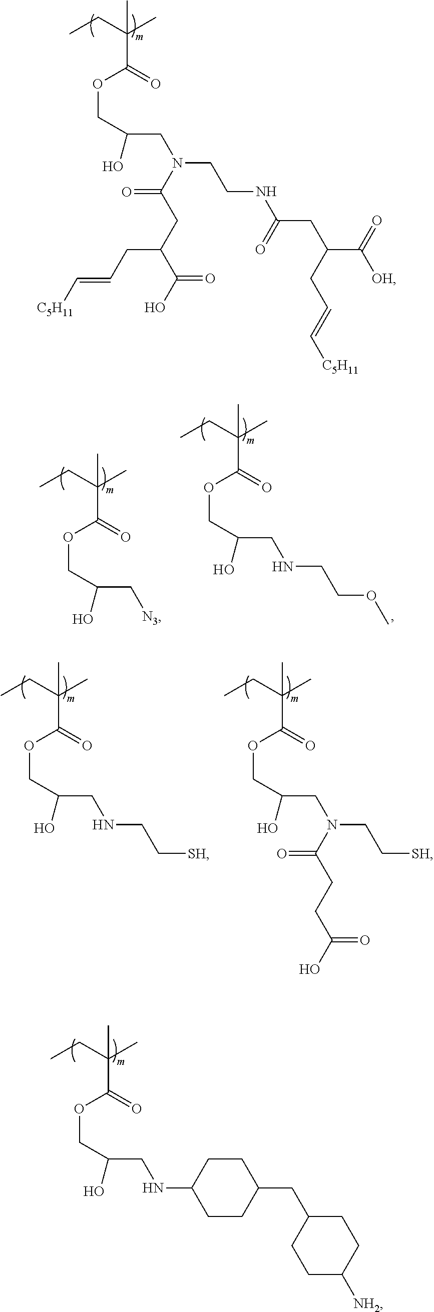

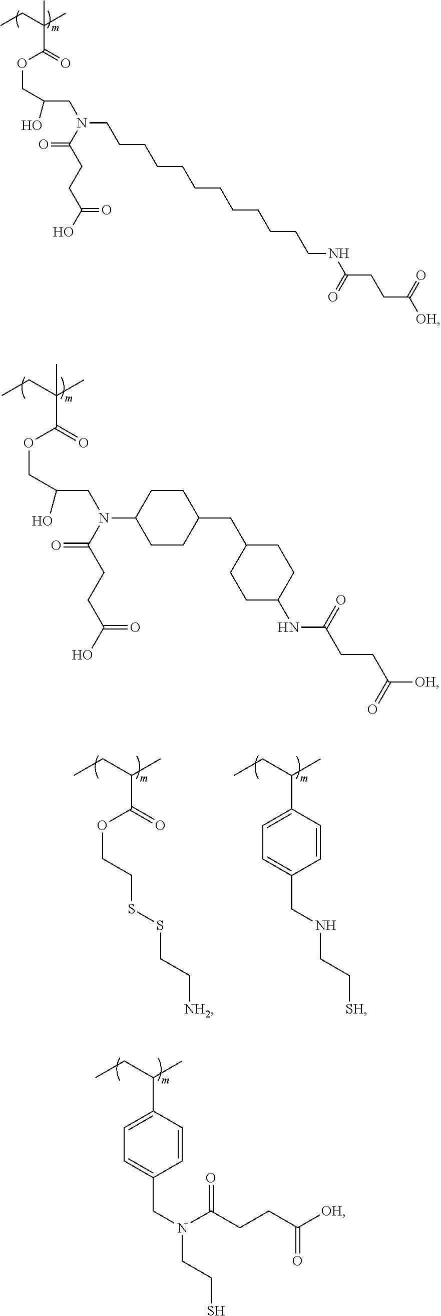

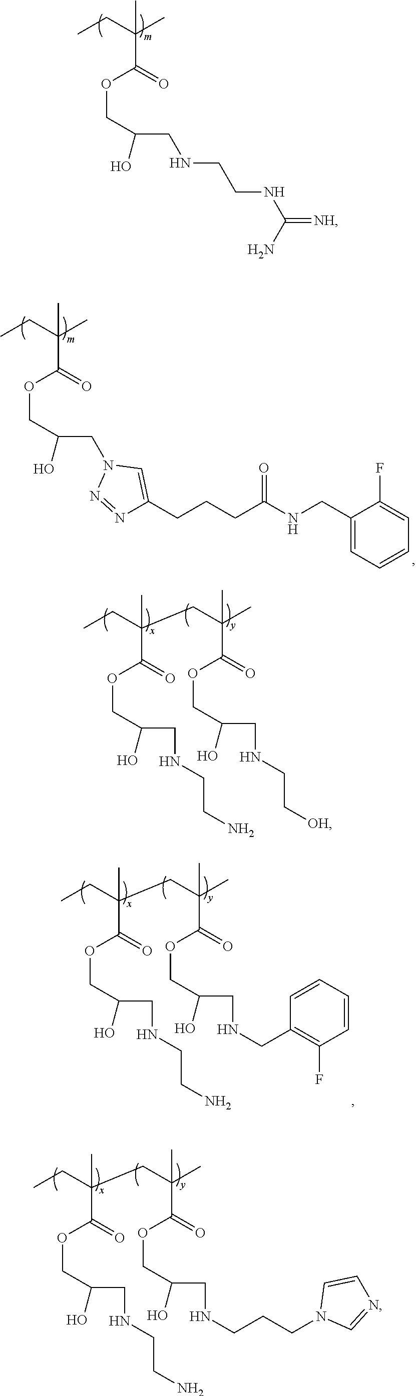

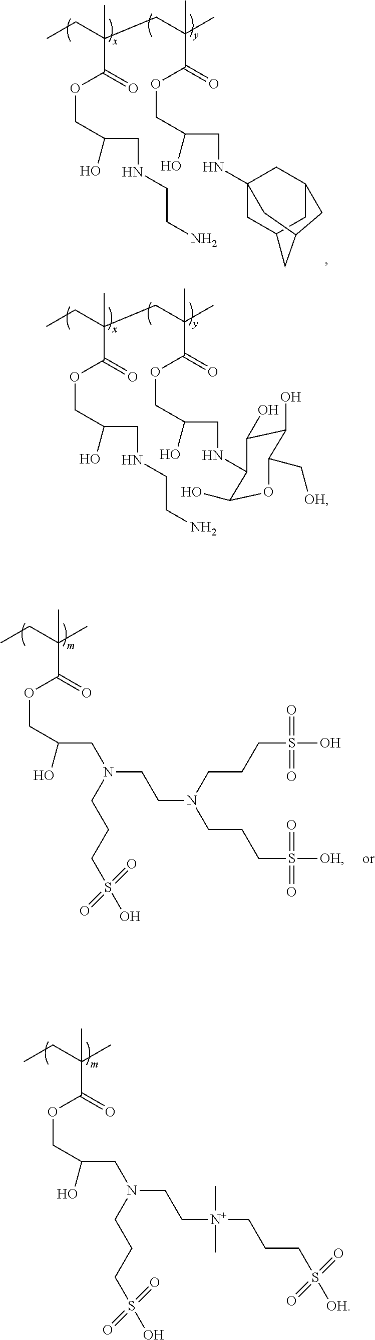

In one aspect, described herein is a macromolecule comprising recurring units of a first component and a second component, wherein the first component comprises a structure of Component (A) and the second component comprises a structure of Component (B):

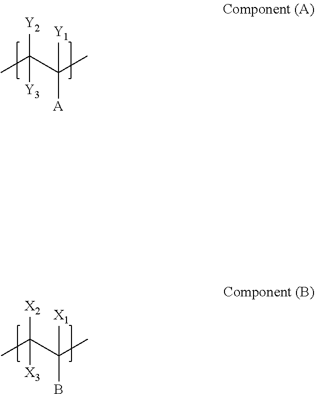

wherein each of Y₁, Y₂, and Y₃ is independently selected from hydrogen or C₁-C₆ alkyl; each of X₁, X₂, and X₃ is independently selected from hydrogen or C₁-C₆ alkyl;
A is

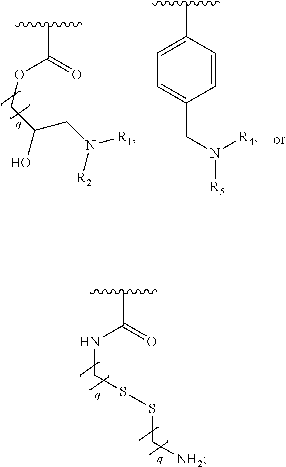

R₁ is hydrogen, optionally substituted succinate, C₁-C₆ alkyl sulfone, phthalate,
R₂ is C₁-C₁₂ amine, C₁-C₆ hydroxy, optionally substituted aryl, optionally substituted heteroaryl, optionally substituted C₃-C₆ heterocycloalkyl, two or more fused 3-6 member rings; optionally substituted C₃-C₆ dicyloalkyl methane, C₁-C₆ alkyl guanidine, C₁-C₆ ether, optionally substituted —C₁-C₆ disulfide, C₁-C₆ thiol, optionally substituted succinate, optionally substituted C₁-C₆ alkylamine, C₁-C₆ alkyl acetamide, C₁-C₆ aminophthalate, a boronic acid, C₁-C₆ thiol, C₁-C₁₁ optionally substituted cycloalkyl, or a monosaccharide; or
R₁ and R₂ are taken together with the nitrogen to which they are attached to form an optionally substituted heterocycle; or
R¹ and R₂ are taken together with the nitrogen to which they are attached to form an azide group;
R₄ is hydrogen or C₁-C₆ thiol;
R₅ is succinate, optionally substituted aryl, or optionally substituted —C₁-C₆ disulfide;
B is

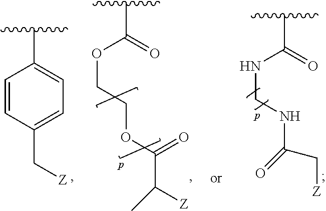

Z is a unit of Monomer (A) or Monomer (B);
each q is independently an integer between 1 and 6; and
p is an integer between 1 and 20.

In some embodiments, provided herein is a macromolecule comprising recurring units of a first component and a second component, wherein the first component comprises a structure of Component (A) and the second component comprises a structure of Component (B′):

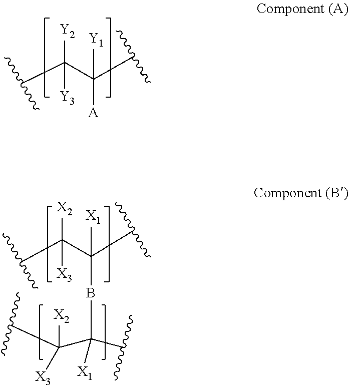

wherein each of Y₁, Y₂, and Y₃ is independently selected from hydrogen or C₁-C₆ alkyl; each of X₁, X₂, and X₃ is independently selected from hydrogen or C₁-C₆ alkyl;
A is

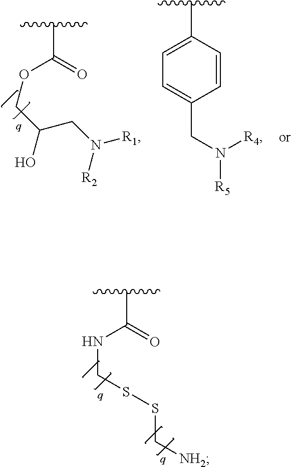

R₁ is hydrogen, nitrogen, optionally substituted succinate, C₁-C₆ alkyl sulfone, phthalate, R₂ is nitrogen, C₁-C₁₂ amine, C₁-C₆ hydroxy, optionally substituted aryl, optionally substituted heteroaryl, optionally substituted C₃-C₆ heterocycloalkyl, two or more fused 3-6 member rings; optionally substituted C₃-C₆ dicyloalkyl methane, C₁-C₆ alkyl guanidine, C₁-C₆ ether, optionally substituted —C₁-C₆ disulfide, C₁-C₆ thiol, optionally substituted succinate, optionally substituted C₁-C₆ alkylamine, C₁-C₆ alkyl acetamide, C₁-C₆ aminophthalate, a boronic acid, or a monosaccharide;
R₄ is hydrogen or C₁-C₆ thiol;
R₅ is succinate or optionally substituted —C₁-C₆ disulfide;
B is

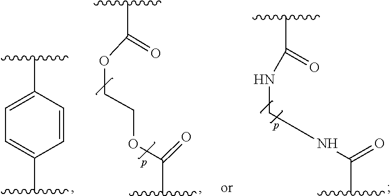

q is an integer between 1 and 6; and
p is an integer between 1 and 20.

In some embodiments, B is

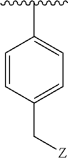

### and A is

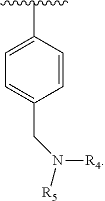

In some embodiments, R₄ is hydrogen and R₅ is optionally substituted —C₁-C₆ disulfide. In some embodiments, R₅ is optionally substituted di-C₁-C₆ alkyl disulfide. In some embodiments, R₅ is —CH₂CH₂—S—S—CH₂CH₂NH₂.

In some embodiments, B is

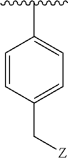

### and A is

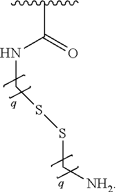

In some embodiments, B is

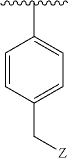

### and A is

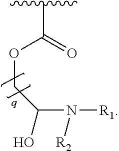

In some embodiments, B is

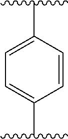 [SMILES: *c1ccc(*)cc1]

In some embodiments, B is

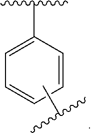

In some embodiments, B is

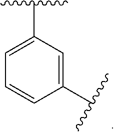 [SMILES: *c1cccc(*)c1]

In some embodiments, B is

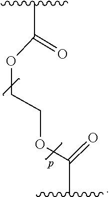

In some embodiments, B is

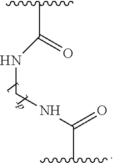

In some embodiments, A is

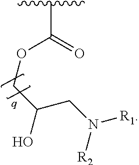

In some embodiments, A is

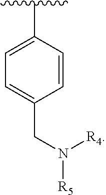

In some embodiments, A is

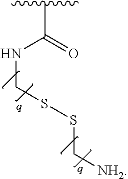

In some embodiments, R₁ is C₂-C₁₂ alkenyl succinate and R₂ is substituted succinate. In some embodiments, R₁ is C₈ alkenyl succinate and R₂ is C₈ alkenyl ethylaminosuccinate. In some embodiments, R₁ is C₁-C₆ alkyl sulfone and R₂ is optionally substituted C₁-C₆ alkylamine. In some embodiments, R₁ is —(CH₂)₃SOOOH and R₂ is —(CH₂)₁₋₆N(CH₃)₂(CH₂CH₂CH₂SOOOH) or —(CH₂)₁₋₆N(CH₂CH₂CH₂SOOOH)₂. In some embodiments, R₂ is —(CH₂)₂N(CH₃)₂(CH₂CH₂CH₂SOOOH).

In some embodiments, R₁ is phthalate and R₂ is C₁-C₆ aminophthalate. In some embodiments, R₂ is C₂-C₆ aminophthalate. In some embodiments, R₂ is C₂ aminophthalate. In some embodiments, R₂ is C₆ aminophthalate.

In some embodiments, R₁ is succinate and R₂ is substituted succinate or optionally substituted aryl. In some embodiments, R₂ is —(CH₂)₁₋₁₂NH(C═O)CH₂CH₂COOH. In some embodiments, R₂ is —(CH₂)₁₋₃NH(C═O)CH₂CH₂COOH. In some embodiments, R₂ is —(CH₂)₂NH(C═O)CH₂CH₂COOH. In some embodiments, R=is optionally substituted aryl. In some embodiments, R₂ is 2-flourotoluene.

In some embodiments, R¹ is hydrogen and R₂ is C₁-C₁₂ amine, optionally substituted C₁-C₆ alkylamine, C₁-C₆ acetamide, optionally substituted heterocyclalkyl, optionally substituted heteroaryl, a monosaccharide, two or more fused 3-6 member rings, optionally substituted aryl, or C₁-C₆ hydroxy.

In some embodiments, R₂ is C₁₋₁₂ amine. In some embodiments, R₂ is C₂ amine. In some embodiments, R₂ is C₆ amine. In some embodiments, R₂ is optionally substituted C₁-C₆ alkylamine. In some embodiments, R₂ is —(CH₂)₁₋₃ dimethylamine. In some embodiments, R₂ is —(CH₂)₂ dimethylamine. In some embodiments, R₂ is C₁-C₆ acetamide. In some embodiments, R₂ is —(CH₂)₂ acetamide. In some embodiments, R₂ is optionally substituted heterocyclalkyl. In some embodiments, R₂ is —(CH₂)₁₋₆ pyrrolidine. In some embodiments, R₂ is —(CH₂)₂ pyrrolidine. In some embodiments, R₂ is optionally substituted heteroaryl. In some embodiments, R₂ is —(CH₂)₁₋₆ imidazole. In some embodiments, R₂ is —(CH₂)₃ imidazole. In some embodiments, R₂ is —(CH₂)₁₋₆ pyridine. In some embodiments, R₂—(CH₂)pyridine. In some embodiments, R₂ is a monosaccharaide. In some embodiments, R₂ is glucose. In some embodiments, R₂ is D-glucose. In some embodiments, R₂ is two or more fused 3-6 member rings. In some embodiments, R₂ is three 6 member rings. In some embodiments, R₂ is optionally substituted aryl. In some embodiments, R₂ is halogenated toluene. In some embodiments, R₂ is 2-flourotoluene. In some embodiments, R₂ is C₁-C₆ hydroxy. In some embodiments, R₂ is C₂-C₆ hydroxy. In some embodiments, R₂ is —(CH₂)₂OH. In some embodiments, R₂ is —(CH₂)₆OH.

In some embodiments, the macromolecule further comprises a second structure of Component (A), wherein a first structure of Component (A) and the second structure of Component (A) are different.

In some embodiments, the first structure of Component (A), R₁ is hydrogen and R₂ is C₂ alkylamine and wherein the second structure of Component (A), R₁ is hydrogen and R₂ is C₁-C₆ hydroxy, optionally substituted aryl, optionally substituted heteroaryl, two or more fused 5 to 6 member rings, or a monosaccharide. In some embodiments, the second structure of Component (A), R₂ is C₁-C₆ hydroxy. In some embodiments, R₂ is C₂ alkylhydroxy.

In some embodiments, the second structure of Component (A), R₂ is optionally substituted aryl. In some embodiments, R₂ is 2-fluorotoluene.

In some embodiments, the second structure of Component (A), R₂ is optionally substituted heteroaryl. In some embodiments, R₂ is 1-propylimidazole.

In some embodiments, the second structure of Component (A), R₂ is two or more fused 5 to 6 member rings. In some embodiments, R₂ is three fused 6-member rings.

In some embodiments, the second structure of Component (A), R₂ is a monosaccharide. In some embodiments, R₂ is d-glucose.

In some embodiments, B is

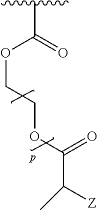

### and A is

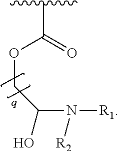

In some embodiments, R₁ is hydrogen and R₂ is selected from C₁-C₁₂ amine, optionally substituted C₃-C₆ dicyloalkyl methane, optionally substituted aryl, optionally substituted heteroaryl, optionally substituted C₃-C₆ heterocycloalkyl, C₁-C₆ hydroxy, C₁-C₆ ether, optionally substituted —C₁-C₆ disulfide, optionally substituted succinate, optionally substituted C₁-C₆ alkylamine, C₁-C₆ alkyl acetamide, a boronic acid, or C₁-C₆ alkyl guanidine.

In some embodiments, R₂ is optionally substituted dicyclohexyl methane. In some embodiments, R₂ is amino dicyclohexylmethane. In some embodiments, R₂ is optionally substituted aryl. In some embodiments, R₂ is halogenated toluene. In some embodiments, R₂ is 2-flourotoluene. In some embodiments, R₂ is a boronic acid. In some embodiments, R₂ is phenylboronic acid. In some embodiments, R₂ is C₁-C₆ hydroxy. In some embodiments, R₂ is —(CH₂)₂OH. In some embodiments, R₂ is C₁-C₆ ether. In some embodiments, R₂ is —CH₂OCH₃, —CH₂CH₂OCH₃, —CH₂CH₂OCH₂CH₃, or —CH₂OCH₂CH₃. In some embodiments, R₂ is —CH₂CH₂OCH₃. In some embodiments, R₂ is optionally substituted di-C₁-C₆ alkyl disulfide. In some embodiments, R₂ is —CH₂CH₂—S—S—CH₂CH₂NH₂. In some embodiments, R₂ is C₁-C₆ thiol. In some embodiments, R₂ is —(CH₂)₂SH. In some embodiments, R₂ is optionally substituted succinate. In some embodiments, R₂ is —(CH₂)₁₋₆ NH(C═O)CH₂CH₂COOH. In some embodiments, R₂ is —(CH₂)₆NH(C═O)CH₂CH₂COOH. In some embodiments, R₂ is C₁-C₁₂ amine. In some embodiments, R₂ is C₆-C₁₂ amine. In some embodiments, R₂ is C₁₂ amine. In some embodiments, R₂ is C₁-C₆ guanidine. In some embodiments, R₂ is —(CH₂)₂ guanidine. In some embodiments, R₂ is optionally substituted heteroaryl. In some embodiments, R₂ is —(CH₂)₁₋₆ imidazole. In some embodiments, R₂ is —(CH₂)₃ imidazole.

In some embodiments, R₁ is succinate and R₂ is optionally substituted C₃-C₆ dicyloalkyl methane, optionally substituted aryl, C₁-C₆ thiol, or optionally substituted succinate. In some embodiments, R₂ is dicyclohexylmethane succinate. In some embodiments, R₂ is —(CH₂)₁₋₁₂NH(C═O)CH₂CH₂COOH. In some embodiments, R₂ is —(CH₂)₁₋₃NH(C═O)CH₂CH₂COOH. In some embodiments, R₂ is —(CH₂)₂NH(C═O)CH₂CH₂COOH. In some embodiments, R₂ is —(CH₂)₁₀₋₁₂NH(C═O)CH₂CH₂COOH. In some embodiments, R₂ is Wherein R₂ is —(CH₂)₁₂NH(C═O)CH₂CH₂COOH. In some embodiments, R₁ is C₂-C₁₂ alkenyl succinate and R₂ is substituted succinate. In some embodiments, R₂ is C₈ alkenyl succinate and R₂ is C₈ alkenyl ethylaminosuccinate.

In some embodiments, each of R₁ and R₂ is nitrogen. In some embodiments, R₁ and R₂ are taken together to form an optionally substituted heterocycle. In some embodiments, the optionally substituted heterocycle is a triazole. In some embodiments, the optionally substituted triazole comprises benzylamide. In some embodiments, the benzylamide is halogenated. In some embodiments, R₁ is C₁-C₆ alkyl sulfone and R₂ is optionally substituted C₁-C₆ alkylamine.

In some embodiments, R₁ is —(CH₂)₃SOOOH and R₂ is —(CH₂)₁₋₆N(CH₂CH₂CH₂SOOOH)₂. In some embodiments, R₂ is —(CH₂)₂N(CH₂CH₂CH₂SOOOH)₂.

In some embodiments, R₁ is phthalate and R₂ is C₁-C₆ aminophthalate. In some embodiments, R₂ is C₂-C₆ aminophthalate. In some embodiments, R₂ is C₂ aminophthalate.

In some embodiments, B is

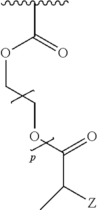

### and A is

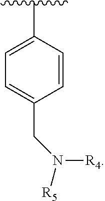

In some embodiments, R₄ is C₁-C₆ thiol and R₅ is succinate. In some embodiments, R₄ is C₁-C₃ thiol. In some embodiments, R₄ is —(CH₂)₂SH. In some embodiments, p is 1.

In some embodiments, B is

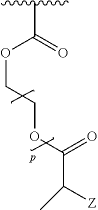

### and A is

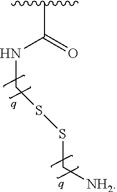

In some embodiments, p is 1. In some embodiments, q is 2 or 3. In some embodiments, q is 2.

In some embodiments, B is

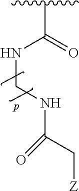

### and A is

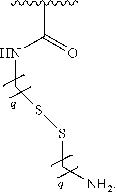

In some embodiments, p is 1. In some embodiments, q is 2 or 3. In some embodiments, q is 2.

In some embodiments, B is

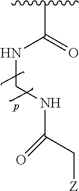

### and A is

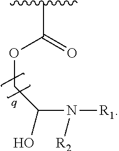

In some embodiments, p is 1.

In some embodiments, R₁ is hydrogen and R₂ is optionally substituted C₁-C₆ alkylamine. In some embodiments, R₂ is —(CH₂)₁₋₃ dimethylamine. In some embodiments, R₂ is —(CH₂)₂ dimethylamine.

In some embodiments, Component (A) comprises about 10 weight percent (wt %) to about 90 wt % of the macromolecule. In some embodiments, Component (A) comprises about 20 wt % to about 80 wt % of the macromolecule. In some embodiments, Component (A) comprises about 40 wt % to about 60 wt % of the macromolecule. In some embodiments, component (A) comprises about 50 wt % of the macromolecule. In some embodiments, Component (B) or Component (B′) comprises about 10 weight percent (wt %) to about 90 wt % of the macromolecule. In some embodiments, Component (B) or Component (B′) comprises about 20 wt % to about 80 wt % of the macromolecule. In some embodiments, Component (B) or Component (B′) comprises about 40 wt % to about 60 wt % of the macromolecule. In some embodiments, Component (B) or Component (B′) comprises about 50 wt % of the macromolecule.

In some embodiments, the structures of Component (A) and Component (B) or Component (B′) are selected from Table 2. In some embodiments, the structures of Component (A) and Component (B) or Component (B′) is selected from

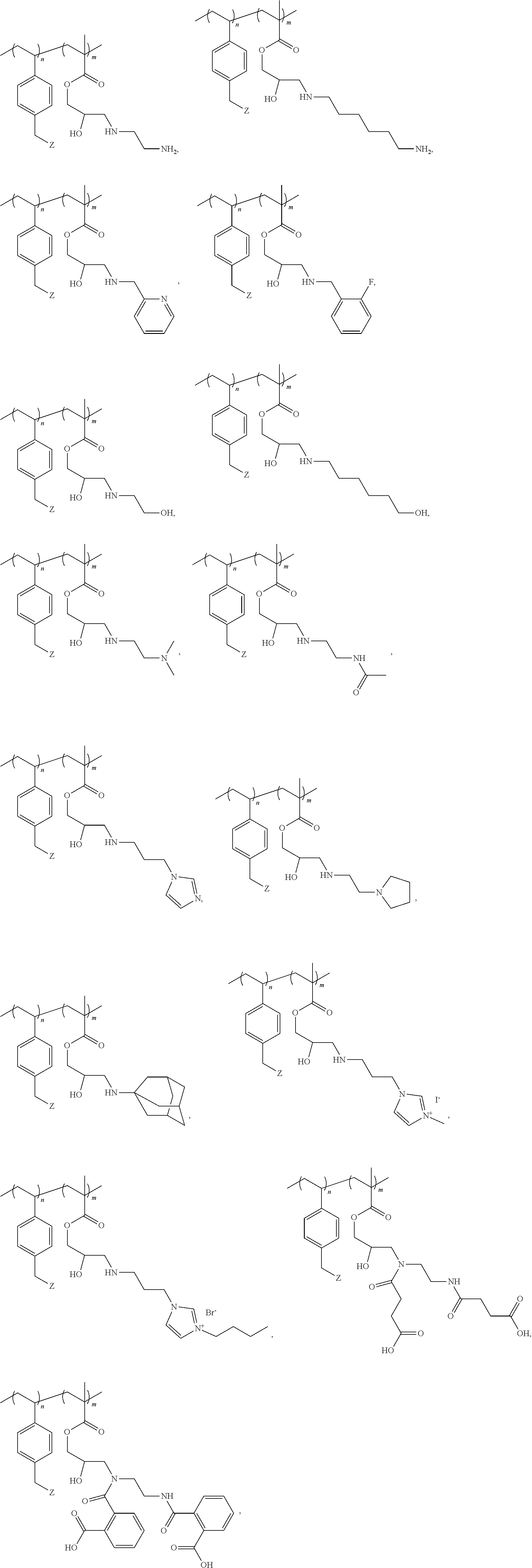

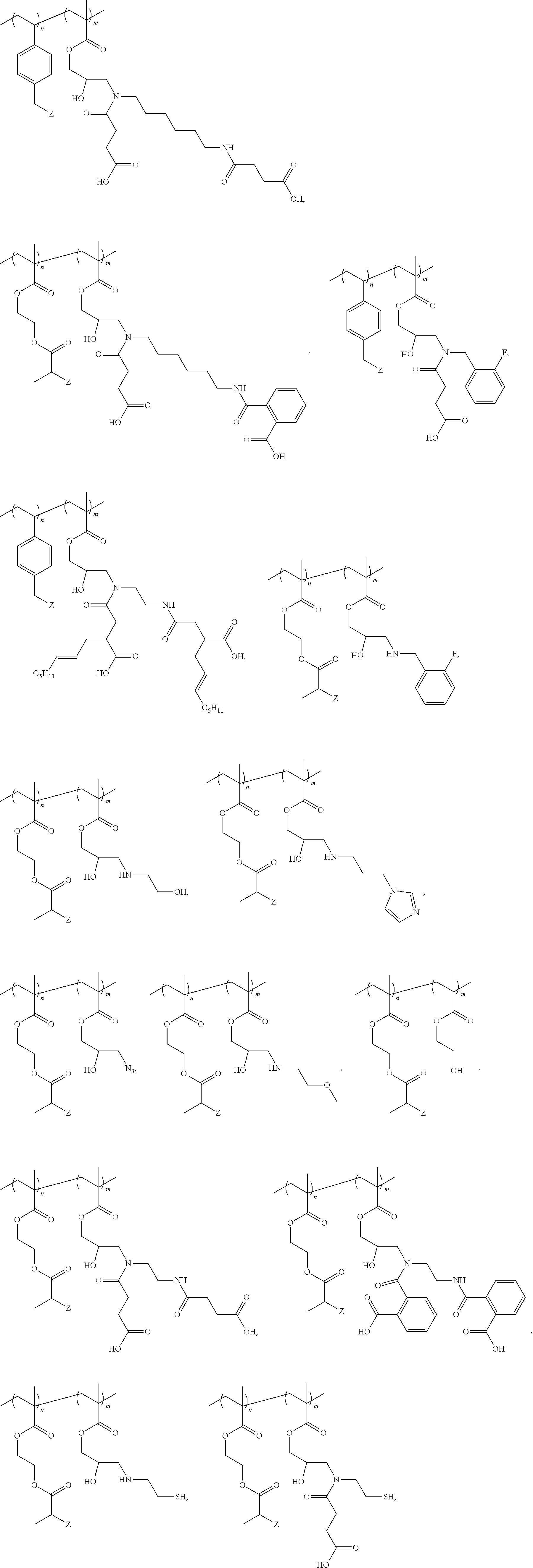

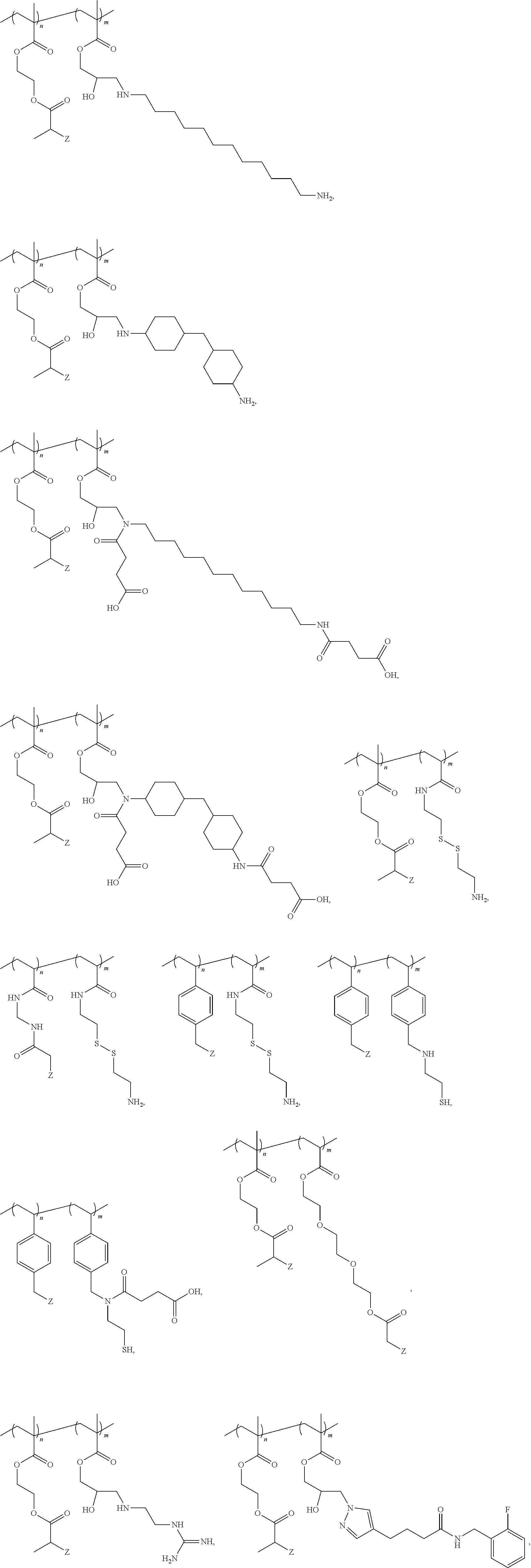

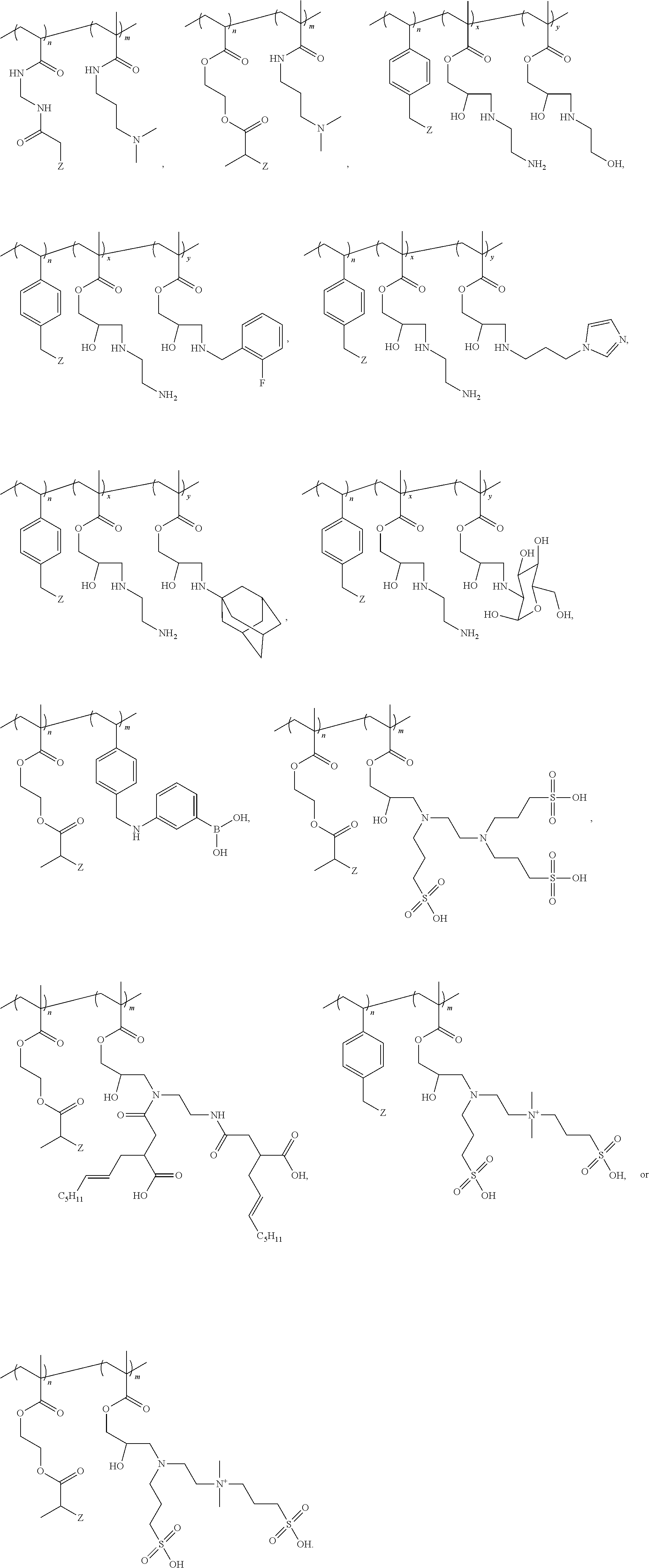

In one aspect, described herein is a surface comprising a moiety of Formula (IV):

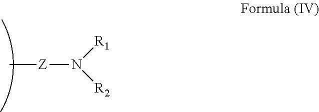

wherein Z is a linking moiety comprising a linear chain with 2 to 20 atoms selected from the group consisting of carbon, oxygen, and nitrogen, and optional substituents on the carbon and any nitrogen atoms in the linear chain;
R₁ is hydrogen, optionally substituted succinate, optionally substituted glutarate, optionally substituted adipate, optionally substituted pimelate, optionally substituted suberate, optionally substituted azelate, or optionally substituted sebacate; and
R₂ is optionally substituted —C₁-C₆ disulfide or optionally substituted C₁-C₆ thiol.

In some embodiments, Z is C₁-C₆ alkyl. In some embodiments, Z is C₃ alkyl. In some embodiments, Z is

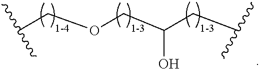

In some embodiments, Z is optionally substituted C₁-C₈ alkoxy. In some embodiments, some embodiments, Z is optionally substituted C₁-C₈ ether. In some embodiments, Z is C₁-C₈ alkoxy substituted with hydroxy. In some embodiments, Z is C₁-C₈ ether substituted with hydroxy. In some embodiments, Z is:

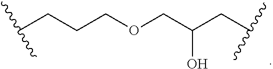 [SMILES: *CCCOCC(O)C*]

In some embodiments, R₁ is hydrogen and R₂ is substituted —C₁-C₆ alkyl disulfide. In some embodiments, R₂ is substituted di-C₁-C₆ alkyl disulfide. In some embodiments, R₂ is —CH₂CH₂—S—S—CH₂CH₂NH₂. In some embodiments, R₁ is succinate and R₂ is C₁-C₆ thiol. In some embodiments, R₂ is —(CH₂)₂SH.

In some embodiments, described herein is macromolecule comprising recurring units of a first component and a cross-linking recurring unit, wherein the first component comprises a structure of Component (A′):

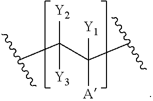

In some embodiments, each of Y₁, Y₂, and Y₃ is independently selected from hydrogen or C₁-C₆ alkyl. In some embodiments, A′ is

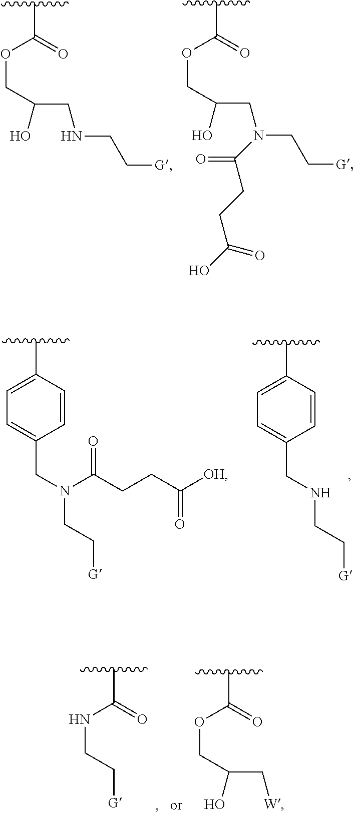

In some embodiments, G′ or W′ comprise Q′. In some embodiments, Q′ is a peptide. In some embodiments, W′ is

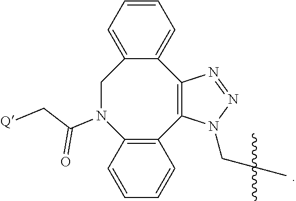

In some embodiments, G′ is

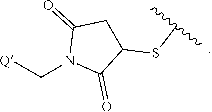

In some embodiments, described herein is a macromolecule comprising recurring units of a first component and a second component, wherein the first component comprises a structure of Component (A′) and the second component comprises a structure of (B). In some embodiments, component (A′) is

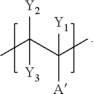

In some embodiments, component (B) is

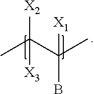

In some embodiments, each of Y₁, Y₂, and Y₃ is independently selected from hydrogen or C₁-C₆ alkyl. In some embodiments, each of X₁, X₂, and X₃ is independently selected from hydrogen or C₁-C₆ alkyl. In some embodiments, A′ is

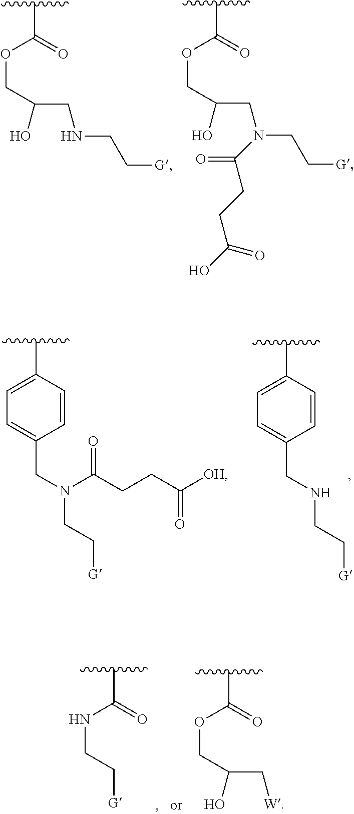

In some embodiments, G′ or W′ comprise Q′. In some embodiments, Q′ is a peptide. In some embodiments, B is

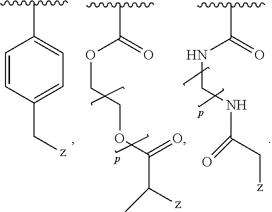

In some embodiments, Z is a unit of Monomer (A′) or (B). In some embodiments, q is an integer between 1 and 6. In some embodiments, p is an integer between 1 and 20. In some embodiments, W′ is

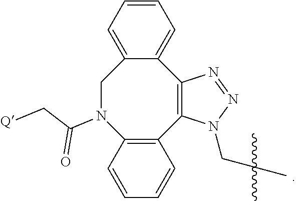

In some embodiments, G′ is

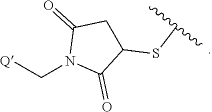

In some embodiments, provided herein is a macromolecule comprising recurring units of a first component and a second component, wherein the first component comprises a structure of Component (A′) and the second component comprises a structure of (B′):

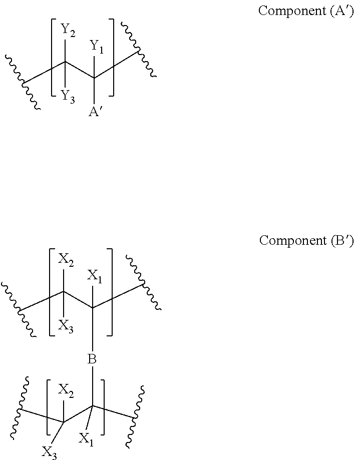

wherein
   each of Y₁, Y₂, and Y₃ is independently selected from hydrogen or C₁-C₆ alkyl;
   each of X₁, X₂, and X₃ is independently selected from hydrogen or C₁-C₆ alkyl;
   A′ is

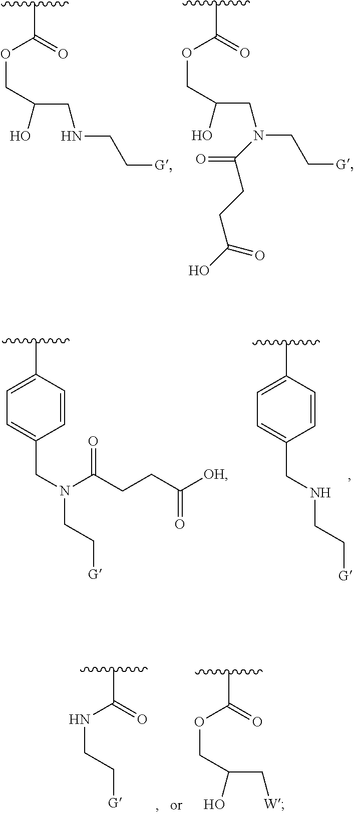

G′ or W′ comprise Q′;
   Q′ is a peptide;
   B is

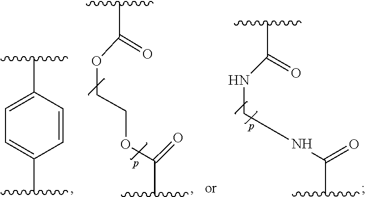

q is an integer between 1 and 6; and
p is an integer between 1 and 20.

In some embodiments, described herein is a surface comprising a moiety of Formula (IV′):

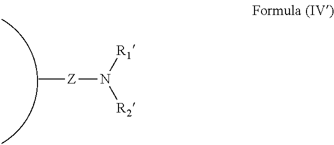

In some embodiments, Z is a linking moiety comprising a linear chain with 2 to 20 atoms selected from the group consisting of carbon, oxygen, and nitrogen and optional substituents on the carbon and any nitrogen atoms in the linear chain. In some embodiments, R₁′ is hydrogen or succinate. In some embodiments, R₂′ is C₁-C₆ alkyl-G′. In some embodiments, G′ comprises Q′. In some embodiments, Q′ is peptide. In specific embodiments, peptide does not comprise cysteine. In some embodiments, G′ is

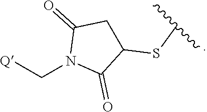

In some embodiments, the peptide comprises at most about 40 amino acids. In some embodiments, the peptide comprise at least about 20 amino acids. In some embodiments, the peptide comprises a synthetic sequence. In some embodiments, the peptide comprises non-natural amino acids.

In some embodiments, the macromolecules described herein may further comprise a peptide. In some embodiments, the peptide is bound to the macromolecule through non-specific adsorption.

In one aspect described herein is a system comprising a surface, a macromolecule described herein comprising recurring units of (A′) and (B) coupled to the surface, wherein the peptide comprises a binding site, and a protein interacting with the peptide at the binding site.

In one aspect described herein is a method comprising contacting a biological sample with a surface described herein; wherein the surface comprises peptides and said peptides are configured to bind to a protein, plurality of biomolecules or a portion thereof from the surface, and identifying at least the plurality of biomolecules or a portion thereof from the surface, wherein the plurality of biomolecules or a portion thereof comprises one or more biomolecules in the at least three different biomolecules in the biological sample. In some embodiments, the biomolecule is a protein. In some embodiments, the protein comprises a targeting protein. In some embodiments, the protein comprises a vacuolar lumen, lysosomal lumen, spliceosomal tri-snRNP complex, U4/U6 xU5 tri-snRNP complex, secretory granule lumen, intracellular organelle lumen, membrane raft, spliceosomal snRNP, complex, spermatoproteasome complex, or Golgi lumen protein.

In one aspect described herein is a surface comprising the macromolecule of any one of recurring units of the disclosure, wherein the macromolecule is immobilized to the modified surface.

In some embodiments, the macromolecule is covalently coupled to the surface. In some embodiments, the macromolecule is electrostatically coupled to the surface. In some embodiments, the macromolecule couples to the surface through a polymerization event. In some embodiments, the polymerization event comprises a reaction with a vinyl group on the surface.

In some embodiments, the surface is a bead or particle. In some embodiments, the surface is a particle. In some embodiments, the particle is a nanoparticle or microparticle. In some embodiments, the particle comprises a diameter of about 200 nanometers (nm) to about 400 nm. In some embodiments, the particle is a superparamagnetic iron oxide particle. In some embodiments, the particle comprises an iron oxide material. In some embodiments, the particle has an iron oxide core. In some embodiments, the particle has iron oxide crystals embedded in a polystyrene core. In some embodiments, the particle comprises an iron oxide core with a silica shell coating.

In some embodiments, the surface is selected from Table 3 or Table 4.

In some embodiments, the surface comprises the structure

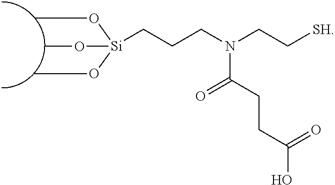

In some embodiments, the surface comprises the structure

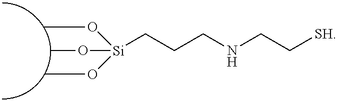

In some embodiments, the surface comprises the structure

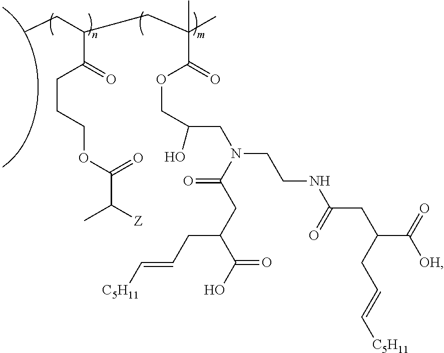

wherein

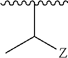

represents an attachment point for a unit of Component (A) or Component (B) when the monomer

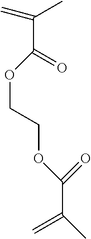

is crosslinked. For example, a structure of

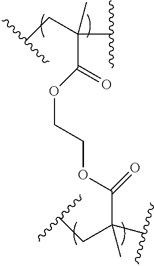

can be represented by

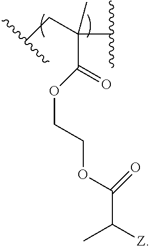

In some embodiments, the surface comprises the structure

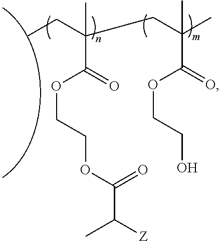

wherein

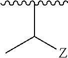

represents an attachment point for a unit of Component (A) or Component (B). In some embodiments, the surface comprises the structure

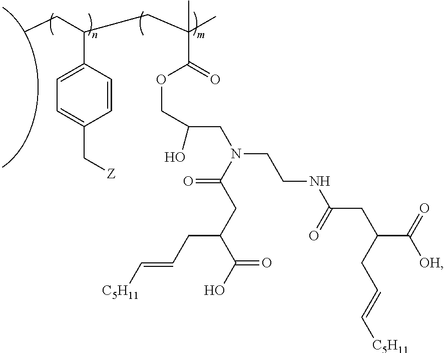

wherein

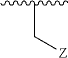

represents an attachment point for a unit of Component (A) or Component B when a divinylbenzene (DVB) monomer is crosslinked. For example, a structure of

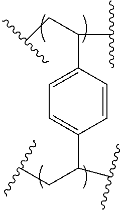

is represented by

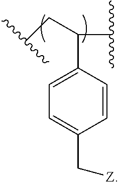

In some embodiments, the surface comprises the structure

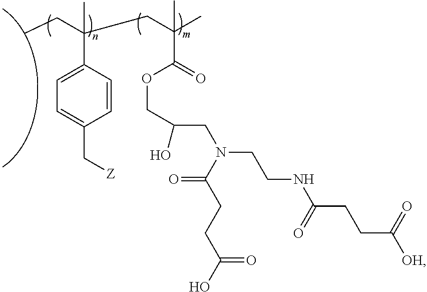

wherein

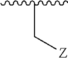

represents an attachment point for a unit of Component (A) or Component (B). In some embodiments, the surface comprises the structure

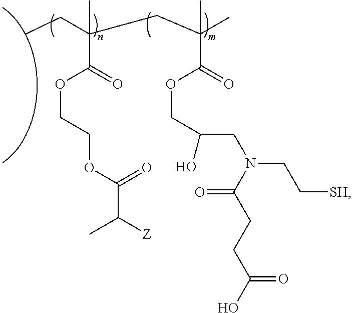

wherein

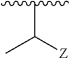

represents an attachment point for a unit of Component (A) or Component B). In some embodiments, the surface comprises the structure

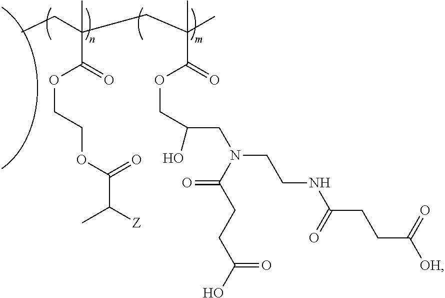

wherein

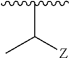

represents an attachment point for a unit of Component (A) or Component (B). In some embodiments, the surface comprises the structure

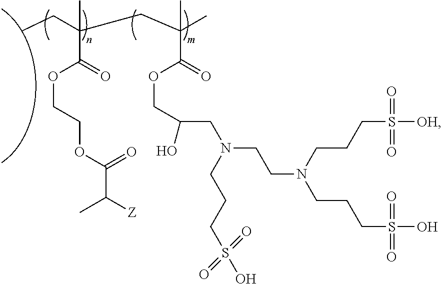

wherein

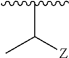

represents an attachment point for a unit of Component (A) or Component (B). In some embodiments, the surface comprises the structure

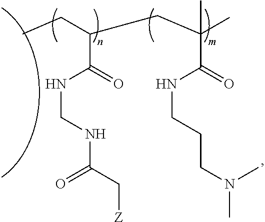

wherein

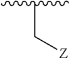

represents an attachment point for a unit of Component (A) or Component (B) when the monomer

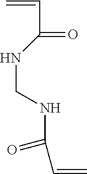

is crosslinked. For example, a structure of

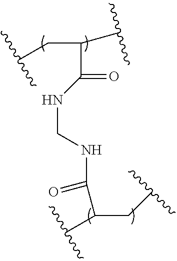

can be represented by

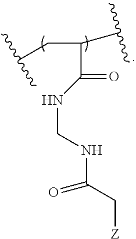

In some embodiments, the surface comprises the structure

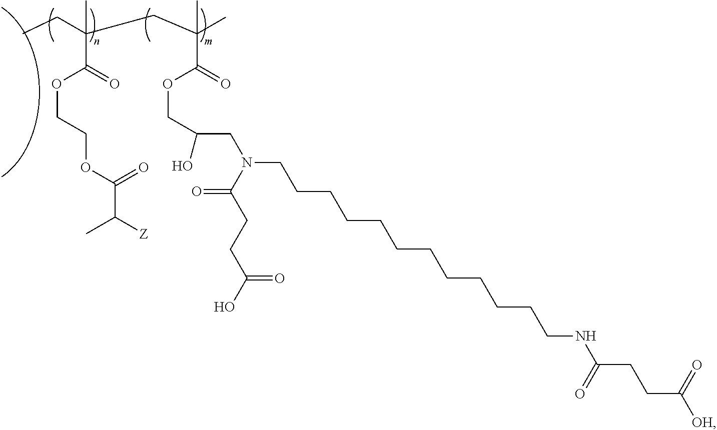

wherein

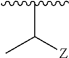

represents an attachment point for a unit of Component (A) or Component (B). In some embodiments, the surface comprises the structure

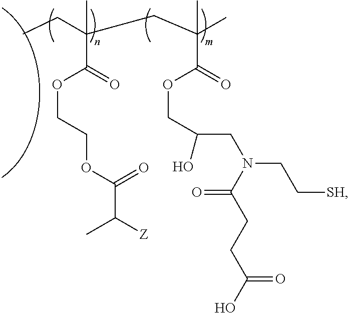

wherein

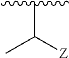

represents an attachment point for a unit of Component (A) or Component (B).

In one aspect, described herein is a method of (a) contacting a biological sample with a surface provided herein; (b) releasing a plurality of biomolecules or a portion thereof from the surface; and (c) identifying at least the plurality of biomolecules or a portion thereof from the surface. In some embodiments, the surface comprises at least two unique surfaces.

In some embodiments, the at least two unique surfaces comprise the structures

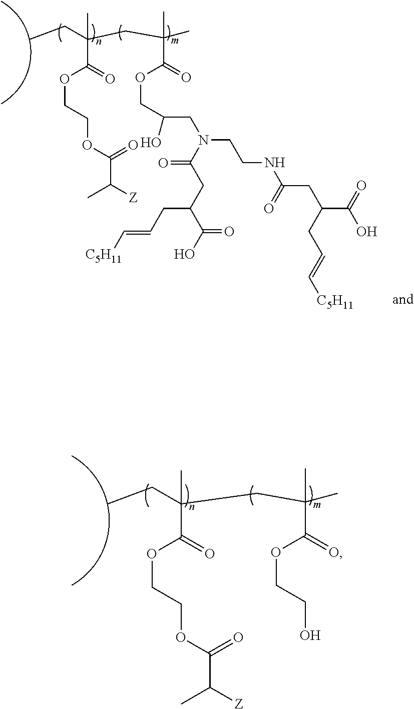

wherein

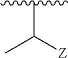

represents an attachment point for a unit of Component (A) or Component (B). In some embodiments, the at least two unique surfaces comprise the structures

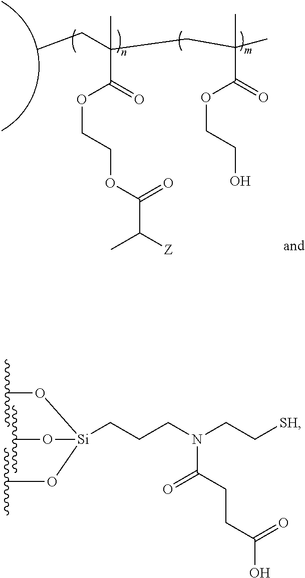

wherein

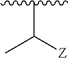

represents an attachment point for a unit of Component (A) or Component (B). In some embodiments, the at least two unique surfaces comprise the structures

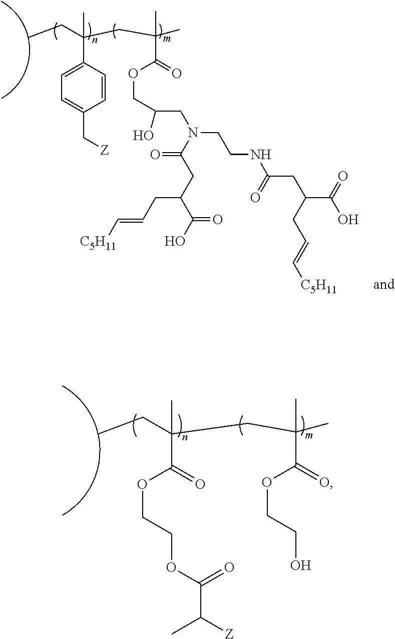

wherein

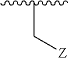

represents an attachment point for a unit of Component (A) or Component (B). In some embodiments, the at least two unique surfaces comprise the structures

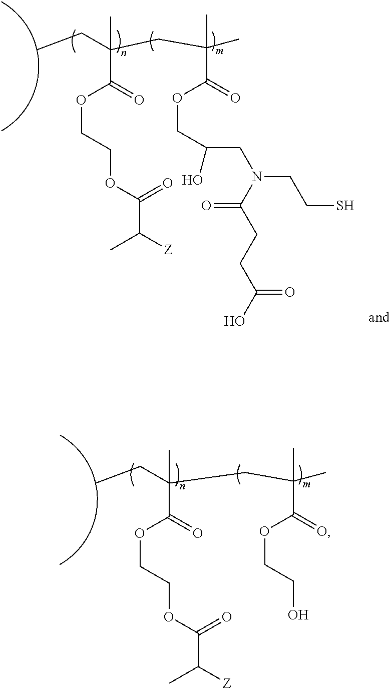

wherein

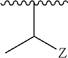

represents an attachment point for a unit of Component (A) or Component (B). In some embodiments, the at least two unique surfaces comprise the structures

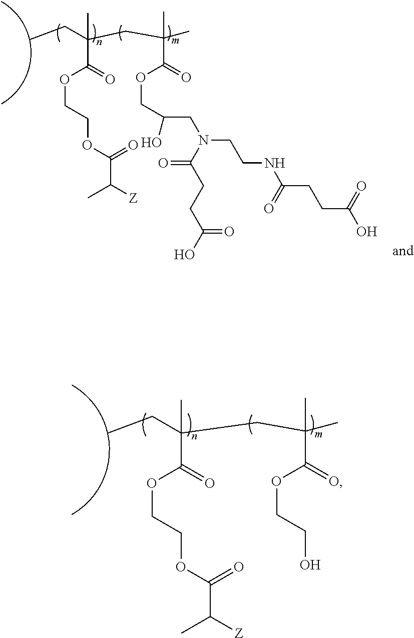

wherein

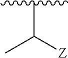

represents an attachment point for a unit of Component (A) or Component (B). In some embodiments, the at least two unique surfaces comprise the structures

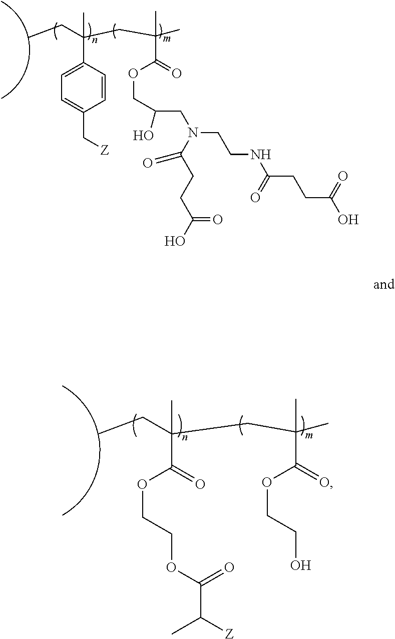

wherein

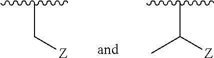

independently represent an attachment point for a unit of Component (A) or Component (B).

In some embodiments, the biomolecule comprises a protein. In some embodiments, the protein comprises a targeting protein. In some embodiments, the protein comprises a vacuolar lumen, lysosomal lumen, spliceosomal tri-snRNP complex, U4/U6 xU5 tri-snRNP complex, secretory granule lumen, intracellular organelle lumen, membrane raft, spliceosomal snRNP, complex, spermatoproteasome complex, or Golgi lumen protein.

In one aspect, described herein is a method of preparing a surface comprising recurring units of a first component and a second component, said method comprising: (a) providing a mixture of monomers in a solvent comprising a first monomer and a second monomer, wherein the first monomer comprises a vinyl group and wherein the second monomer comprises an epoxide group; (b) contacting a surface and the mixture of monomers, thereby producing a reaction mixture; (c) initiating free radical polymerization to produce a macromolecule immobilized to the surface; (d) contacting the macromolecule immobilized to the surface and an amine, thereby producing an aminated macromolecule; and (e) optionally contacting the aminated macromolecule with a compound comprising succinate, phthalate, or propanesulfone.

In some embodiments, the surface is a bead or particle. In some embodiments, the surface is a particle. In some embodiments, the particle is a nanoparticle or microparticle. In some embodiments, the particle comprises a diameter of about 200 nanometers (nm) to about 400 nm. In some embodiments, the particle is a superparamagnetic iron oxide particle. In some embodiments, the particle comprises an iron oxide material. In some embodiments, the particle has an iron oxide core. In some embodiments, the particle has iron oxide crystals embedded in a polystyrene core. In some embodiments, the particle comprises an iron oxide core with a silica shell coating.

In some embodiments, solvent is a polar solvent. In some embodiments, the solvent is acetonitrile, THF, or DMF

In some embodiments, free radical polymerization is initiated with a radical initiator. In some embodiments, the radical initiator is AIBN.

In some embodiments, the method comprises, subsequent to (c) and prior to (d), contacting the macromolecule immobilized to the surface with a quenching agent. In some embodiments, the quenching agent is introduced to the reaction mixture when the macromolecule immobilized to the surface comprises a diameter of about 300 nm to about 500 nm. In some embodiments, the diameter is about 325 nm to about 375 nm. In some embodiments, the quenching agent comprises benzoquinone.

In some embodiments, the method further comprises purifying the macromolecule immobilized to the surface. In some embodiments, purifying comprises washing the macromolecule immobilized to the surface with THF or ethanol

In some embodiments, the first monomer is divinylbenzene (DVB), ethyleneglycol dimethacrylate (EGDMA), or N,N′-methylenebisacrylamide (MBA). In some embodiments, the second monomer comprises glycidyl methacrylate. In some embodiments, the amine is C₁-C₁₂ alkylamine, C₁-C₆ hydroxyamine, C₁-C₆ alkoxyethylamine. In some embodiments, the C₁-C₁₂ alkylamine is diethylamine. In some embodiments, the C₁-C₆hydroxyamine is ethanolamine or hexanolamine. In some embodiments, the C₁-C₆ alkoxyethylamine is methoxyethylamine.

In some embodiments, the method comprises (e) contacting the aminated macromolecule with a compound comprising succinate, phthalate, thiol, or propylsulfone. In some embodiments, the compound comprises a succinate. In some embodiments, the succinate is C₈ alkenyl succinate or C₈ alkenyl ethylaminosuccinate.

In some embodiments, the compound comprises a thiol. In some embodiments, thiol is C₂ alkyl thiol.

In some embodiments, the compound comprises a phthalate. In some embodiments, the phthalate is C₁-C₆ aminophthalate.

In some embodiments, the compound comprises a propylsulfone. In some embodiments, the propylsulfone is dipropylsulfone ethylamine.

In some embodiments, the first monomer comprises about 10 weight percent (wt %) to about 90 wt % of the mixture of monomers. In some embodiments, the first monomer comprises about 20 wt % to about 80 wt % of the mixture of monomers. In some embodiments, the first monomer comprises about 40 wt % to about 60 wt % of the mixture of monomers. In some embodiments, the first monomer comprises about 50 wt % of the mixture of monomers. In some embodiments, the second monomer comprises about 10 weight percent (wt %) to about 90 wt % of the mixture of monomers. In some embodiments, the second monomer comprises about 20 wt % to about 80 wt % of the mixture of monomers. In some embodiments, the second monomer comprises about 40 wt % to about 60 wt % of the mixture of monomers. In some embodiments, the second monomer comprises about 50 wt % of the mixture of monomers.

In one aspect, described herein is a method of preparing a surface comprising recurring units of a first component and second component, the method comprising: (a) providing a mixture of monomers in a solvent comprising a first monomer and a second monomer, wherein the first monomer comprises a vinyl group and the second monomer comprises an epoxide group; (b) contacting a surface and the mixture of monomers, thereby producing a reaction mixture; (c) initiating a free radical polymerization to produce a macromolecule immobilized to a surface; (d) contacting the macromolecule immobilized to the surface with an azide salt, thereby producing an azide containing macromolecule; and (e) optionally contacting the azide-containing macromolecule with an alkyne-containing molecule to form a triazole-containing macromolecule.

In some embodiments, the surface is a bead or particle. In some embodiments, the surface is a particle. In some embodiments, the particle is a nanoparticle or microparticle. In some embodiments, the particle comprises a diameter of about 200 nanometers (nm) to about 400 nm. In some embodiments, the particle is a superparamagnetic iron oxide particle. In some embodiments, the particle comprises an iron oxide material. In some embodiments, the particle has an iron oxide core. In some embodiments, the particle has iron oxide crystals embedded in a polystyrene core. In some embodiments, the particle comprises an iron oxide core with a silica shell coating.

In some embodiments, solvent is a polar solvent. In some embodiments, the solvent is acetonitrile, THF, or DMF

In some embodiments, free radical polymerization is initiated with a radical initiator. In some embodiments, the radical initiator is AIBN.

In some embodiments, the method comprises, subsequent to (c) and prior to (d), contacting the macromolecule immobilized to the surface with a quenching agent. In some embodiments, the quenching agent is introduced to the reaction mixture when the macromolecule immobilized to the surface comprises a diameter of about 300 nm to about 500 nm. In some embodiments, the diameter is about 325 nm to about 375 nm. In some embodiments, the quenching agent comprises benzoquinone.

In some embodiments, the method further comprises purifying the macromolecule immobilized to the surface. In some embodiments, purifying comprises washing the macromolecule immobilized to the surface with THF or ethanol

In one aspect, described herein is a kit for identifying biomolecules in a biological sample, the kit comprising one or more surfaces of the disclosure.

In one aspect, described herein is a composition for identifying biomolecules in a biological sample, the composition comprising one or more surfaces of the disclosure and a biological sample in contact with the surfaces.

In some embodiments, the biological sample is plasma, serum or blood.

In some embodiments, the one or more surfaces comprises at least 2 distinct surfaces, at least 3 distinct surfaces, at least 4 distinct surfaces, at least 5 distinct surfaces, at least 6 distinct surfaces, at least 7 distinct surfaces, at least 8 distinct surfaces, at least 9 distinct surfaces, at least 10 distinct surfaces, at least 11 distinct surfaces, at least 12 distinct surfaces, at least 13 distinct surfaces, at least 14 distinct surfaces, at least 15 distinct surfaces, at least 20 distinct surfaces, at least 25 surfaces, or at least 30 distinct surfaces.

In some embodiments, the one or more surfaces comprises a first distinct surface and a second distinct surface, wherein the first distinct surface and the second distinct surface share at least one physicochemical property and differ by at least one physicochemical property, such that the first distinct surface and the second distinct surface are different.

In some embodiments, the one or more surfaces comprises a first distinct surface and a second distinct surface, wherein the first distinct surface and the second distinct surface share at least two physicochemical properties and differ by at least two physicochemical properties, such that the first distinct surface and the second distinct surface are different.

In some embodiments, the one or more surfaces comprises a first distinct surface and a second distinct surface, wherein the first distinct surface and the second distinct surface share at least one physicochemical property and differ by at least two physicochemical properties, such that the first distinct surface and the second distinct surface are different.

In some embodiments, the one or more surfaces comprises a first distinct surface and a second distinct surface, wherein the first distinct surface and the second distinct surface share at least two physicochemical properties and differ by at least one physicochemical property, such that the first distinct surface and the second distinct surface are different.

In some embodiments, the physicochemical property comprises size, charge, core material, shell material, porosity, or surface hydrophobicity.

In some embodiments, the size is diameter or radius, as measured by dynamic light scattering, SEM, TEM, or any combination thereof.

In some embodiments, the one or more surfaces comprises a first distinct surface and a second distinct surface, wherein the first distinct surface and the second distinct surface comprise a carboxylate material, wherein the first distinct particle is a microparticle, and wherein the second distinct surface is a nanoparticle.

In some embodiments, the one or more surfaces comprises a first distinct surface and a second distinct surface, wherein the first distinct surface and the second distinct surface comprise a surface charge of from 0 mV and −50 mV, wherein the first distinct surface has a diameter of less than 200 nm, and wherein the second distinct surface has a diameter of greater than 200 nm.

In some embodiments, the one or more surfaces comprises a first distinct surface and a second distinct surface, wherein the first distinct surface and the second distinct surface comprise a diameter of 100 to 400 nm, wherein the first distinct surface has a positive surface change, and wherein the second distinct surface has a neutral surface charge.

In some embodiments, the one or more surfaces comprises a first distinct surface and a second distinct surface, wherein the first distinct surface and the second distinct surface are nanoparticles, wherein the first distinct surface has a surface change less than −20 mV and the second distinct surface has a surface charge greater than −20 mV.

In some embodiments, the one or more surfaces comprises a first distinct surface and a second distinct surface, wherein the first distinct surface and the second distinct surface are microparticles, wherein the first distinct surface has a negative surface charge, and wherein the second distinct surface has a positive surface charge.

In some embodiments, the one or more surfaces comprises a subset of negatively charged nanoparticles, wherein each particle of the subset differ by at least one surface chemical group.

In some embodiments, the one or more surfaces comprises a first distinct surface, a second particle, and a third distinct surface, wherein the first distinct surface, the second distinct surface, and the third distinct surface comprise iron oxide cores, polymer shells, and are less than about 500 nm in diameter and wherein the first distinct surface comprises a negative charge, the second distinct surface comprises a positive charge, and the third distinct surface comprises a neutral charge, wherein the diameter is a mean diameter as measured by dynamic light scattering.

In some embodiments, at least one distinct surface of the one or more surfaces is a superparamagnetic iron oxide particle. In some embodiments, each surface of the one or more surfaces comprise an iron oxide material. In some embodiments, at least one distinct surface of the one or more surfaces has an iron oxide core. In some embodiments, at least one distinct surface of the one or more surfaces has iron oxide crystals embedded in a polystyrene core. In some embodiments, each distinct surface of the one or more surfaces is a superparamagnetic iron oxide particle. In some embodiments, each distinct surface of the one or more surfaces comprises an iron oxide core. In some embodiments, each one distinct surface of the one or more surfaces has iron oxide crystals embedded in a polystyrene core. In some embodiments, at least one surface of the one or more surfaces comprises an iron oxide core with a silica shell coating.

In one aspect, described herein is a system for identifying biomolecules in a biological sample, the composition comprising: (i) a macromolecule immobilized to a surface of the disclosure; (ii) a suspension solution; (iii) a biological sample comprising a concentration of proteins; and (iv) an automated system comprising a network of units with differentiated functions in distinguishing states of a complex biological sample using a plurality of surfaces with different physiochemical properties, and wherein the automated system is programed to perform a series of steps.

In some embodiments, the network of units comprises:
a first unit comprises a multichannel fluid transfer instrument for transferring fluids between units within the system;
a second unit comprises a support for storing a plurality of biological samples;
a third unit comprises a support for a sensor array plate possessing partitions that comprise the plurality of particles having surfaces with different physiochemical properties for detecting a binding interaction between a population of analytes within the complex biological sample and the plurality of particles;
a fourth unit comprises supports for storing a plurality of reagents;
a fifth unit comprises supports for storing a reagent to be disposed of; and
a sixth unit comprises supports for storing consumables used by the multichannel fluid transfer instrument

In some embodiments, the series of steps comprises: (i) contacting the biological sample with a specified partition of the sensor array; (ii) incubating the biological sample with the plurality of particles contained within the partition of the sensor array plate; (iii) removing all components from a partition except the plurality of particles and a population of analytes interacting with a particle; and (iv) preparing a sample for mass spectrometry.

In some embodiments, i.-iii. are incubated at a temperature of about 20 degrees Celsius to about 100 degrees Celsius.

In some embodiments, the suspension solution comprises Tris EDTA 150 mM KCl, 0.05% CHAPS buffer. In some embodiments, the suspension solution comprises 10 mM Tris HCl pH 7.4, 1 mM EDTA.

In one aspect, described herein is a method of identifying proteins in a sample, the method comprising: (a) incubating one or more surfaces of the disclosure with a biological sample comprising biomolecules to form a biomolecule corona; (b) isolating at least a portion of the biomolecules in the biomolecule corona; and (c) assaying the biomolecule corona.

In some embodiments, the assaying is capable of identifying from 1 to 20,000 protein groups. In some embodiments, the assaying is capable of identifying from 1000 to 10,000 protein groups. In some embodiments, the assaying is capable of identifying from 1,000 to 5,000 protein groups. In some embodiments, the assaying is capable of identifying from 1,200 to 2,200 protein groups.

In some embodiments, the protein group comprises a peptide sequence having a minimum length of 7 amino acid residues.

In some embodiments, the assaying is capable of identifying from 1,000 to 10,000 proteins. In some embodiments, the assaying is capable of identifying from 1,800 to 5,000 proteins.

In some embodiments, the sample comprises a plurality of samples. In some embodiments, the plurality of samples comprises at least two or more spatially isolated samples.

In some embodiments, incubating comprises contacting the at least two or more spatially isolated samples with the one or more surfaces at the same time.

In some embodiments, isolating comprises magnetically isolating the one or more surfaces from unbound protein in the at least two or more spatially isolated samples of the plurality of samples at the same time.

In some embodiments, the assaying comprises assaying a plurality of biomolecule coronas to identify proteins in the at least two or more spatially isolated samples at the same time.

In some embodiments, the method further comprises repeating the methods of any one of claims y-yy, wherein, when repeated, the incubating, isolating, and assaying yields a percent quantile normalized coefficient (QNCV) of variation of 20% or less, as determined by comparing a peptide mass spectrometry feature from at least three full-assay replicates for each surface in the one or more surfaces.

In some embodiments, when repeated, the incubating, isolating, and assaying yields a percent quantile normalized coefficient (QNCV) of variation of 10% or less, as determined by comparing a peptide mass spectrometry feature from at least three full-assay replicates for each surface in the one or more surfaces.

In some embodiments, the assaying is capable of identifying proteins over a dynamic range of at least 7, at least 8, at least 9, or at least 10.

In some embodiments, the method further comprises washing the one or more surfaces at least one time or at least two times after magnetically isolating the one or more surfaces from the unbound protein.

In some embodiments, after the assaying the method further comprises lysing the proteins in the plurality of biomolecule coronas.

In some embodiments, the method further comprises digesting the proteins in the plurality of biomolecule coronas to generate digested peptides. In some embodiments, the method further comprises purifying the digested peptides.

In some embodiments, the assaying comprises using mass spectrometry to identify proteins in the sample.

In some embodiments, the assaying is performed in about 2 to about 4 hours.

In some embodiments, the method is performed in about 1 to about 20 hours. In some embodiments, the method is performed in about 2 to about 10 hours. In some embodiments, the method is performed in about 4 to about 6 hours.

In some embodiments, the isolating takes no more than about 30 minutes, no more than about 15 minutes, no more than about 10 minutes, no more than about 5 minutes, or no more than about 2 minutes.

In some embodiments, the plurality of samples comprises at least 10 spatially isolated samples, at least 50 spatially isolated samples, at least 100 spatially isolated samples, at least 150 spatially isolated samples, at least 200 spatially isolated samples, at least 250 spatially isolated samples, or at least 300 spatially isolated samples.

In some embodiments, the plurality of samples comprises at least 96 samples.

In some embodiments, the one or more surfaces comprises at least 2 distinct surfaces, at least 3 distinct surfaces, at least 4 distinct surfaces, at least 5 distinct surfaces, at least 6 distinct surfaces, at least 7 distinct surfaces, at least 8 distinct surfaces, at least 9 distinct surfaces, at least 10 distinct surfaces, at least 11 distinct surfaces, at least 12 distinct surfaces, at least 13 distinct surfaces, at least 14 distinct surfaces, at least 15 distinct surfaces, at least 20 distinct surfaces, at least 25 surfaces, or at least 30 distinct surfaces.

In some embodiments, the one or more surfaces comprises at least 10 distinct surfaces.

In some embodiments, the at least two spatially isolated samples differ by at least one physicochemical property.

In some embodiments, the one or more surfaces comprises a first distinct surface and a second distinct surface, wherein the first distinct surface and the second distinct surface share at least one physicochemical property and differ by at least one physicochemical property, such that the first distinct surface and the second distinct surface are different.

In some embodiments, the one or more surfaces comprises a first distinct surface and a second distinct surface, wherein the first distinct surface and the second distinct surface share at least two physicochemical properties and differ by at least two physicochemical properties, such that the first distinct surface and the second distinct surface are different.

In some embodiments, the one or more surfaces comprises a first distinct surface and a second distinct surface, wherein the first distinct surface and the second distinct surface share at least one physicochemical property and differ by at least two physicochemical properties, such that the first distinct surface and the second distinct surface are different.

In some embodiments, the one or more surfaces comprises a first distinct surface and a second distinct surface, wherein the first distinct surface and the second distinct surface share at least two physicochemical properties and differ by at least one physicochemical property, such that the first distinct surface and the second distinct surface are different.

In some embodiments, the physicochemical property comprises size, charge, core material, shell material, porosity, or surface hydrophobicity.

In some embodiments, the size is diameter or radius, as measured by dynamic light scattering, SEM, TEM, or any combination thereof.

In some embodiments, the one or more surfaces comprises a first distinct surface and a second distinct surface, wherein the first distinct surface and the second distinct surface comprise a carboxylate material, wherein the first distinct particle is a microparticle, and wherein the second distinct surface is a nanoparticle.

In some embodiments, the one or more surfaces comprises a first distinct surface and a second distinct surface, wherein the first distinct surface and the second distinct surface comprise a surface charge of from 0 mV and −50 mV, wherein the first distinct surface has a diameter of less than 200 nm, and wherein the second distinct surface has a diameter of greater than 200 nm.

In some embodiments, the one or more surfaces comprises a first distinct surface and a second distinct surface, wherein the first distinct surface and the second distinct surface comprise a diameter of 100 to 400 nm, wherein the first distinct surface has a positive surface change, and wherein the second distinct surface has a neutral surface charge.

In some embodiments, the one or more surfaces comprises a first distinct surface and a second distinct surface, wherein the first distinct surface and the second distinct surface are nanoparticles, wherein the first distinct surface has a surface change less than −20 mV and the second distinct surface has a surface charge greater than −20 mV.

In some embodiments, the one or more surfaces comprises a first distinct surface and a second distinct surface, wherein the first distinct surface and the second distinct surface are microparticles, wherein the first distinct surface has a negative surface charge, and wherein the second distinct surface has a positive surface charge.

In some embodiments, the one or more surfaces comprises a subset of negatively charged nanoparticles, wherein each particle of the subset differ by at least one surface chemical group.

In some embodiments, the one or more surfaces comprises a first distinct surface, a second particle, and a third distinct surface, wherein the first distinct surface, the second distinct surface, and the third distinct surface comprise iron oxide cores, polymer shells, and are less than about 500 nm in diameter and wherein the first distinct surface comprises a negative charge, the second distinct surface comprises a positive charge, and the third distinct surface comprises a neutral charge, wherein the diameter is a mean diameter as measured by dynamic light scattering.

In some embodiments, at least one distinct surface of the one or more surfaces is a nanoparticle. In some embodiments, at least one distinct surface of the one or more surfaces is a microparticle. In some embodiments, at least one distinct surface of the one or more surfaces is a superparamagnetic iron oxide particle. In some embodiments, each particle of the one or more surfaces comprise an iron oxide material. In some embodiments, at least one distinct surface of the one or more surfaces has an iron oxide core. In some embodiments, at least one distinct surface of the one or more surfaces has iron oxide crystals embedded in a polystyrene core. In some embodiments, each distinct surface of the one or more surfaces is a superparamagnetic iron oxide particle. In some embodiments, each distinct surface of the one or more surfaces comprises an iron oxide core. In some embodiments, each one distinct surface of the one or more surfaces has iron oxide crystals embedded in a polystyrene core. In some embodiments, at least one distinct surface of one or more surfaces comprises a carboxylated polymer, an aminated polymer, a zwitterionic polymer, or any combination thereof. In some embodiments, at least one surface of the one or more surfaces comprises an iron oxide core with a silica shell coating.

In some embodiments, at least one distinct surface of the one or more surfaces comprises a negative surface charge. In some embodiments, at least one distinct surface of the one or more surfaces comprises a positive surface charge. In some embodiments, at least one distinct surface of the one or more surfaces comprises a neutral surface charge.

In one aspect, described herein is a use of the macromolecule of the disclosure in a method for identifying proteins in biological sample.

In one aspect, described herein is a use of the macromolecule of the disclosure to adsorb proteins in biological sample.

In one aspect, described herein is a use of the macromolecule of the disclosure in a method for identifying proteins in biological sample.

In one aspect, described herein is a use of the macromolecule of the disclosure to adsorb proteins in biological sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the general design space of the epoxidated nanoparticle platform following addition of a functionalizing amine.

FIG. 2 shows the conversion schemes available after epoxidation of nanoparticles including reaction with glycidyls and azides to obtain diverse functionalities.

FIG. 3 shows a diagram of a nanoparticle coated with SiO₂ followed by coating with cross-linked polymer to obtain diverse functionalities.

FIG. 4 shows the number of protein groups adhered to different surfaces and the Jaccard index representing the difference between two surfaces in terms of protein groups adhered.

FIG. 5 shows the number of protein groups adhered to different surface and the Jaccard index representing the difference between two surfaces in terms of protein groups adhered.

FIG. 6A-C shows a Cy7-Maleimide functionalizing agent (FIG. 6A), relative fluorescence units as a function of concentration to determine the number of thiols per nanoparticle (FIG. 6B), and the chemical structure of the thiolated nanoparticle which was functionalized (FIG. 6C).

FIG. 7A-C shows a Cy7-DBCO functionalizing agent (FIG. 7A), relative fluorescence units as a function of concentration to determine the number of azides per nanoparticle (FIG. 7B), and the chemical structure of the azide modified nanoparticle which was functionalized (FIG. 7C).

FIG. 8 shows a general reaction scheme for the functionalization of a thiolated nanoparticle with a peptide. Figure discloses SEQ ID NO: 3.

FIG. 9A-B shows thermogravimetric analysis (TGA) of surfaces before peptide modification (FIG. 9A) and after peptide modification (FIG. 9B).

FIG. 10 shows the chemical structure of the thiolated nanoparticle which was functionalized with SMCC-MagaininII.

FIG. 11 shows a reaction scheme for modification of a thiolated surface with an SMCC modified peptide. Figure discloses SEQ ID NOS 5, 4, and 4, respectively, in order of appearance.

FIG. 12 shows a reaction scheme for modification of a azide modified surface with a DBCO modified peptide. Figure discloses SEQ ID NOS 6, 7, and 7, respectively, in order of appearance.

FIG. 13A-B shows a diagram of a modular unit which may be used for peptide synthesis, in accordance with some embodiments (FIG. 13A) and an example of a binding molecule comprising modular units and configured for intermolecular or intramolecular exchange, in accordance with some embodiments (FIG. 13B).

FIG. 14 shows examples of linear and cyclic peptides which may be coupled to a particle surface, in accordance with some embodiments. Figure discloses SEQ ID NOS 8-10, respectively, in order of appearance.

FIG. 15 shows examples of a reaction scheme for obtaining of a surface functionalized with thiol and carboxylic acid.

### DETAILED DESCRIPTION

While various embodiments of the disclosure have been shown and described herein, it will be obvious to those skilled in the art that such embodiments are provided by way of example only. Numerous variations, changes, and substitutions can occur to those skilled in the art without departing from the disclosure. It should be understood that various alternatives to the embodiments of the disclosure described herein can be employed. The section headings used herein are for organizational purposes only and are not to be construed as limiting the subject matter described.

### Definitions

Unless defined otherwise, all technical and scientific terms used herein have the same meaning as is commonly understood by one of skill in the art to which this disclosure belongs. All patents and publications referred to herein are incorporated by reference.

“Alkyl” refers to an optionally substituted straight-chain, or optionally substituted branched-chain saturated hydrocarbon mono-radical, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). Whenever it appears herein, a numerical range such as “C₁-C₃ alkyl” means that the alkyl group consists of 1 carbon atom, 2 carbon atoms, or 3 carbon atoms. In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). In other embodiments, examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. The alkyl is attached to the rest of the molecule by a single bond. Unless stated otherwise specifically in the specification, an alkyl group is optionally substituted, for example, with oxo, halogen, amino, nitrile, nitro, hydroxyl, sulfone, mercapto, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, —NO₂, or —C≡CH. In some embodiments, the alkyl is optionally substituted with oxo, halogen, —CN, —CF₃, —OH, or —OMe. In some embodiments, the alkyl is optionally substituted with halogen such as F. In some embodiments, the alkyl is unsubstituted.

As used herein, C₁-Cₓ (or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages. Also, by way of example, “C₁-C₆ hydroxy” indicates that there are one to six carbon atoms in the alkyl group which is substituted by hydroxy (—OH).

As used herein, C₁-Cₓ (or C₁₋ₓ) hydroxy includes C₁-Cₓ alkyl substituted with hydroxy (—OH). As used herein, C₁-Cₓ (or C₁₋ₓ) heterocycloalkyl includes C₁-Cₓ alkyl substituted with heterocycloalkyl. As used herein, C₁-Cₓ (or C₁₋ₓ) dicycloalkyl methane includes C₁-Cₓ alkyl substituted with dicycloalkyl methane. As used herein, C₁-Cₓ (or C₁₋ₓ) alkyl guanidine includes C₁-Cₓ alkyl substituted with alkyl guanidine. As used herein, C₁-Cₓ (or C₁₋ₓ) ether includes C₁-Cₓ alkyl substituted with an —O— within alkyl chain (e.g., —(CH₂)ₓ—O—(CH₂)_{y}—). As used herein, C₁-Cₓ (or C₁₋ₓ) disulfide includes C₁-Cₓ alkyl substituted with an —S—S— within the alkyl chain (e.g., —(CH₂)ₓ—S—S—(CH₂)_{y}—). As used herein C₁-Cₓ (or C₁₋ₓ) thiol includes C₁-Cₓ (or C₁₋ₓ) alkyl substituted with thiol. As used herein, C₁-Cₓ (or C₁₋ₓ) alkylamine includes C₁-Cₓ (or C₁₋ₓ) alkyl substituted with alkylamine. As used herein, C₁-Cₓ (or C₁₋ₓ) alkyl acetamide includes C₁-Cₓ (or C₁₋ₓ) alkyl substituted with alkyl acetamide. As used herein C₁-Cₓ (or C₁₋ₓ) aminophthalate includes C₁-Cₓ (or C₁₋ₓ) alkyl substituted with aminophthalate. As used herein, C₁-Cₓ (or C₁₋ₓ) alkyl sulfone includes C₁-Cₓ (or C₁₋ₓ) alkyl substituted with sulfone (e.g., —(CH₂)₃SOOOH or —(CH₂)₃S(═O)₂OH). As used herein, C₁-Cₓ (or C₁₋ₓ) alkylamine includes C₁-Cₓ (or C₁₋ₓ) alkyl substituted with an amine within the alkyl chain (e.g., —(CH₂)ₓN(R)(CH₂)_{y}—) and can include secondary, tertiary, or quartary amines. As used herein C₁-Cₓ (or C₁₋ₓ) alkylamine also includes C₁-Cₓ (or C₁₋ₓ) alkyl substituted with amines e.g., —CH₂CH₂NH₂.

“Alkoxy” refers to a radical bonded through an oxygen atom of the formula —O-alkyl, where alkyl is an alkyl chain as defined above. Unless stated otherwise specifically in the specification, an alkoxy group can be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen. In some embodiments, the alkoxy is unsubstituted.

“Alkenyl” refers to an optionally substituted straight or branched hydrocarbon chain radical group containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises four to eight carbon atoms (i.e., C₄-C₆ alkenyl). In other embodiments, an alkenyl comprises six to eight carbon atoms (i.e., C₆-C₈ alkenyl). In certain embodiments, an alkenyl comprises at least one double bond at the end of a carbon chain. In other embodiments, an alkenyl comprises at least one double bond in the middle of a carbon chain. The group can be in either the cis or trans configuration about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group can consist of 2 carbon atoms, 3 carbon atoms, 4 carbon atoms, 5 carbon atoms, or 6 carbon atoms. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen. In some embodiments, the alkenyl is unsubstituted.

“Alkynyl” refers to an optionally substituted straight or branched hydrocarbon chain radical group containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). Whenever it appears herein, a numerical range such as “C₂-C₆ alkynyl” means that the alkynyl group can consist of 2 carbon atoms, 3 carbon atoms, 4 carbon atoms, 5 carbon atoms, or 6 carbon atoms. The alkynyl is attached to the rest of the molecule by a single bond, for example, ethynyl, propynyl, butynyl, pentynyl, hexynyl, 2-propynyl, 2-butynyl, 1,3-butadiynyl, and the like. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen. In some embodiments, the alkynyl is unsubstituted.

“Alkylene” or “alkylene chain” refers to an optionally substituted straight or branched divalent hydrocarbon chain linking the rest of the molecule to a radical group containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene group can be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. In some embodiments, the alkylene is unsubstituted.

“Aryl” refers to a radical derived from a hydrocarbon ring system comprising at least one aromatic ring. In some embodiments, an aryl comprises hydrogens and 5 to 30 carbon atoms. The aryl radical can be a monocyclic, bicyclic, tricyclic, or tetracyclic ring system, which can include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, indane, indene, naphthalene, phenalene, phenanthrene, pleiadene, pyrene, and triphenylene. In some embodiments, the aryl is phenyl. Unless stated otherwise specifically in the specification, an aryl can be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂. In some embodiments, the aryl is unsubstituted.

“Aralkyl” refers to a radical of the formula —R^{c}-aryl where R^{c} is an alkylene chain as defined above, for example, methylene, ethylene, and the like.

“Aralkenyl” refers to a radical of the formula —R^{d}-aryl where R^{d} is an alkenylene chain as defined above. “Aralkynyl” refers to a radical of the formula —R^{e}-aryl, where R^{c} is an alkynylene chain as defined above.

“Carbocycle” refers to a saturated, unsaturated or aromatic rings in which each atom of the ring is carbon. Carbocycle can include 3- to 10-membered monocyclic rings and 6- to 12-membered bicyclic rings (such as spiro, fused, or bridged rings). Each ring of a bicyclic carbocycle can be selected from saturated, unsaturated, and aromatic rings. An aromatic ring, e.g., phenyl, can be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. In an exemplary embodiment, an aromatic ring, e.g., phenyl, can be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-5 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene. The term “saturated cyclaroalkyl” as used herein refers to a saturated carbocycle. Exemplary carbocycles include cyclopropyl, cyclopentyl, cyclohexyl, cyclohexenyl, adamantyl, phenyl, indanyl, norborane, and naphthyl. Carbocycles can be optionally substituted by one or more substituents such as those substituents described herein.

“Cycloalkyl” refers to a stable, partially or fully saturated, monocyclic or polycyclic carbocyclic ring, which can include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen. In some embodiments, the cycloalkyl is unsubstituted.

“Cycloalkylalkyl” refers to a radical of the formula —R^{c}-cycloalkyl where R^{c} is an alkylene chain as described above.

“Cycloalkylalkoxy” refers to a radical bonded through an oxygen atom of the formula —O—R^{c}-cycloalkyl where R^{c} is an alkylene chain as described above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di-and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., C₁, Br, F, I, etc.). When an alkyl group is substituted with more than one halogen radicals, each halogen can be independently selected e.g., 1-chloro, 2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl. As used herein, “C₁-C₆ hydroxy” refers to C₁-C₆ hydroxyalkyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Disulfide” refers to two sulfur atoms bonded to each other, where each sulfur comprises an optionally substituted alkyl chain. In some embodiments a disulfide may be R—S—S—R′. In some embodiments, R and R′ may be identical. In some embodiments, R and R′ are different. Each R and R′ may be independently selected from C₁-C₁₂ alkyl. In certain embodiments, R or R′ may be substituted with an amine, sulfone, or carboxylic acid.

The term “heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen. In some embodiments, the heteroalkyl is unsubstituted.

“Heterocycloalkyl” refers to a stable 3- to 24-membered partially or fully saturated ring radical comprising 2 to 23 carbon atoms and at least one ring heteroatoms. In some embodiments, a heterocycloalkyl contains from one to 8 heteroatoms selected from the group consisting of nitrogen, oxygen, phosphorous, and sulfur. Unless stated otherwise specifically in the specification, the heterocycloalkyl radical can be a monocyclic, bicyclic, tricyclic, or tetracyclic ring system, which can include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical can be optionally oxidized; the nitrogen atom can be optionally quaternized.

Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen. In some embodiments, the heterocycloalkyl is unsubstituted.

“Heterocycle” or “heterocyclyl” refers to a saturated, unsaturated or aromatic ring comprising one or more ring heteroatoms. Exemplary heteroatoms include N, O, Si, P, B, and S atoms. Heterocycles include e.g., 3- to 10-membered monocyclic rings and 6- to 12-membered bicyclic rings (such as spiro, fused, or bridged rings). Unless stated otherwise specifically in the specification, the heterocyclyl radical is a monocyclic, bicyclic, tricyclic or tetracyclic ring system, which optionally includes fused, bridged, or spirocyclic ring systems. The heteroatoms in the heterocyclyl radical are optionally oxidized. One or more nitrogen atoms, if present, are optionally quaternized. The heterocyclyl radical can be partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents. For example, a heterocyclyl can be optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—CN, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroaryl” or “aromatic heterocycle” refers to a ring system radical comprising carbon atom(s) and one or more ring heteroatoms (e.g., selected from the group consisting of nitrogen, oxygen, phosphorous, silicon, and sulfur), and at least one aromatic ring. In some embodiments, a heteroaryl is a 5- to 14-membered ring system radical comprising one to thirteen carbon atoms, one to six heteroatoms selected from the group consisting of nitrogen, oxygen, phosphorous, and sulfur. The heteroaryl radical can be a monocyclic, bicyclic, tricyclic, or tetracyclic ring system, which can include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical can be optionally oxidized; the nitrogen atom can be optionally quatemized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen. In some embodiments, the heteroaryl is unsubstituted.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen can have hydrogen substituents and/or any permissible substituents of organic compounds described herein which satisfy the valences of the heteroatoms.

In some embodiments, substituents can include any substituents described herein, for example: halogen, hydroxy, amino, oxo (═O), thioxo (═S), cyano (—CN), nitro (—NO₂), imino (═N—H), oximo (═N—OH), hydrazino (═N—NH₂), —R^{b}—OR, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R b-C(O)OR^{a}, —R^{b}—C(O)N(R^{a})₂, —R^{b}—O—R^{c}—C(O)N(R^{a})₂, —R^{b}—N(R^{a})C(O)OW, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which can be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), SF⁵, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, can be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R b-C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

As used in the specification and claims, the singular form “a”, “an” and “the” includes plural references unless the context clearly dictates otherwise.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group can be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 can comprise 1 to 10, 1 to 20, 1 to 30, and 1 to 40 in one direction, or 50 to 40, 50 to 30, 50 to 20, and 50 to 10 in the other direction.

The compounds and structures provided herein may be stereoisomeric. In some cases, a compound or structure of the disclosure may form a stereoisomer. In some cases, the stereoisomer may be a diastereomer (e.g., a cis/trans isomer, E/Z isomer, conformer, or rotamer). In some cases, the stereoisomer may be an enantiomer (R,S enantiomers or +/−enantiomers). In some cases, the compound or structure of the disclosure may be enantiopure (e.g., 100% pure). In some cases, the compound or structure may form a racemic mixture of enantiomers (e.g., 50% pure). In some cases, a compound or structure of the disclosure may stabilize as a stereoisomer, where the compound or structure of the disclosure comprises at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 98.5%, about 99%, about 99.1%, about 99.2%, about 99.3%, about 99.4%, about 99.5%, about 99.6%, about 99.7%, about 99.8%, about 99.9%, or more of a mixture of the compound or structure and the corresponding stereoisomer.

The compounds and structures provided herein are intended to include all ionized forms of the compounds and structures provided herein. The compounds and structures provided herein are also intended to include all salt forms of the compounds and structures provided herein.

### Compounds of the Disclosure

Disclosed herein are macromolecules comprising one or more recurring units.

In one aspect, described herein, the macromolecule comprises a recurring unit of Formula (I):

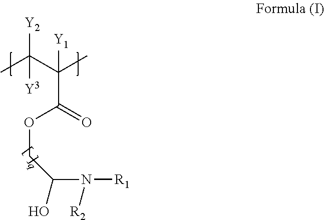

wherein each of Y₁, Y₂, and Y₃ is independently selected from hydrogen or C₁-C₆ alkyl;
R₁ is hydrogen, nitrogen, optionally substituted succinate, C₁-C₆ alkyl sulfone, or phthalate;
R₂ is nitrogen, C₁-C₆ hydroxy, optionally substituted aryl, optionally substituted heteroaryl, optionally substituted C₃-C₆ heterocycloalkyl, optionally substituted C₃-C₆ dicyloalkyl methane, C₁-C₆ alkyl guanidine, C₁-C₆ ether, optionally substituted —C₁-C₆ disulfide, C₁-C₆ thiol, optionally substituted succinate, optionally substituted C₁-C₆ alkylamine, C₁-C₆ alkyl acetamide, or C₁-C₆ aminophthalate; and
q is an integer between 1 and 6,
wherein if R₁ and R₂ are each nitrogen, then R₁ and R₂ are optionally taken together with the atom to which they are attached to form an optionally substituted heterocycle.

In one aspect, described herein, the macromolecule comprises a recurring unit of Formula (I-A):

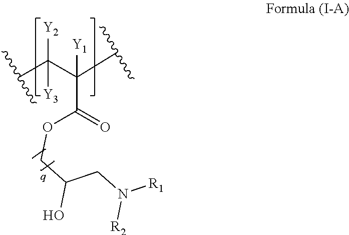

wherein each of Y₁, Y₂, and Y₃ is independently selected from hydrogen or C₁-C₆ alkyl;
R¹ is hydrogen, optionally substituted succinate, C₁-C₆ alkyl sulfone, or phthalate;
R₂ is C₁-C₆ hydroxy, optionally substituted aryl, optionally substituted heteroaryl, optionally substituted C₃-C₆ heterocycloalkyl, C₁-C₁₂ amine, optionally substituted C₃-C₆ dicyloalkyl methane, C₁-C₆ alkyl guanidine, C₁-C₆ ether, optionally substituted —C₁-C₆ disulfide, C₁-C₆ thiol, optionally substituted succinate, optionally substituted C₁-C₁₂ alkylamine, C₁-C₆ alkyl acetamide, C₅-C₁₁ cycloalkyl, or C₁-C₆ aminophthalate; or
R₁ and R₂ are taken together with the nitrogen to which they are attached to form an optionally substituted heterocycle; or
R₁ and R₂ are taken together with the nitrogen to which they are attached to form an azide group; and
q is an integer between 1 and 6. In some embodiments, provided herein is a polymer derived from a monomer of Formula (I-A′), wherein the substituents are as defined in Formula (I) else wherein herein:

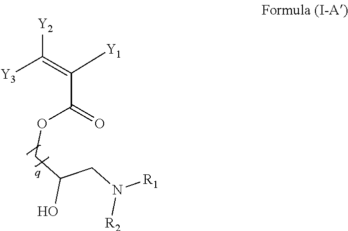

In some embodiments of Formula (I), (I-A), and (I-A′) each of Y₁, Y₂, and Y₃ is independently selected from hydrogen or C₁-C₆ alkyl. In some embodiments, Y₁ is hydrogen. In some embodiments, Y₁ is C₁-C₆ alkyl. In some embodiments, Y₂ is hydrogen. In some embodiments, Y₂ is C₁-C₆ alkyl. In some embodiments, Y₃ is hydrogen. In some embodiments, Y₃ is C₁-C₆ alkyl. In some embodiments, Y₁ is C₁-C₃ alkyl and each of Y₂ and Y₃ are hydrogen. In some embodiments, Y₁ is C₁ alkyl (e.g., —CH₃) and each of Y₂ and Y₃ are hydrogen.

In some embodiments of Formula (I), (I-A), and (I-A′), R₁ is hydrogen, nitrogen, optionally substituted succinate, C₁-C₆ alkyl sulfone, or phthalate. In some embodiments of Formula (I), R₁ is hydrogen, optionally substituted succinate, C₁-C₆ alkyl sulfone, or phthalate. In some embodiments, R₁ is hydrogen. In some embodiments, R₁ is nitrogen. In some embodiments, R₁ is optionally substituted succinate. In some embodiments, R₁ is C₁-C₆ alkyl sulfone. In some embodiments, R₁ is phthalate.

In some embodiments of Formula (I), (I-A), and (I-A′), R₂ is nitrogen, C₁-C₆ hydroxy, optionally substituted aryl, optionally substituted heteroaryl, optionally substituted C₃-C₆ heterocycloalkyl, optionally substituted C₃-C₆ dicyloalkyl methane, C₁-C₆ alkyl guanidine, C₁-C₆ ether, optionally substituted —C₁-C₆ disulfide, C₁-C₆ thiol, optionally substituted succinate, optionally substituted C₁-C₆ alkylamine, C₁-C₆ alkyl acetamide, or C₁-C₆ aminophthalate. In some embodiments of Formula (I), R₂ is C₁-C₆ hydroxy, optionally substituted aryl, optionally substituted heteroaryl, optionally substituted C₃-C₆ heterocycloalkyl, C₁-C₁₂ amine, optionally substituted C₃-C₆ dicyloalkyl methane, C₁-C₆ alkyl guanidine, C₁-C₆ ether, optionally substituted —C₁-C₆ disulfide, C₁-C₆ thiol, optionally substituted succinate, optionally substituted C₁-C₁₂ alkylamine, C₁-C₆ alkyl acetamide, C₅-C₁₁ cycloalkyl, or C₁-C₆ aminophthalate. In some embodiments, R₂ is nitrogen (e.g., azide). In some embodiments, R₂ is azide (e.g., N₃). In some embodiments, R₂ is C₁-C₁₂ amine. In some embodiments, R₂ is C₅-C₁₁ cycloalkyl. In some embodiments, R₂ is C₁-C₆ hydroxy. In some embodiments, R₂ is optionally substituted aryl. In some embodiments, R₂ is optionally substituted heteroaryl. In some embodiments, R₂ is optionally substituted C₃-C₆ heterocycloalkyl. In some embodiments, R₂ is optionally substituted C₃-C₆ dicyloalkyl methane. In some embodiments, R₂ is C₁-C₆ alkyl guanidine. In some embodiments, R₂ is C₁-C₆ ether. In some embodiments, R₂ is optionally substituted —C₁-C₆ disulfide. In some embodiments, R₂ is C₁-C₆ thiol. In some embodiments, R₂ is optionally substituted succinate. In some embodiments, R₂ is optionally substituted C₁-C₁₂ alkylamine. In some embodiments, R₂ is C₁-C₆ alkyl acetamide. In some embodiments, R₂ is C₁-C₆ aminophthalate. In some embodiments,

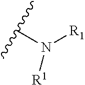

is taken together to form an —N₃.

In some embodiments, R₁ is selected from hydrogen,

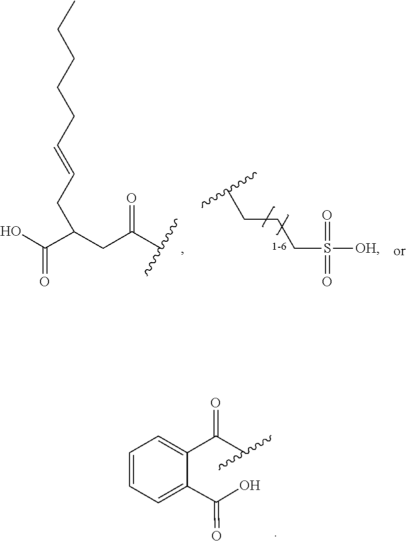

In some embodiments R₂ is selected from

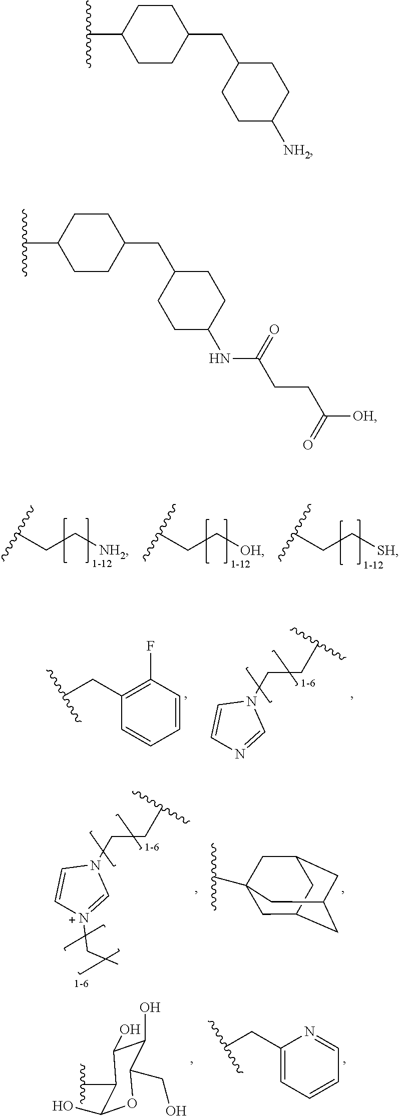

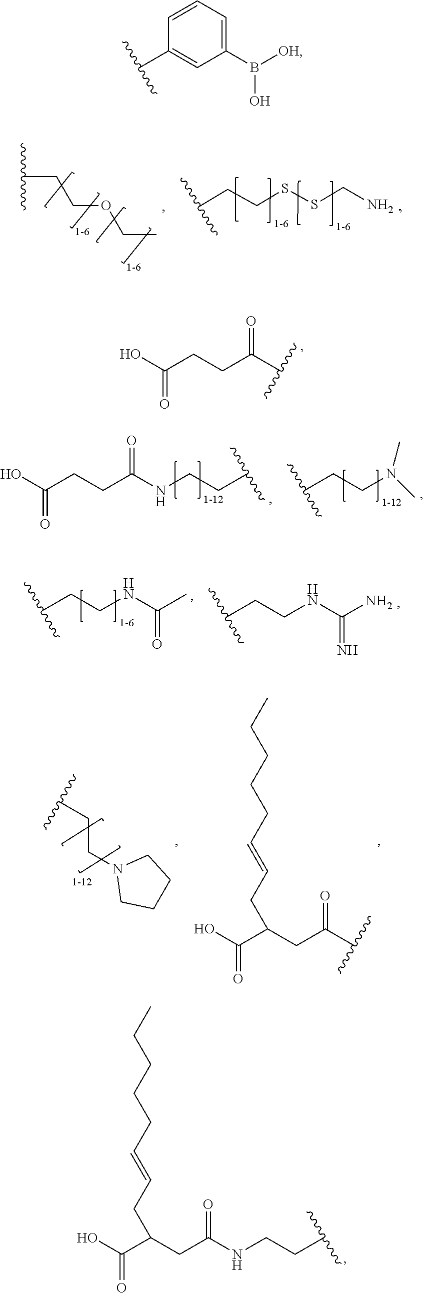

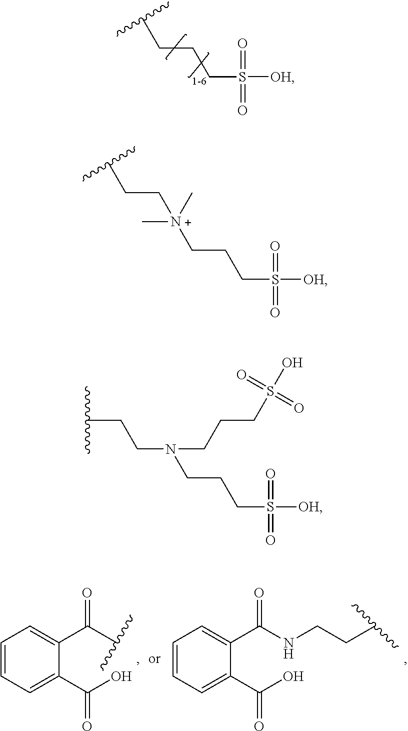

In some embodiments, R₁ and R₂ are taken together with the nitrogen to which they are attached to form an optionally substituted heterocycle (e.g., a nitrogen containing heterocycle).

In some embodiments, R₁ and R₂ are taken together with the nitrogen to which they are attached to form an azide group.

In some embodiments of Formula (I), (I-A), and (I-A′), R₁ is hydrogen and R₂ is selected from optionally substituted C₃-C₆ dicyloalkyl methane, optionally substituted aryl, optionally substituted heteroaryl, optionally substituted C₃-C₆ heterocycloalkyl, C₁-C₆ hydroxy, C₁-C₆ ether, optionally substituted —C₁-C₆ disulfide, optionally substituted succinate, optionally substituted C₁-C₆ alkylamine, C₁-C₆ alkyl acetamide, or C₁-C₆ alkyl guanidine. In some embodiments, when R₂ is optionally substituted C₃-C₆ heterocycloalkyl, it is attached to the rest of molecule via a substituent of the heterocycloalkyl. In some embodiments, R₁ is hydrogen and R₂ is optionally substituted dicyclohexyl methane. In some embodiments, R₁ is hydrogen and R₂ is amino dicyclohexyl methane (e.g.,

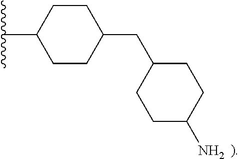 [SMILES: *C1CCC(CC2CCC(N)CC2)CC1]

In some embodiments, R₁ is hydrogen and R₂ is optionally substituted aryl. In some embodiments, R₁ is hydrogen and R₂ is halogenated toluene. In some embodiments R₁ is hydrogen and R₂ is 2-fluorotoluene (e.g.,

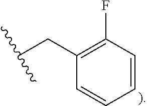 [SMILES: *Cc1ccccc1F]

In some embodiments, R₁ is hydrogen and R₂ is C₁-C₆ hydroxy. In some embodiments, R₁ is hydrogen and R₂ is C₃-C₆ hydroxy. In some embodiments, R₁ is hydrogen and R₂ is —(CH₂)₆OH (e.g.,

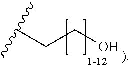

In some embodiments, R₁ is hydrogen and R₂ is C₁-C₆ ether. In some embodiments, R₁ is hydrogen and R₂ is selected from —CH₂OCH₃—, —CH₂CH₂OCH₃, —CH₂CH₂OCH₂CH₃, or —CH₂OCH₂CH₃. In some embodiments R₁ is hydrogen and R₂ is —CH₂CH₂OCH₃. In some embodiments, R₁ is hydrogen and R₂ is C₁-C₆ acetamide. In some embodiments, R₁ is hydrogen and R₂ is —(CH₂)₂ acetamide (e.g.,

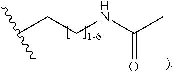

In some embodiments, R₁ is hydrogen and R₂ is optionally substituted di-C₁-C₆ alkyl disulfide. In some embodiments, R₁ is hydrogen and R₂ is —CH₂CH₂—S—S—CH₂CH₂NH₂ (e.g.,

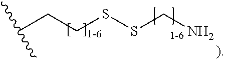

In some embodiments, R₁ is hydrogen and R₂ is optionally substituted succinate. In some embodiments, R₁ is hydrogen and R₂ is —(CH₂)₁₋₆ NH(C═O)CH₂CH₂COOH. In some embodiments, R₁ is hydrogen and R₂ is —(CH₂)₆NH(C═O)CH₂CH₂COOH (e.g.,

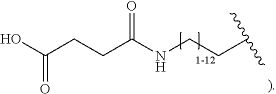

In some embodiments, R₁ is hydrogen and R₂ is optionally substituted heteroaryl. In some embodiments, when R₂ is optionally substituted heteroaryl, it is attached to the rest of molecule via a substituent of the heteroaryl. In some embodiments, the optionally substituted heteroaryl is —(CH₂)₁₋₆ imidazole. In some embodiments, R₁ is hydrogen and R₂ is —(CH₂)₁₋₆ imidazole. In some embodiments, R₁ is hydrogen and R₂ is —(CH₂)₃ imidazole (e.g.,

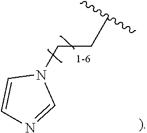

In some embodiments, R₁ is hydrogen and R₂ is disubstituted C₂-C₄ imidazole. In some embodiments, R₁ is hydrogen and R₂ is dipropyl imidazole (e.g.,

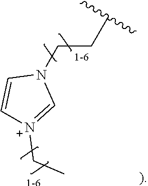

In some embodiments, R₁ is hydrogen and R₂ is optionally substituted heterocycloalkyl. In some embodiments, R₁ is hydrogen and R₂ is —(CH₂)₁₋₆ pyrrolidine. In some embodiments, R₁ is hydrogen and R₂ is —(CH₂)₂ pyrrolidine (e.g.,

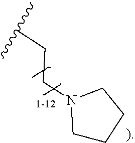

In some embodiments, R₁ is hydrogen and R₂ is optionally substituted C₁-C₆ alkylamine. In some embodiments, R₁ is hydrogen and R₂ is —(CH₂)₁₋₃ dimethylamine. In some embodiments, R₁ is hydrogen and R₂ is —(CH₂)₂ dimethylamine (e.g.,

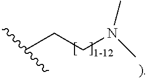

In some embodiments, R₁ is hydrogen and R₂ is C₁-C₆ guanidine. In some embodiments R₁ is hydrogen and R₂ is —(CH₂)₂ guanidine (e.g.,

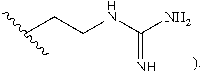 [SMILES: *CCNC(=N)N]

In some embodiments, each of R₁ and R₂ is nitrogen and are taken together to form an optionally substituted heterocycle. In some embodiments, each of R₁ and R₂ is nitrogen and are taken together to form an optionally substituted heterocycle which comprises a triazole. In some embodiments, each of R₁ and R₂ is nitrogen and are taken together to form an optionally substituted triazole comprising benzylamide. In some embodiments, each of R₁ and R₂ is nitrogen and are taken together to form an optionally substituted triazole comprising benzylamide wherein the benzylamide is halogenated. In some embodiments, R₁ and R₂ are taken together with the nitrogen to which they are attached to form an optionally substituted heterocycle. In some embodiments, R₁ and R₂ are taken together with the nitrogen to which they are attached to form an optionally substituted heteroaryl. In some embodiments, R₁ and R₂ are taken together with the nitrogen to which they are attached to form an azide group.

In some embodiments R₁ is optionally substituted succinate and R₂ is selected from optionally substituted C₃-C₆ dicycloalkyl methane, optionally substituted aryl, C₁-C₆ thiol, or optionally substituted succinate. In some embodiments R₁ is succinate and R₂ is optionally substituted C₃-C₆ dicyloalkyl methane, optionally substituted aryl, C₁-C₆ thiol, or optionally substituted succinate. In some embodiments R₁ is succinate and R₂ is dicyclohexylmethane succinate (e.g.,

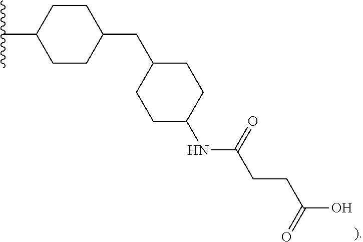 [SMILES: *C1CCC(CC2CCC(NC(=O)CCC(=O)O)CC2)CC1]

In some embodiments, R₂ is optionally substituted C₃-C₆ dicyloalkyl methane (e.g.,

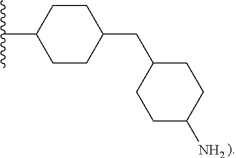 [SMILES: *C1CCC(CC2CCC(N)CC2)CC1]

In some embodiments, R₁ is succinate and R₂ is optionally substituted aryl. In some embodiments R₁ is succinate and R₂ is 2-fluorotoluene (e.g.,

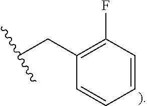 [SMILES: *Cc1ccccc1F]

In some embodiments, R₁ is succinate and R₂ is C₁-C₆ thiol. In some embodiments, R₁ is succinate and R₂ is —(CH₂)₂SH (e.g.,

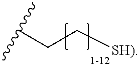

In some embodiments, R₁ is succinate and R₂ is optionally substituted succinate. In some embodiments, R₁ is succinate and R₂ is —(CH₂)₁₋₁₂NH(C═O)CH₂CH₂COOH. In some embodiments, R₁ is succinate and R₂ is —(CH₂)₂NH(C═O)CH₂CH₂COOH (e.g.

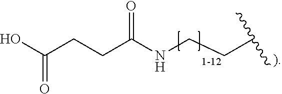

In some embodiments, R₁ is succinate and R₂ is —(CH₂)₁₀₋₁₂NH(C═O)CH₂CH₂COOH. In some embodiments, R₁ is succinate and R₂ is —(CH₂)₁₂NH(C═O)CH₂CH₂COOH (e.g.,

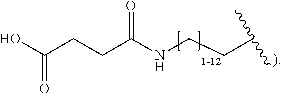

In some embodiments, R₁ is C₂-C₁₂ alkenyl succinate and R₂ is substituted succinate. In some embodiments R₁ is C₈ alkenyl succinate and R₂ is C₈ alkenyl ethylamino succinate (e.g.,

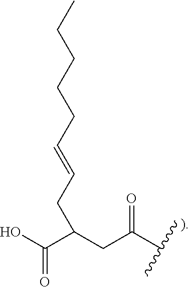 [SMILES: *C(=O)CC(C/C=C/CCCCC)C(=O)O]

In some embodiments, R₁ is C₁-C₆ alkyl sulfone and R₂ is optionally substituted C₁-C₆ alkylamine. In some embodiments, the C₁-C₆ alkyl sulfone is —(CH₂)₃SOOOH. In some embodiments R₁ is —(CH₂)₃SOOOH (e.g.,

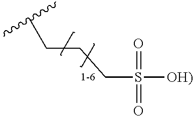

and R₂ is selected from —(CH₂)₁₋₆N(CH₃)₂(CH₂CH₂CH₂SOOOH) (e.g.,

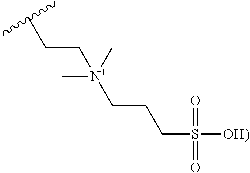

or —(CH₂)₁₋₆N(CH₂CH₂CH₂SOOOH)₂ (e.g.,

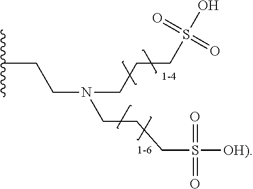

In some embodiments, R₁ is —(CH₂)₃SOOOH (e.g.,

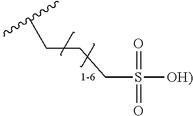

and R₂ is —(CH₂)₂N(CH₃)₂(CH₂CH₂CH₂SOOOH) (e.g.,

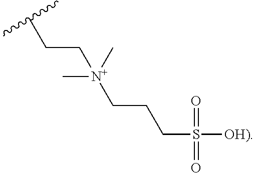

In some embodiments, R₁ is —(CH₂)₃SOOOH (e.g.,

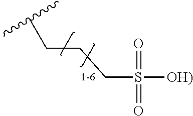

and R₂ is —(CH₂)₂N(CH₂CH₂CH₂SOOOH)₂(e.g.,

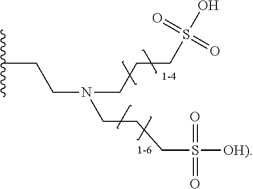

In some embodiments, R₁ is phthalate (e.g.,

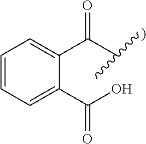 [SMILES: *C(=O)c1ccccc1C(=O)O]

and R₂ is C₁-C₆ aminophthalate. In some embodiments, R₁ is phthalate and R₂ is C₂-C₆ aminophthalate. In some embodiments R₁ is phthalate and R₂ is C₁-C₁₂ aminophthalate (e.g.,

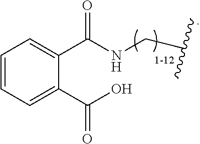

In some embodiments, R₁ is phthalate and R₂ is C₆ aminophthalate (e.g.,

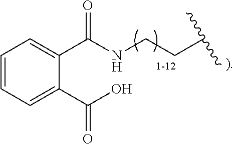

In some embodiments, R₂ is C₂ aminophthalate (e.g.,

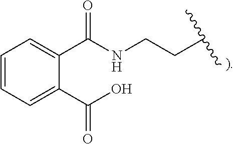 [SMILES: *CCNC(=O)c1ccccc1C(=O)O]

In some embodiments of Formula (I), (I-A), and (I-A′), R₁ and R₂ are each nitrogen. In some embodiments of Formula (I), R₁ and R₂ are each nitrogen and taken together with the atom to which they are attached to form an optionally substituted heterocycle. In some embodiments, the optionally substituted heterocycle is

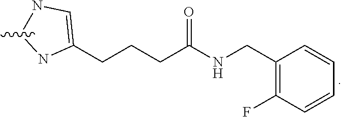

In some embodiments of Formula (I), (I-A), and (I-A′), q is an integer between 1 and 6. In some embodiments, q is an integer between 1 and 3. In some embodiments, q is 1. In some embodiments, q is 2. In some embodiments, q is 3. In some embodiments, q is 4. In some embodiments, q is 5. In some embodiments, q is 6.

In one aspect, the macromolecule comprises a recurring unit of formula (II):

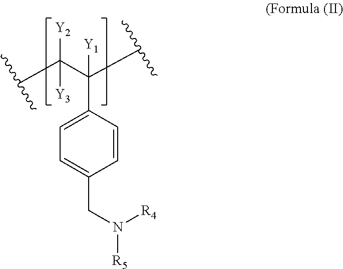

wherein each of Y₁, Y₂, and Y₃ is independently selected from hydrogen or C₁-C₆ alkyl;
R₄ is hydrogen or C₁-C₆ thiol; and
R₅ is succinate, C₁-C₆ thiol, optionally substituted aryl, or optionally substituted —C₁-C₆ disulfide.

In some embodiments,

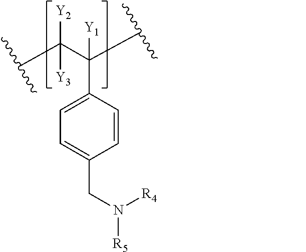

may refer interchangeably to

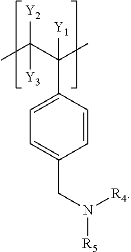

In some embodiments, provided herein is a polymer derived from a monomer of Formula (II′), wherein the substituent groups are defined as in Formula (II) as described elsewhere herein:

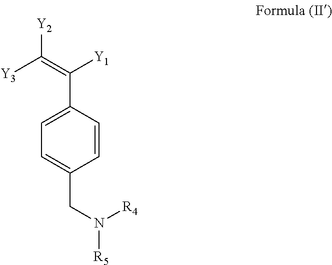

In some embodiments of Formula (II) and (II′), each of Y₁, Y₂, and Y₃ is independently selected from hydrogen or C₁-C₆ alkyl. In some embodiments Y₁ is hydrogen. In some embodiments Y₁ is C₁-C₆ alkyl. In some embodiments, Y₂ is hydrogen. In some embodiments, Y₂ is C₁-C₆ alkyl. In some embodiments, Y₃ is hydrogen. In some embodiments, Y₃ is C₁-C₆ alkyl.

In some embodiments of Formula (II) and (II′), R₄ is hydrogen or C₁-C₆ thiol. In some embodiments R₄ is hydrogen. In some embodiments, R₄ is C₁-C₆ thiol. In some embodiments R₄ is C₁-C₃ thiol.

In some embodiments, R₅ is succinate or optionally substituted —C₁-C₆ disulfide.

In some embodiments, R₅ is succinate or optionally substituted —C₁-C₆ disulfide. In some embodiments, R₅ is succinate, C₁-C₆ thiol, or optionally substituted —C₁-C₆ disulfide. In some embodiments, R₅ is succinate. In some embodiments, R₅ is optionally substituted —C₁-C₆ disulfide. In some embodiments, R₅ is C₁-C₆ thiol.

In some embodiments Y₁ is C₁-C₃ alkyl. In some embodiments Y₁ is C₁ alkyl (e.g. —CH₃). In some embodiments, each of Y₂ and Y₃ is hydrogen. In some embodiments R₄ is selected from hydrogen, C₁-C₃ thiol, or —(CH₂)₂SH (e.g.,

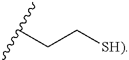

In some embodiments R₅ is selected from di-C₁-C₆ alkyl disulfide or —CH₂CH₂—S—S—CH₂CH₂NH₂ (e.g.,

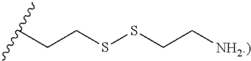

In some embodiments, R₄ is hydrogen and R₅ is optionally substituted —C₁-C₆ disulfide. In some embodiments R₄ is hydrogen and R₅ is optionally disubstituted di-C₁-C₆ alkyl disulfide. In some embodiments R₄ is hydrogen and R₅ is —CH₂CH₂—S—S—CH₂CH₂NH₂ (e.g.,

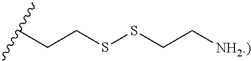

In some embodiments, R₄ is C₁-C₆ thiol and R₅ is succinate. In some embodiments, R₄ is C₁-C₃ thiol and R₅ is succinate. In some embodiments R₄ is —(CH₂)₂SH (e.g.,

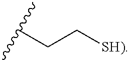

And R₅ is succinate.

In some embodiments, R₄ is

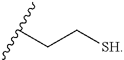 [SMILES: *CCS]

In some embodiments, R₅ is

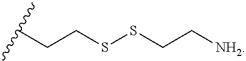 [SMILES: *CCSSCCN]

In one aspect, the macromolecule comprises a recurring unit of Formula (III):

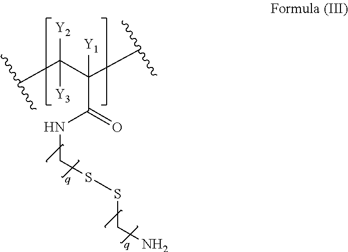

wherein each of Y₁, Y₂, and Y₃ is independently selected from hydrogen or C₁-C₆ alkyl; and each q is independently an integer between 1 and 6.

In some embodiments,

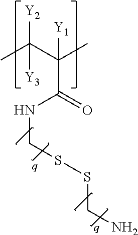

refers interchangeably to

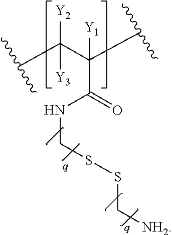

In some embodiments, provided herein is a macromolecule comprising a recurring unit of Formula (III′):

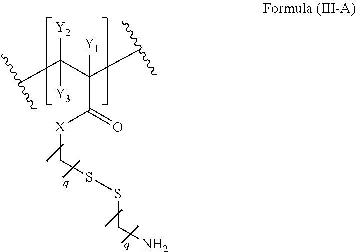

In some embodiments, X is O or NH. In some embodiments, X is O. In some embodiments, X is NH. In some embodiments, the recurring unit of Formula (III-A) is the recurring unit of Formula (III). In some embodiments, the substituents Y₁, Y₂, and Y₃ are defined as in Formula (III) herein.

In some embodiments, provided herein is a polymer derived from a monomer of Formula (III′), wherein the substituent groups are defined as in Formula (III) as described elsewhere herein:

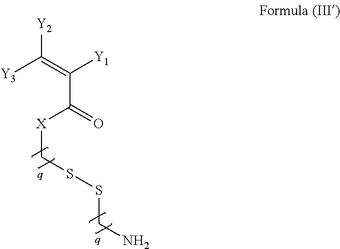

In some embodiments of Formula (III), (III-A), or (III′), each of Y₁, Y₂, and Y₃ is independently selected from hydrogen or C₁-C₆ alkyl. In some embodiments, Y₁ is hydrogen. In some embodiments, Y₁ is C₁-C₆ alkyl. In some embodiments, Y₂ is hydrogen. In some embodiments, Y₂ is C₁-C₆ alkyl. In some embodiments, Y₃ is hydrogen. In some embodiments, Y₃ is C₁-C₆ alkyl. In some embodiments, Y₁ is C₁-C₃ alkyl. In some embodiments, Y₁ is C₁ alkyl (e.g. —CH₃). In some embodiments, each of Y₂ and Y₃ is hydrogen. In some embodiments, each q is 2 or 3. In some embodiments, each q is 2.

In some embodiments, the macromolecule may comprise at least 5% by weight of the recurring unit of Formula (I), (I-A), (I-A′), (II), (II′), (III), (III′), or (III-A). In some embodiments, the macromolecule may comprise at least 10% by weight of the recurring unit of Formula (I), (I-A), (I-A′), (II), (II′), (III), (III′), or (III-A). In some embodiments, the macromolecule may comprise at least 25% by weight of the recurring unit of Formula (I), (I-A), (I-A′), (II), (II′), (III), (III′), or (III-A). In some embodiments, the macromolecule may comprise at least 50% by weight of the recurring unit of Formula (I), (I-A), (I-A′), (II), (II′), (III), (III′), or (III-A). In some embodiments, the macromolecule may comprise at least 75% by weight of the recurring unit of Formula (I), (I-A), (I-A′), (II), (II′), (III), (III′), or (III-A). In some embodiments, the macromolecule may comprise 5% to 95% by weight of the recurring unit of Formula (I), (I-A), (II), (III), or (III-A). In some embodiments, the macromolecule may comprise 20% to 80% by weight of the recurring unit of Formula (I), (I-A), (I-A′), (II), (II′), (III), (III′), or (III-A). In some embodiments, the macromolecule may comprise 40% to 60% by weight of the recurring unit of Formula (I), (I-A), (I-A′), (II), (II′), (III), (III′), or (III-A).

In some embodiments, a macromolecule described herein is quaternized. In some embodiments, described herein are macromolecules that are N-quatemized derivatives of a macromolecule described herein.

In some embodiments, recurring units of Formula (I), (I-A), (I-A′), (II), (II′), (III), (III′), (III-A), or (IV) may comprise the structures shown in Table 1, where m is an integer of 2 or more:

TABLE 1

| Compound | Structure |
|---|---|
| 103 | 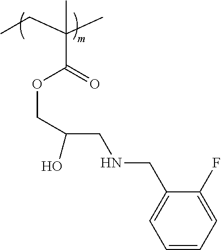 |
| 105 | 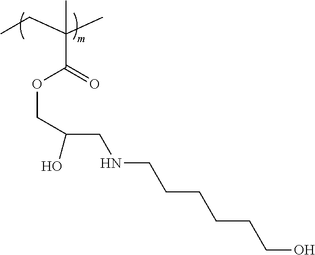 |
| 106 | 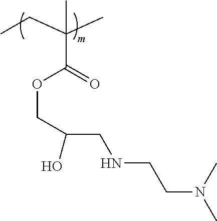 |
| 107 | 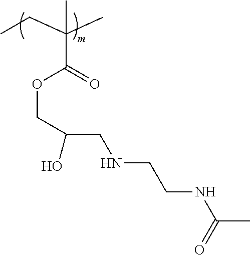 |
| 108 | 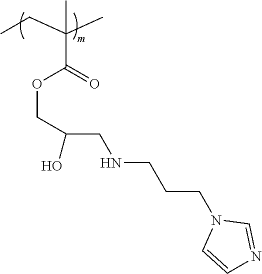 |
| 109 | 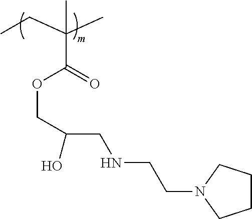 |
| 112 | 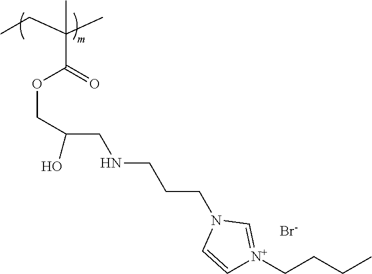 |
| 113 | 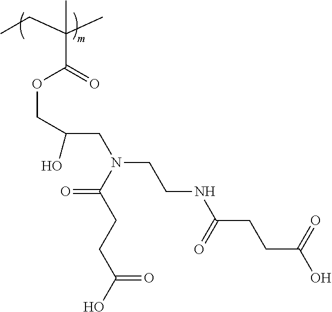 |
| 114 | 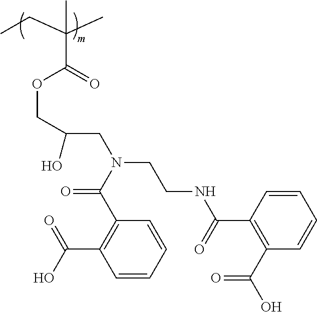 |
| 115 | 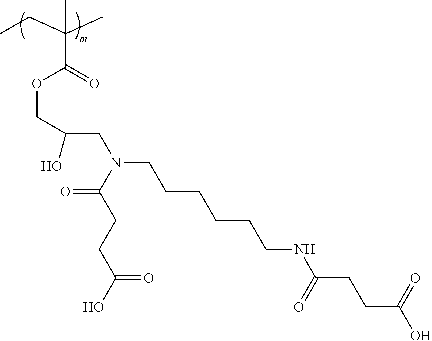 |
| 116 | 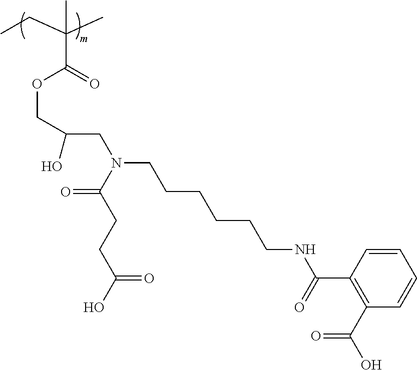 |
| 117 | 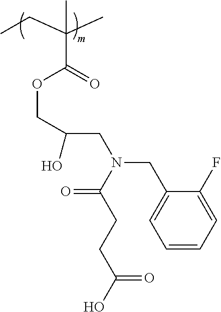 |
| 118 | 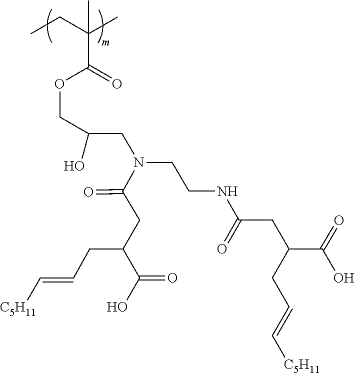 |
| 122 | 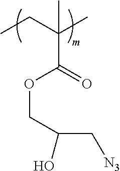 |
| 123 | 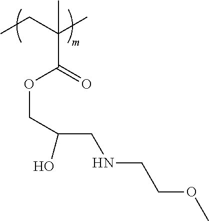 |
| 127 | 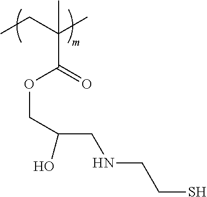 |
| 128 | 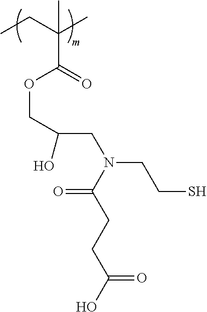 |
| 130 | 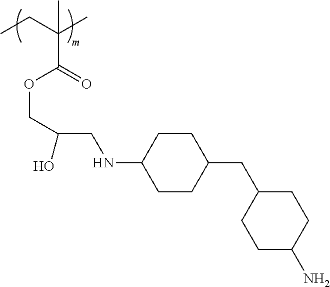 |
| 131 | 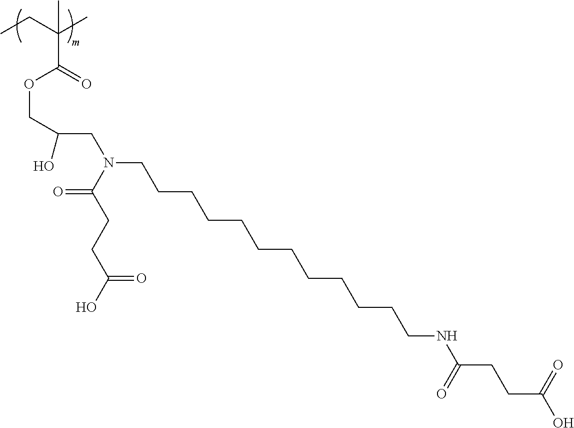 |
| 132 | 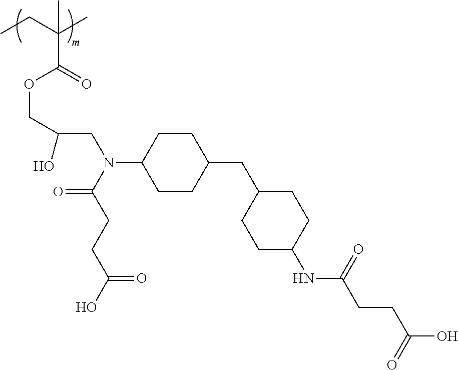 |
| 133 | 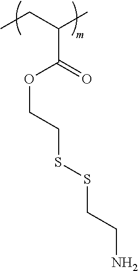 |
| 136 | 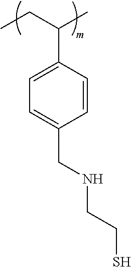 |
| 137 | 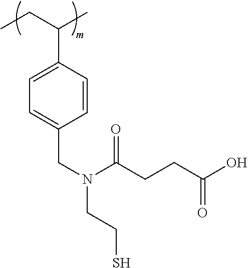 |
| 139 | 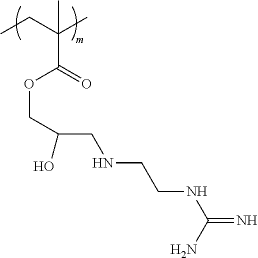 |
| 140 | 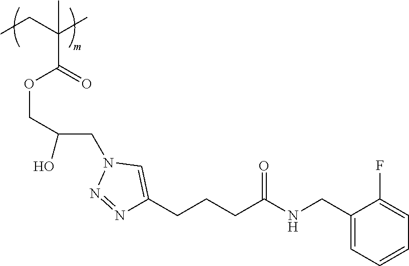 |
| 143 | 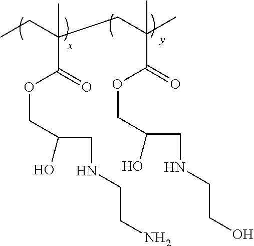 |
| 144 | 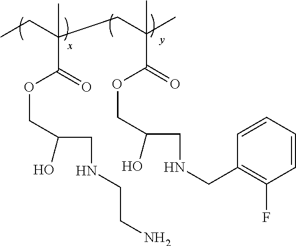 |
| 145 | 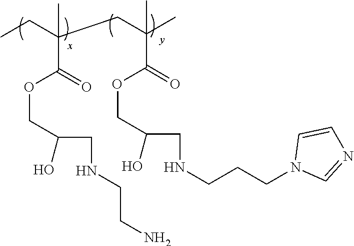 |
| 146 | 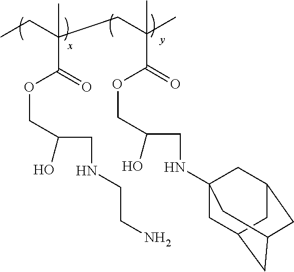 |
| 147 | 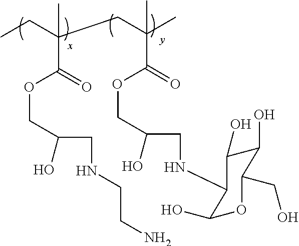 |
| 149 | 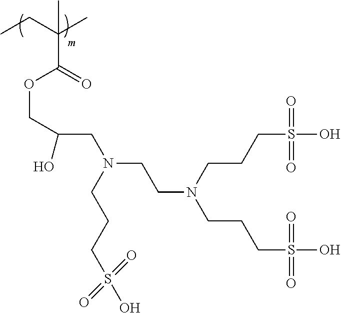 |
| 151 | 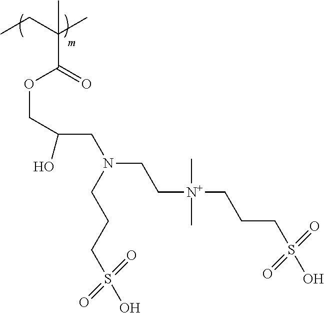 |

In some embodiments, the macromolecule further comprises a cross-linking recurring unit. For example, the cross-linking recurring unit may result from free radical polymerization of a monomer having two vinyl groups, such as divinyl benzene (DVB), ethyleneglycol dimethacrylate (EGDMA), poly(ethylene glycol) dimethacrylate (PEGDMA) (e.g., Diethylene glycol dimethacrylate (DEGDMA), Triethylene glycol dimethacrylate (TEGDMA), and the like) N,N′-alkylenebisacrylamide (e.g., N,N′-methylenebisacrylamide (MBA), N,N′-ethylenebisacrylamide, N,N′-butylenebisacrylamide, and the like), or derivative thereof. In some embodiments, the macromolecule may comprise at least 5% by weight of the cross-linking recurring unit. In some embodiments, the macromolecule may comprise at least 25% by weight of the cross-linking recurring unit. In some embodiments, the macromolecule may comprise at least 40% by weight of the cross-linking recurring unit. In some embodiments, the macromolecule may comprise no more than 90% by weight of the cross-linking recurring unit. In some embodiments, the macromolecule may comprise no more than 75% by weight of the cross-linking recurring unit. In some embodiments, the macromolecule may comprise no more than 60% by weight of the cross-linking recurring unit. In some embodiments, the macromolecule may comprise no more than 50% by weight of the cross-linking recurring unit. In some embodiments, the macromolecule may comprise no more than 25% by weight of the cross-linking recurring unit. In some embodiments, the macromolecule may comprise 5% to 95% by weight of the cross-linking recurring unit. In some embodiments, the macromolecule may comprise 20% to 80% by weight of the cross-linking recurring unit. In some embodiments, the macromolecule may comprise 40% to 60% by weight of the cross-linking recurring unit.

In one aspect, the macromolecule comprises recurring units of a first component and a second component, wherein the first component comprises a structure of Component (A) and the second component comprises a structure of Component (B).

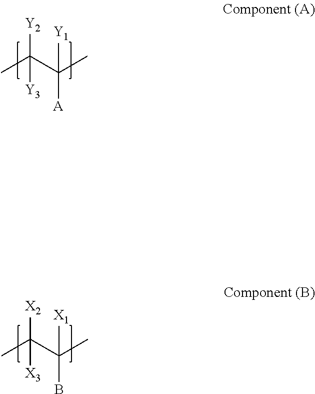

wherein each of Y₁, Y₂, and Y₃ is independently selected from hydrogen or C₁-C₆ alkyl;
each of X₁, X₂, and X₃ is independently selected from hydrogen or C₁-C₆ alkyl;
A is

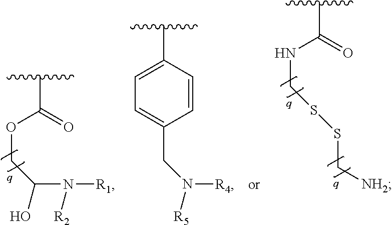

R¹ is hydrogen, nitrogen, optionally substituted succinate, C₁-C₆ alkyl sulfone, phthalate,
R₂ is nitrogen, C₁-C₁₂ amine, C₁-C₆ hydroxy, optionally substituted aryl, optionally substituted heteroaryl, optionally substituted C₃-C₆ heterocycloalkyl, two or more fused 3-6 member rings; optionally substituted C₃-C₆ dicyloalkyl methane, C₁-C₆ alkyl guanidine, C₁-C₆ ether, optionally substituted —C₁-C₆ disulfide, C₁-C₆ thiol, optionally substituted succinate, optionally substituted C₁-C₆ alkylamine, C₁-C₆ alkyl acetamide, C₁-C₆ aminophthalate, a boronic acid, or a monosaccharide;
R₄ is hydrogen or C₁-C₆ thiol;
R₅ is succinate or optionally substituted —C₁-C₆ disulfide;
B is

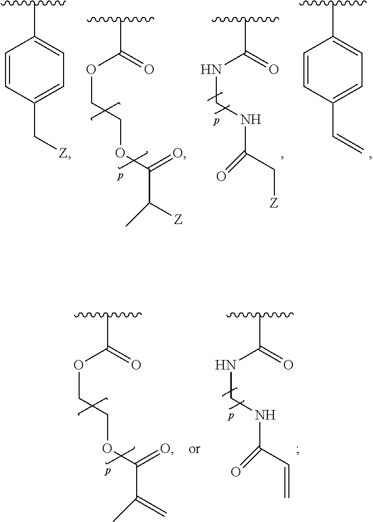

Z is a chain within the macromolecule;
q is an integer between 1 and 6; and
p is an integer between 1 and 20.

As used herein,

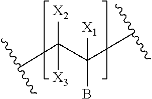

may refer interchangeably to

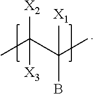

As used herein,

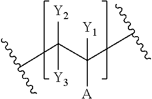

may refer interchangeably to

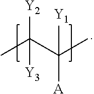

In some embodiments, each of Y₁, Y₂, and Y₃ is independently selected from hydrogen or C₁-C₆ alkyl. In some embodiments, Y₁ is hydrogen. In some embodiments, Y₁ is C₁-C₆ alkyl. In some embodiments, Y₂ is hydrogen. In some embodiments, Y₂ is C₁-C₆ alkyl. In some embodiments, Y₃ is hydrogen. In some embodiments, Y₃ is C₁-C₆ alkyl. In some embodiments, Y₁ is C₁-C₃ alkyl and each of Y₂ and Y₃ are hydrogen. In some embodiments, Y₁ is C₁ alkyl (e.g., —CH₃) and each of Y₂ and Y₃ are hydrogen.

In some embodiments, each of X₁, X₂, and X₃ is independently selected from hydrogen or C₁-C₆ alkyl. In some embodiments, X₁ is hydrogen. In some embodiments, X₁ is C₁-C₆ alkyl. In some embodiments, X₂ is hydrogen. In some embodiments, X₂ is C₁-C₆ alkyl. In some embodiments, X₃ is hydrogen. In some embodiments, X₃ is C₁-C₆ alkyl. In some embodiments, X₁ is C₁-C₃ alkyl and each of X₂ and X₃ are hydrogen. In some embodiments, X₁ is C₁ alkyl (e.g., —CH₃) and each of X₂ and X₃ are hydrogen.

In some embodiments, Z is a chain within the macromolecule. In some embodiments, the chain may comprise a unit of Monomer (A). In some embodiments, the chain may comprise a unit of Monomer (B).

In some embodiments, q is an integer between 1 and 6. In some embodiments, q is an integer between 1 and 3. In some embodiments, q is 1. In some embodiments, q is 2. In some embodiments, q is 3. In some embodiments, q is 4. In some embodiments, q is 5. In some embodiments, q is 6.

In some embodiments p is an integer between 1 and 20. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12. In some embodiments, p is 13. In some embodiments, p is 14. In some embodiments, p is 15. In some embodiments, p is 16. In some embodiments, p is 17. In some embodiments, p is 18. In some embodiments, p is 19. In some embodiments, p is 20.

In some embodiments, B is

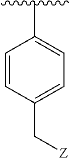

### and A is

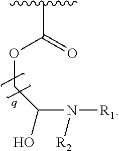

In some embodiments, B is

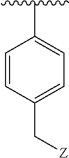

### and A is

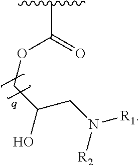

In some embodiments, B is

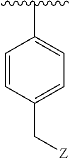

### and A is

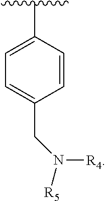

In some embodiments, B is

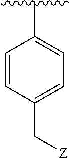

### and A is

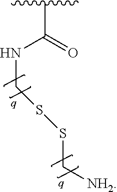

In some embodiments, B is

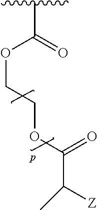

### and A is

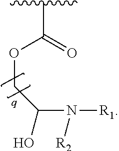

In some embodiments, B is

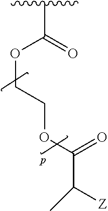

### and A is

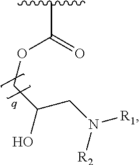

In some embodiments, B is

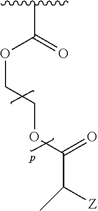

### and A is

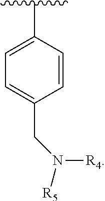

In some embodiments, B is

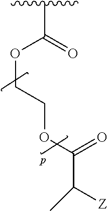

### and A is

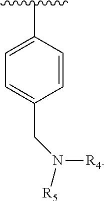

In some embodiments, B is

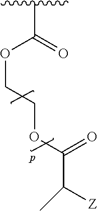

### and A is

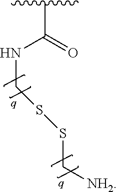

In some embodiments, B is

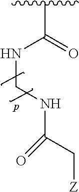

### and A is

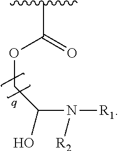

In some embodiments, B is

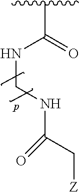

### and A is

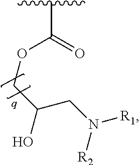

In some embodiments, B is

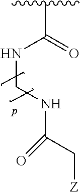

### and A is

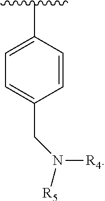

In some embodiments, B is

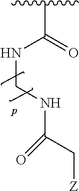

### and A is

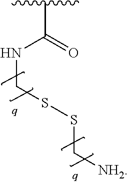

In some embodiments, the combinations of R₁, R₂, R₄ and R₅, are as described elsewhere herein or as disclosed in Table 1. In some embodiments, B is

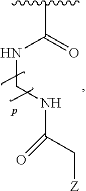

### A is

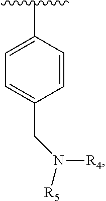

p is 1, R₁ is hydrogen, and R₂ is optionally substituted C₁-C₆ alkylamine (e.g., alkyl substituted dimethylamine or —(CH₂)₃dimethylamine). In some embodiments, B is

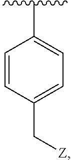

### A is

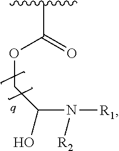

R₁ is hydrogen and R₂ is C₁₋₁₂ alkylamine (e.g., —CH₂CH₂NH₂ or —(CH₂)₆NH₂). In some embodiments, B is

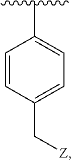

### A is

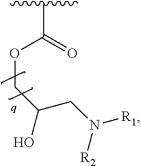

R₁ is hydrogen and R₂ is C₁₋₁₂ alkylamine (e.g., —CH₂CH₂NH₂ or —(CH₂)₆NH₂). In some embodiments, B is

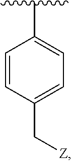

### A is

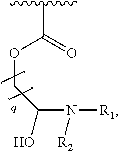

R₁ is hydrogen and R₂ is three fused 6-membered cycloalkyl rings. In some embodiments, B is

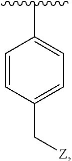

### A is

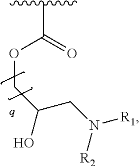

R₁ is hydrogen and R₂ is three fused 6-membered cycloalkyl rings. In some embodiments, B is

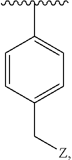

### A is

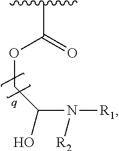

R₁ is hydrogen and R₂ is —(CH₂)₁₋₆ pyridine (e.g., —(CH₂)pyridine). In some embodiments, B is

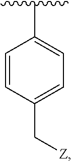

### A is

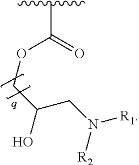

R₁ is hydrogen and R₂ is —(CH₂)₁₋₆ pyridine (e.g., —(CH₂)pyridine). In some embodiments, B is

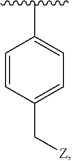

### A is

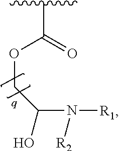

R₁ is hydrogen and R₂ is C₁-C₆ hydroxy (e.g., —(CH₂)₂OH). In some embodiments, B is

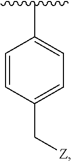

### A is

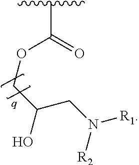

R₁ is hydrogen and R₂ is C₁-C₆ hydroxy (e.g., —(CH₂)₂OH). In some embodiments, B is

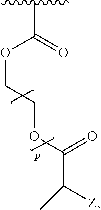

### A is

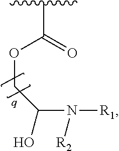

R₁ is hydrogen and R₂ is C₁₋₁₂ alkylamine (e.g., —(CH₂)₁₂NH₂). In some embodiments, B is

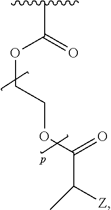

### A is

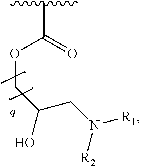

R₁ is hydrogen and R₂ is C₁₋₁₂ alkylamine (e.g., —(CH₂)₁₂NH₂). In some embodiments, B is

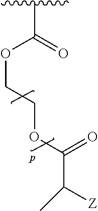

### A is

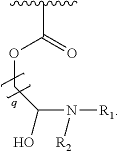

R₁ is hydrogen and R₂ is a boronic acid (e.g., phenylboronic acid). In some embodiments, B is

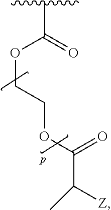

### A is

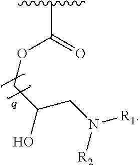

R₁ is hydrogen and R₂ is a boronic acid (e.g., phenylboronic acid). In some embodiments, B is

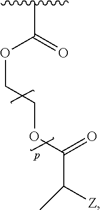

### A is

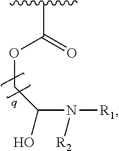

R₁ is hydrogen and R₂ is C₁-C₆ hydroxy (e.g., —(CH₂)₂OH). In some embodiments, B is

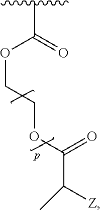

### A is

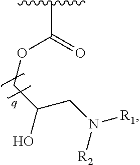

R₁ is hydrogen and R₂ is C₁-C₆ hydroxy (e.g., —(CH₂)₂OH).

In one aspect, the macromolecule comprises recurring units of a first component and a second component, wherein the first component comprises a structure of Component (A) and the second component comprises a structure of Component (B):

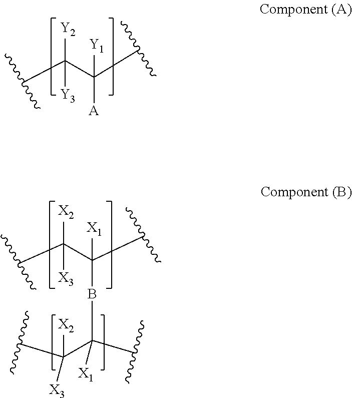

As provided herein,

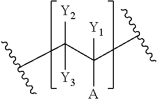

may refer interchangeably with

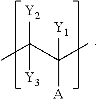

In some embodiments, each of Y₁, Y₂, and Y₃ is independently selected from hydrogen or C₁-C₆ alkyl. In some embodiments, Y₁ is hydrogen. In some embodiments, Y₁ is C₁-C₆ alkyl. In some embodiments, Y₂ is hydrogen. In some embodiments, Y₂ is C₁-C₆ alkyl. In some embodiments, Y₃ is hydrogen. In some embodiments, Y₃ is C₁-C₆ alkyl. In some embodiments, Y₁ is C₁-C₃ alkyl and each of Y₂ and Y₃ are hydrogen. In some embodiments, Y₁ is C₁ alkyl (e.g., —CH₃) and each of Y₂ and Y₃ are hydrogen.

In some embodiments, each of X₁, X₂, and X₃ is independently selected from hydrogen or C₁-C₆ alkyl. In some embodiments, X₁ is hydrogen. In some embodiments, X₁ is C₁-C₆ alkyl. In some embodiments, X₂ is hydrogen. In some embodiments, X₂ is C₁-C₆ alkyl. In some embodiments, X₃ is hydrogen. In some embodiments, X₃ is C₁-C₆ alkyl. In some embodiments, X₁ is C₁-C₃ alkyl and each of X₂ and X₃ are hydrogen. In some embodiments, X₁ is C₁ alkyl (e.g., —CH₃) and each of X₂ and X₃ are hydrogen.

In some embodiments, A is

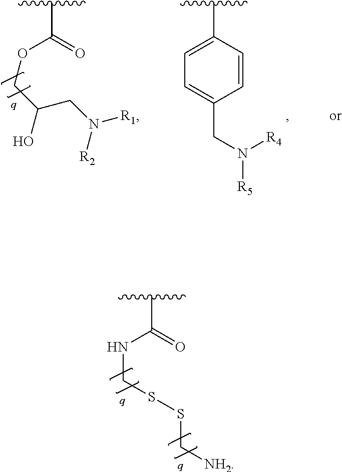

In some embodiments, A is

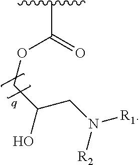

In some embodiments, A is

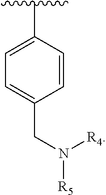

In some embodiments, A is

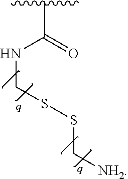

In some embodiments, the substituents of A (e.g., R₁, R₂, R₄, R₅) are described elsewhere herein.

In some embodiments, B is

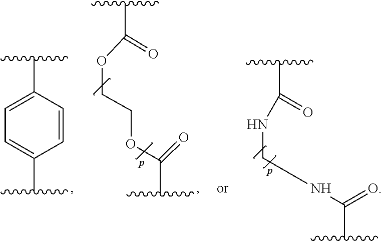

In some embodiments, B is

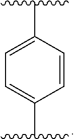 [SMILES: *c1ccc(*)cc1]

In some embodiments, B is

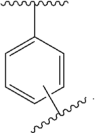

In some embodiments, B is

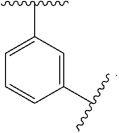 [SMILES: *c1cccc(*)c1]

In some embodiments, B is

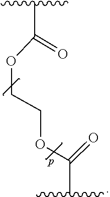

In some embodiments, B is

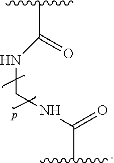

In some embodiments, q is an integer between 1 and 6. In some embodiments, q is an integer between 1 and 3. In some embodiments, q is 1. In some embodiments, q is 2. In some embodiments, q is 3. In some embodiments, q is 4. In some embodiments, q is 5. In some embodiments, q is 6.

In some embodiments p is an integer between 1 and 20. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12. In some embodiments, p is 13. In some embodiments, p is 14. In some embodiments, p is 15. In some embodiments, p is 16. In some embodiments, p is 17. In some embodiments, p is 18. In some embodiments, p is 19. In some embodiments, p is 20.

In some embodiments, the macromolecule may further comprise a second structure of Component (A). In some embodiments, a first structure of Component (A) and a second structure of Component (A) may be different. In some embodiments, the first structure of component (A) may be

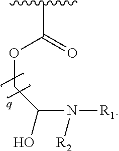

In some embodiments, the first structure of component (A) may be

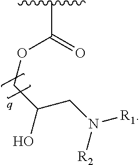

In some embodiments, the first structure of Component (A) may comprise R₁ as hydrogen and R₂ as —CH₂CH₂NH₂. In some embodiments, the second structure of Component (A) may comprise R₁ as hydrogen and R₂ as C₁-C₆ hydroxy (e.g., —CH₂CH₂OH), optionally substituted aryl (e.g., 2-fluorotoluene), optionally substituted heteroaryl (e.g., 1-propylimidazole), two or more fused 5 to 6 member rings (e.g., three fused 6 membered rings), or a monosaccharide (e.g., d-glucose).

Also provided herein are macromolecules comprising repeating units of component (A) such as repeating units of poly(ethylene)glycol dimethacrylate.

In some embodiments, the component (A) may comprise a weight percent of the mixture of monomers of about 10% to about 90%. In some embodiments, the component (A) may comprise a weight percent of the mixture of monomers of about 20 to about 80%. In some embodiments, the component (A) may comprise a weight percent of the mixture of monomers of about 40% to about 60%. In some embodiments, the component (A) may comprise a weight percent of the mixture of monomers of about 50%. In some embodiments, the Component (B) or Component (B′) may comprise a weight percent of the mixture of monomers of about 10% to about 90%. In some embodiments, the Component (B) or Component (B′) may comprise a weight percent of the mixture of monomers of about 20 to about 80%. In some embodiments, the Component (B) or Component (B′) may comprise a weight percent of the mixture of monomers of about 40% to about 60%. In some embodiments, the Component (B) or Component (B′) may comprise a weight percent of the mixture of monomers of about 50%.

As used herein,

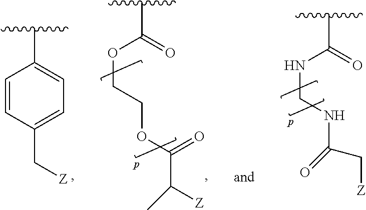

represent the incorporation of divinylbenzene, ethyleneglycol dimethacrylate, and methylene(bisacrylamide), respectively, into the macromolecule chain when both vinyl groups have undergone a polymerization reaction to cross-link macromolecule chains. Accordingly, as shown in Tables 1-4,

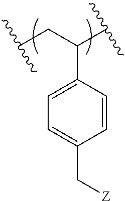

represents a crosslinked divinylbenzene. Accordingly, as shown in Table 1-4,

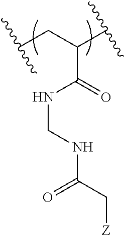

represents a crosslinked N,N-Methylenebis(acrylamide). Accordingly, as shown in Tables 1-4,

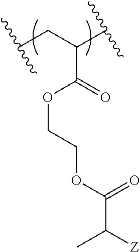

represents a crosslinked Ethylene glycol dimethacrylate.

In some embodiments, the incorporation of

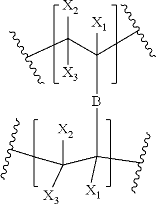

refers to incorporation of component (B) as a cross-linker in the macromolecule.

In some embodiments, the macromolecule may comprise recurring units as shown in Table 2, where n, m, x, and y indicate a recurring unit. In some embodiments, m is an integer greater than 2. In some embodiments, m is an integer between 2 and 50,000 (e.g., between 10 and 10,000, between 25 and 2,500, or between 50 and 1,000). In some embodiments, n is an integer greater than 2. In some embodiments, n is an integer between 2 and 50,000 (e.g., between 10 and 10,000, between 25 and 2,500, or between 50 and 1,000). In some embodiments, the recurring units denoted by n and m are randomly copolymerized. In some embodiments x is an integer greater than 2. In some embodiments, x is an integer between 2 and 50,000 (e.g., between 10 and 10,000, between 25 and 2,500, or between 50 and 1,000). In some embodiments y is an integer greater than 2. In some embodiments, y is an integer between 2 and 50,000 (e.g., between 10 and 10,000, between 25 and 2,500, or between 50 and 1,000).

TABLE 2

| Compound | Structure |
|---|---|
| 200 | 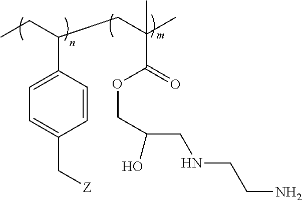 |
| 201 | 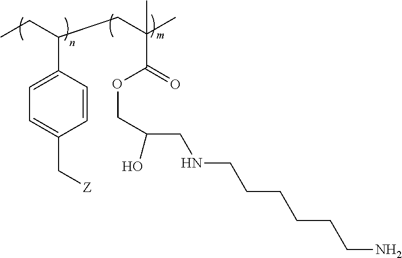 |
| 202 | 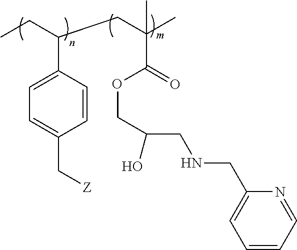 |
| 203 | 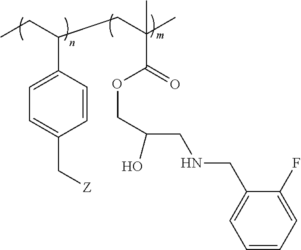 |
| 204 | 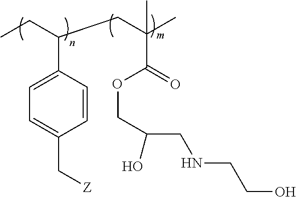 |
| 205 | 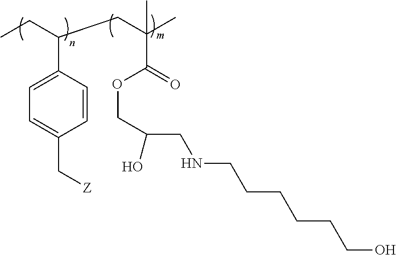 |
| 206 | 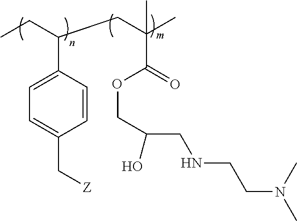 |
| 207 | 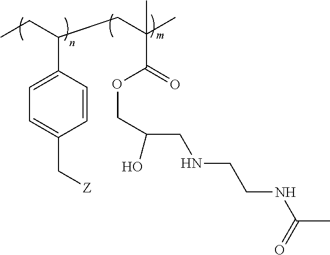 |
| 208 | 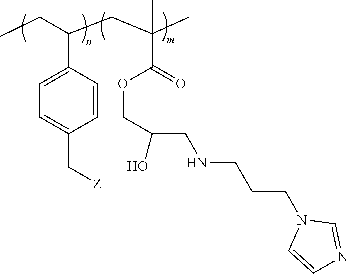 |
| 209 | 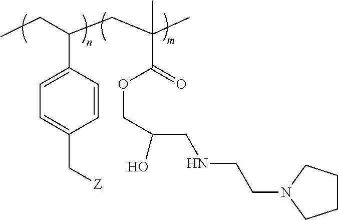 |
| 210 | 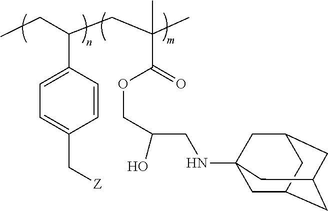 |
| 211 | 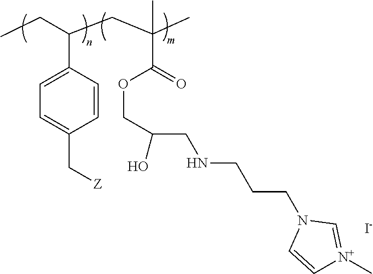 |
| 212 | 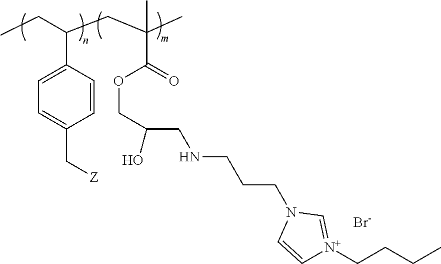 |
| 213 | 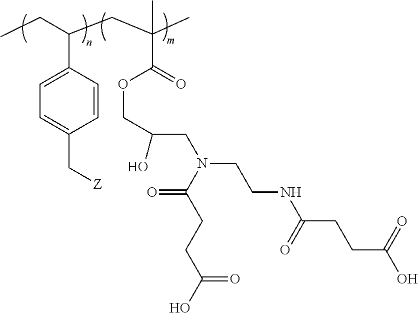 |
| 214 | 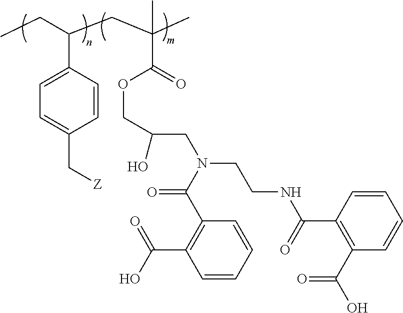 |
| 215 | 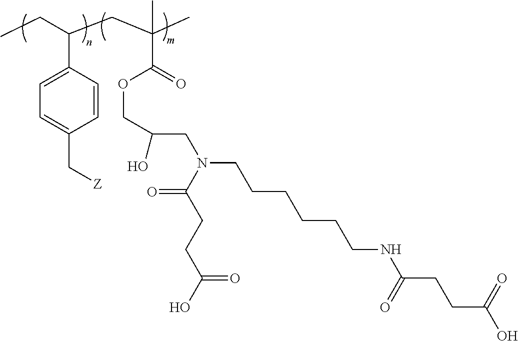 |
| 216 | 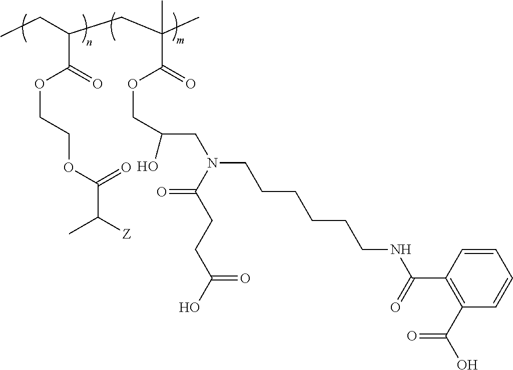 |
| 217 | 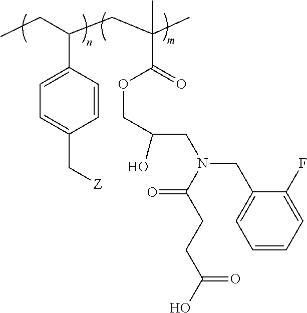 |
| 218 | 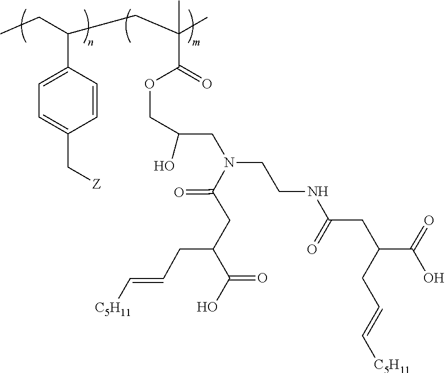 |
| 219 | 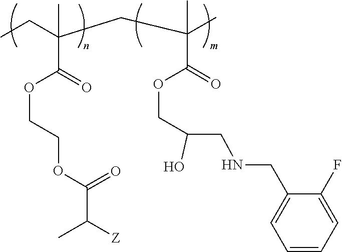 |
| 220 | 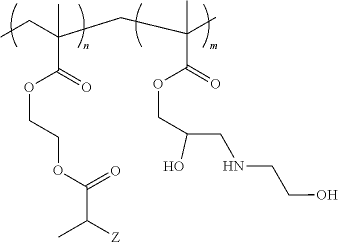 |
| 221 | 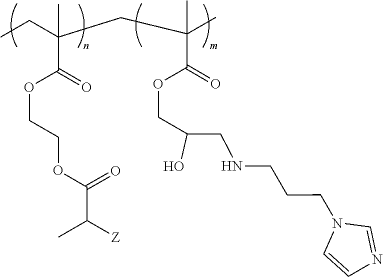 |
| 222 | 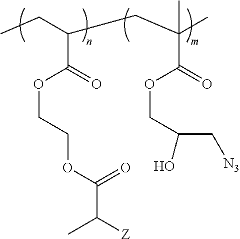 |
| 223 | 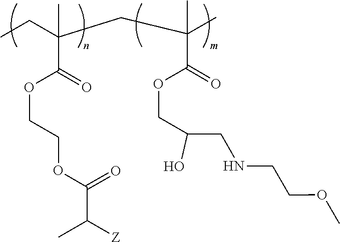 |
| 224 | 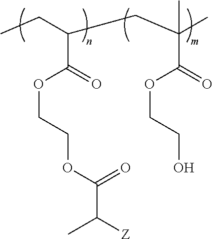 |
| 225 | 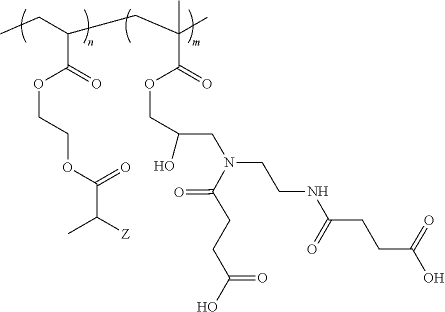 |
| 226 | 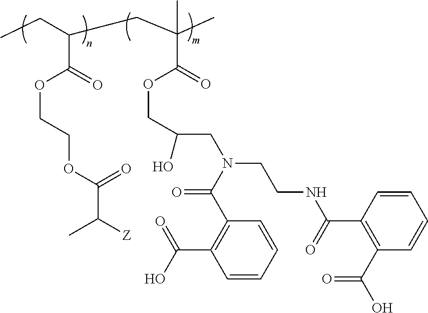 |
| 227 | 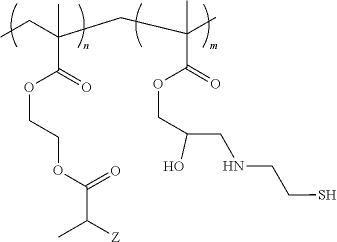 |
| 228 | 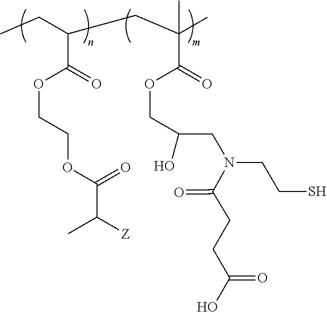 |
| 229 | 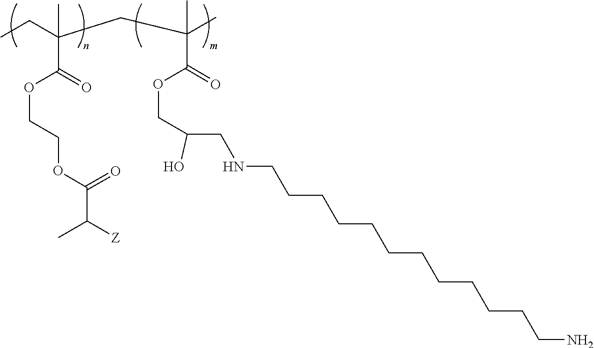 |
| 230 | 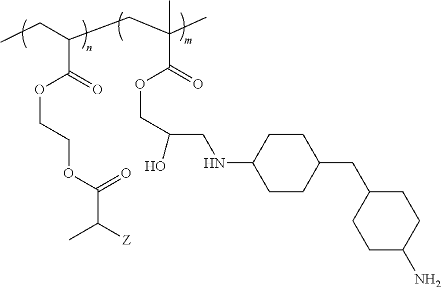 |
| 231 | 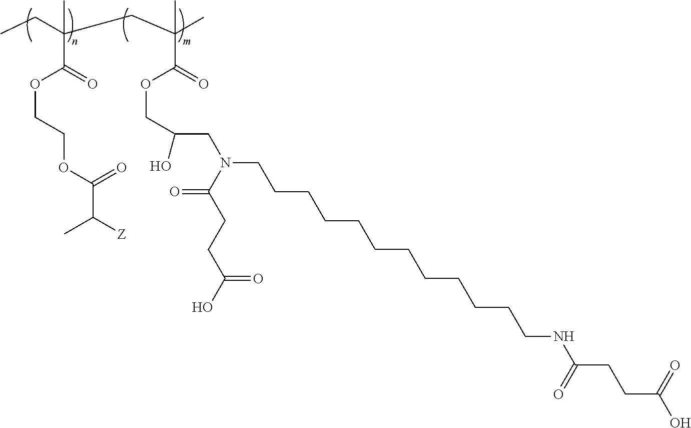 |
| 232 | 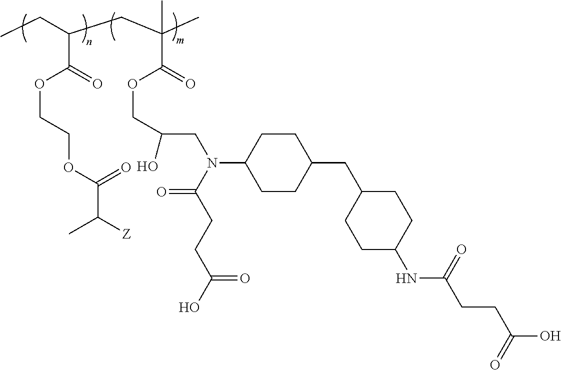 |
| 233 | 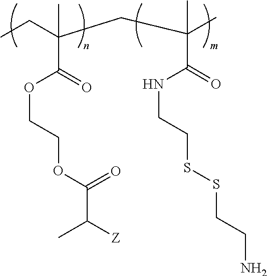 |
| 234 | 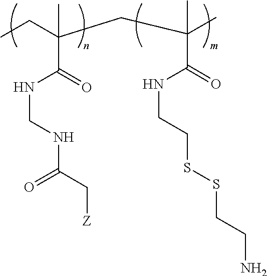 |
| 235 | 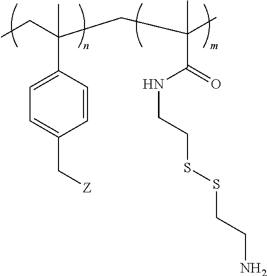 |
| 236 | 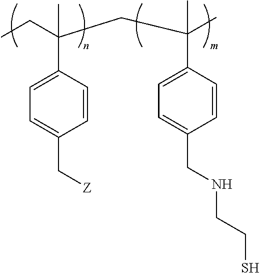 |
| 237 | 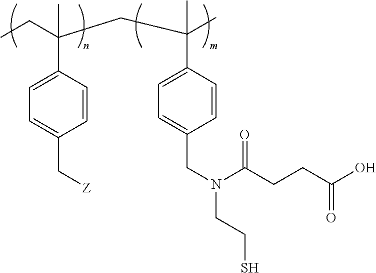 |
| 238 | 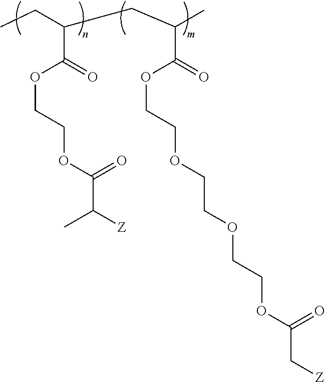 |
| 239 | 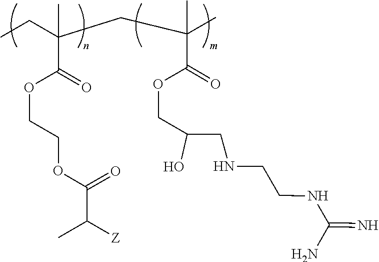 |
| 240 | 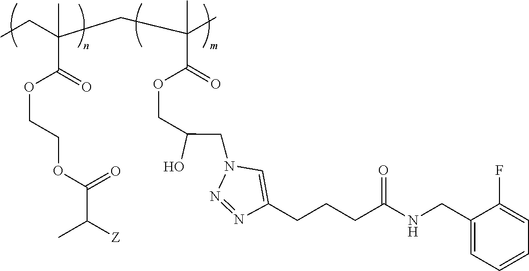 |
| 241 | 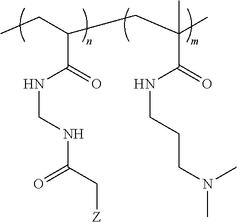 |
| 242 | 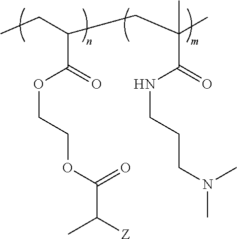 |
| 243 | 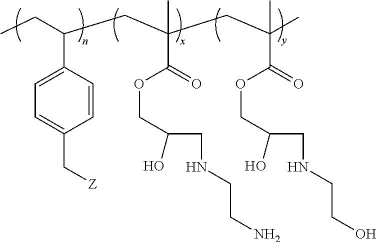 |
| 244 | 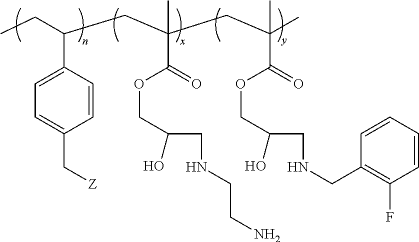 |
| 245 | 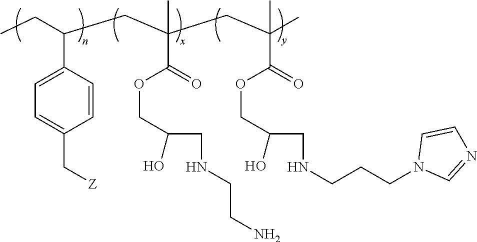 |
| 246 | 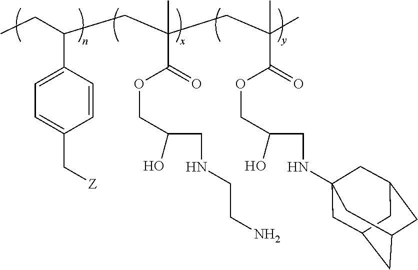 |
| 247 | 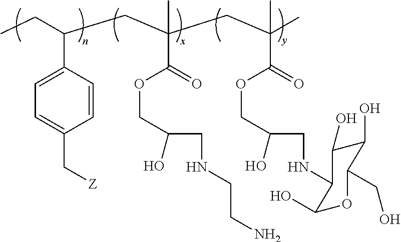 |
| 248 | 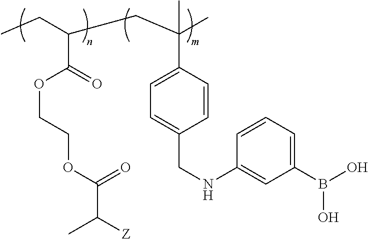 |
| 249 | 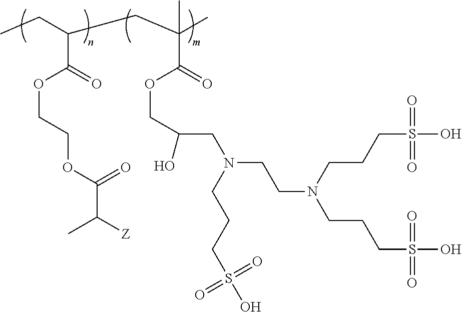 |
| 250 | 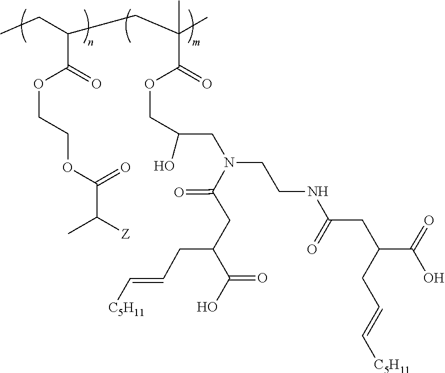 |
| 251 | 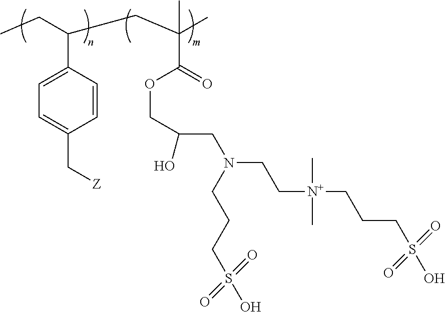 |
| 252 | 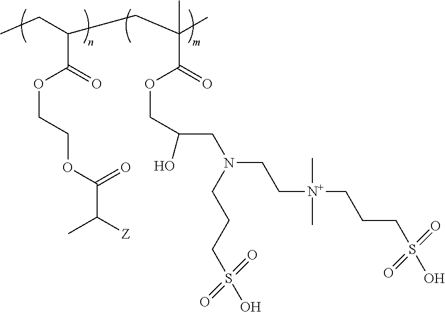 |

As used herein,

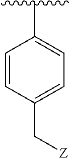

represents the incorporation of divinyl benzene into the macromolecule chain when both vinyl groups have undergone a polymerization reaction to cross-link macromolecule chains. In some embodiments,

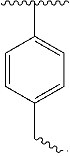

Is represented as

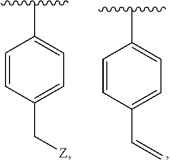

or mixtures thereof. In some embodiments,

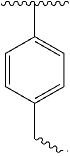

Is represented as

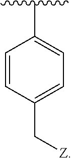

As used herein,

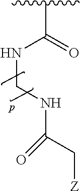

represents the incorporation of N,N′-methylenebisacrylamide (MBA) into the macromolecule chain when both vinyl groups have undergone a polymerization reaction to cross-link macromolecule chains. In some embodiments,

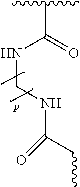

is represented as

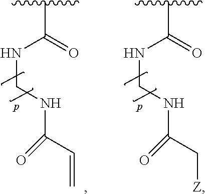

or mixtures thereof. In some embodiments,

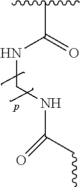

is represented as

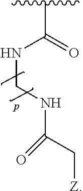

As used herein,

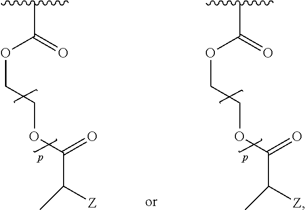

when p is 1, represents the incorporation of ethyleneglycol dimethacrylate (EGDMA) into the macromolecule chain when both vinyl groups have undergone a polymerization reaction to cross-link macromolecule chains.

In any of the embodiments provided herein,

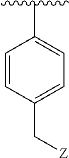

may refer to a (polymerized) divinylbenzene. In some instances, the divinylbenzene is a p-divinylbenzene. In some instances, the divinylbenzene is o-divinylbenzene. In some instances, the divinylbenzene is m-divinylbenzene. In some instances, the structure of

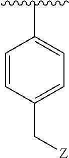

may refer interchangeably to

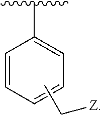

In some instances,

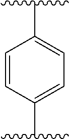 [SMILES: *c1ccc(*)cc1]

as used herein may refer to

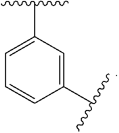 [SMILES: *c1cccc(*)c1]

In some instances,

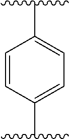 [SMILES: *c1ccc(*)cc1]

as used herein may refer interchangeably to

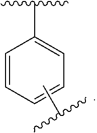

In one aspect, described herein is a surface comprising a moiety of Formula (IV):

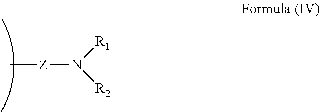

wherein Z is a linking moiety comprising a linear chain with 2 to 20 atoms selected from the group consisting of carbon, oxygen, and nitrogen, and optional substituents on the carbon and any nitrogen atoms in the linear chain;
R₁ is hydrogen or succinate; and
R₂ is optionally substituted —C₁-C₆ disulfide or C₁-C₆ thiol.

In some embodiments, Z is a linear chain with 2 to 20 atoms. In some embodiments, Z is a linear chain with 2 to 12 atoms. In some embodiments, Z is a linear chain with 2 to 6 atoms. In some embodiments, Z is a linear chain with 2 atoms. In some embodiments, Z is a linear chain with 3 atoms. In some embodiments, Z is a linear chain with 4 atoms. In some embodiments, Z is a linear chain with 5 atoms. In some embodiments, Z is a linear chain with 6 atoms. In some embodiments Z comprises carbon only. In some embodiments, Z is a C₂-C₆ alkyl chain. In some embodiments, Z is C₃ alkyl. In some embodiments Z comprises oxygen, nitrogen, carbon, or a combination thereof. In some embodiments, Z comprises substituents on the linear chain.

In some embodiments, Z comprises a C₂-C₈ heteroalkyl, optionally substituted (e.g., with hydroxy). In some embodiments, Z comprises a C₂-C₈ alkoxy, optionally substituted (e.g., with hydroxy).

In some embodiments, R₁ is hydrogen, optionally substituted succinate, optionally substituted glutarate, optionally substituted adipate, optionally substituted pimelate, optionally substituted suberate, optionally substituted azelate, or optionally substituted sebacate. In some embodiments, R₁ is optionally substituted glutarate. In some embodiments, R₁ is optionally substituted adipate. In some embodiments, R₁ is optionally substitute pimelate. In some embodiments, R₁ is optionally substituted suberate. In some embodiments, R₁ is optionally substitute azelate. In some embodiments, R₁ is optionally substitute sebacate. In some embodiments, R₁ is hydrogen. In some embodiments, R₁ is optionally substituted C₁-C₆ alkyl. In some embodiments, R₁ is substituted with one or more oxo and —COOH. In some embodiments, R¹ is succinate. In some embodiments, R₁ is optionally substituted succinate. In some embodiments, R₂ is optionally substituted —C₁-C₆ disulfide. In some embodiments, R₂ is C₁-C₆ thiol. In some embodiments, R₂ is substituted —C₁-C₆ alkyl disulfide. In some embodiments, R₂ is substituted di-C₁-C₆ alkyl disulfide. In some embodiments, R₂ is —CH₂CH₂—S—S—CH₂CH₂NH₂. In some embodiments, R₂ is C₂ thiol (e.g., —(CH₂)₂SH). In some embodiments, Z is attached to a surface. In some embodiments, Z is covalently attached is a surface.

In some embodiments, a structure of Formula (IV) comprises

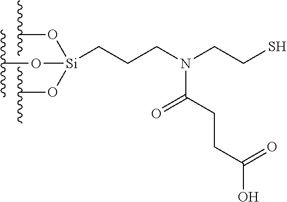 [SMILES: *O[Si](CCCN(CCS)C(=O)CCC(=O)O)(O*)O*]

(Compound 508). In some embodiments, a structure of Formula (IV) comprises

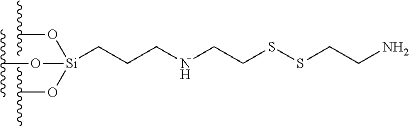 [SMILES: *O[Si](CCCNCCSSCCN)(O*)O*]

(Compound 507). In some embodiments, a structure of Formula (IV) comprises

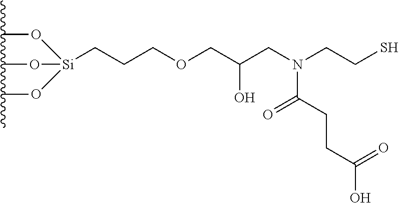

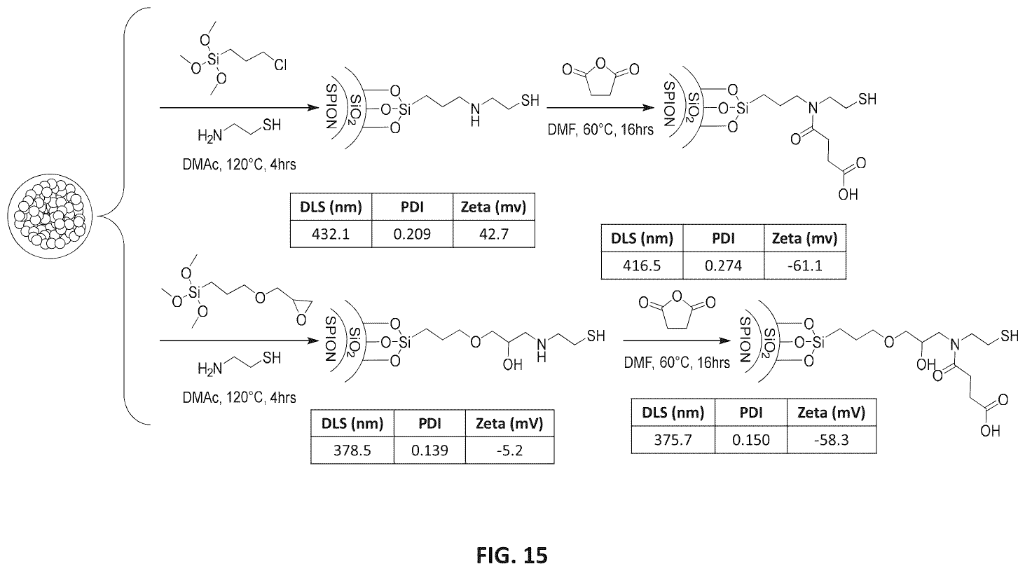

(Compound 509). FIG. 15 shows a reaction scheme for preparation of Compound 508 and 509 as well as particle size and surface potential.

In one aspect, described herein in a surface comprising the macromolecule or moieties of the disclosure as described elsewhere herein, wherein the macromolecule or moiety is immobilized to the surface. In some embodiments, the structures as disclosed in Table 3 and Table 4 are immobilized to a surface, where n, m, x, y, and surfaces are described elsewhere herein.

TABLE 3

| Compound | Structure |
|---|---|
| 300 | 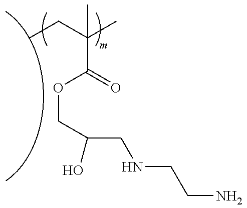 |
| 301 | 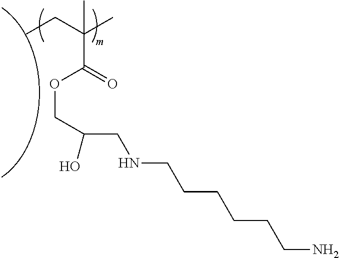 |
| 302 | 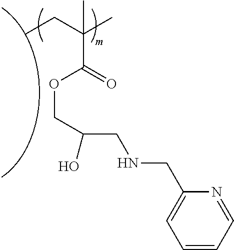 |
| 303 | 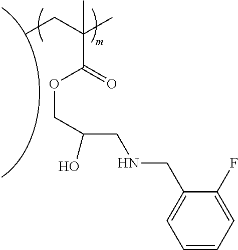 |
| 304 | 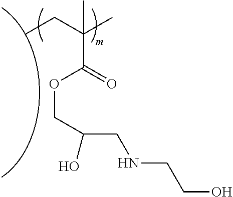 |
| 305 | 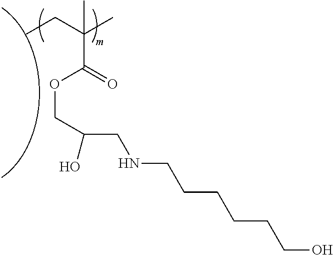 |
| 306 | 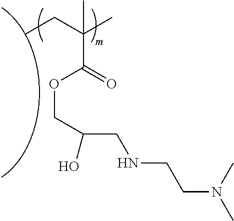 |
| 307 | 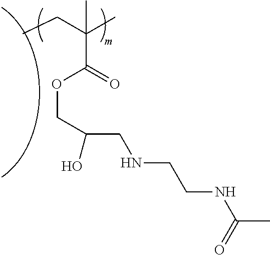 |
| 308 | 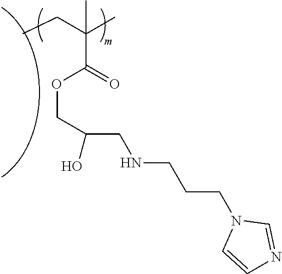 |
| 309 | 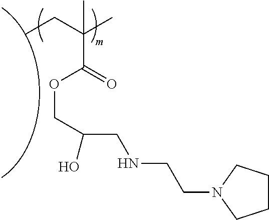 |
| 310 | 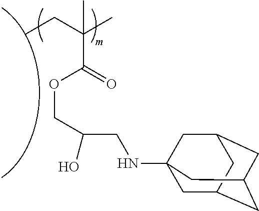 |
| 311 | 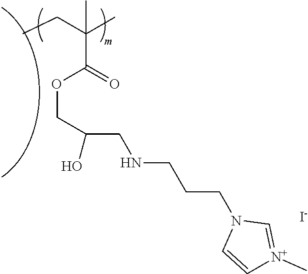 |
| 312 | 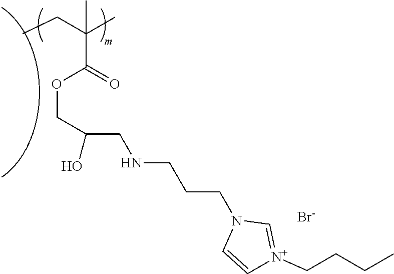 |
| 313 | 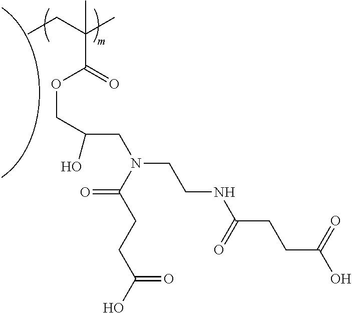 |
| 314 | 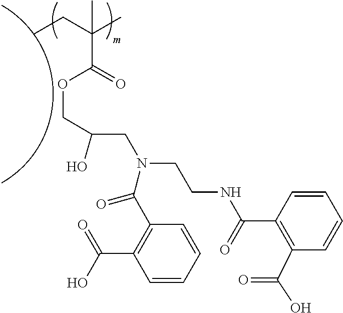 |
| 315 | 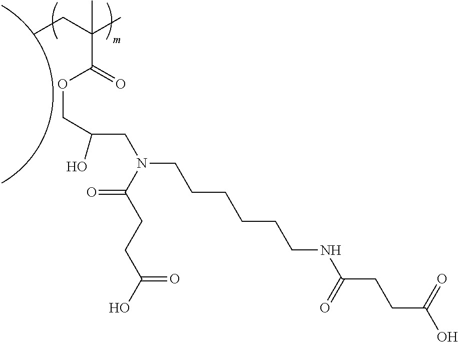 |
| 316 | 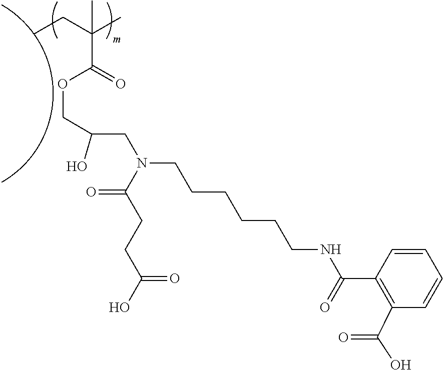 |
| 317 | 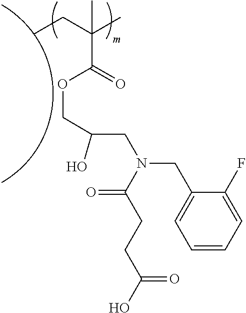 |
| 318 | 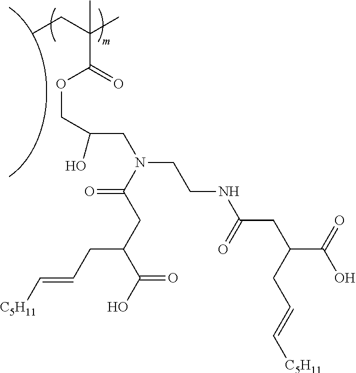 |
| 322 | 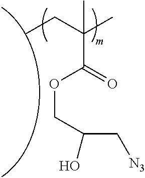 |
| 323 | 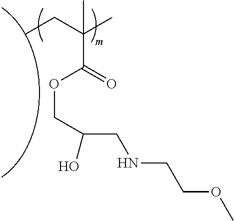 |
| 324 | 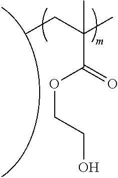 |
| 327 | 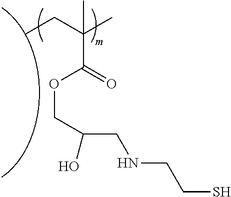 |
| 328 | 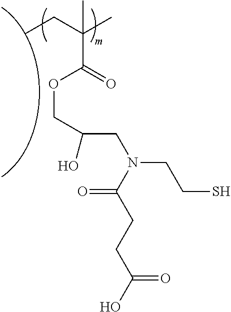 |
| 329 | 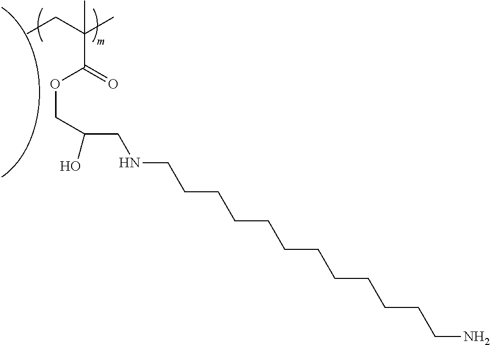 |
| 330 | 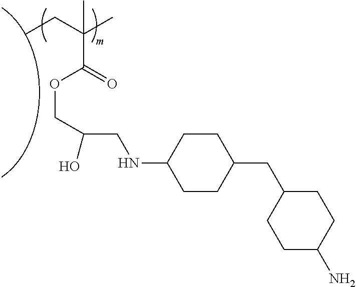 |
| 331 | 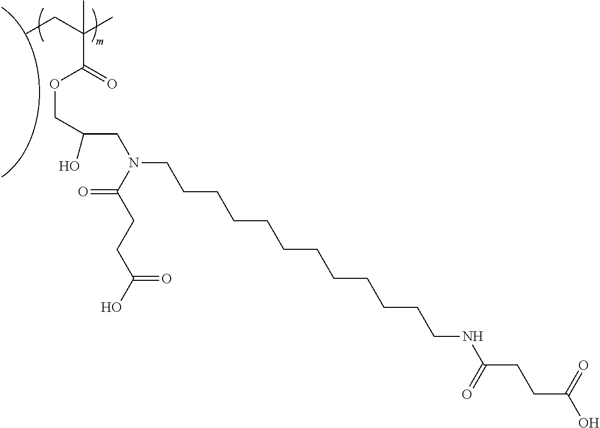 |
| 332 | 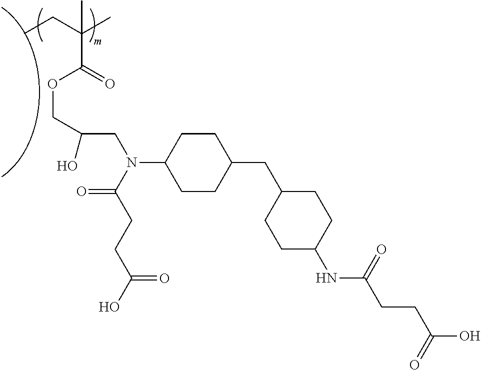 |
| 333 | 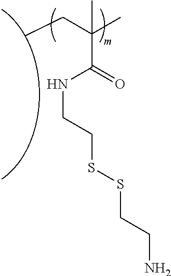 |
| 336 | 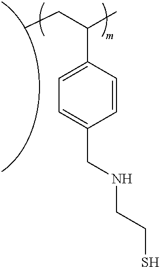 |
| 337 | 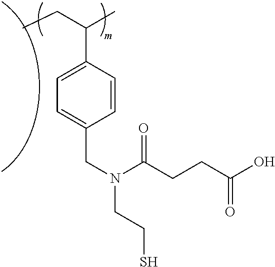 |
| 338 | 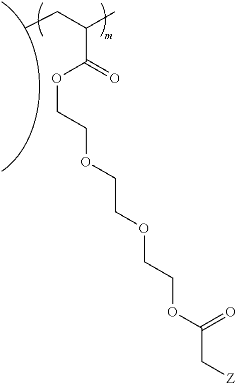 |
| 339 | 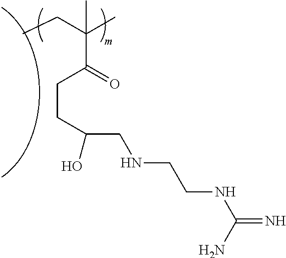 |
| 340 | 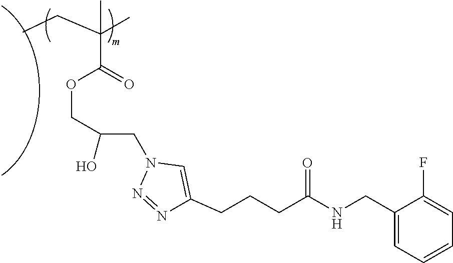 |
| 343 | 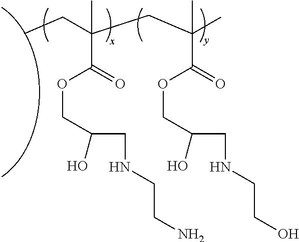 |
| 344 | 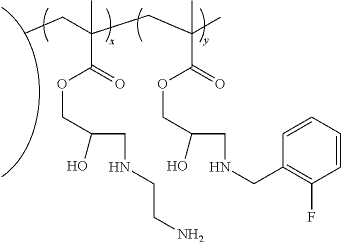 |
| 345 | 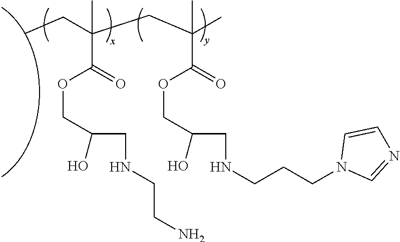 |
| 346 | 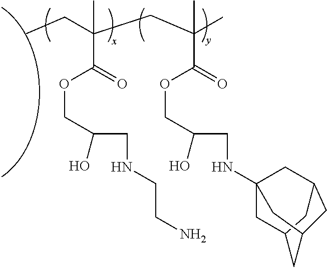 |
| 347 | 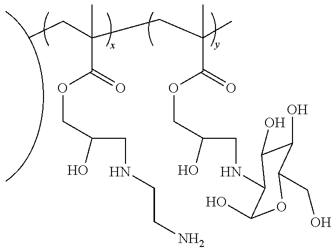 |
| 348 | 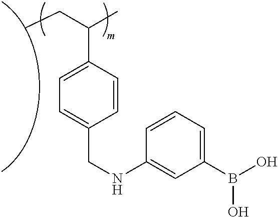 |
| 349 | 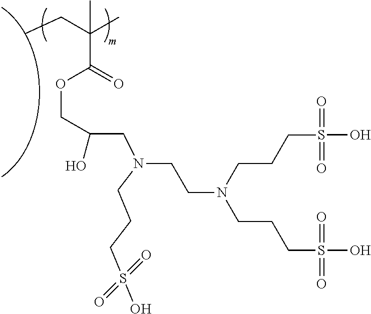 |
| 351 | 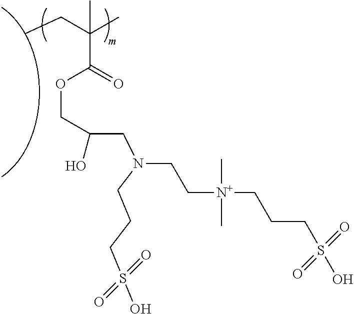 |
| 354 | 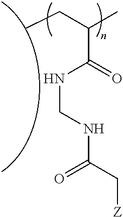 |

TABLE 4

| Compound | Structure |
|---|---|
| 400 | 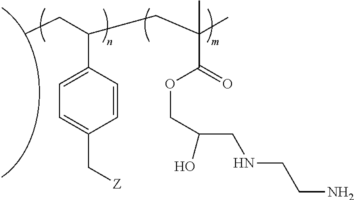 |
| 401 | 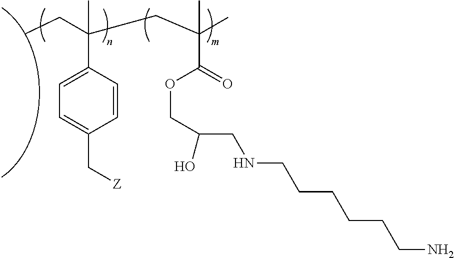 |
| 402 | 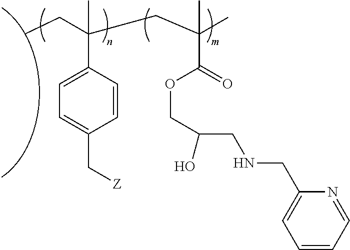 |
| 403 | 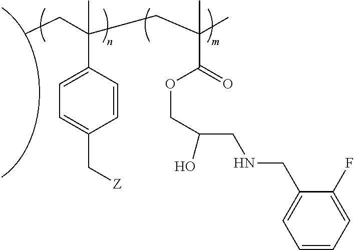 |
| 404 | 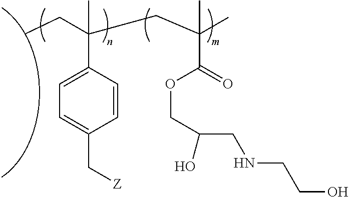 |
| 405 | 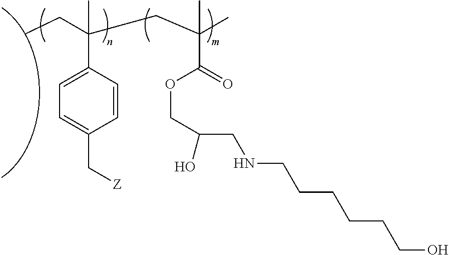 |
| 406 | 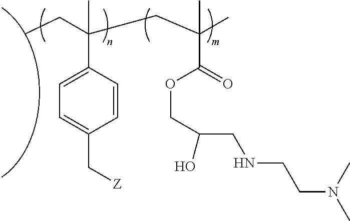 |
| 407 | 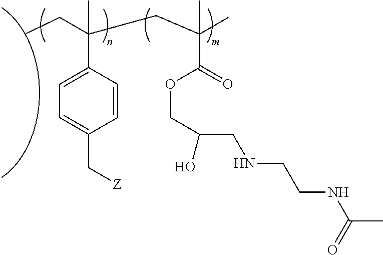 |
| 408 | 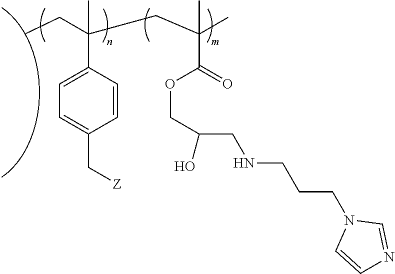 |
| 409 | 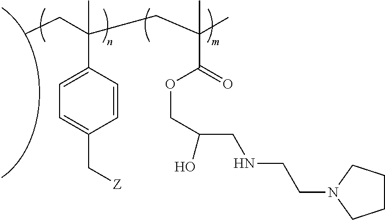 |
| 410 | 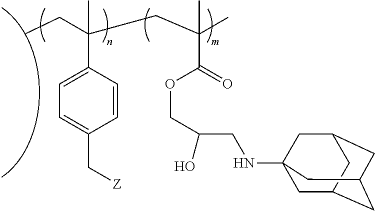 |
| 411 | 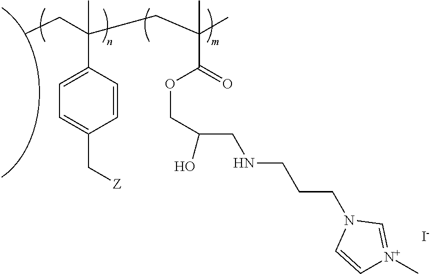 |
| 412 | 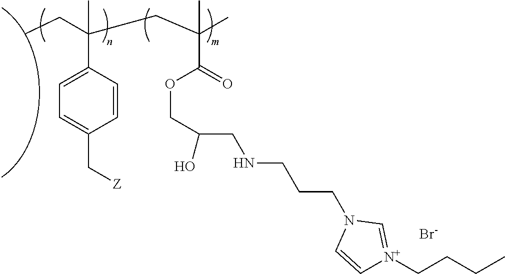 |
| 413 | 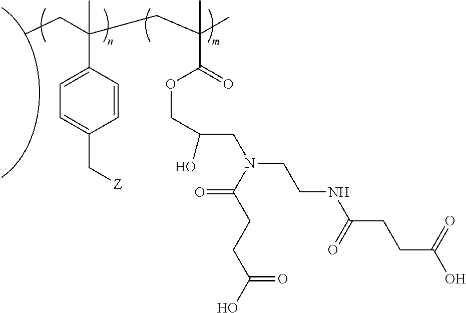 |
| 414 | 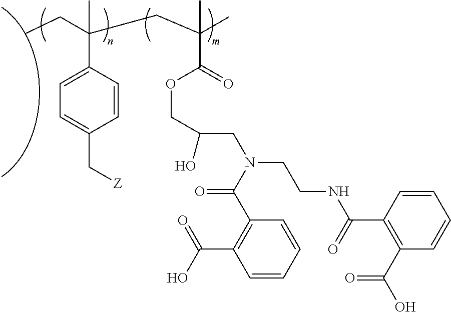 |
| 415 | 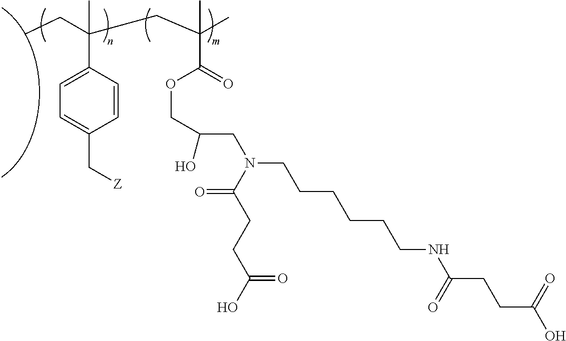 |
| 416 | 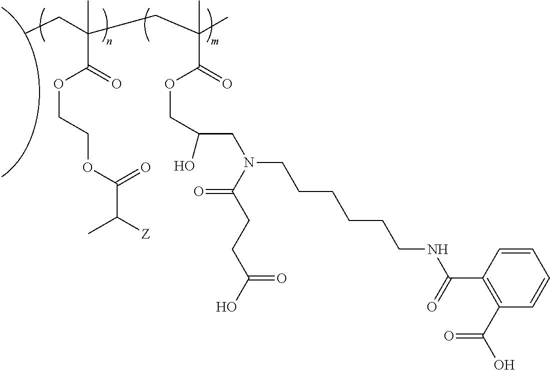 |
| 417 | 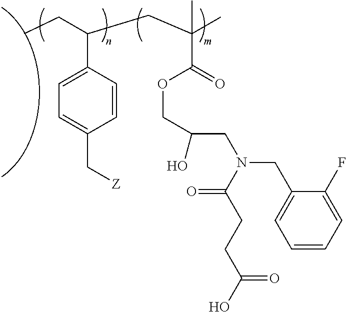 |
| 418 | 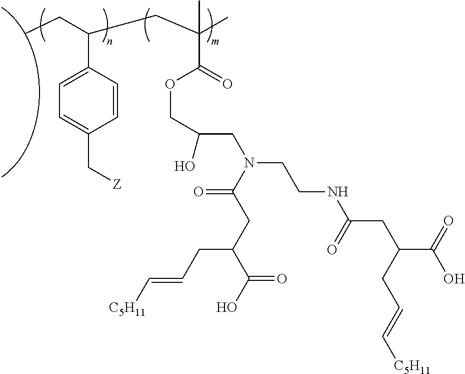 |
| 419 | 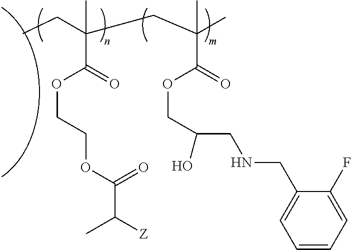 |
| 420 | 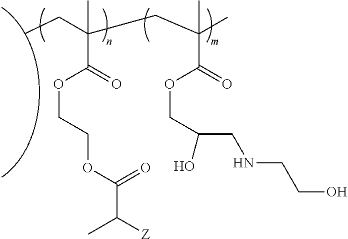 |
| 421 | 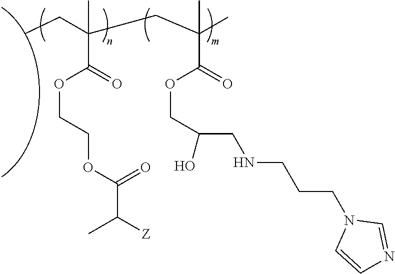 |
| 422 | 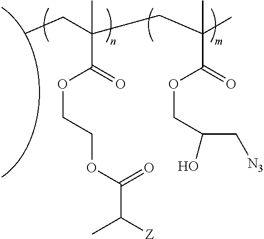 |
| 423 | 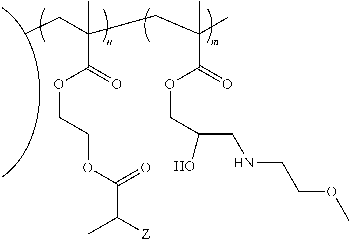 |
| 424 | 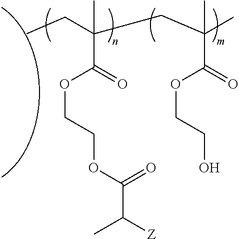 |
| 425 | 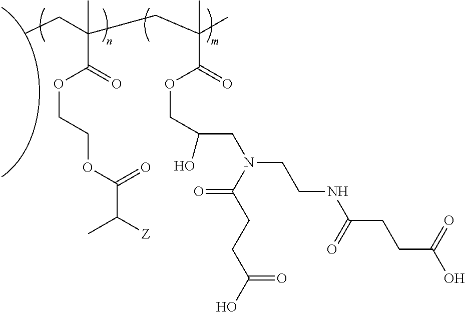 |
| 426 | 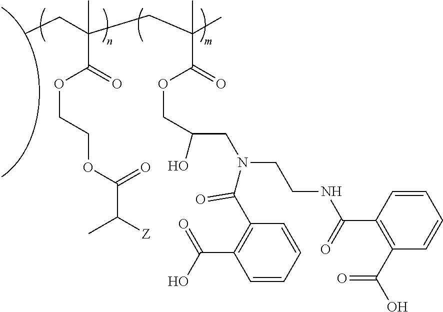 |
| 427 | 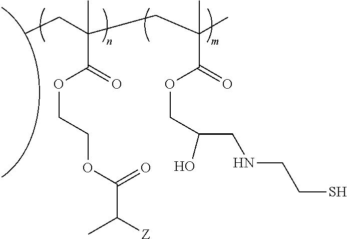 |
| 428 | 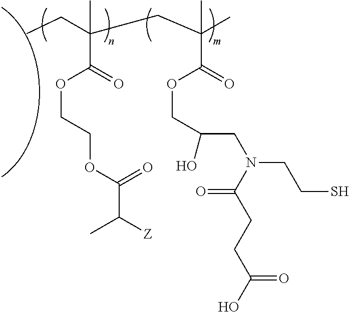 |
| 429 | 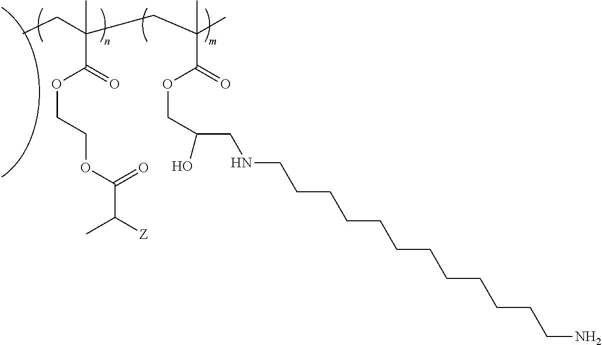 |
| 430 | 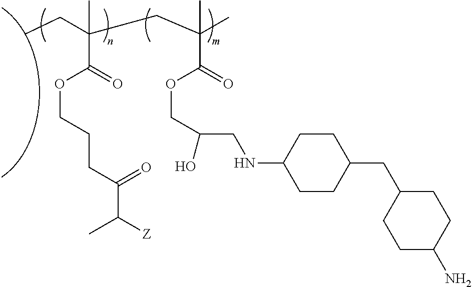 |
| 431 | 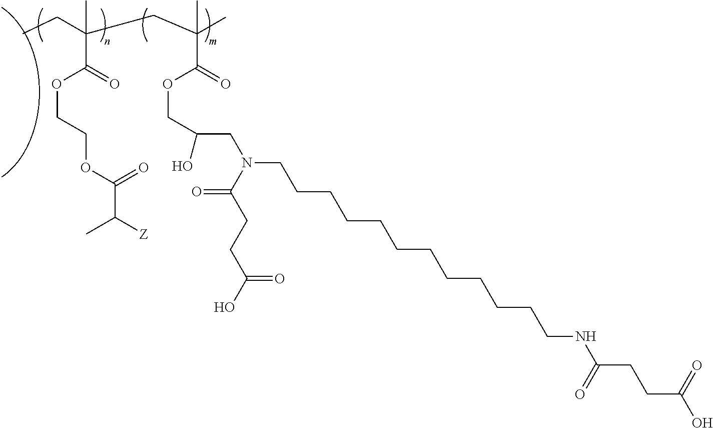 |
| 432 | 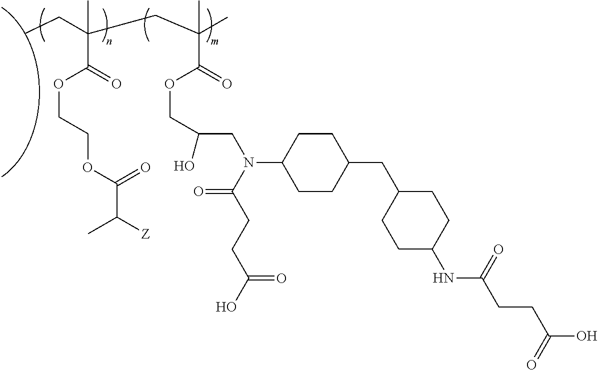 |
| 433 | 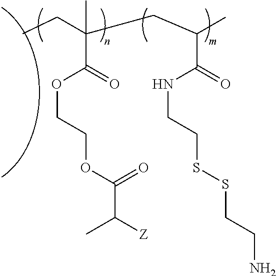 |
| 434 | 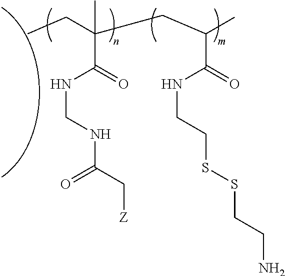 |
| 435 | 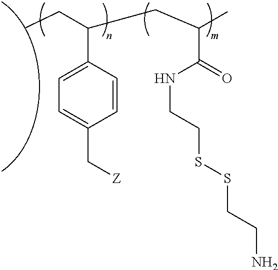 |
| 436 | 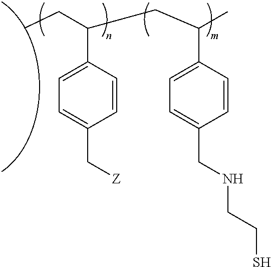 |
| 437 | 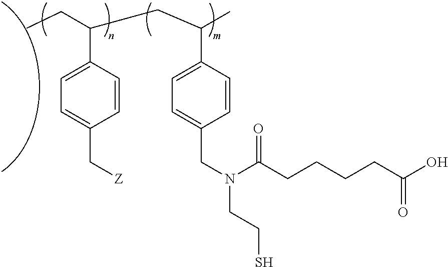 |
| 438 | 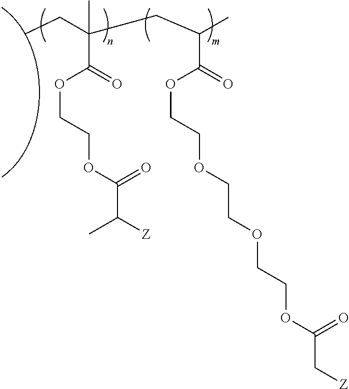 |
| 439 | 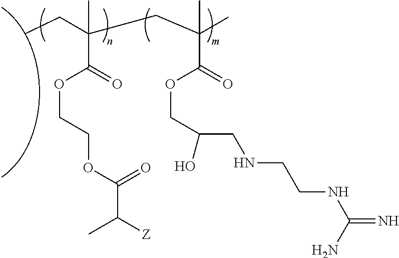 |
| 440 | 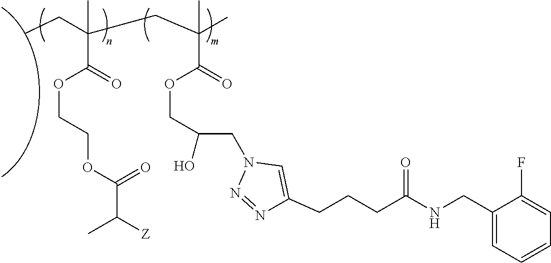 |
| 441 | 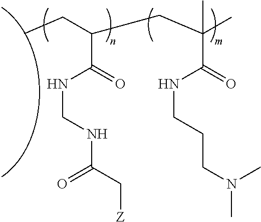 |
| 442 | 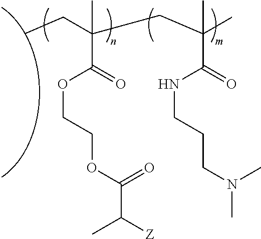 |
| 443 | 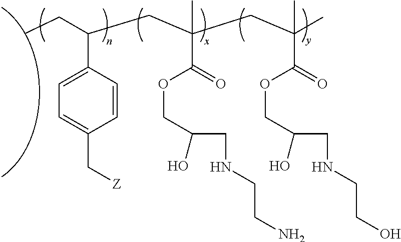 |
| 444 | 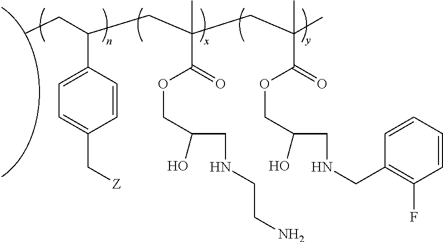 |
| 445 | 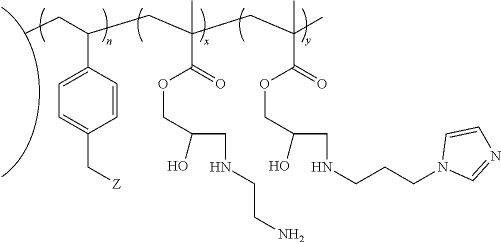 |
| 446 | 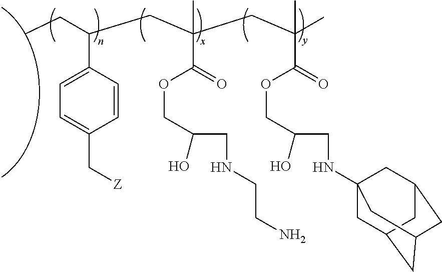 |
| 447 | 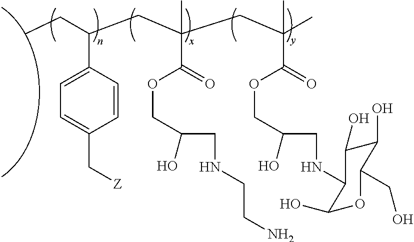 |
| 448 | 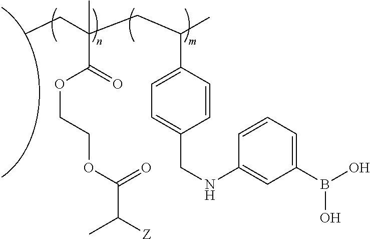 |
| 449 | 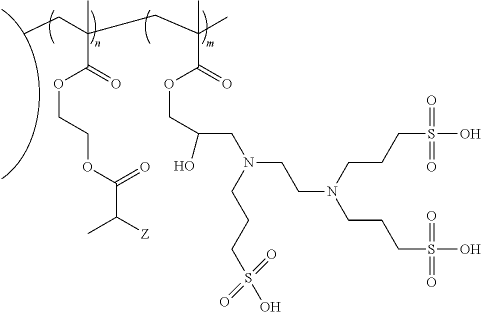 |
| 450 | 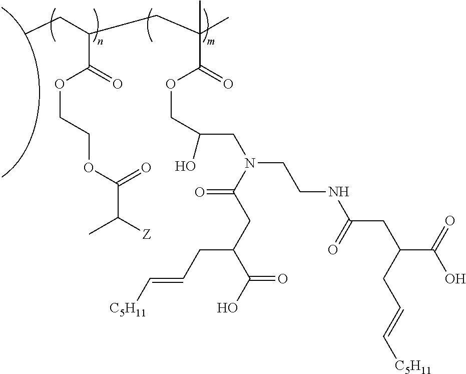 |
| 451 | 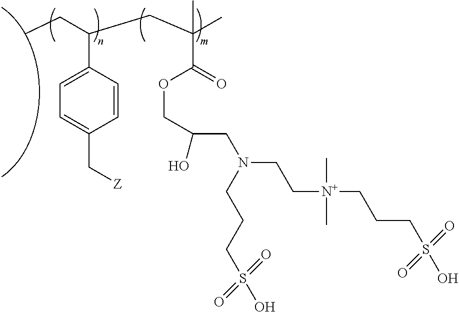 |
| 452 | 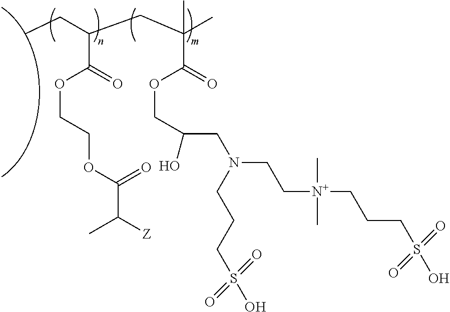 |
| 453 | 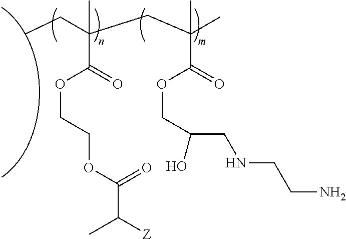 |

In one aspect, described herein in a surface comprising the macromolecule or moieties of the disclosure as described elsewhere herein, wherein the macromolecule or moiety is immobilized to the surface. In some embodiments, the structures are immobilized to a surface and described in Table 5, where n, m, and surfaces are described elsewhere herein.

TABLE 5

| Compound | Structure |
|---|---|
| 500 | 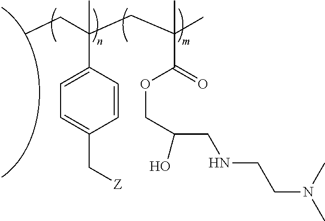 |
| 501 | 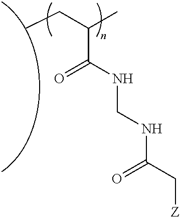 |
| 502 | 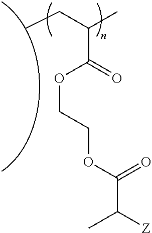 |
| 503 | 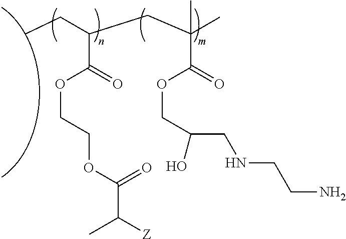 |
| 504 | 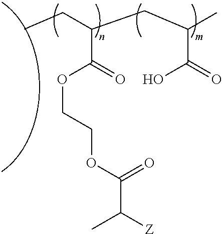 |
| 505 | 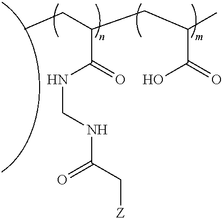 |
| 506 | 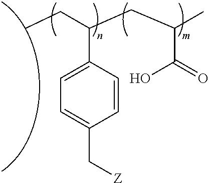 |

In some embodiments,

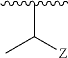

as represented in the compounds herein (e.g., such as in Tables 1-5) represent an attachment point for a unit of Component (A) or Component (B). In some embodiments,

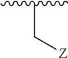

as represented in the compounds herein (e.g., such as in Tables 1-5) represent an attachment point for a unit of Component (A) or Component (B). In some embodiments,

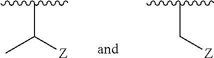

represent attachment points for crosslinking with a monomer of

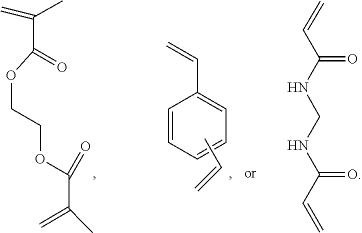

As used herein,

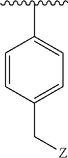

represents the incorporation of divinyl benzene into the macromolecule chain when both vinyl groups have undergone a polymerization reaction to cross-link macromolecule chains. In some embodiments,

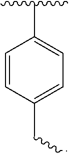

Is represented as

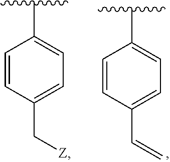

or mixtures thereof. In some embodiments,

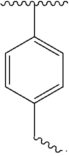

Is represented as

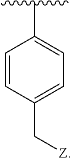

As used herein,

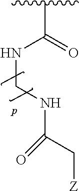

represents the incorporation of N,N′-methylenebisacrylamide (MBA) into the macromolecule chain when both vinyl groups have undergone a polymerization reaction to cross-link macromolecule chains. In some embodiments,

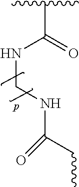

is represented as

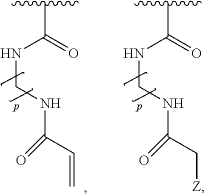

or mixtures thereof. In some embodiments,

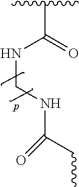

is represented as

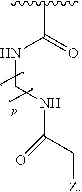

As used herein

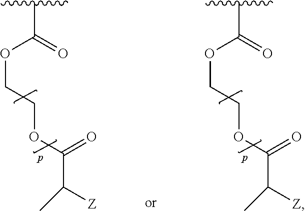

when p is 1, represents the incorporation of ethyleneglycol dimethacrylate (EGDMA) into the macromolecule chain when both vinyl groups have undergone a polymerization reaction to cross-link macromolecule chains.

In any of the embodiments provided herein,

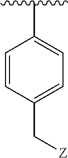

may refer to a (polymerized) divinylbenzene. In some instances, the divinylbenzene is a p-divinylbenzene. In some instances, the divinylbenzene is o-divinylbenzene. In some instances, the divinylbenzene is m-divinylbenzene. In some instances, the structure of

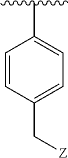

may refer interchangeably to

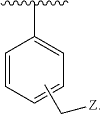

In some instances,

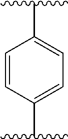 [SMILES: *c1ccc(*)cc1]

as used herein may refer to

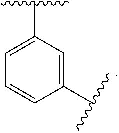 [SMILES: *c1cccc(*)c1]

In some instances,

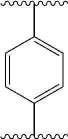 [SMILES: *c1ccc(*)cc1]

as used herein may refer interchangeably to

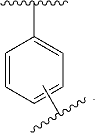

In some embodiments, the macromolecule or moieties are covalently coupled to a surface. In some embodiments, the macromolecule or moieties are electrostatically coupled to a surface. In some embodiments, the macromolecule or moieties are coupled to a surface via a polymerization event. In some embodiments, the polymerization event may comprise a reaction with a vinyl group on the surface. In some embodiments, the surface comprises silica and the vinyl group is attached to the surface using a silane coupling agent. In some embodiments, the surface may be a bead. In some embodiments, the surface may be a particle. In some embodiments, the particle may be a nanoparticle or microparticle with dimensions and properties as described elsewhere herein. In some embodiments, the particle may comprise features and properties (e.g., comprise iron oxide) as described elsewhere herein.

In some embodiments of Formula (I), (I-A), (I-A′), (II), (II′), (III), (III′), or (III-A) and (IV), each of R₁, R₂, R₄, R₅ is independently selected from hydrogen, nitrogen,

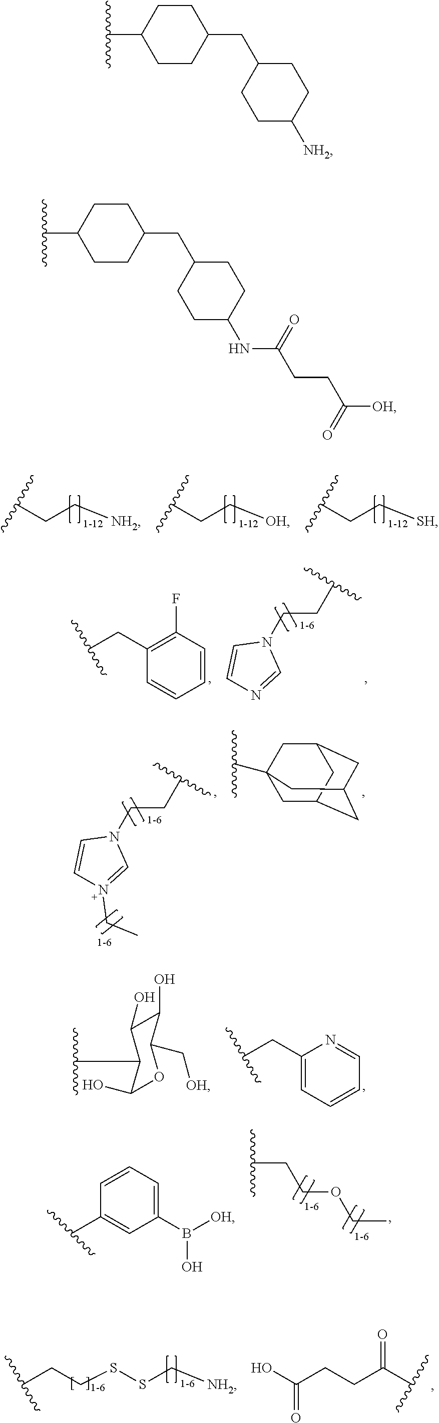

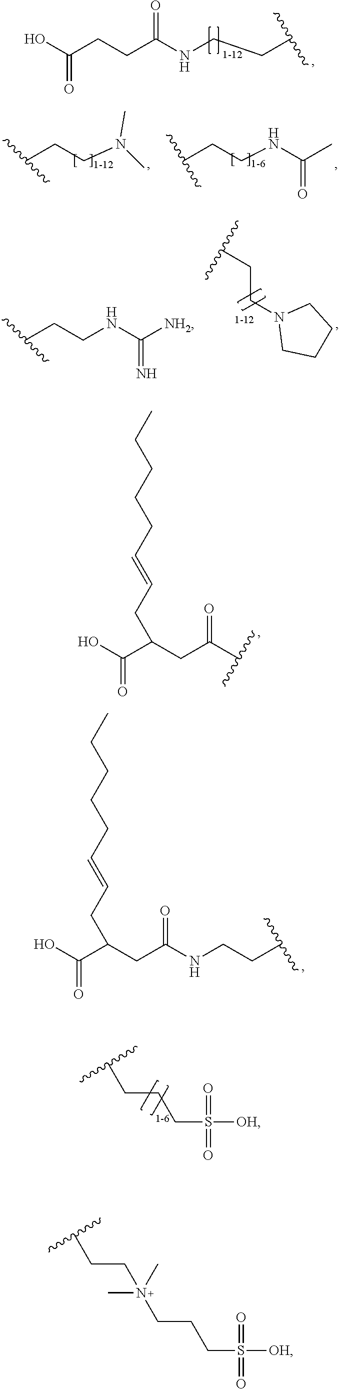

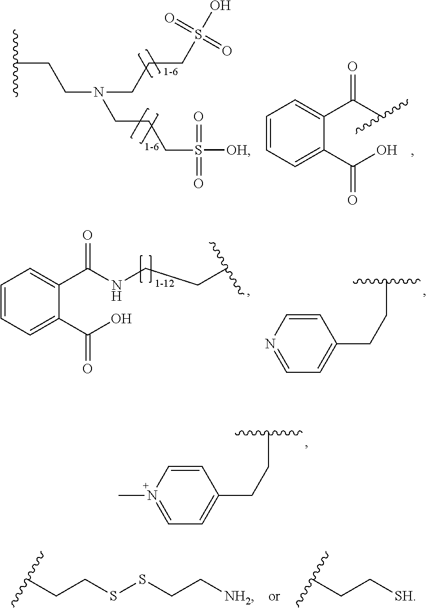

In some embodiments, R₁, R₂, R₄, and R₅ may comprise a charge. In some embodiments, R₁, R₂, R₄, and R₅ may comprise a positive charge. In such cases, R₂, R₂, R₄, and R₅ may comprise a counterion comprising a negative charge. In some cases, a counterion comprising a negative charge may be a halogen (e.g., fluoride, chloride, bromide, or iodide) In some embodiments, R₁, R₂, R₄, and R₅ may comprise a negative charge. In such cases, R₁, R₂, R₄, and R₅ may comprise a counterion comprising a positive charge (e.g., sodium ion, lithium ion, etc).

In some embodiments, the surface is the surface of compound 450 (e.g., of Table 4).

In some embodiments, the surface is the surface of compound 424 (e.g., of Table 4).

In some embodiments, the surface is the surface of compound:

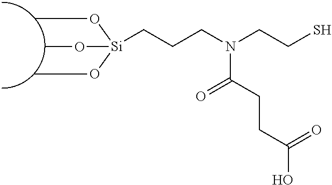 [SMILES: CO[Si](CCCN(CCS)C(=O)CCC(=O)O)(OC)OC]

In some embodiments, the surface is the surface of compound 418 (e.g., of Table 4).

In some embodiments, the surface is the surface of compound 413 (e.g., of Table 4).

In some embodiments, the surface is the surface of compound 428 (e.g., of Table 4).

In some embodiments, the surface is the surface of compound 425 (e.g., of Table 4).

In some embodiments, the surface is the surface of

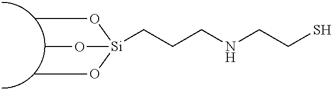

In some embodiments, the surface is the surface of compound 449 (e.g., of Table 4).

In some embodiments, the surface is the surface of compound 413 (e.g., of Table 4).

In some embodiments, the surface is the surface of compound 441 (e.g., of Table 4).

In some embodiments, the surface is the surface of compound 425 (e.g., of Table 4).

In some embodiments, the surface is the surface of compound 424 (e.g., of Table 4).

In some embodiments, the surface is the surface of compound 431 (e.g., of Table 4).

In some embodiments, the surface is the surface of compound 428 (e.g., of Table 4).

In some embodiments, the surface is the surface of compound 507.

In some embodiments, Z represents an attachment point for a unit of Component (A) or Component (B).

In some embodiments, the macromolecule (e.g., bound to a surface) is iron oxide nanoparticle.

In some embodiments, the macromolecule is a silica coated iron oxide nanoparticle.

In some instances, multiple macromolecules are used in combination. In some instances, the use of one or more unique macromolecules (or macromolecules bound to surfaces) enhances the ability of the surfaces to for example, identify proteins, peptides, or protein groups.

In some embodiments, the surfaces provided herein may be used in combination with one or more other unique surfaces provided herein, such as in any method provided herein. In some instances, when used in combination, surfaces may exhibit enhanced performance, such as when identifying proteins, peptides, or protein groups. In some embodiments, two or more surfaces provided herein may be used in combination, such as in any method provided herein. In some embodiments, three or more surfaces provided herein may be used in combination, such as in any method provided herein. In some embodiments, two unique surfaces provided herein may be provided in combination, such as in any method provided herein.

In some embodiments, the surfaces of compound 450 and compound 424 (e.g., of Table 4) may be provided together in a method provided herein.

In some embodiments, the surfaces of compound 424 (e.g., of Table 4) and compound 509 may be provided together in a method provided herein.

In some embodiments, the surfaces of compound 418 and compound 424 (e.g., of Table 4) may be provided together in a method provided herein.

In some embodiments, the surfaces of compound 428 and compound 424 (e.g., of Table 4) may be provided together in a method provided herein.

In some embodiments, the surfaces of compound 425 and compound 424 (e.g., of Table 4) may be provided together in a method provided herein.

In some embodiments, the surfaces of compound 413 and compound 424 (e.g., of Table 4) may be provided together in a method provided herein.

In some embodiments, the surfaces of compound 413 and compound 507 may be provided together in a method provided herein.

In some embodiments, the surfaces of compound 508 and compound 424 may be provided together in a method provided herein.

Some embodiments disclosed here include a surface for adsorbing biomolecules from a biological sample, wherein the surface is functionalized with a carboxylic acid and a thiol, and wherein the functionalization facilitates the adsorption of biomolecules when contacted with the biological sample. As a non-limiting example, the surface may be compound 328, compound 508, or compound 509. Such surfaces may be advantageous, for example, to adsorb a large number of different proteins in a biological sample, they effectively compress dynamic range of biomolecule in the biological sample, exhibit optimal adsorption properties at biological pH, and/or provide improved reproducibility for biological assays.

In some embodiments, the molar ratio of the carboxylic acid to thiol functionalization may be about 3:1 to about 1:3. In some embodiments, the molar ratio of the carboxylic acid to thiol functionalization may be about 2:1 to about 1:2. In some embodiments, the molar ratio of the carboxylic acid to thiol functionalization may be about 3:2 to about 2:3. In some embodiments, the molar ratio of the carboxylic acid to thiol functionalization may be about 4:3 to about 3:4.

In some embodiments, the carboxylic acid and thiol are covalently coupled to the surface. In some embodiments, the surface comprises a polymer functionalized with the carboxylic acid and thiol. In some embodiments, the polymer comprising a recurring unit functionalized with both carboxylic acid and thiol.

In some embodiments, the surface is functionalized with the following moiety:

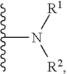

where R¹ is optionally substituted succinate, optionally substituted glutarate, optionally substituted adipate, optionally substituted pimelate, optionally substituted suberate, optionally substituted azelate, or optionally substituted sebacate; and R₂ is optionally substituted C₁-C₆ thiol. In some embodiments, R¹ is succinate and R₂ is ethylthiol. An appropriate tethering moiety may be covalently linked to the nitrogen atom to covalently couple the moiety to the surface.

In some embodiments, the surface is functionalized with following moiety:

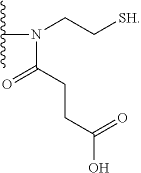 [SMILES: *N(CCS)C(=O)CCC(=O)O]

An appropriate tethering moiety may be covalently linked to the nitrogen atom to covalently coupled the moiety to the surface.

The surfaces functionalized with the carboxylic acid and thiol may be used in any of the systems, methods, and kits disclosed herein.

### Surfaces

The surfaces disclosed herein can be used, in some embodiments, to identifying a number of proteins, peptides, or protein groups using the Proteograph™ workflows (MS analysis of biomolecule coronas corresponding to distinct surfaces in the surface) described in WO2020/096631, which is incorporated herein by reference in its entirety. Feature intensities refer to the intensity of a discrete spike (“feature”) seen on a plot of mass to charge ratio versus intensity from a mass spectrometry run of a sample. These features can correspond to variably ionized fragments of peptides and/or proteins. Using the data analysis methods described herein, feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct surfaces in a surface, yields a high number of feature intensities. This number decreases as feature intensities are processed into distinct peptides, further decreases as distinct peptides are processed into distinct proteins, and further decreases as peptides are grouped into protein groups (two or more proteins that share a distinct peptide sequence).

The surfaces disclosed herein can be used to identify at least at least 100 proteins, at least 200 proteins, at least 300 proteins, at least 400 proteins, at least 500 proteins, at least 600 proteins, at least 700 proteins, at least 800 proteins, at least 900 proteins, at least 1000 proteins, at least 1100 proteins, at least 1200 proteins, at least 1300 proteins, at least 1400 proteins, at least 1500 proteins, at least 1600 proteins, at least 1700 proteins, at least 1800 proteins, at least 1900 proteins, at least 2000 proteins, at least 2100 proteins, at least 2200 proteins, at least 2300 proteins, at least 2400 proteins, at least 2500 proteins, at least 2600 proteins, at least 2700 proteins, at least 2800 proteins, at least 2900 proteins, at least 3000 proteins, at least 3100 proteins, at least 3200 proteins, at least 3300 proteins, at least 3400 proteins, at least 3500 proteins, at least 3600 proteins, at least 3700 proteins, at least 3800 proteins, at least 3900 proteins, at least 4000 proteins, at least 4100 proteins, at least 4200 proteins, at least 4300 proteins, at least 4400 proteins, at least 4500 proteins, at least 4600 proteins, at least 4700 proteins, at least 4800 proteins, at least 4900 proteins, at least 5000 proteins, at least 10000 proteins, at least 20000 proteins, at least 50000 proteins, at least 100000 proteins, from 100 to 5000 proteins, from 200 to 4700 proteins, from 300 to 4400 proteins, from 400 to 4100 proteins, from 500 to 3800 proteins, from 600 to 3500 proteins, from 700 to 3200 proteins, from 800 to 2900 proteins, from 900 to 2600 proteins, from 1000 to 2300 proteins, from 1000 to 3000 proteins, from 3000 to 4000 proteins, from 4000 to 5000 proteins, from 5000 to 6000 proteins, from 6000 to 7000 proteins, from 7000 to 8000 proteins, from 8000 to 9000 proteins, from 9000 to 10000 proteins, from 10000 to 11000 proteins, from 11000 to 12000 proteins, from 12000 to 13000 proteins, from 13000 to 14000 proteins, from 14000 to 15000 proteins, from 15000 to 16000 proteins, from 16000 to 17000 proteins, from 17000 to 18000 proteins, from 18000 to 19000 proteins, from 19000 to 20000 proteins, from 20000 to 25000 proteins, from 25000 to 30000 proteins, from 10000 to 20000 proteins, from 10000 to 50000 proteins, from 20000 to 100000 proteins, from 2000 to 20000 proteins, from 1800 to 20000 proteins, or from 10000 to 100000 proteins.

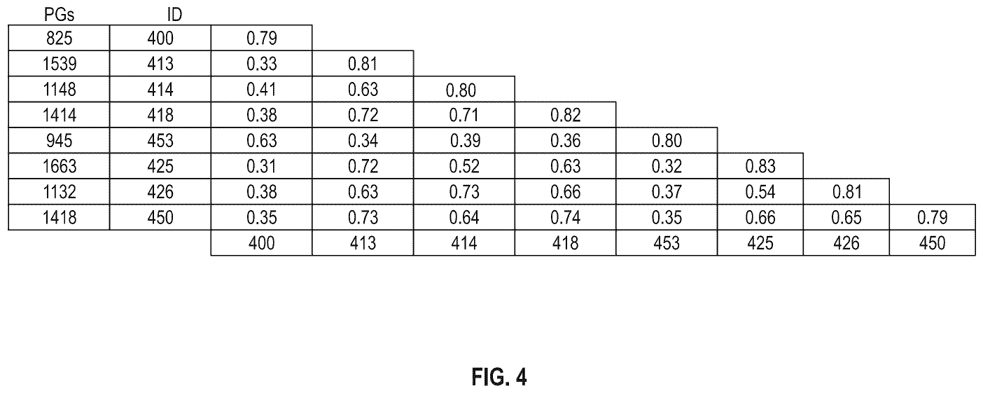

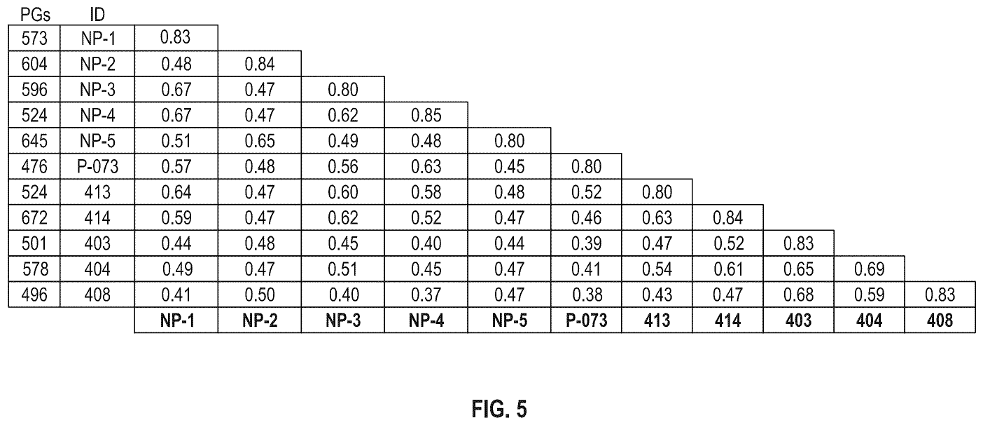

The surfaces disclosed herein can be used to identify at least at least 100 protein groups, at least 200 protein groups, at least 300 protein groups, at least 400 protein groups, at least 500 protein groups, at least 600 protein groups, at least 700 protein groups, at least 800 protein groups, at least 900 protein groups, at least 1000 protein groups, at least 1100 protein groups, at least 1200 protein groups, at least 1300 protein groups, at least 1400 protein groups, at least 1500 protein groups, at least 1600 protein groups, at least 1700 protein groups, at least 1800 protein groups, at least 1900 protein groups, at least 2000 protein groups, at least 2100 protein groups, at least 2200 protein groups, at least 2300 protein groups, at least 2400 protein groups, at least 2500 protein groups, at least 2600 protein groups, at least 2700 protein groups, at least 2800 protein groups, at least 2900 protein groups, at least 3000 protein groups, at least 3100 protein groups, at least 3200 protein groups, at least 3300 protein groups, at least 3400 protein groups, at least 3500 protein groups, at least 3600 protein groups, at least 3700 protein groups, at least 3800 protein groups, at least 3900 protein groups, at least 4000 protein groups, at least 4100 protein groups, at least 4200 protein groups, at least 4300 protein groups, at least 4400 protein groups, at least 4500 protein groups, at least 4600 protein groups, at least 4700 protein groups, at least 4800 protein groups, at least 4900 protein groups, at least 5000 protein groups, at least 10000 protein groups, at least 20000 protein groups, at least 100000 protein groups, from 100 to 5000 protein groups, from 200 to 4700 protein groups, from 300 to 4400 protein groups, from 400 to 4100 protein groups, from 500 to 3800 protein groups, from 600 to 3500 protein groups, from 700 to 3200 protein groups, from 800 to 2900 protein groups, from 900 to 2600 protein groups, from 1000 to 2300 protein groups, from 1000 to 3000 protein groups, from 3000 to 4000 protein groups, from 4000 to 5000 protein groups, from 5000 to 6000 protein groups, from 6000 to 7000 protein groups, from 7000 to 8000 protein groups, from 8000 to 9000 protein groups, from 9000 to 10000 protein groups, from 10000 to 11000 protein groups, from 11000 to 12000 protein groups, from 12000 to 13000 protein groups, from 13000 to 14000 protein groups, from 14000 to 15000 protein groups, from 15000 to 16000 protein groups, from 16000 to 17000 protein groups, from 17000 to 18000 protein groups, from 18000 to 19000 protein groups, from 19000 to 20000 protein groups, from 20000 to 25000 protein groups, from 25000 to 30000 protein groups, from 10000 to 20000 protein groups, from 10000 to 50000 protein groups, from 20000 to 100000 protein groups, from 2000 to 20000 protein groups, from 1800 to 20000 protein groups, or from 10000 to 100000 protein groups. For example, FIG. 4 shows the number of protein groups adhered to different surfaces of the disclosure and the Jaccard index representing the difference between two surfaces in terms of protein groups adhered. In another example, FIG. 5 shows the number of protein groups adhered to different surfaces of the disclosure and the Jaccard index representing the difference between two surfaces in terms of protein groups adhered; NP-1 through NP-5 are nanoparticles found in commercially available Proteograph™ V1.2 kit and P-073 is a dextran-functionalized nanoparticle.

The surfaces disclosed herein can be used to identify the number of distinct proteins disclosed herein, and/or any of the specific proteins disclosed herein, over a wide dynamic range. For example, the surfaces disclosed herein comprising distinct surfaces, can enrich for proteins in a sample, which can be identified using the Proteograph workflow, over the entire dynamic range at which proteins are present in a sample (e.g., a plasma sample). In some embodiments, a surface including any number of distinct surfaces disclosed herein, enriches and identifies proteins over a dynamic range of at least 2. In some embodiments, a surface including any number of distinct surfaces disclosed herein, enriches and identifies proteins over a dynamic range of at least 3. In some embodiments, a surface including any number of distinct surfaces disclosed herein, enriches and identifies proteins over a dynamic range of at least 4. In some embodiments, a surface including any number of distinct surfaces disclosed herein, enriches and identifies proteins over a dynamic range of at least 5. In some embodiments, a surface including any number of distinct surfaces disclosed herein, enriches and identifies proteins over a dynamic range of at least 6. In some embodiments, a surface including any number of distinct surfaces disclosed herein, enriches and identifies proteins over a dynamic range of at least 7. In some embodiments, a surface including any number of distinct surfaces disclosed herein, enriches and identifies proteins over a dynamic range of at least 8. In some embodiments, a surface including any number of distinct surfaces disclosed herein, enriches and identifies proteins over a dynamic range of at least 9. In some embodiments, a surface including any number of distinct surfaces disclosed herein, enriches and identifies proteins over a dynamic range of at least 10. In some embodiments, a surface including any number of distinct surfaces disclosed herein, enriches and identifies proteins over a dynamic range of at least 11. In some embodiments, a surface including any number of distinct surfaces disclosed herein, enriches and identifies proteins over a dynamic range of at least 12. In some embodiments, a surface including any number of distinct surfaces disclosed herein, enriches and identifies proteins over a dynamic range of at least 13. In some embodiments, a surface including any number of distinct surfaces disclosed herein, enriches and identifies proteins over a dynamic range of at least 14. In some embodiments, a surface including any number of distinct surfaces disclosed herein, enriches and identifies proteins over a dynamic range of at least 15. In some embodiments, a surface including any number of distinct surfaces disclosed herein, enriches and identifies proteins over a dynamic range of from 2 to 15. In some embodiments, a surface including any number of distinct surfaces disclosed herein, enriches and identifies proteins over a dynamic range of from 6 to 15. In some embodiments, a surface including any number of distinct surfaces disclosed herein, enriches and identifies proteins over a dynamic range of from 8 to 12. In some embodiments, a surface including any number of distinct surfaces disclosed herein, enriches and identifies proteins over a dynamic range of from 2 to 5. In some embodiments, a surface including any number of distinct surfaces disclosed herein, enriches and identifies proteins over a dynamic range of from 5 to 10.

In some embodiments, panels can have more than one surfaces. Increasing the number of surfaces in a panel can be a method for increasing the number of proteins that can be identified in a given sample. For example, panel size of one particle type may identify at least 400 unique proteins, a panel size of two surfaces may identify at least 550 proteins, a panel size of three surfaces may identify at least 700 proteins, a panel size of four surfaces may identify at least 800 proteins, a panel size of five surfaces may identify at least 900 proteins, a panel size of six surfaces may identify at least 1000 proteins, a panel size of seven surfaces may identify at least 1050 proteins, a panel size of eight surfaces may identify at least 1100 proteins, a panel size of nine surfaces may identify at least 1175 proteins, a panel size of 10 surfaces may identify 1200 proteins, a panel size of eleven surfaces may identify at least 1250 proteins, and a panel size of twelve surfaces identified at least 1300 proteins. The surfaces may include nanosurfaces. In some embodiments, the surfaces are particles, such microparticles and nanoparticles.

In some embodiments, a panel size of one surface type is capable of identifying 400 to 100 unique proteins. In some embodiments, a panel size of two surfaces is capable of identifying 800 to 1500 unique proteins. In some embodiments, a panel size of three surfaces is capable of identifying 1000 to 2000 unique proteins. In some embodiments, a panel size of four surfaces is capable of identifying 1500 to 3500 unique proteins. In some embodiments, a panel size of five surfaces is capable of identifying 2500 to 4500 unique proteins. In some embodiments, a panel size of six surfaces is capable of identifying 4000 to 5500 unique proteins. In some embodiments, a panel size of seven surfaces is capable of identifying 4500 to 6000 unique proteins. In some embodiments, a panel size of eight surfaces is capable of identifying 5000 to 6500 unique proteins. In some embodiments, a panel size of nine surfaces is capable of identifying 5500 to 7000 unique proteins. In some embodiments, a panel size of 10 surfaces is capable of identifying 6000 to 7500 unique proteins.

The present disclosure provides for distinct surfaces comprising at least one physicochemical property that differs between a first surface and a second surface. For example, the present disclosure provides a surface having at least 2 distinct surfaces, at least 3 distinct surfaces, at least 4 distinct surfaces, at least 5 distinct surfaces, at least 6 distinct surfaces, at least 7 distinct surfaces, at least 8 distinct surfaces, at least 9 distinct surfaces, at least 10 distinct surfaces, at least 11 distinct surfaces, at least 12 distinct surfaces, at least 13 distinct surfaces, at least 14 distinct surfaces, at least 15 distinct surfaces, at least 20 distinct surfaces, at least 25 distinct surfaces, at least 30 distinct surfaces, at least 35 distinct surfaces, at least 40 distinct surfaces, at least 45 distinct surfaces, at least 50 distinct surfaces, at least 100 distinct surfaces, at least 150 distinct surfaces, at least 200 distinct surfaces, at least 250 distinct surfaces, at least 300 distinct surfaces, at least 350 distinct surfaces, at least 400 distinct surfaces, at least 450 distinct surfaces, at least 500 distinct surfaces, from 2 to 500 distinct surfaces, from 2 to 5 distinct surfaces, from 5 to 10 distinct surfaces, from 10 to 15 distinct surfaces, from 15 to 20 distinct surfaces, from 20 to 40 distinct surfaces, from 40 to 60 distinct surfaces, from 60 to 80 distinct surfaces, from 80 to 100 distinct surfaces, from 100 to 500 distinct surfaces, from 4 to 15 distinct surfaces, or from 2 to 20 distinct surfaces. The surfaces may include nanosurfaces.

In some embodiments, the present disclosure provide a panel size of at least 1 particle distinct type, at least 2 distinct surfaces, at least 3 distinct surfaces, at least 4 distinct surfaces, at least 5 distinct surfaces, at least 6 distinct surfaces, at least 7 distinct surfaces, at least 8 distinct surfaces, at least 9 distinct surfaces, at least 10 distinct surfaces, at least 11 distinct surfaces, at least 12 distinct surfaces, at least 13 distinct surfaces, at least 14 distinct surfaces, at least 15 distinct surfaces, at least 16 distinct surfaces, at least 17 distinct surfaces, at least 18 distinct surfaces, at least 19 distinct surfaces, at least 20 distinct surfaces, at least 25 distinct surfaces, at least 30 distinct surfaces, at least 35 distinct surfaces, at least 40 distinct surfaces, at least 45 distinct surfaces, at least 50 distinct surfaces, at least 55 distinct surfaces, at least 60 distinct surfaces, at least 65 distinct surfaces, at least 70 distinct surfaces, at least 75 distinct surfaces, at least 80 distinct surfaces, at least 85 distinct surfaces, at least 90 distinct surfaces, at least 95 distinct surfaces, at least 100 distinct surfaces, from 1 to 5 distinct surfaces, from 5 to 10 distinct surfaces, from 10 to 15 distinct surfaces, from 15 to 20 distinct surfaces, from 20 to 25 distinct surfaces, from 25 to 30 distinct surfaces, from 30 to 35 distinct surfaces, from 35 to 40 distinct surfaces, from 40 to 45 distinct surfaces, from 45 to 50 distinct surfaces, from 50 to 55 distinct surfaces, from 55 to 60 distinct surfaces, from 60 to 65 distinct surfaces, from 65 to 70 distinct surfaces, from 70 to 75 distinct surfaces, from 75 to 80 distinct surfaces, from 80 to 85 distinct surfaces, from 85 to 90 distinct surfaces, from 90 to 95 distinct surfaces, from 95 to 100 distinct surfaces, from 1 to 100 distinct surfaces, from 20 to 40 distinct surfaces, from 5 to 10 distinct surfaces, from 3 to 7 distinct surfaces, from 2 to 10 distinct surfaces, from 6 to 15 distinct surfaces, or from 10 to 20 distinct surfaces. In particular embodiments, the present disclosure provides a panel size of from 3 to 10 surfaces. In particular embodiments, the present disclosure provides a panel size of from 4 to 11 distinct surfaces. In particular embodiments, the present disclosure provides a panel size of from 5 to 15 distinct surfaces. In particular embodiments, the present disclosure provides a panel size of from 5 to 15 distinct surfaces. In particular embodiments, the present disclosure provides a panel size of from 8 to 12 distinct surfaces. In particular embodiments, the present disclosure provides a panel size of from 9 to 13 distinct surfaces. In particular embodiments, the present disclosure provides a panel size of from 2 to 20 distinct surfaces. In particular embodiments, the present disclosure provides a panel size of 10 distinct surfaces. The surfaces may include nanosurfaces. In some embodiments, the surfaces are particles, such microparticles and nanoparticles.

For example, the present disclosure provides a surface having at least 2 distinct surfaces, at least 3 different surface chemistries, at least 4 different surface chemistries, at least 5 different surface chemistries, at least 6 different surface chemistries, at least 7 different surface chemistries, at least 8 different surface chemistries, at least 9 different surface chemistries, at least 10 different surface chemistries, at least 11 different surface chemistries, at least 12 different surface chemistries, at least 13 different surface chemistries, at least 14 different surface chemistries, at least 15 different surface chemistries, at least 20 different surface chemistries, at least 25 different surface chemistries, at least 30 different surface chemistries, at least 35 different surface chemistries, at least 40 different surface chemistries, at least 45 different surface chemistries, at least 50 different surface chemistries, at least 100 different surface chemistries, at least 150 different surface chemistries, at least 200 different surface chemistries, at least 250 different surface chemistries, at least 300 different surface chemistries, at least 350 different surface chemistries, at least 400 different surface chemistries, at least 450 different surface chemistries, at least 500 different surface chemistries, from 2 to 500 different surface chemistries, from 2 to 5 different surface chemistries, from 5 to 10 different surface chemistries, from 10 to 15 different surface chemistries, from 15 to 20 different surface chemistries, from 20 to 40 different surface chemistries, from 40 to 60 different surface chemistries, from 60 to 80 different surface chemistries, from 80 to 100 different surface chemistries, from 100 to 500 different surface chemistries, from 4 to 15 different surface chemistries, or from 2 to 20 different surface chemistries.

The present disclosure provides a surface having at least 2 different surface chemistries, at least 3 different surface chemistries, at least 4 different surface chemistries, at least 5 different surface chemistries, at least 6 different surface chemistries, at least 7 different surface chemistries, at least 8 different surface chemistries, at least 9 different surface chemistries, at least 10 different surface chemistries, at least 11 different surface chemistries, at least 12 different surface chemistries, at least 13 different surface chemistries, at least 14 different surface chemistries, at least 15 different surface chemistries, at least 20 different surface chemistries, at least 25 different surface chemistries, at least 30 different surface chemistries, at least 35 different surface chemistries, at least 40 different surface chemistries, at least 45 different surface chemistries, at least 50 different surface chemistries, at least 100 different surface chemistries, at least 150 different surface chemistries, at least 200 different surface chemistries, at least 250 different surface chemistries, at least 300 different surface chemistries, at least 350 different surface chemistries, at least 400 different surface chemistries, at least 450 different surface chemistries, at least 500 different surface chemistries, from 2 to 500 different surface chemistries, from 2 to 5 different surface chemistries, from 5 to 10 different surface chemistries, from 10 to 15 different surface chemistries, from 15 to 20 different surface chemistries, from 20 to 40 different surface chemistries, from 40 to 60 different surface chemistries, from 60 to 80 different surface chemistries, from 80 to 100 different surface chemistries, from 100 to 500 different surface chemistries, from 4 to 15 different surface chemistries, or from 2 to 20 different surface chemistries.

In some embodiments, the panel comprises a panel of particles having different sizes. For example, the panel may include a first particle with a diameter of about 100 nm, a second particle with a diameter of about 3 microns, and a third particle with a diameter of about 4 microns. The particles may otherwise have the same surface chemistry. In some embodiments, the different particles sizes in the panel may be separately incubated with a biofluid. In some embodiments, the different particles sizes in the panel may incubated in a single mixture with a biofluid. As used herein, a mixture of particles have a polydispersity index (PDI) greater than 0.5 (e.g., greater than 0.75) shall be considered to have two or more different particle sizes.

The present disclosure provides a surface having at least 2 different physical properties, at least 3 different physical properties, at least 4 different physical properties, at least 5 different physical properties, at least 6 different physical properties, at least 7 different physical properties, at least 8 different physical properties, at least 9 different physical properties, at least 10 different physical properties, at least 11 different physical properties, at least 12 different physical properties, at least 13 different physical properties, at least 14 different physical properties, at least 15 different physical properties, at least 20 different physical properties, at least 25 different physical properties, at least 30 different physical properties, at least 35 different physical properties, at least 40 different physical properties, at least 45 different physical properties, at least 50 different physical properties, at least 100 different physical properties, at least 150 different physical properties, at least 200 different physical properties, at least 250 different physical properties, at least 300 different physical properties, at least 350 different physical properties, at least 400 different physical properties, at least 450 different physical properties, at least 500 different physical properties, from 2 to 500 different physical properties, from 2 to 5 different physical properties, from 5 to 10 different physical properties, from 10 to 15 different physical properties, from 15 to 20 different physical properties, from 20 to 40 different physical properties, from 40 to 60 different physical properties, from 60 to 80 different physical properties, from 80 to 100 different physical properties, from 100 to 500 different physical properties, from 4 to 15 different physical properties, or from 2 to 20 different physical properties.

In some embodiments, the panel comprises a panel of particles having different sizes. For example, the panel may include a first particle with a diameter about 100 nm, a second particle with a diameter of about 3 microns, and a third particle with a diameter of about 4 microns. The particles may otherwise have the same surface chemistry. In some embodiments, the different particles sizes in the panel may be separately incubated with a biofluid. In some embodiments, the different particles sizes in the panel may incubated in a single mixture with a biofluid. As used herein, a mixture of particles have a polydispersity index (PDI) greater than 0.5 (e.g., greater than 0.75, greater than 1.0, greater than 1.5, or greater than 2.0) shall be considered to have two or more different particle sizes.

In some embodiments, panels that identity proteins and associate biomarkers with diseases include panels selected from the surfaces described in TABLE 3 and TABLE 4. Surfaces especially suitable to identifying high numbers of proteins (e.g., greater than 1500 proteins) in a sample include from 5 to 10 distinct surfaces in an assay. The number of distinct surfaces included in a surface can be tuned for a specific application (e.g., detection of a particular subset of proteins or detection of a group of markers associated with a particular disease). In some embodiments, panels with physicochemically distinct surfaces that optimally identify proteins and associate biomarkers with diseases include silica-coated SPIONs, acrylamide-based SPIONs, and acrylate-based SPIONS. For example, a panel of surfaces disclosed herein that generates information rich proteomic data via their protein coronas, which can be associated with biomarkers and diseases with high sensitivity and specificity include silica-coated SPIONs (SP-003), poly(N-(3-(dimethylamino)propyl) methacrylamide) (PDMAPMA)-coated SPIONs (SP-007), and poly(oligo(ethylene glycol) methyl ether methacrylate) (POEGMA)-coated SPIONs (SP-011).

In some embodiments, the entire assay time from a single pooled plasma, including sample preparation and LC-MS, can be about 8 hours. In some embodiments, the entire assay time from a single pooled plasma, including sample preparation and LC-MS, can be about at least 1 hour, at least 2 hours, at least 3 hours, at least 4 hours, at least 5 hours, at least 6 hours, at least 7 hours, at least 8 hours, at least 9 hours, at least 10 hours, under 20 hours, under 19 hours, under 18 hours, under 17 hours, under 16 hours, under 15 hours, under 14 hours, under 13 hours, under 12 hours, under 11 hours, under 10 hours, under 9 hours, under 8 hours, under 7 hours, under 6 hours, under 5 hours, under 4 hours, under 3 hours, under 2 hours, under 1 hour, at least 5 min to 10 min, at least 10 min to 20 min, at least 20 min to 30 min, at least 30 min to 40 min, at least 40 min to 50 min, at least 50 min to 60 min, at least 1 hour to 1.5 hours, at least 1.5 hour to 2 hours, at least 2 hour to 2.5 hours, at least 2.5 hour to 3 hours, at least 3 hour to 3.5 hours, at least 3.5 hour to 4 hours, at least 4 hour to 4.5 hours, at least 4.5 hour to 5 hours, at least 5 hour to 5.5 hours, at least 5.5 hour to 6 hours, at least 6 hour to 6.5 hours, at least 6.5 hour to 7 hours, at least 7 hour to 7.5 hours, at least 7.5 hour to 8 hours, at least 8 hour to 8.5 hours, at least 8.5 hour to 9 hours, at least 9 hour to 9.5 hours, or at least 9.5 hour to 10 hours.

### Biosamples

The surfaces of the present disclosure can be used to generate proteomic data from protein coronas and subsequently associated with any of the biological states described herein. Samples consistent with the present disclosure include biological samples from a subject. The subject may be a human or a non-human animal. Biological samples may be a biofluid. For example, the biofluid may be plasma, serum, CSF, urine, tear, cell lysates, tissue lysates, cell homogenates, tissue homogenates, nipple aspirates, fecal samples, synovial fluid and whole blood, or saliva. The biological sample may be medium from a cell culture. Samples can also be non-biological samples, such as water, milk, solvents, or anything homogenized into a fluidic state. In some embodiments, the biological sample may be a cell-free biological sample (e.g., plasma or serum). In some embodiments, the biological sample may comprise extracellular vesicles. Said biological samples can contain a plurality of proteins or proteomic data, which may be analyzed after adsorption of proteins to the surface of the various surfaces in a panel and subsequent digestion of protein coronas. Proteomic data can comprise nucleic acids, peptides, or proteins. Any of the samples herein can contain a number of different analytes, which can be analyzed using the compositions and methods disclosed herein. The analytes can be proteins, peptides, small molecules, nucleic acids, metabolites, lipids, or any molecule that could potentially bind or interact with the surface of a particle type.

Disclosed herein are compositions and methods for multi-omic analysis. “Multi-omic(s)” or “multiomic(s)” can refer to an analytical approach for analyzing biomolecules at a large scale, wherein the data sets are multiple ones, such as proteome, genome, transcriptome, lipidome, and metabolome. Non-limiting examples of multi-omic data includes proteomic data, genomic data, lipidomic data, glycomic data, transcriptomic data, or metabolomics data. “Biomolecule” in “biomolecule corona” can refer to any molecule or biological component that can be produced by, or is present in, a biological organism. Non-limiting examples of biomolecules include proteins (protein corona), polypeptides, polysaccharides, a sugar, a lipid, a lipoprotein, a metabolite, an oligonucleotide, a nucleic acid (DNA, RNA, micro RNA, plasmid, single stranded nucleic acid, double stranded nucleic acid), metabolome, as well as small molecules such as primary metabolites, secondary metabolites, and other natural products, or any combination thereof. In some embodiments, the biomolecule is selected from the group of proteins, nucleic acids, lipids, and metabolomes.

In some embodiments, a sample of the present disclosure can be a plurality of samples. At least two samples of the plurality of samples can be spatially isolated. Spatially isolated refers to samples that are contained in separate volumes. For example, spatially isolated samples can refer to samples that are in separate wells in a plate or separate tubes. Spatially isolated samples can refer to samples that are in separate wells in a plate or separate tubes and assayed together on the same instrument. In some embodiments, the present disclosure provides surfaces and methods of use thereof that are compatible with analyzing a plurality of samples, such as at least 2 spatially isolated samples, at least 5 spatially isolated samples, at least 10 spatially isolated samples, at least 15 spatially isolated samples, at least 20 spatially isolated samples, at least 25 spatially isolated samples, at least 30 spatially isolated samples, at least 35 spatially isolated samples, at least 40 spatially isolated samples, at least 45 spatially isolated samples, at least 50 spatially isolated samples, at least 55 spatially isolated samples, at least 60 spatially isolated samples, at least 65 spatially isolated samples, at least 70 spatially isolated samples, at least 75 spatially isolated samples, at least 80 spatially isolated samples, at least 85 spatially isolated samples, at least 90 spatially isolated samples, at least 95 spatially isolated samples, at least 96 spatially isolated samples, at least 100 spatially isolated samples, at least 120 spatially isolated samples, at least 140 spatially isolated samples, at least 160 spatially isolated samples, at least 180 spatially isolated samples, at least 200 spatially isolated samples, at least 220 spatially isolated samples, at least 240 spatially isolated samples, at least 260 spatially isolated samples, at least 280 spatially isolated samples, at least 300 spatially isolated samples, at least 320 spatially isolated samples, at least 340 spatially isolated samples, at least 360 spatially isolated samples, at least 380 spatially isolated samples, at least 400 spatially isolated samples, at least 420 spatially isolated samples, at least 440 spatially isolated samples, at least 460 spatially isolated samples, at least 480 spatially isolated samples, at least 500 spatially isolated samples, at least 600 spatially isolated samples, at least 700 spatially isolated samples, at least 800 spatially isolated samples, at least 900 spatially isolated samples, at least 1000 spatially isolated samples, at least 1100 spatially isolated samples, at least 1200 spatially isolated samples, at least 1300 spatially isolated samples, at least 1400 spatially isolated samples, at least 1500 spatially isolated samples, at least 1600 spatially isolated samples, at least 1700 spatially isolated samples, at least 1800 spatially isolated samples, at least 1900 spatially isolated samples, at least 2000 spatially isolated samples, at least 5000 spatially isolated samples, at least 10000 spatially isolated samples, from 2 to 10 spatially isolated samples, from 2 to 100 spatially isolated samples, from 2 to 200 spatially isolated samples, from 2 to 300 spatially isolated samples, from 50 to 150 spatially isolated samples, from 10 to 20 spatially isolated samples, from 20 to 30 spatially isolated samples, from 30 to 40 spatially isolated samples, from 40 to 50 spatially isolated samples, from 50 to 60 spatially isolated samples, from 60 to 70 spatially isolated samples, from 70 to 80 spatially isolated samples, from 80 to 90 spatially isolated samples, from 90 to 100 spatially isolated samples, from 100 to 150 spatially isolated samples, from 150 to 200 spatially isolated samples, from 200 to 250 spatially isolated samples, from 250 to 300 spatially isolated samples, from 300 to 350 spatially isolated samples, from 350 to 400 spatially isolated samples, from 400 to 450 spatially isolated samples, from 450 to 500 spatially isolated samples, from 500 to 600 spatially isolated samples, from 600 to 700 spatially isolated samples, from 700 to 800 spatially isolated samples, from 800 to 900 spatially isolated samples, from 900 to 1000 spatially isolated samples, from 1000 to 2000 spatially isolated samples, from 2000 to 3000 spatially isolated samples, from 3000 to 4000 spatially isolated samples, from 4000 to 5000 spatially isolated samples, from 5000 to 6000 spatially isolated samples, from 6000 to 7000 spatially isolated samples, from 7000 to 8000 spatially isolated samples, from 8000 to 9000 spatially isolated samples, or from 9000 to 10000 spatially isolated samples.

The methods disclosed herein include isolating a surface from one or more than one sample. The surfaces may be superparamagnetic for rapid isolation or separation from the sample by applying a magnetic field. Moreover, multiple samples that are spatially isolated can be processed in parallel. Thus, the methods disclosed herein provide for isolating or separating a surface from unbound protein in a plurality of spatially isolated panels at the same time, by using a magnet. For example, surfaces may be incubated with a plurality of spatially isolated samples, wherein each spatially isolated sample is in a well in a well plate (e.g., a 96-well plate). After incubation, the surfaces in each of the wells of the well plate can be separated from unbound protein present in the spatially isolated samples by placing the entire plate on a magnet. This simultaneously pulls down the superparamagnetic particles in the surface. The supernatant in each well can be removed to remove the unbound protein. These steps (incubate, pull down using a magnet) can be repeated to effectively wash the particles, thus removing residual background unbound protein that may be present in a sample. This is one example, but one of skill in the art could envision numerous other scenarios in which superparamagnetic particles are rapidly isolated from one or more than one spatially isolated samples at the same time.

In some embodiments, the panels of the present disclosure provides identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 μg/mL. A protein of moderate abundance may be present in a sample at concentrations between about 10 ng/mL and about 10 μg/mL. Examples of proteins that are highly abundant proteins include albumin, IgG, and the top 14 proteins in abundance that contribute 95% of the mass in plasma. Additionally, any proteins that may be purified using a conventional depletion column may be directly detected in a sample using the surfaces disclosed herein. Examples of proteins may be any protein listed in published databases such as Keshishian et al. (Mol Cell Proteomics. 2015 September; 14(9):2375-93. Doi: 10.1074/mcp.M114.046813. Epub 2015 February 27.), Farr et al. (J Proteome Res. 2014 Jan. 3; 13(1):60-75. Doi: 10.1021/pr4010037. Epub 2013 December 6.), or Pememalm et al. (Expert Rev Proteomics. 2014 August; 11(4):431-48. Doi: 10.1586/14789450.2014.901157. Epub 2014 March 24.).

In some embodiments, examples of proteins that can be measured and identified using the surfaces disclosed herein include albumin, IgG, lysozyme, CEA, HER-2/neu, bladder tumor antigen, thyroglobulin, alpha-fetoprotein, PSA, CA125, CA19.9, CA 15.3, leptin, prolactin, osteopontin, IGF-II, CD98, fascin, sPigR, 14-3-3 eta, troponin I, B-type natriuretic peptide, BRCA1, c-Myc, IL-6, fibrinogen. EGFR, gastrin, PH, G-CSF, desmin. NSE, FSH, VEGF, P21, PCNA, calcitonin, PR, CA125, LH, somatostatin. S100, insulin. Alpha-prolactin, ACTH, Bcl-2, ER alpha, Ki-67, p53, cathepsin D, beta catenin. VWF, CD15, k-ras, caspase 3, EPN, CD10, FAS, BRCA2. CD30L, CD30, CGA, CRP, prothrombin, CD44, APEX, transferrin, GM-CSF, E-cadherin, IL-2, Bax, IFN-gamma, beta-2-MG, TNF alpha, c-erbB-2, trypsin, cyclin D1, MG B, XBP-1, HG-1, YKL-40, S-gamma, NESP-55, netrin-1, geminin, GADD45A, CDK-6, CCL21, BrMS1, 17betaHDI, PDGFRA, Pcaf, CCL5, MMP3, claudin-4, and claudin-3. In some embodiments, other examples of proteins that can be measured and identified using the surfaces disclosed herein are any proteins or protein groups listed in the open targets database for a particular disease indication of interest (e.g., prostate cancer, lung cancer, or Alzheimer's disease).

### Methods

In one aspect, described herein is a method of preparing a surface comprising recurring units of a first component and a second component, wherein the method may comprise (a) providing a mixture of monomers in a solvent comprising a first monomer and a second monomer, wherein the first monomer may comprise a vinyl group and wherein the second monomer may comprise an epoxide group; (b) contacting a surface and the mixture of monomers, thereby producing a reaction mixture; (c) initiating free radical polymerization to produce a macromolecule immobilized to the surface; (d) contacting the macromolecule immobilized to the surface with an amine, thereby producing an aminated macromolecule; and (e) optionally contacting the aminated macromolecule with a compound comprising succinate, phthalate, or propanesulfone. In one aspect, described herein is a method of preparing a surface comprising recurring units of a first component and a second component, wherein the method may comprise (a) providing a mixture of monomers in a solvent comprising a first monomer and a second monomer, wherein the first monomer may comprise a vinyl group and wherein the second monomer may comprise an epoxide group; (b) contacting a surface and the mixture of monomers, thereby producing a reaction mixture; (c) initiating free radical polymerization to produce a macromolecule immobilized to the surface; (d) contacting the macromolecule immobilized to the surface with an azide salt, thereby producing an azide-containing macromolecule; and (e) optionally contacting the azide-containing macromolecule with a alkyne-containing molecule to form a triazole-containing macromolecule. In some embodiments, the surface comprising recurring units of a first component and a second component is a macromolecule immobilized to a surface of the disclosure as described elsewhere herein. In some embodiments, the surface comprising recurring units of a first component and a second component is a macromolecule immobilized to a surface of the disclosure as described elsewhere herein.

The surface may comprise features or properties as described elsewhere herein. For example, the surface may be a particle comprising an iron oxide core and a silica layer. In some embodiments, the solvent may be a polar solvent. In some embodiments, the solvent may be a nonpolar solvent. In some embodiments, the solvent may comprise ethanol, water, acetonitrile, tetrahydrofuran, dimethylformamide, or a combination thereof. In some embodiments, polymerization may be initiated using a free radical initiator. In some embodiments, the free radical initiator is azobisisobutyronitrile (AIBN).

In some embodiments, the method may comprise, subsequent to (c) and prior to (d), contacting the macromolecule immobilized to the surface with a quenching agent. The quenching agent may be contacted with the macromolecule at any suitable time to obtain a desired size for the macromolecule and/or surface. In some embodiments, the quenching agent may be introduced to the reaction mixture when the macromolecule immobilized to the surface comprises a diameter of about 100 nanometers (nm) to about 600 nm. In some embodiments, the reaction may be quenched when the surface comprises a diameter of about 200 nm to about 500 nm. In some embodiments, the reaction may be quenched when the surface comprises a diameter of about 250 nm to about 400 nm. In some embodiments, the reaction may be quenched when the surface comprises a diameter of about 250 nm to about 350 nm. In some embodiments, the reaction may be quenched when the surface comprises a diameter of about 325 nm to about 375 nm. In some embodiments, the quenching agent may comprise glycol or acrylate. In some embodiments, the quenching agent may comprise benzoquinone.

In some embodiments, the method may further comprise purifying the macromolecule immobilized to a surface. In some embodiments, purifying may comprise washing the macromolecule immobilized to a surface with a solvent. In some embodiments, the solvent may comprise ethanol, water, acetonitrile, tetrahydrofuran, dimethylformamide, or a combination thereof. In some embodiments, the solvent may comprise ethanol or tetrahydrofuran.

In some embodiments, the first monomer may comprise a vinyl group. In some embodiments, the first monomer may comprises at least two vinyl groups. In some embodiments, the first monomer may comprises two vinyl groups. In some embodiments, the first monomer is a cross-linking agent. In some embodiments, the first monomer may be divinyl benzene (DVB), ethyleneglycol dimethacrylate (EGDMA), poly(ethylene glycol) dimethacrylate (PEGDMA) (e.g., Diethylene glycol dimethacrylate (DEGDMA), Triethylene glycol dimethacrylate (TEGDMA), and the like) N,N′-alkylenebisacrylamide (e.g., N,N′-methylenebisacrylamide (MBA), N,N′-ethylenebisacrylamide, N,N′-butylenebisacrylamide, and the like), or derivatives thereof. In some embodiments, the second monomer comprises a vinyl group. In some embodiments, the second monomer may comprise glycidyl methacrylate or glycidyl acrylate. In some embodiments, the amine may be a C₁-C₁₂ alkylamine, C₁-C₆ hydroxyamine, or C₁-C₆ alkoxyethylamine. In some embodiments, the amine may be diethylamine, ethanolamine, hexanolamine, methoxyethylamine, or any other amine of the disclosure. In some embodiments, the amine may be a diethylamine. In some embodiments, the amine is a diamine, such as C₁-C₂₀ alkylene diamine. Non-limiting example of suitable diamines include ethylenediamine, propylenediamine, butylenediamine, hexylenediamine, dodecylenediamine, and the like. In some embodiments, the diamine may react with two epoxide-containing monomer units of the macromolecule. In some embodiments, the diamine may react, and crosslink, with two monomer units of the macromolecule that both comprise an epoxide. In some embodiments, the diamine may react with one epoxy-containing monomer unit of the macromolecule. In some embodiments, the diamine may react with one epoxy-containing monomer unit leaving an unreacted terminal amine group. In some embodiments, a portion of the diamines react with one epoxy-containing monomer unit and a portion of the diamines react with two epoxy-containing monomer units. In some embodiments, the azide salt is an alkali metal salt. In some embodiments, the azide salt is sodium azide. In some embodiments, the azide salt is lithium azide.

In some embodiments, the first monomer may comprise a weight percent of the mixture of monomers of about 10% to about 90%. In some embodiments, the first monomer may comprise a weight percent of the mixture of monomers of about 20 to about 80%. In some embodiments, the first monomer may comprise a weight percent of the mixture of monomers of about 40% to about 60%. In some embodiments, the first monomer may comprise a weight percent of the mixture of monomers of about 50%. In some embodiments, the second monomer may comprise a weight percent of the mixture of monomers of about 10% to about 90%. In some embodiments, the second monomer may comprise a weight percent of the mixture of monomers of about 20 to about 80%. In some embodiments, the second monomer may comprise a weight percent of the mixture of monomers of about 40% to about 60%. In some embodiments, the second monomer may comprise a weight percent of the mixture of monomers of about 50%.

In some embodiments, the method comprises (e) contacting the aminated macromolecule with a compound comprising succinate, phthalate, thiol, or propylsulfone. In some embodiments, the compound may comprise succinate (e.g., C₈ alkenyl succinate or C₈ alkenyl ethylaminosuccinate). In some embodiments, the compound may comprise thiol (e.g., C₂ alkyl thiol). In some embodiments, the compound may comprise a phthalate (e.g., C₁-C₆ aminophthalate). In some embodiments, the compound may comprise a propylsulfone (e.g., dipropylsulfone ethylamine).

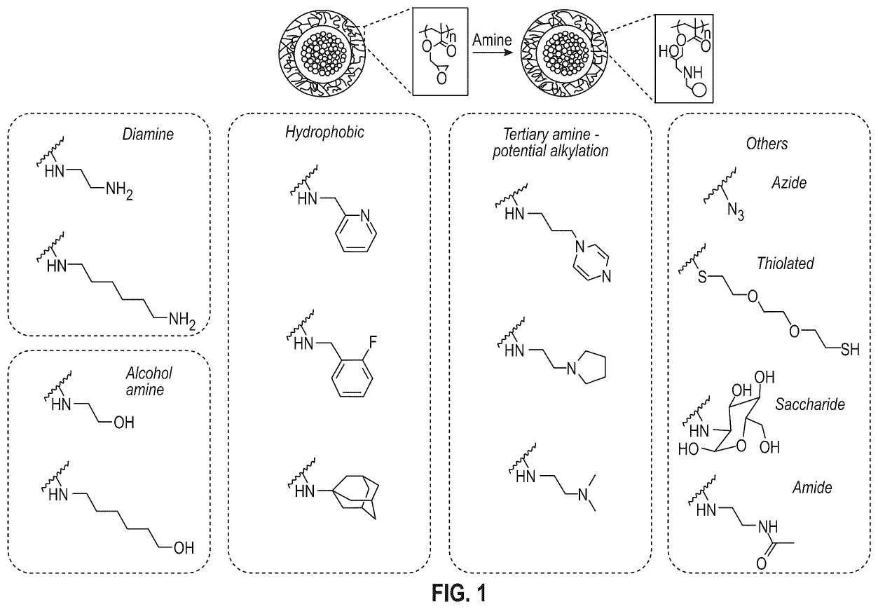

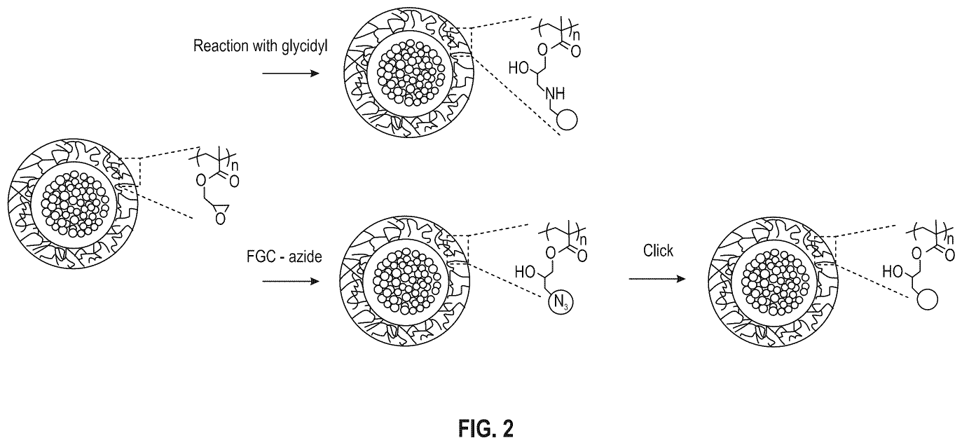

In some embodiments, the method may result in a macromolecule immobilized to surface as described herein. For example, the method may result in a macromolecule immobilized to a surface (e.g. particle), wherein the macromolecule comprises a recurring unit according to Formula (I), (I-A), (I-A′), (II), (II′), (III), (III′), or (III-A) or any recurring unit in Table 1. As another example, the method may result in a macromolecule immobilized to a surface, wherein the macromolecule comprises recurring units of the first component and the second component, or any of the recurring units in Table 2. As an example, FIG. 1 shows the general design space of the epoxidated nanoparticle platform following addition of a functionalizing amine, while FIG. 2 shows the conversion schemes available after epoxidation of nanoparticles including reaction with glycidyls and azides to obtain diverse functionalities.

In one aspect, described herein is a method of identifying proteins in a sample, wherein the method comprises (a) incubating one or more surfaces with a biological sample comprising biomolecules to form a biomolecule corona; (b) isolating at least a portion of the biomolecules in the biomolecule corona; and (c) assaying the biomolecule corona.

In some embodiments, the one or more surfaces are selected from the surfaces disclosed elsewhere herein. In some embodiments, the surfaces are selected from Table 3 and/or Table 4.

In some embodiments, assaying the biomolecule corona may be capable of identifying from 1 to 50,000 protein groups or proteins. In some embodiments, 1 to 20,000 protein groups or proteins may be identified. In some embodiments, at least 100 protein groups may be identified. In some embodiments, at least 300 protein groups may be identified. In some embodiments, at least 500 protein groups may be identified. In some embodiments, 1,000 to 10,000 protein groups or proteins may be identified. In some embodiments, 1,000 to 5,000 protein groups or proteins may be identified. In some embodiments, 1,800 to 5,000 protein groups or proteins may be identified. In some embodiments, 1,200 to 2,200 protein groups or proteins may be identified. In some embodiments, a protein group or proteins may comprise a peptide sequence having a minimum length of 2 amino acid residues. In some embodiments, a protein group may comprise a peptide sequence having a minimum length of 2 amino acid residues. In some embodiments, a protein group may comprise a peptide sequence having a minimum length of 5 amino acid residues. In some embodiments, a protein group may comprise a peptide sequence having a minimum length of 7 amino acid residues. In some embodiments, a protein group may comprise a peptide sequence having a minimum length of 8 amino acid residues. In some embodiments, a protein group may comprise a peptide sequence having a minimum length of 9 amino acid residues. In some embodiments, a protein group may comprise a peptide sequence having a minimum length of 10 amino acid residues. In some embodiments, the method may further comprise lysing the proteins of the biomolecule corona. In some embodiments, the method may further comprise digesting the proteins of the biomolecule corona. In some embodiments, the digested proteins may be purified. In some embodiments, the particles and the proteins of the biomolecule corona are together incubated with a protease to digest the proteins.

In some embodiments, the method further comprises repeating the methods described herein, wherein, when repeated, the incubating, isolating, and assaying yields a percent quantile normalized coefficient (QNCV) of variation of 30% or less, as determined by comparing a peptide mass spectrometry feature from at least three full-assay replicates for each surface in the one or more surfaces. In some embodiments, when repeated, the incubating, isolating, and assaying yields a percent quantile normalized coefficient (QNCV) of variation of 25% or less, as determined by comparing a peptide mass spectrometry feature from at least three full-assay replicates for each surface in the one or more surfaces. In some embodiments, when repeated, the incubating, isolating, and assaying yields a percent quantile normalized coefficient (QNCV) of variation of 20% or less, as determined by comparing a peptide mass spectrometry feature from at least three full-assay replicates for each surface in the one or more surfaces. In some embodiments, the assaying is capable of identifying proteins over a dynamic range of at least 7, at least 8, at least 9, or at least 10. In some embodiments, the assaying is capable of identifying proteins over a dynamic range of no more than 12, no more than 11, no more than 10, no more than 9, or no more than 8.

In some embodiments, the method may further comprise washing one or more surfaces at least one time after isolating the one or more surfaces from an unbound protein. In some embodiments, the method may further comprise washing one or more surfaces at least two times after isolating the one or more surfaces from an unbound protein. In some embodiments, the method may further comprise washing one or more surfaces at least three times after isolating the one or more surfaces from an unbound protein. The isolation may be performed, for example, using magnetic isolation or centrifugation.

In some embodiments, the method may further comprise solubilizing the proteins of the biomolecule corona. In some embodiments, the method may further comprise denaturing the proteins of the biomolecule corona.

In some embodiments, the assaying comprises using mass spectrometry to identify proteins in the sample. In some embodiments, the assaying comprises using tandem mass spectrometry. In some embodiments, the assaying comprises using liquid chromatography tandem mass spectrometry. The assaying may comprise, in some embodiments, ELISA, Edman Degradation, immunoaffinity techniques, single-molecule protein sequencing, and the like. In some embodiments, the assaying is performed in about 2 to about 4 hours. In some embodiments, the method is performed in about 1 to about 20 hours. In some embodiments, the method is performed in about 2 to about 10 hours. In some embodiments, the method is performed in about 4 to about 6 hours. In some embodiments, the isolating takes no more than about 30 minutes, no more than about 15 minutes, no more than about 10 minutes, no more than about 5 minutes, or no more than about 2 minutes. In some embodiments, a plurality of spatially isolated samples are processed according to the method. In some embodiments, the plurality of samples comprises at least 10 spatially isolated samples, at least 50 spatially isolated samples, at least 100 spatially isolated samples, at least 150 spatially isolated samples, at least 200 spatially isolated samples, at least 250 spatially isolated samples, or at least 300 spatially isolated samples. In further embodiments, the plurality of samples comprises at least 96 samples.

In some embodiments, the one or more surfaces comprises a first distinct surface and a second distinct surface, wherein both the first distinct surface and the second distinct surface each comprise a macromolecule as described herein (e.g., a macromolecule comprising a recurring represent by Formula (I), (I-A), (I-A′), (II), (II′), (III), (III′), (III-A), (IV), or described in Tables 1-4). In some embodiments, the one or more surfaces comprises a first distinct surface and a second distinct surface, wherein the first distinct surface comprises a macromolecule as described herein and the second distinct surface is a non-polymeric surface. In some embodiments, the non-polymeric surface is a carboxyl-containing non-polymeric surface. In some embodiments, the non-polymeric surface is a amine-containing non-polymeric surface. In some embodiments, the non-polymeric surface is a silica surface. In some embodiments, the non-polymeric surface comprises the moiety of Formula (IV).

In some embodiments, the one or more surfaces comprises a first distinct surface and a second distinct surface, wherein both the first distinct surface and the second distinct surface each comprise a macromolecule selected from Tables 1 and/or 2. In some embodiments, the one or more surfaces comprises a first distinct surface and a second distinct surface, wherein the first distinct surface comprises a macromolecule selected from Tables 1 and/or 2 and the second distinct surface is a non-polymeric surface. In some embodiments, the non-polymeric surface is a carboxyl-containing non-polymeric surface. In some embodiments, the non-polymeric surface is a amine-containing non-polymeric surface. In some embodiments, the non-polymeric surface is a silica surface. In some embodiments, the non-polymeric surface comprises the moiety of Formula (IV).

In some embodiments, the one or more surfaces comprises a first distinct surface and a second distinct surface, wherein both the first distinct surface and the second distinct surface each comprise a surface selected from Tables 3 and/or 4. In some embodiments, the one or more surfaces comprises a first distinct surface and a second distinct surface, wherein the first distinct surface comprise a surface selected from Tables 3 and/or 4, and wherein the second distinct surface is non-polymeric. In some embodiments, the non-polymeric surface is a carboxyl-containing non-polymeric surface. In some embodiments, the non-polymeric surface is a amine-containing non-polymeric surface. In some embodiments, the non-polymeric surface is a silica surface. In some embodiments, the non-polymeric surface comprises the moiety of Formula (IV).

In some embodiments, the one or more surfaces comprises a first distinct surface and a second distinct surface, wherein the first distinct surface and the second distinct surface share at least one physicochemical property and differ by at least one physicochemical property, such that the first distinct surface and the second distinct surface are different. As an example, the first surface may comprise the macromolecule S-337 and the second surface may comprise the macromolecule S-370. Both surfaces may have polymeric properties, but can exhibit different surface charges as measured by zeta potential analysis. In some embodiments, the one or more surfaces comprises a first distinct surface and a second distinct surface, wherein the first distinct surface and the second distinct surface share at least two physicochemical properties and differ by at least two physicochemical properties, such that the first distinct surface and the second distinct surface are different. In some embodiments, the one or more surfaces comprises a first distinct surface and a second distinct surface, wherein the first distinct surface and the second distinct surface share at least one physicochemical property and differ by at least two physicochemical properties, such that the first distinct surface and the second distinct surface are different.

In some embodiments, the one or more surfaces comprises a first distinct surface and a second distinct surface, wherein the first distinct surface and the second distinct surface share at least two physicochemical properties and differ by at least one physicochemical property, such that the first distinct surface and the second distinct surface are different. In further embodiments, the physicochemical property comprises size, charge, core material, shell material, porosity, or surface hydrophobicity. In further embodiments, size is diameter or radius, as measured by dynamic light scattering, SEM, TEM, or any combination thereof.

In some embodiments, a surface may be a particle. Particles that are consistent with the present disclosure can be made and used in methods of forming protein coronas after incubation in a biofluid at a wide range of sizes. For example, the particles disclosed herein can have a diameter of at least 10 nm, at least 100 nm, at least 200 nm, at least 300 nm, at least 400 nm, at least 500 nm, at least 600 nm, at least 700 nm, at least 800 nm, at least 900 nm, at least 1000 nm, at least 1100 nm, at least 1200 nm, at least 1300 nm, at least 1400 nm, at least 1500 nm, at least 1600 nm, at least 1700 nm, at least 1800 nm, at least 1900 nm, at least 2000 nm, at least 2100 nm, at least 2200 nm, at least 2300 nm, at least 2400 nm, at least 2500 nm, at least 2600 nm, at least 2700 nm, at least 2800 nm, at least 2900 nm, at least 3000 nm, at least 3100 nm, at least 3200 nm, at least 3300 nm, at least 3400 nm, at least 3500 nm, at least 3600 nm, at least 3700 nm, at least 3800 nm, at least 3900 nm, at least 4000 nm, at least 4100 nm, at least 4200 nm, at least 4300 nm, at least 4400 nm, at least 4500 nm, at least 4600 nm, at least 4700 nm, at least 4800 nm, at least 4900 nm, at least 5000 nm, at least 5100 nm, at least 5200 nm, at least 5300 nm, at least 5400 nm, at least 5500 nm, at least 5600 nm, at least 5700 nm, at least 5800 nm, at least 5900 nm, at least 6000 nm, at least 6100 nm, at least 6200 nm, at least 6300 nm, at least 6400 nm, at least 6500 nm, at least 6600 nm, at least 6700 nm, at least 6800 nm, at least 6900 nm, at least 7000 nm, at least 7100 nm, at least 7200 nm, at least 7300 nm, at least 7400 nm, at least 7500 nm, at least 7600 nm, at least 7700 nm, at least 7800 nm, at least 7900 nm, at least 8000 nm, at least 8100 nm, at least 8200 nm, at least 8300 nm, at least 8400 nm, at least 8500 nm, at least 8600 nm, at least 8700 nm, at least 8800 nm, at least 8900 nm, at least 9000 nm, at least 9100 nm, at least 9200 nm, at least 9300 nm, at least 9400 nm, at least 9500 nm, at least 9600 nm, at least 9700 nm, at least 9800 nm, at least 9900 nm, at least 10000 nm or from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm, from 10 to 100 nm, from 100 to 200 nm, from 200 to 300 nm, from 300 to 400 nm, from 400 to 500 nm, from 500 to 600 nm, from 600 to 700 nm, from 700 to 800 nm, from 800 to 900 nm, from 900 to 1000 nm, from 1000 to 1100 nm, from 1100 to 1200 nm, from 1200 to 1300 nm, from 1300 to 1400 nm, from 1400 to 1500 nm, from 1500 to 1600 nm, from 1600 to 1700 nm, from 1700 to 1800 nm, from 1800 to 1900 nm, from 1900 to 2000 nm, from 2000 to 2100 nm, from 2100 to 2200 nm, from 2200 to 2300 nm, from 2300 to 2400 nm, from 2400 to 2500 nm, from 2500 to 2600 nm, from 2600 to 2700 nm, from 2700 to 2800 nm, from 2800 to 2900 nm, from 2900 to 3000 nm, from 3000 to 3100 nm, from 3100 to 3200 nm, from 3200 to 3300 nm, from 3300 to 3400 nm, from 3400 to 3500 nm, from 3500 to 3600 nm, from 3600 to 3700 nm, from 3700 to 3800 nm, from 3800 to 3900 nm, from 3900 to 4000 nm, from 4000 to 4100 nm, from 4100 to 4200 nm, from 4200 to 4300 nm, from 4300 to 4400 nm, from 4400 to 4500 nm, from 4500 to 4600 nm, from 4600 to 4700 nm, from 4700 to 4800 nm, from 4800 to 4900 nm, from 4900 to 5000 nm, from 5000 to 5100 nm, from 5100 to 5200 nm, from 5200 to 5300 nm, from 5300 to 5400 nm, from 5400 to 5500 nm, from 5500 to 5600 nm, from 5600 to 5700 nm, from 5700 to 5800 nm, from 5800 to 5900 nm, from 5900 to 6000 nm, from 6000 to 6100 nm, from 6100 to 6200 nm, from 6200 to 6300 nm, from 6300 to 6400 nm, from 6400 to 6500 nm, from 6500 to 6600 nm, from 6600 to 6700 nm, from 6700 to 6800 nm, from 6800 to 6900 nm, from 6900 to 7000 nm, from 7000 to 7100 nm, from 7100 to 7200 nm, from 7200 to 7300 nm, from 7300 to 7400 nm, from 7400 to 7500 nm, from 7500 to 7600 nm, from 7600 to 7700 nm, from 7700 to 7800 nm, from 7800 to 7900 nm, from 7900 to 8000 nm, from 8000 to 8100 nm, from 8100 to 8200 nm, from 8200 to 8300 nm, from 8300 to 8400 nm, from 8400 to 8500 nm, from 8500 to 8600 nm, from 8600 to 8700 nm, from 8700 to 8800 nm, from 8800 to 8900 nm, from 8900 to 9000 nm, from 9000 to 9100 nm, from 9100 to 9200 nm, from 9200 to 9300 nm, from 9300 to 9400 nm, from 9400 to 9500 nm, from 9500 to 9600 nm, from 9600 to 9700 nm, from 9700 to 9800 nm, from 9800 to 9900 nm, from 9900 to 10000 nm. The diameter can be measured by dynamic light scattering (DLS) as an indirect measure of size. The DLS measurement can be an ‘intensity-weighted’ average, which means the size distribution that the mean is calculated from can be weighted by the sixth power of radius. This can be referred to herein as ‘z-average’ or ‘intensity-mean’.

Alternatively, particles disclosed herein can have a radius of at least 10 nm, at least 100 nm, at least 200 nm, at least 300 nm, at least 400 nm, at least 500 nm, at least 600 nm, at least 700 nm, at least 800 nm, at least 900 nm, at least 1000 nm, at least 1100 nm, at least 1200 nm, at least 1300 nm, at least 1400 nm, at least 1500 nm, at least 1600 nm, at least 1700 nm, at least 1800 nm, at least 1900 nm, at least 2000 nm, at least 2100 nm, at least 2200 nm, at least 2300 nm, at least 2400 nm, at least 2500 nm, at least 2600 nm, at least 2700 nm, at least 2800 nm, at least 2900 nm, at least 3000 nm, at least 3100 nm, at least 3200 nm, at least 3300 nm, at least 3400 nm, at least 3500 nm, at least 3600 nm, at least 3700 nm, at least 3800 nm, at least 3900 nm, at least 4000 nm, at least 4100 nm, at least 4200 nm, at least 4300 nm, at least 4400 nm, at least 4500 nm, at least 4600 nm, at least 4700 nm, at least 4800 nm, at least 4900 nm, at least 5000 nm, at least 5100 nm, at least 5200 nm, at least 5300 nm, at least 5400 nm, at least 5500 nm, at least 5600 nm, at least 5700 nm, at least 5800 nm, at least 5900 nm, at least 6000 nm, at least 6100 nm, at least 6200 nm, at least 6300 nm, at least 6400 nm, at least 6500 nm, at least 6600 nm, at least 6700 nm, at least 6800 nm, at least 6900 nm, at least 7000 nm, at least 7100 nm, at least 7200 nm, at least 7300 nm, at least 7400 nm, at least 7500 nm, at least 7600 nm, at least 7700 nm, at least 7800 nm, at least 7900 nm, at least 8000 nm, at least 8100 nm, at least 8200 nm, at least 8300 nm, at least 8400 nm, at least 8500 nm, at least 8600 nm, at least 8700 nm, at least 8800 nm, at least 8900 nm, at least 9000 nm, at least 9100 nm, at least 9200 nm, at least 9300 nm, at least 9400 nm, at least 9500 nm, at least 9600 nm, at least 9700 nm, at least 9800 nm, at least 9900 nm, at least 10000 nm or from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm, from 10 to 100 nm, from 100 to 200 nm, from 200 to 300 nm, from 300 to 400 nm, from 400 to 500 nm, from 500 to 600 nm, from 600 to 700 nm, from 700 to 800 nm, from 800 to 900 nm, from 900 to 1000 nm, from 1000 to 1100 nm, from 1100 to 1200 nm, from 1200 to 1300 nm, from 1300 to 1400 nm, from 1400 to 1500 nm, from 1500 to 1600 nm, from 1600 to 1700 nm, from 1700 to 1800 nm, from 1800 to 1900 nm, from 1900 to 2000 nm, from 2000 to 2100 nm, from 2100 to 2200 nm, from 2200 to 2300 nm, from 2300 to 2400 nm, from 2400 to 2500 nm, from 2500 to 2600 nm, from 2600 to 2700 nm, from 2700 to 2800 nm, from 2800 to 2900 nm, from 2900 to 3000 nm, from 3000 to 3100 nm, from 3100 to 3200 nm, from 3200 to 3300 nm, from 3300 to 3400 nm, from 3400 to 3500 nm, from 3500 to 3600 nm, from 3600 to 3700 nm, from 3700 to 3800 nm, from 3800 to 3900 nm, from 3900 to 4000 nm, from 4000 to 4100 nm, from 4100 to 4200 nm, from 4200 to 4300 nm, from 4300 to 4400 nm, from 4400 to 4500 nm, from 4500 to 4600 nm, from 4600 to 4700 nm, from 4700 to 4800 nm, from 4800 to 4900 nm, from 4900 to 5000 nm, from 5000 to 5100 nm, from 5100 to 5200 nm, from 5200 to 5300 nm, from 5300 to 5400 nm, from 5400 to 5500 nm, from 5500 to 5600 nm, from 5600 to 5700 nm, from 5700 to 5800 nm, from 5800 to 5900 nm, from 5900 to 6000 nm, from 6000 to 6100 nm, from 6100 to 6200 nm, from 6200 to 6300 nm, from 6300 to 6400 nm, from 6400 to 6500 nm, from 6500 to 6600 nm, from 6600 to 6700 nm, from 6700 to 6800 nm, from 6800 to 6900 nm, from 6900 to 7000 nm, from 7000 to 7100 nm, from 7100 to 7200 nm, from 7200 to 7300 nm, from 7300 to 7400 nm, from 7400 to 7500 nm, from 7500 to 7600 nm, from 7600 to 7700 nm, from 7700 to 7800 nm, from 7800 to 7900 nm, from 7900 to 8000 nm, from 8000 to 8100 nm, from 8100 to 8200 nm, from 8200 to 8300 nm, from 8300 to 8400 nm, from 8400 to 8500 nm, from 8500 to 8600 nm, from 8600 to 8700 nm, from 8700 to 8800 nm, from 8800 to 8900 nm, from 8900 to 9000 nm, from 9000 to 9100 nm, from 9100 to 9200 nm, from 9200 to 9300 nm, from 9300 to 9400 nm, from 9400 to 9500 nm, from 9500 to 9600 nm, from 9600 to 9700 nm, from 9700 to 9800 nm, from 9800 to 9900 nm, from 9900 to 10000 nm.

In certain examples, the particles disclosed herein have a diameter of 100 nm to 400 nm. In other examples, the particles disclosed herein have a radius of 100 nm to 400 nm. Particle size can be determined by a number of techniques, such as dynamic light scattering or electron microscopy (e.g., SEM, TEM). Particles disclosed herein can be nanoparticles or microparticles.

Additionally, particles can have a homogenous size distribution or a heterogeneous size distribution. Polydispersity index (PDI), which can be measured by techniques such as dynamic light scattering, is a measure of the size distribution. A low PDI indicates a more homogeneous size distribution and a higher PDI indicates a more heterogeneous size distribution. For example, particles disclosed herein can have a PDI of less than 0.5, less than 0.4, less than 0.3, less than 0.2, less than 0.15, or less than 0.1. In particular embodiments, the particles disclosed herein have a PDI of less than 0.1. In some embodiments, the particles may have a PDI of at least 0.5, at least 0.75, at least 1.0, at least 1.5, or at least 2.0.

Particles disclosed herein can have a range of different surface charges. Particles can be negatively charged, positively charged, or neutral in charge. In some embodiments, particles have a surface charge of −150 mV to −100 mV, −100 mV to −90 mV, −90 mV to −80 mV, −80 mV to −70 mV, −70 mV to −60 mV, −60 mV to −50 mV, −50 mV to −40 mV, −40 mV to −30 mV, −30 mV to −20 mV, −20 mV to −10 mV, −10 mV to 0 mV, 0 mV to 10 mV, 10 mV to 20 mV, 20 mV to 30 mV, 30 mV to 40 mV, 40 mV to 50 mV, 50 mV to 60 mV, 60 mV to 70 mV, 70 mV to 80 mV, 80 mV to 90 mV, 90 mV to 100 mV, 100 mV to 110 mV, 110 mV to 120 mV, 120 mV to 130 mV, 130 mV to 140 mV, 140 mV to 150 mV, −150 my to −100 mV, −100 my to 0 mV, 0 my to 100 mV, 100 my to 150 mV. In particular examples, particles disclosed herein have a surface charge of −60 mV to 60 mV. The surface charge may be evaluated by zeta potential analysis at neutral pH using an appropriate buffer.

In some embodiments, the one or more surfaces comprises a first distinct surface and a second distinct surface, wherein the first distinct surface and the second distinct surface comprise a carboxylate material, wherein the first distinct, the second distinct surface, or both are a nanoparticle. In some embodiments, the one or more surfaces comprises a first distinct surface and a second distinct surface, wherein the first distinct surface and the second distinct surface comprise a surface charge of from 0 mV and −50 mV, wherein the first distinct surface, the second distinct surface, or both have a diameter of less than 400 nm. In some embodiments, the one or more surfaces comprises a first distinct surface and a second distinct surface, wherein the first distinct surface and the second distinct surface comprise a diameter of 100 to 400 nm, wherein the first distinct surface has a positive surface change, and wherein the second distinct surface has a negative surface charge.

In some embodiments, the one or more surfaces comprises a first distinct surface and a second distinct surface, wherein the first distinct surface and the second distinct surface are nanoparticles, wherein the first distinct surface has a surface change less than −20 mV and the second distinct surface has a surface charge greater than 20 mV. In some embodiments, the one or more surfaces comprises a first distinct surface and a second distinct surface, wherein the first distinct surface and the second distinct surface are microparticles, wherein the first distinct surface has a negative surface charge, and wherein the second distinct surface has a positive surface charge. In some embodiments, the one or more surfaces comprises a subset of negatively charged nanoparticles, wherein each surface of the subset differ by at least one surface chemical group. In some embodiments, the one or more surfaces comprises a first distinct surface, a second distinct surface, and a third distinct surface, wherein the first distinct surface, the second distinct surface, and the third distinct surface comprise iron oxide cores, and are less than about 500 nm in diameter, and wherein the first distinct surface comprises a negative charge of less −40 mV, the second distinct surface comprises a positive charge of more than 20 mV, and the third distinct surface comprises a negative charge of −20 mV to −40 mV.

In some embodiments, the three or more distinct magnetic surfaces comprise a nanoparticle. In some embodiments, the three or more distinct magnetic surfaces comprises a microparticle. In some embodiments, at least one distinct surface of the three or more distinct magnetic surfaces is a superparamagnetic iron oxide particle. In some embodiments, at least one distinct surface of the three or more distinct magnetic surfaces comprise an iron oxide material. In some embodiments, at least one distinct surface of the three or more distinct magnetic surfaces has an iron oxide core. In some embodiments, at least one distinct surface of the three or more distinct magnetic surfaces has iron oxide crystals embedded in a polystyrene core.

In some embodiments, each distinct surface of the three or more distinct magnetic surfaces is a superparamagnetic iron oxide particle. In some embodiments, each distinct surface of the three or more distinct magnetic surfaces comprise an iron oxide core. In some embodiments, each one distinct surface of the three or more distinct magnetic surfaces has iron oxide crystals embedded in a polystyrene core. In some embodiments, at least one surface of the three or more distinct magnetic surfaces comprises a polymer coating.

In some embodiments, the three or more distinct magnetic surfaces comprise a carboxylated polymer, an aminated polymer, or any combination thereof. In some embodiments, at least one surface of the three or more distinct magnetic surfaces comprises an iron oxide core with a silica shell coating. In some embodiments, at least one surface of the three or more distinct magnetic surfaces comprises an iron oxide core with a poly(N-(3-(dimethylamino)propyl) methacrylamide) (PDMAPMA) coating. In some embodiments, at least one surface of the three or more distinct magnetic surfaces comprises a carboxyl-containing outer surface. In some embodiments, at least one surface of the three or more distinct magnetic surfaces comprises a amine-containing outer surface. In some embodiments, at least one surface of the three or more distinct magnetic surfaces comprises an iron oxide core with a poly(oligo(ethylene glycol) methyl ether methacrylate) (POEGMA) coating.

In some embodiments, at least one surface of the three or more distinct magnetic surfaces comprises a negative surface charge. In some embodiments, at least one surface of the three or more distinct magnetic surfaces comprises a positive surface charge. In some embodiments, at least one surface of the three or more distinct magnetic surfaces comprises a neutral surface charge.

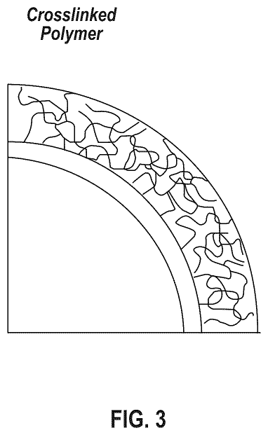

In some aspects, the present disclosure provides a method of determining the biological state of a sample from a subject, comprising: exposing a biological sample to a panel comprising a plurality of surfaces, thereby generating a plurality of protein coronas; generating proteomic data from the plurality of protein coronas; determining a protein profile of the plurality of protein coronas; and associating the protein profile to a biological state, wherein the panel comprises at least two different surfaces. The surfaces can be any of the surfaces described herein. For example, the surfaces may be a magnetic nanoparticles having an outer layer comprising any of the macromolecules in Tables 1 and/or 2. In an example, FIG. 3 shows a diagram of a nanoparticle coated with SiO₂ followed by coating with cross-linked polymer to obtain diverse functionalities.

In some embodiments, the panel comprises at least three different surfaces. In some embodiments, the method associates the protein profile to the biological state with at least 90% accuracy. In some embodiments, the plurality of surfaces comprises at least one iron oxide nanoparticle.

### Peptide Decorated Macromolecules and Surfaces

In some aspects, the present disclosure provides that some macromolecules and surfaces described herein may further comprise a peptide as described in PCT/US2022/027080, which is incorporated herein by reference in its entirety. In some embodiments, the macromolecule of Formula (I), (I-A), (I-A′), (II), (II′), (III), (III′), (III-A), or (IV) may be modified to further comprise a peptide. In some embodiments, the peptide may be bound to the surface via a specific interaction or a non-specific interaction. In some embodiments, the macromolecule of Formula (I) may further comprise a peptide. In some embodiments, the macromolecule of Formula (I-A) may further comprise a peptide. In some embodiments, the macromolecule of Formula (II) may further comprise a peptide. In some embodiments, the macromolecule of Formula (III) may further comprise a peptide. In some embodiments, the macromolecule of Formula (III-A) may further comprise a peptide. In some embodiments, the macromolecule of Formula (IV) may further comprise a peptide.

In some aspects, the macromolecule of (I), (I-A), (I-A′), (II), (II′), (III), (III′), (III-A), or (IV) may further comprise a peptide and said peptide may be covalently bonded to the macromolecule via a linker. In some aspects, disclosed herein is a macromolecule comprising recurring units of a first component and a cross-linking recurring unit, wherein the first component comprises a structure of Component (A′):

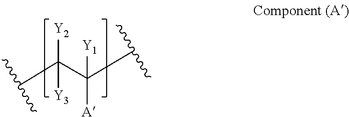

wherein
   each of Y₁, Y₂, and Y₃ is independently selected from hydrogen or C₁-C₆ alkyl;
   each of X₁, X₂, and X₃ is independently selected from hydrogen or C₁-C₆ alkyl;
   A′ is

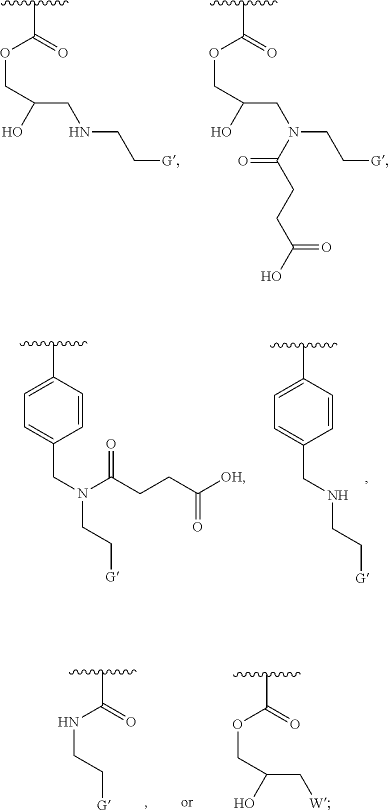

G′ or W′ comprise Q′;
   Q′ is a peptide.

The structure of

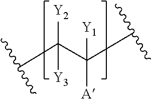

may refer interchangeably to

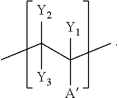

In some embodiments, each of Y₁, Y₂, and Y₃ independently selected from hydrogen or C₁-C₆ alkyl. In some embodiments, Y₁ is hydrogen or C₁-C₆ alkyl. In some embodiments, Y₁ is hydrogen. In some embodiments, Y₁ is C₁-C₆ alkyl. In some embodiments, Y₂ is hydrogen or C₁-C₆ alkyl. In some embodiments, Y₂ is hydrogen. In some embodiments, Y₂ is C₁-C₆ alkyl. In some embodiments, Y₃ is hydrogen or C₁-C₆ alkyl. In some embodiments, Y₃ is hydrogen. In some embodiments, Y₃ is C₁-C₆ alkyl.

In some embodiments, A′ is

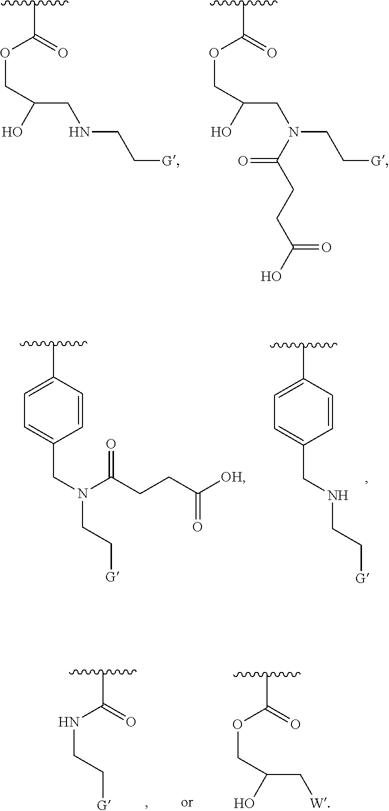

In some embodiments, A′ is

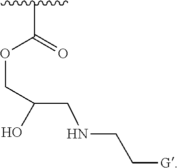

In some embodiments, A′ is

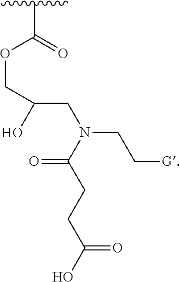

In some embodiments, A′ is

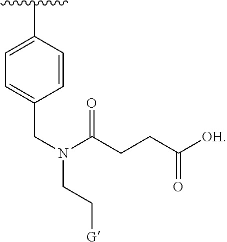

In some embodiments, A′ is

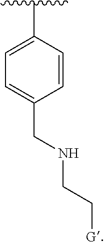

In some embodiments, A′ is

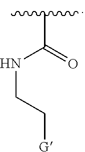

In some embodiments, A′ is

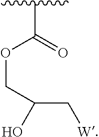

In some embodiments G′ or W′ comprise Q′. In some embodiments, G′ comprises Q′. In some embodiments, W′ comprises Q′. In some embodiments, Q′ comprises a peptide. In some embodiments, W′ is

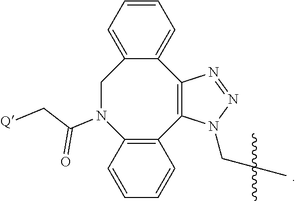

In some embodiments, G′ is

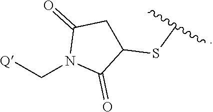

In some embodiments, a cross-linking recurring unit can comprise a polymerizable unit. In some embodiments, a cross-linking recurring unit can be randomly distributed throughout the macromolecule. In some embodiments, a cross-linking recurring unit can be distributed throughout the macromolecule in a controlled manner. In some embodiments, a cross-linking recurring unit can cross-link with another cross-linking recurring unit. In some embodiments, a cross-linking recurring unit can cross-link with component (A′).

In some aspects, disclosed herein is a macromolecule comprising recurring units of a first component and a second component, wherein the first component comprises a structure of Component (A′) and the second component comprises a structure of (B):

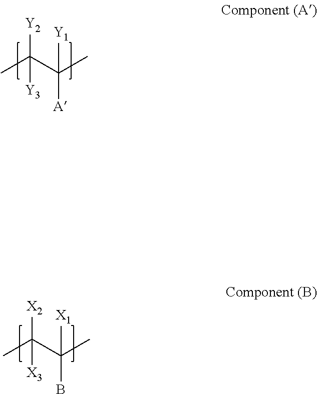

wherein
   each of Y₁, Y₂, and Y₃ is independently selected from hydrogen or C₁-C₆ alkyl;
   each of X₁, X₂, and X₃ is independently selected from hydrogen or C₁-C₆ alkyl;
   A′ is

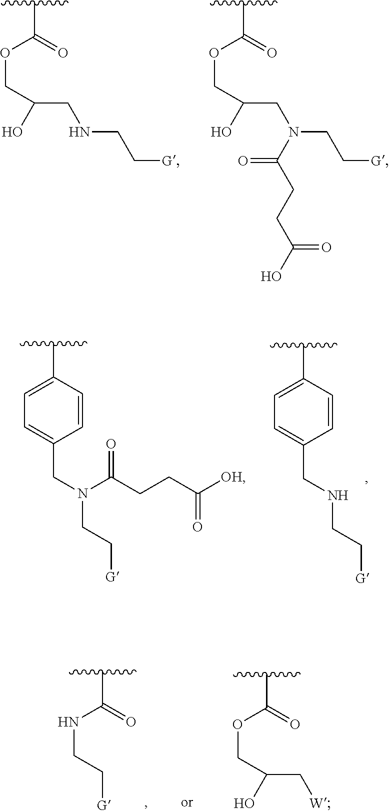

G′ or W′ comprise Q′;
   Q′ is a peptide;
   B is

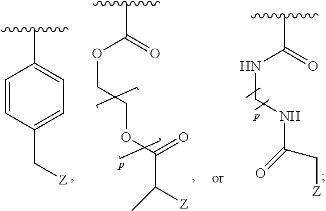

Z is a unit of Monomer (A′) or Monomer (B);
   q is an integer between 1 and 6; and
   p is an integer between 1 and 20.

In some aspects, disclosed herein is a macromolecule comprising recurring units of a first component and a second component, wherein the first component comprises a structure of Component (A′) and the second component comprises a structure of (B′):

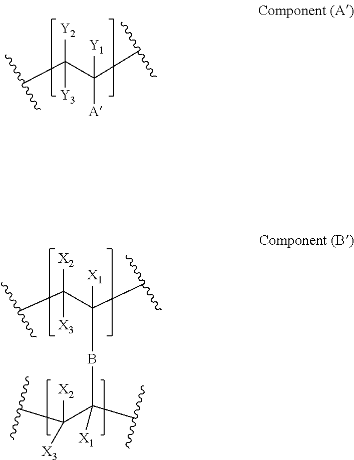

In some embodiments, each of Y₁, Y₂, and Y₃ independently selected from hydrogen or C₁-C₆ alkyl. In some embodiments, Y₁ is hydrogen or C₁-C₆ alkyl. In some embodiments, Y₁ is hydrogen. In some embodiments, Y₁ is C₁-C₆ alkyl. In some embodiments, Y₂ is hydrogen or C₁-C₆ alkyl. In some embodiments, Y₂ is hydrogen. In some embodiments, Y₂ is C₁-C₆ alkyl. In some embodiments, Y₃ is hydrogen or C₁-C₆ alkyl. In some embodiments, Y₃ is hydrogen. In some embodiments, Y₃ is C₁-C₆ alkyl. In some embodiments, X₁, X₂, and X₃ is independently selected from hydrogen or C₁-C₆ alkyl. In some embodiments, X₁ is hydrogen or C₁-C₆ alkyl. In some embodiments, X₁ is hydrogen. In some embodiments, X₁ is C₁-C₆ alkyl. In some embodiments, X₂ is hydrogen or C₁-C₆ alkyl. In some embodiments, X₂ is hydrogen. In some embodiments, X₂ is C₁-C₆ alkyl. In some embodiments, X₃ is hydrogen or C₁-C₆ alkyl. In some embodiments, X₃ is hydrogen. In some embodiments, X₃ is C₁-C₆ alkyl.

In some embodiments, A′ is

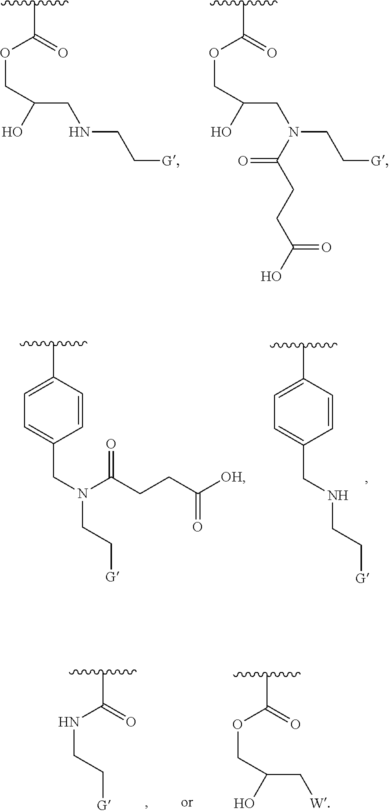

In some embodiments, A′ is

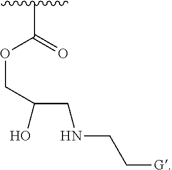

In some embodiments, A′ is

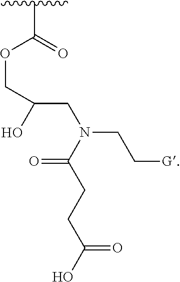

In some embodiments, A′ is

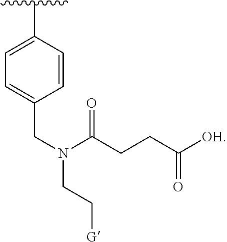

In some embodiments, A′ is

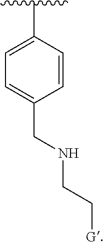

In some embodiments, A′ is

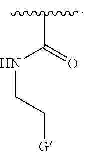

In some embodiments, A′ is

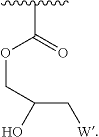

In some embodiments G′ or W′ comprise Q′. In some embodiments, G′ comprises Q′. In some embodiments, W′ comprises Q′. In some embodiments, Q′ comprises a peptide.

In some embodiments, B is

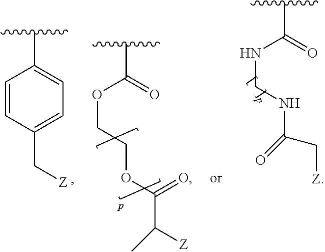

In some embodiments, B is

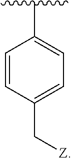

In some embodiments, B is

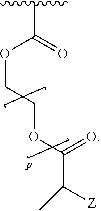

In some embodiments, B is

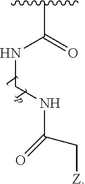

In some embodiments, Z is a unit of Monomer (A′) or Monomer (B). In some embodiments, Z is a unit of Monomer (A′). In some embodiments Z is a unit of Monomer (B).

In some embodiments, B is

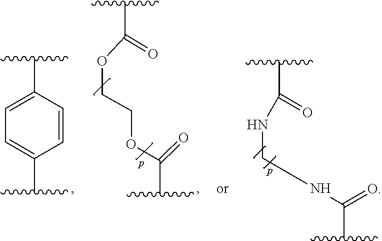

In some embodiments, B is

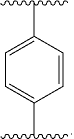 [SMILES: *c1ccc(*)cc1]

In some embodiments, B is

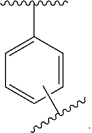

In some embodiments, B is

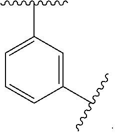 [SMILES: *c1cccc(*)c1]

In some embodiments, B is

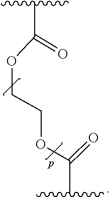

In some embodiments B is

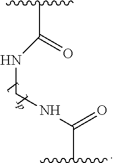

In some embodiments, q is an integer between 1 and 6. In some embodiments, p is an integer between 1 and 20. In some embodiments, the macromolecule is immobilized on a surface.

In some embodiments, W′ is

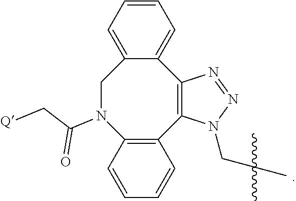

In some embodiments, G′ is

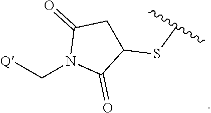

In some aspects, provided herein is a macromolecule comprising a recurring unit of a first component, wherein the first component comprises a structure of Component (A′) as described above. In some embodiments, the macromolecule may further comprise a cross-linking recurring unit. For example, the cross-linking recurring unit may result from free radical polymerization of a monomer having two vinyl groups, such as divinyl benzene (DVB), ethyleneglycol dimethacrylate (EGDMA), poly(ethylene glycol) dimethacrylate (PEGDMA) (e.g., Diethylene glycol dimethacrylate (DEGDMA), Triethylene glycol dimethacrylate (TEGDMA), and the like) N,N′-alkylenebisacrylamide (e.g., N,N′-methylenebisacrylamide (MBA), N,N′-ethylenebisacrylamide, N,N′-butylenebisacrylamide, and the like), or derivative thereof. In some embodiments, the macromolecule may comprise at least 5% by weight of the cross-linking recurring unit. In some embodiments, the macromolecule may comprise at least 25% by weight of the cross-linking recurring unit. In some embodiments, the macromolecule may comprise at least 40% by weight of the cross-linking recurring unit. In some embodiments, the macromolecule may comprise no more than 90% by weight of the cross-linking recurring unit. In some embodiments, the macromolecule may comprise no more than 75% by weight of the cross-linking recurring unit. In some embodiments, the macromolecule may comprise no more than 60% by weight of the cross-linking recurring unit. In some embodiments, the macromolecule may comprise no more than 50% by weight of the cross-linking recurring unit. In some embodiments, the macromolecule may comprise no more than 25% by weight of the cross-linking recurring unit. In some embodiments, the macromolecule may comprise 5% to 95% by weight of the cross-linking recurring unit. In some embodiments, the macromolecule may comprise 20% to 80% by weight of the cross-linking recurring unit. In some embodiments, the macromolecule may comprise 40% to 60% by weight of the cross-linking recurring unit.

In some embodiments, the macromolecule may comprise at least 5% by weight of the recurring unit of the first component. In some embodiments, the macromolecule may comprise at least 25% by weight of the recurring unit of the first component. In some embodiments, the macromolecule may comprise at least 40% by weight of the recurring unit of the first component.

In some aspects, provided herein is a surface comprising a moiety of Formula (IV′):

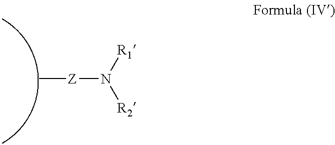

wherein
   Z is a linking moiety comprising a linear chain with 2 to 20 atoms selected from the group consisting of carbon, oxygen, and nitrogen and optional substituents on the carbon and any nitrogen atoms in the linear chain;
   R₁′ is hydrogen or succinate; and
   R₂′ is C₁-C₆ alkyl-G′;
   G′ comprises Q′;
   Q′ is peptide;
   wherein peptide does not comprise cysteine.

In some embodiments, Z is a linking moiety comprising a linear chain with 2 to 20 atoms selected from the group consisting of carbon, oxygen, and nitrogen and optional substituents on the carbon and any nitrogen atoms in the linear chain. In some embodiments, R₁′ is hydrogen or succinate. In some embodiments, R₁′ is hydrogen. In some embodiments, R₁′ is succinate. In some embodiments, R₂′ is C₁-C₆ alkyl-G′. In some embodiments, G′ comprises Q′. In some embodiments, Q′ is a peptide. In specific embodiments, peptide does not comprise cysteine. In some embodiments, G′ is

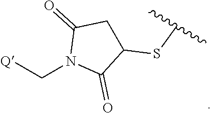

In some embodiments, Z is a linear chain with 2 to 20 atoms. In some embodiments, Z is a linear chain with 2 to 12 atoms. In some embodiments, Z is a linear chain with 2 to 6 atoms. In some embodiments, Z is a linear chain with 2 atoms. In some embodiments, Z is a linear chain with 3 atoms. In some embodiments, Z is a linear chain with 4 atoms. In some embodiments, Z is a linear chain with 5 atoms. In some embodiments, Z is a linear chain with 6 atoms. In some embodiments Z comprises carbon only. In some embodiments, Z is a C₂-C₆ alkyl chain. In some embodiments, Z is C₃ alkyl. In some embodiments Z comprises oxygen, nitrogen, carbon, or a combination thereof. In some embodiments, Z comprises substituents on the linear chain.

In some embodiments, the peptide comprises at most about 40 amino acids. In some embodiments, the peptide comprise at least about 20 amino acids. In some embodiments, the peptide comprises a synthetic sequence. In some embodiments, the peptide comprises non-natural amino acids.

In some aspects, the present disclosure provides a macromolecule modified with a peptide. In some embodiments, the macromolecules that may be modified comprises a thiol or azide. In some aspects, described herein is a surface comprising the macromolecule modified with a peptide immobilized to a surface. In some embodiments, the macromolecule is covalently coupled to the surface. In some embodiments, the macromolecule is electrostatically coupled to the surface. In some embodiments, the macromolecule is coupled to the surface through a polymerization event. In some embodiments, the polymerization event comprises a reaction with a vinyl group on the surface. In some aspects, described herein is a peptide functionalized surface. In some embodiments, the peptide comprises at most about 40 amino acids. In some aspects, the surface is a bead or a particle. In some embodiments, the surface is a particle. In some aspects, the particle comprises an iron oxide core with a silica shell coating.

The macromolecules modified with peptides may be used in the systems, methods, and composition in the same way as the other macromolecules disclosed herein. Provided herein are systems and methods of using macromolecules modified with peptides and said macromolecules immobilized on surfaces for binding or enrichment of biomolecules (e.g., proteins). In some aspects, provided herein is a system comprising a surface, a macromolecule modified with a peptide, wherein the peptide comprises a binding site, and a protein interacting with the peptide at the binding site. Provided herein, is a method of identifying at least the plurality of biomolecules or a portion thereof from the surface, the method comprising contacting a biological sample with a surface comprising the macromolecule modified with a peptide, wherein the peptides are configured to bind to a protein, releasing the plurality of biomolecules or a portion thereof from the surface, and identifying at least the plurality of biomolecules or a portion thereof from the surface. In some embodiments, the biomolecule is a protein.

In some embodiments, the methods provided herein comprise contacting a biological sample with at least two unique surfaces (e.g., macromolecules bound to surfaces) provided elsewhere herein. In some embodiments, the methods provided herein comprise contacting a biological sample with two unique surfaces (e.g., two unique macromolecules bound to surfaces).

In some embodiments, the at least two unique surfaces comprise the structures

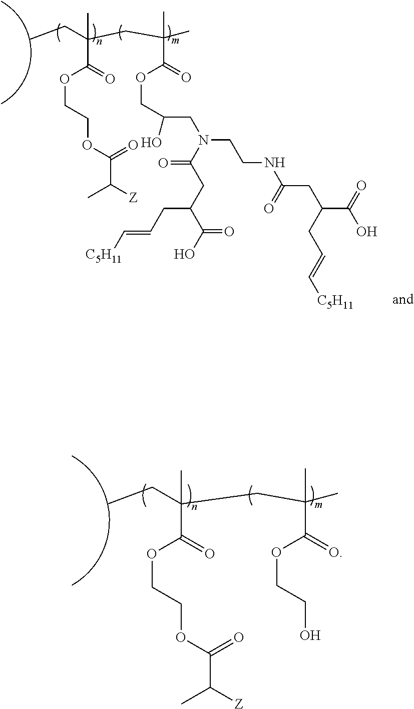

In some embodiments, the at least two unique surfaces comprise the structures

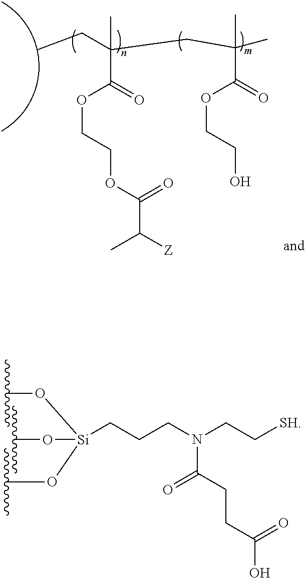

In some embodiments, the at least two unique surfaces comprise the structures

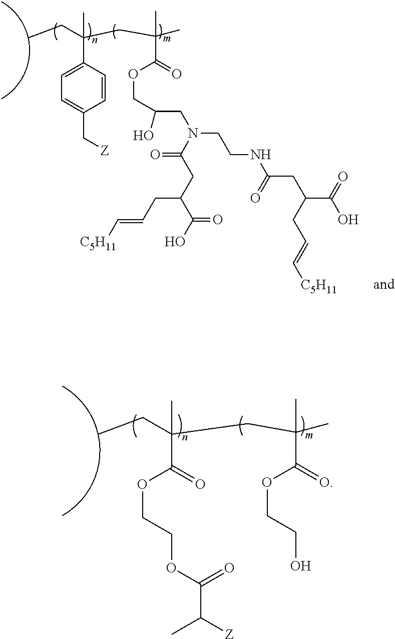

In some embodiments, the at least two unique surfaces comprise the structures

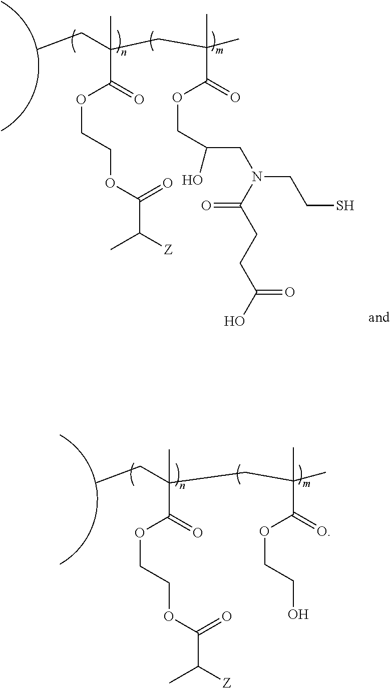

In some embodiments, the at least two unique surfaces comprise the structures

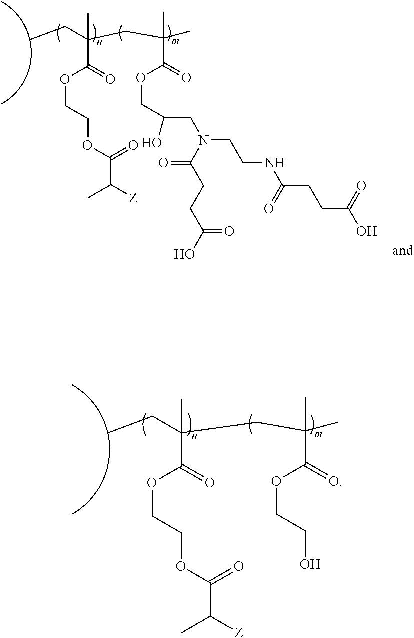

In some embodiments, the at least two unique surfaces comprise the structures

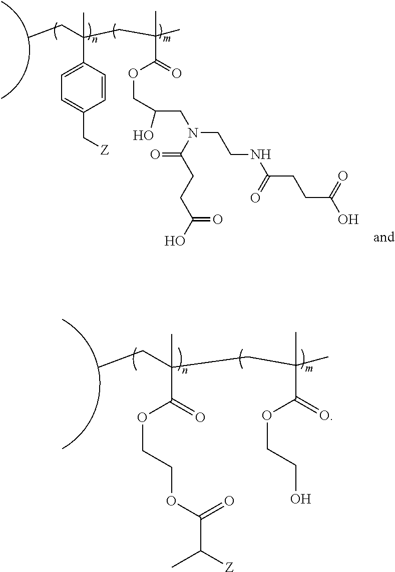

In some embodiments,

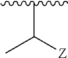

represents an attachment point for a unit of Component (A) or Component (B) as described elsewhere herein. In some embodiments,

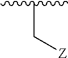

represents an attachment point for a unit of Component (A) or Component (B) as described elsewhere herein. In some embodiments, provided herein are compositions comprising at least two unique surfaces as provided elsewhere herein, such as the combination of two unique surfaces as provided herein.

In some aspects, provided herein, is a system comprising a surface, a macromolecule coupled to the surface comprising a peptide, wherein the peptide comprises a binding site, and a protein interacting with the peptide at the binding site. In some embodiments, the surface is a particle. In some embodiments, the particle is a superparamagnetic iron oxide nanoparticle. In some embodiments, the particle comprises an iron oxide material. In some embodiments, the particle has an iron oxide core. In some embodiments, the particle has iron oxide crystals embedded in a polystyrene core. In some embodiments, the particle comprises an iron oxide core with a silica shell coating.

In some embodiments, the peptide is coupled to the surface at a density of at least 1 peptide per 5 nanometers squared, 1 peptide per 50 nanometers squared, or 1 peptide per 500 nanometers squared. In some embodiments, the surface further comprises a plurality of peptides coupled thereto, wherein each peptide in the plurality of peptides is configured to bind to at least three different proteins. In some embodiments, at least two different protein are specifically bound to the peptide. In some embodiments, the system further comprises a plurality of biomolecules adsorbed on the surface. In some embodiments, the plurality of biomolecules comprises at least about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 proteins not specifically bound to the peptide. In some embodiments, the plurality of biomolecules comprises at a dynamic range of at least about 5, 6, 7, 8, 9, 10, 11, 12, 13, 14, 15, 16, 17, 18, 19, or 20. In some embodiments, the surface is provided in a solution with at least about 0.05, 0.1, 0.2, 0.3, 0.4, or 0.5 cm² in surface area of the surface per μL of the solution. In some embodiments, a plurality of biomolecules are captured on the surface such that a ratio of abundance between at least two biomolecules in the plurality of biomolecules in solution is changed for the at least two biomolecules in the plurality of biomolecules captured on the surface. In some embodiments, a plurality of biomolecules is increased in visibility in a downstream assay. In some embodiments, the visibility of a biomolecule in the plurality of biomolecules is measurable by an intensity as measured by mass spectrometry. In some embodiments, the protein comprises a targeting protein. In some embodiments, the protein comprises a vacuolar protein, lysosomal lumen, spliceosomal tri-snRNP complex, U4/U6 xU5 tri-snRNP complex, secretory granule lumen, intracellular organelle lumen, membrane raft, spliceosomal snRNP, complex, spermatoproteasome complex, or Golgi lumen protein.

In some aspects provided herein is a method of contacting a biological sample with a surface as described herein, wherein the surface comprises peptides, and said peptides are configured to bind to a protein, releasing the plurality of biomolecules or a portion thereof from the surface, and identifying at least the plurality of biomolecules or a portion thereof from the surface wherein the plurality of biomolecules or a portion thereof comprises one or more biomolecules in the at least three different biomolecules in the biological sample. In some embodiments, the biomolecule comprises a protein. In some embodiments, the biomolecule comprises a targeting protein. In some embodiments, the protein comprises a vacuolar lumen, lysosomal lumen, spliceosomal tri-snRNP complex, U4/U6 xU5 tri-snRNP complex, secretory granule lumen, intracellular organelle lumen, membrane raft, spliceosomal snRNP, complex, spermatoproteasome complex, or Golgi lumen protein.

### Oligopeptide Functionalizations

A binding molecule may comprise a peptide. Peptides are an extensive and diverse set of biomolecules which may comprise a wide range of physical and chemical properties. Depending on its composition, sequence, and chemical modification, a peptide may be hydrophilic, hydrophobic, amphiphilic, lipophilic, lipophobic, positively charged, negatively charged, zwitterionic, neutral, chaotropic, antichaotropic, reactive, redox active, inert, acidic, basic, rigid, flexible, or any combination thereof. Accordingly, a peptide surface functionalization may confer a range of physicochemical properties to a particle.

A particle may comprise a single peptide surface functionalization or a plurality of peptide surface functionalizations. A single peptide surface functionalization may comprise a plurality of identical or sequence-sharing peptides bound to a particle in a uniform fashion. For example, a particle comprising a single peptide surface functionalization may comprise about 3×10⁵ peptides of the sequence alanine-valine-tyrosine-proline-histidine-phosphotyrosine-hydroxyproline-phenylalanine-tryptophan-alanine-arginine (SEQ ID NO: 1), each coupled by its C-terminal arginine to the particle surface.

A plurality of peptide surface functionalizations may comprise a plurality of peptides sharing a common sequence but bound to a particle in a plurality of fashions. For example, a plurality of identical peptides sharing a common sequence of alanine-lysine-alanine-lysine-alanine-lysine-proline (SEQ ID NO: 2) may provide a plurality of surface functionalizations to a single particle when separately coupled to the particle through any one of the lysine residues. A plurality of peptide surface functionalizations may comprise a plurality of peptides sharing common sequence but bearing different chemical modifications. For example, a particle may comprise a plurality of peptide surface functionalizations sharing a common sequence but differing in N-terminal functionalization. A plurality of peptide surface functionalizations may comprise peptides with different lengths or sequences.

A particle may comprise any number of peptide surface functionalizations. A particle may comprise a single peptide surface functionalization. A particle may comprise at least 2, at least 3, at least 4, at least 5, at least 6, at least 8, at least 10, at least 12, at least 15, at least 20, at least 25, at least 30, at least 40, at least 50, at least 80, at least 100, at least 150, at least 200, at least 250, at least 500, at least 10³, at least 2×10³, at least 5×10³, at least 10⁴, at least 5×10⁴, at least 10⁵, at least 5×10⁵, or at least 10⁶ types of peptide surface functionalizations. A particle may comprise at most 10⁶, at most 5×10⁵, at most 10⁵, at most 5×10⁴, at most 10⁴, at most 5×10³, at most 10³, at most 500, at most 250, at most 200, at most 150, at most 100, at most 80, at most 50, at most 40, at most 30, at most 25, at most 20, at most 15, at most 12, at most 10, at most 8, at most 6, at most 5, at most 4, at most 3, or at most 2 types of peptide surface functionalizations. In some embodiments, each peptide surface functionalization of a particle is unique. Such a diversity of surface functionalizations may be achieved, for example, through relatively facile combinatorial peptide synthesis. As peptide sequence diversity grows exponentially with peptide length, a library of even relatively short oligopeptides may comprise sufficient diversity to statistically ensure unique peptide functionalization over a particle surface. For example, a peptide comprising of ten amino acids selected from a set of 20 proteinogenic amino acids may comprise more than 10¹³ different sequences, so that a particle comprising 10⁶ random sequence peptides of length 10 has less than a 10-2% chance of containing two identical peptides.

Peptide surface functionalizations may be distributed over a particle surface in a random or an ordered fashion. In some embodiments, a plurality of peptide surface functionalizations on a single particle may be spatially separated, such that a first region of the particle comprises a first peptide surface functionalization and a second region of the particle comprises a second surface functionalization.

A peptide surface functionalization may comprise a range of peptide masses or lengths. In some embodiments, a peptide surface functionalization is an amino acid dimer surface functionalization, an amino acid trimer surface functionalization, an oligopeptide surface functionalization (e.g., comprising a length between about 2 and about 30 amino acids), a polypeptide surface functionalization (e.g., comprising a length of greater than about 30 amino acids), or a protein surface functionalization (e.g., a peptide comprising a defined structure). A plurality of peptide surface functionalizations may comprise peptides of identical lengths. A plurality of peptide surface functionalizations comprise peptides of different lengths. A plurality of peptide surface functionalizations may be a plurality of oligopeptide surface functionalizations. A plurality of peptide surface functionalizations may comprise peptides of between 5 and 12 amino acids in length. A plurality of peptide surface functionalizations may comprise peptides of between 4 and 8, between 4 and 10, between 4 and 12, between 4 and 15, between 4 and 20, between 4 and 25, between 5 and 8, between 5 and 10, between 5 and 12, between 5 and 15, between 5 and 20, between 5 and 25, between 6 and 8, between 6 and 10, between 6 and 12, between 6 and 15, between 6 and 20, between 6 and 25, between 7 and 10, between 7 and 12, between 7 and 15, between 7 and 20, between 7 and 25, between 8 and 10, between 8 and 12, between 8 and 15, between 8 and 20, between 8 and 25, between 10 and 12, between 10 and 15, between 10 and 20, between 10 and 25, between 12 and 15, between 12 and 20, between 12 and 25, between 15 and 20, between 15 and 25, between 20 and 25, or between 20 and 30 amino acids in length.

A peptide surface functionalization may comprise a subset of amino acid types. As different amino acid types confer different properties to peptides, the properties of a peptide surface functionalization may be at least partially determined by constructing the peptide surface functionalization from a limited number of amino acid types. For example, glutamic acid and aspartic acid tend to lower the isoelectric point of peptides to which they are attached, while histidine, lysine, and arginine tend to raise peptide isoelectric points while simultaneously providing nucleophilic character. Peptide surface functionalization properties, and thus particle physicochemical surface properties, can be regulated by controlling the types and ratios between types of constituent amino acids. In many cases, peptide surface functionalizations will lack cysteine residues to prevent thiol nucleophilic behavior, redox activity, and crosslinking. In many cases, peptide surface functionalizations will comprise a defined ratio or range of ratios of acidic and basic sidechains as a means for controlling isoelectric point and charge. A peptide surface functionalization may be free of at least one of cysteine, methionine, tryptophan, tyrosine, phenylalanine, and derivatives thereof. A peptide functionalization may be free of at least two of cysteine, methionine, tryptophan, tyrosine, phenylalanine, and derivatives thereof. A peptide functionalization may be free of at least three of cysteine, methionine, tryptophan, tyrosine, phenylalanine, and derivatives thereof. A peptide functionalization may be free of at least four of cysteine, methionine, tryptophan, tyrosine, phenylalanine, and derivatives thereof. A peptide functionalization may be free of cysteine, methionine, tryptophan, tyrosine, phenylalanine, and derivatives thereof. A peptide may comprise amino acids selected from the group consisting of alanine, arginine, aspartic acid, glutamic acid, glutamine, glycine, histidine, isoleucine, leucine, lysine, proline, serine, asparagine, threonine, valine, and derivatives thereof. A peptide may comprise amino acids selected from the group consisting of alanine, arginine, aspartic acid, cysteine, glutamic acid, glutamine, glycine, histidine, isoleucine, leucine, lysine, methionine, phenylalanine, proline, serine, asparagine, threonine, tryptophan, tyrosine, valine, and derivatives thereof. A plurality of peptides may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, or at most 20 types of amino acids. A peptide may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, or at most 20 types of amino acids. A peptide may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 types of amino acids. A plurality of peptides may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 types of amino acids.

A peptide functionalization may comprise a non-proteinogenic amino acid. The non-proteinogenic amino acid may be a chemically modified form of a proteinogenic amino acid. Such a chemical modification may include acylation, alkylation, amidation, deamidation, carbamylation, carbonylation, carboxylation, decarboxylation, citrullination, flavination, glycosylation, halogenation, hydroxylation, nitrosylation, oxidation, phosphorylation, prenylation, racemization, reduction, succinylation, sulfation, or any combination thereof. The non-proteinogenic amino acid may comprise a post-translational modification.

A peptide functionalization may provide a specific isoelectric point to a particle. A particle may comprise an isoelectric point within 2, within 1.5, within 1, within 0.75, within 0.5, within 0.25, or within 0.1 pH units of a peptide functionalization coupled to its surface. A method may comprise forming a biomolecule corona at a pH in which a particle and an analyte comprise opposite charges, or in which the particle and analyte are both neutral. A method may comprise separating a plurality of particles by their isoelectric points (e.g., by isoelectric focusing). A peptide functionalization may comprise an isoelectric point between 4 and 10. A peptide functionalization may comprise an isoelectric point between 7 and 10. A peptide functionalization may comprise an isoelectric point between 4 and 7. A peptide functionalization may comprise an isoelectric point between 5 and 10. A peptide functionalization may comprise an isoelectric point between 6 and 8. A particle may comprise a plurality of peptide functionalizations with different isoelectric points. A peptide functionalization or a plurality of peptide functionalizations may provide a plurality of pka values to a particle.

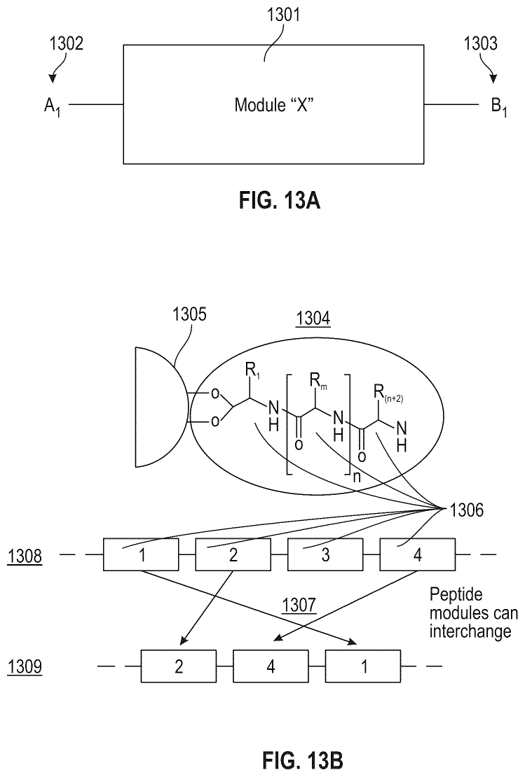

A peptide functionalization or a library of peptide functionalizations may be constructed from modular units. The modular units may comprise individual amino acids, oligomeric units, such as oligopeptides comprising between 2 and 6 amino acid units, or non-peptidic chemical or material units, such as succinyl linkers. For example, a plurality of modular units may comprise oligopeptides having about 1 to about 6 amino acids. A modular unit may be biodegradable (e.g., configured for metabolization and mineralization by a biological system). A diagram of a modular unit structure consistent with the present disclosure is provided in FIG. 13A. A modular unit may comprise an amino acid, an oligopeptide, a non-peptide moiety (e.g., a nucleotide), or any combination thereof 1301. A modular unit may be linear or branched. A modular unit may comprise a first reactive handle 1302 and a second reactive handle 1303. Each reactive handle may comprise a different coupling specificity. For example, a first reactive handle may be configured to only couple with a third type of reactive handle, while a second reactive handle may be configured to couple only with a fourth type of reactive handle. Conversely, two reactive handles may share a specificity. For example, a first reactive handle may be configured to couple with a third type or a fourth type of reactive handle, while a second reactive handle may be configured to couple with the fourth type of reactive handle and a fifth type of reactive handle. In other cases, a first reactive handle is identical to a second reactive handle.

A modular unit may be individually addressable from among a plurality of modular units (e.g., may comprise a unique chemical reactivity or physical property from among the modular units of a peptide). Two or more modular units may be connected by a linking element. Such a linking element may be non-peptidic, and for example may comprise a saccharide, lipid, nucleic acid, or alkyl backbone for incorporation into a peptide functionalization. Separate modular units may be configured to undergo positional exchange, such that a first modular unit from a first peptide functionalization exchanges positions with a second modular unit from the first peptide functionalization or from a second peptide functionalization. Furthermore, an individual modular unit may be configured for removal, modification (e.g., functional group coupling, oxidation, or reduction), or substitution by a separate modular unit. Modifiable and substitutable modular unit building blocks may be used to rapidly generate diverse peptide functionalization libraries. Such a peptide functionalization may comprise between 7 and 20 amino acid residues.

An example of such a modular peptide binding molecule is provided in FIG. 13B. The peptide binding molecule 1304 may be coupled to a particle 1305. The peptide binding molecule may comprise a plurality of modular units 1306. The units may be capable of intermolecular (e.g., with another peptide binding molecule) or intramolecular exchange 1307, thereby providing a mechanism for a first peptide binding molecule 1308 to generate a second peptide binding molecule 1309 comprising a common modular unit.

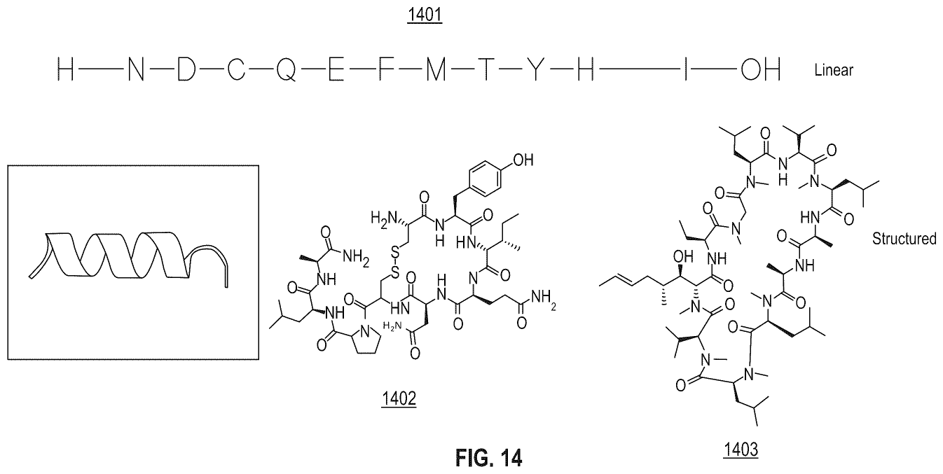

FIG. 14 provides examples of peptide binding molecules with different structural configurations. A peptide binding molecule may comprise a linear structure, for example that of 1401. A peptide binding molecule may also comprise a branched or cyclic structure, as is illustrated in 1402 and 1403.

### Analysis and Automated Systems

In one aspect, described herein is a system for identifying biomolecules in a biological sample, wherein the system may comprise (i) a macromolecule immobilized to one or more surfaces as described elsewhere herein; (ii) a suspension solution; (iii) a biological sample comprising proteins; and (iv) an automated system comprising a network of units with differentiated functions for isolating protein adsorbed to the one or more surfaces, and wherein the automated system is programed to perform a series of steps.

In some embodiments, the macromolecule immobilized to a surface (or a surface of the disclosure) as described elsewhere herein, the suspension solution, and the biological sample comprising a concentration of protein may be incubated at a temperature of about 10 degrees Celsius (° C.) to about 100° C. In some embodiments, one or more components of the composition may be incubated at a temperature of about 20° C. to about 90° C. In some embodiments, one or more components of the composition may be incubated at a temperature of about 20° C. to about 50° C. In some embodiments, the incubation may be for at least 10 minutes, at least 15 minutes, at least 20 minutes, at least 25 minutes, at least 30 minutes or at least 45 minutes. In some embodiments, the incubation may be for no more than 3 hours, no more than 2 hours, no more than 90 minutes, or no more than 1 hour. In some embodiments, the incubation may be for about 20 to about 90 minutes.

In some embodiments, the suspension may comprise one or more buffers. For example, the pH may be modified as disclosed in PCT/US2023/068457, filed on Jun. 14, 2023, which is hereby incorporated herein by reference in its entirety. The pH may be, for example about 5, about 6, about 7, about 8, about 9, about 10, about 5 to about 7, or about 9 to about 10. In some embodiments, the suspension solution may comprise Tris, EDTA, and CHAPS buffer. For example, the suspension solution may be Tris, EDTA in 150 millimolar (mM) KCl and 0.05% CHAPS buffer. In another example, the suspension solution may be 10 mM Tris HCl pH 7.4, 1 mM EDTA. In some embodiments, the suspension comprises tris(hydroxymethyl)aminomethane. In some embodiments, the suspension comprises tris(hydroxymethyl)aminomethane at pH of about 9.5.

In some aspects, the present disclosure provides an automated system comprising a network of units as described in WO2021/026172, which is incorporated herein by reference in its entirety. In some embodiments, the network of units may comprise differentiated functions in distinguishing states of a complex biological sample using a plurality of particles having surfaces with different physicochemical properties wherein: a first unit comprises a multichannel fluid transfer instrument for transferring fluids between units within the system; a second unit comprises a support for storing a plurality of biological samples; a third unit comprises a support for a sensor array plate possessing partitions that comprise the plurality of particles having surfaces with different physicochemical properties for binding a population of analytes within the complex biological sample; a fourth unit comprises supports for storing a plurality of reagents; a fifth unit comprises supports for storing a reagent to be disposed of; a sixth unit comprises supports for storing consumables used by the multichannel fluid transfer instrument; and wherein the system is programed to perform a series of steps comprising: contacting the complex biological sample with a specified partition of the sensor array; incubating the complex biological sample with the plurality of particles contained within the partition of the sensor array plate; removing components from a partition except the plurality of particles and a population of analytes interacting with a particle; and optionally preparing the population of analytes for analysis, such as mass spectrometry.

In some embodiments, the first unit comprises a degree of mobility that enables access to all other units within the system. In some embodiments, the first unit comprises a capacity to perform pipetting functions.

In some embodiments, the support of the second and/or third unit comprises support for a single plate, a 6 well plate, a 12 well plate, a 96 well plate, or a rack of microtubes. In some embodiments, the second and/or unit comprises a thermal unit capable of modulating the temperature of said support and a sample. In some embodiments, the second and/or third unit comprises a rotational unit capable of physically agitating and/or mixing a sample.

In some embodiments, the plurality of particles having surfaces with different physicochemical properties for binding a population of analytes within the biological sample are immobilized to a surface within a partition of the sensory array. In some embodiments, the plurality of particles comprises a plurality of magnetic nanoparticles with different physicochemical properties for binding a population of analytes within the complex biological sample. In some embodiments, the system comprises a step wherein the sensor array plate is transferred to an additional seventh unit that comprises a magnetized support and a thermal unit capable of modulating the temperature of said support and a sample and incubated for an additional amount of time.

In some embodiments, the fourth unit comprises a set of reagents for: generating the sensor array plate; washing an unbound sample; and/or preparing a sample for mass spectrometry. In some embodiments, contacting the biological sample with a specified partition of the sensor array comprises pipetting a specified volume of the biological sample into the specific partition of the sensor array. In some embodiments, contacting the biological sample with a specified partition of the sensor array comprises pipetting a volume corresponding to a 1:1, 1:2:1:3, 1:4, 1:5, 1:6, 1:7, 1:8, 1:9, 1:10, 1:15, or 1:20 ratio of a plurality of particles in a solution to the biological sample.

In some embodiments, contacting the biological sample with a specified partition of the sensor array comprises pipetting a volume of at least 10 microliters, at least 20 microliters at least 50 microliters, at least 100 microliters, at least 250 microliters, at least 500 microliters, or at least 1000 microliters of the biological sample into the specific partition of the sensor array. In some embodiments, contacting the biological sample with a specified partition of the sensor array comprises pipetting a volume of no more than 1000 microliters, no more than 500 microliters, nor more than 250 microliters, no more than 150 microliters, no more than 100 microliters, no more than 75 microliters, no more than 50 microliters, or no more than 30 microliters.

In some embodiments, incubating the biological sample with the plurality of particles contained within the partition of the sensor array plate comprises an incubation time of at least about 10 seconds, at least about 15 seconds, at least about 20 seconds, at least about 25 seconds, at least about 30 seconds, at least about 40 seconds, at least about 50 seconds, at least about 60 seconds, at least about 90 seconds, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 15 minutes, at least about 20 minutes, at least about 25 minutes, at least about 30 minutes, at least about 45 minutes, at least about 50 minutes, at least about 60 minutes, at least about 90 minutes, at least about 2 hours, at least about 3 hours, at least about 4 hours, at least about 5 hours, at least about 6 hours, at least about 7 hours, at least about 8 hours, at least about 9 hours, at least about 10 hours, at least about 12 hours, at least about 14 hours, at least about 15 hours, at least about 16 hours, at least about 17 hours, at least about 18 hours, at least about 19 hours, at least about 20 hours, or at least about 24 hours. In some embodiments, incubating the biological sample with the plurality of particles contained within the partition of the sensor array plate comprises an incubation time of no more than 24 hours, no more than 12 hours, no more than 6 hours, no more than 3 hours, no more than 2 hours, no more than 90 minutes, no more than 75 minutes, no more than 60 minutes, no more than 45 minutes, or no more than 30 minutes. In some embodiments, incubating the biological sample with the plurality of particles contained within the partition of the sensor array plate comprises an incubation time of 30 minutes and 3 hours.

In some embodiments, incubating the biological sample with the plurality of particles contained within the partition of the substrate comprises an incubation temperature between about 4° C. to about 40° C. Incubating the biological sample with the plurality of particles contained within the partition of the substrate may comprise an incubation temperature between about 4° C. to about 37° C. Incubating the biological sample with the plurality of particles contained within the partition of the substrate may comprise an incubation temperature between about 20° C. to about 50° C. Incubating the biological sample with the plurality of particles contained within the partition of the substrate may comprise an incubation temperature between about 4° C. to about 100° C.

In some embodiments, removing all components from a partition except the plurality of particles and a population of analytes interacting with a particle comprises a series of wash steps.

In some embodiments, the second unit can facilitate a transfer of the sample for mass spectrometry to a mass spectrometry unit.

In some aspects, the present disclosure provides an automated apparatus to identify proteins in a biological sample, the automated apparatus comprising: a sample preparation unit; a substrate comprising a plurality of channels; a plurality of pipettes; a plurality of solutions, a plurality of surfaces as described herein, and wherein the automated apparatus is configured to form a protein corona and digest the protein corona.

In some embodiments, the automated apparatus further comprises a magnetic source. In some embodiments, the automated apparatus is configured for BCA, gel, or trypsin digestion of the protein corona.

In some embodiments, the automated apparatus is enclosed. In some embodiments, the automated apparatus is sterilized before use. In some embodiments, the automated apparatus is configured to a mass spectrometry. In some embodiments, the automated apparatus is temperature controlled.

The proteomic data of the sample can be identified, measured, and quantified using a number of different analytical techniques. For example, proteomic data can be analyzed using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, and methods disclosed in EP3548652, WO2019083856, WO2019133892, each of which is incorporated herein by reference in its entirety, and other protein separation techniques

In some aspects, the present disclosure provides an automated apparatus for generating a subset of biomolecules from a biological sample, comprising: a substrate comprising a plurality of partitions, a first unit comprising the biological sample, and a loading unit that is movable across the substrate and is capable of transferring a volume (e.g., a volume of buffer) between different units of the apparatus. In some cases, the substrate is a multi-well plate.

The plurality of partitions may comprise a plurality of sensor elements. The plurality of sensor elements may comprise surfaces. The plurality of sensor elements may be surfaces (e.g., particles) as disclosed herein. For example, the sensor elements may include a first distinct nanoparticle and a second distinct nanoparticle, wherein the first distinct nanoparticle comprises a surface selected from Tables 3 and/or 4.

A partition from among the plurality of partitions may comprise 1 to 100 types of sensor elements (e.g., distinct surfaces). A partition from among the plurality of partitions may comprise 2 to 50 types of sensor elements. A partition from among the plurality of partitions may comprise 2 to 20 types of sensor elements. A partition from among the plurality of partitions may comprise 2 to 5 types of sensor elements. A partition from among the plurality of partitions may comprise 3 to 8 types of sensor elements. A partition from among the plurality of partitions may comprise 4 to 10 types of sensor elements. A partition from among the plurality of partitions may comprise 5 to 12 types of sensor elements. A partition from among the plurality of partitions may comprise 6 to 15 types of sensor elements. A partition from among the plurality of partitions may comprise 8 to 20 types of sensor elements. A partition from among the plurality of partitions may comprise 2 types of sensor elements. A partition from among the plurality of partitions may comprise 3 types of sensor elements. A partition from among the plurality of partitions may comprise 4 types of sensor elements.

Two or more partitions from among the plurality of partitions may comprise different quantities of sensor elements. Two or more partitions from among the plurality of partitions may comprise different types of sensor elements. A partition amongst a plurality of partitions may comprise a combination of types and/or quantities of sensor element(s) that differs from other partitions in the plurality. A subset of partitions in a plurality of partitions may each contain a combination of distinct sensor elements that is distinct from other partitions in the plurality.

Sensor elements may be stored in dry form inside of or within the partitions. Dry sensor elements may be reconstituted or rehydrated prior to use. Sensor elements may also be stored within solutions. For example, a substrate partition may comprise a solution comprising a high concentration of surfaces.

Partitions from among the plurality of partitions comprise different concentrations or amounts (e.g., by mass/molar amount per unit volume of sample) of sensor elements. A partition from among the plurality of partitions may comprise from 1 pM to 100 nM of sensor elements. A partition from among the plurality of partitions comprise may from 1 pM to 500 pM of sensor elements. A partition from among the plurality of partitions may comprise from 10 pM to 1 nM of sensor elements. A partition from among the plurality of partitions may comprise from 100 pM to 10 nM of sensor elements. A partition from among the plurality of partitions may comprise from 500 pM to 100 nM of sensor elements. A partition from among the plurality of partitions may comprise from 50 μg/ml to 300 μg/ml of sensor elements. A partition from among the plurality of partitions may comprise from 100 μg/ml to 500 μg/ml of sensor elements. A partition from among the plurality of partitions may comprise from 250 μg/ml to 750 μg/ml of sensor elements. A partition from among the plurality of partitions may comprise from 400 μg/ml to 1 mg/ml of sensor elements. A partition from among the plurality of partitions may comprise from 600 μg/ml to 1.5 mg/ml of sensor elements. A partition from among the plurality of partitions may comprise from 800 μg/ml to 2 mg/ml of sensor elements. A partition from among the plurality of partitions may comprise from 1 mg/ml to 3 mg/ml of sensor elements. A partition from among the plurality of partitions may comprise from 2 mg/ml to 5 mg/ml of sensor elements. A partition from among the plurality of partitions may comprise more than 5 mg/ml of sensor elements.

The loading unit may be configured to move between and transfer volumes (e.g., a volume of a solution or a powder) between any units, compartments, or partitions within the apparatus. The loading unit may be configured to move precise volumes (e.g., within 0.1%, 0.01%, 0.001% of the specified volume). The loading unit may be configured to collect a volume from the substrate or a compartment or partition within the substrate, and dispense the volume back into the substrate or compartment or partition within the substrate, or to dispense the volume or a portion of the volume into a different unit, compartment, or partition. The loading unit may be configured to move multiple volumes simultaneously, such as 2 to 400 separate volumes. The loading unit may comprise a plurality of pipette tips.

The loading unit may be configured to move a volume of a liquid. The volume may be about 0.1 μl, 0.2 μl, 0.3 μl, 0.4 μl, 0.5 μl, 0.6 μl, 0.7 μl, 0.8 μl, 0.9 μl, 1 μl, 2 μl, 3 μl, 4 μl, 5 μl, 6 μl, 7 μl, 8 μl, 9 μl, 10 μl, 12 μl, 15 μl, 20 μl, 25 μl, 30 μl, 40 μl, 50 μl, 60 μl, 70 μl, 80 μl, 90 μl, 100 μl, 120 μl, 150 μl, 180 μl, 200 μl, 250 μl, 300 μl, 400 μl, 500 μl, 600 μl, 800 μl, 1 ml, or more than 1 ml. The liquid may be a biological sample or a solution.

In some cases, the solution comprises a wash solution, a resuspension solution, a denaturing solution, a buffer, a reagent (e.g., a reducing reagent), or any combination thereof. In some cases, the solution comprises a biological sample.

In part owing to these functionalities, the loading unit can be capable of partitioning a sample. In some embodiments, this comprises dividing a sample into a number of partitions. A sample can be divided into at least 2, 3, 4, 5, 6, 7, 8, 9, 10, 12, 15, 20, 25, 30, 40, 50, 60, 70, 80, 90, 100, 120, 150, 180, 200, 250, 300, 350, 400, 500, or more partitions. A sample can be divided into 96, 192, or 384 partitions. The automated apparatus can comprise multiple substrates comprising partitions. The automated apparatus may comprise 1, 2, 3, 4, 5, or more substrates comprising partitions. In some cases, the loading unit loads different volumes of the biological sample into different partitions. In some cases, the loading unit loads identical volumes into two or more partitions. The volume of biological sample loaded into a partition may be about 0.1 μl, 0.2 μl, 0.3 μl, 0.4 μl, 0.5 μl, 0.6 μl, 0.7 μl, 0.8 μl, 0.9 μl, 1 μl, 2 μl, 3 μl, 4 μl, 5 μl, 6 μl, 7 μl, 8 μl, 9 μl, 10 μl, 12 μl, 15 μl, 20 μl, 25 μl, 30 μl, 40 μl, 50 μl, 60 μl, 70 μl, 80 μl, 90 μl, 100 μl, 120 μl, 150 μl, 180 μl, 200 μl, 250 μl, 300 μl, 400 μl, 500 μl, 600 μl, 800 μl, 1 ml, or more than 1 ml. The volume of biological sample loaded into a partition may be about 10 μl to 400 μl. The volume of biological sample loaded into a partition may be about 5 μl to 150 μl. The volume of biological sample loaded into a partition may be about 35 μl to 80 μl. In some cases, the loading unit may partition two or more biological samples. For example, a sample storage unit may comprise two biological samples that the system partitions into one well plate. In some embodiments, the loading unit can facilitate a transfer of the sample for mass spectrometry to a mass spectrometry unit.

The system may be configured to perform a dilution on a sample or a sample partition. A sample or sample partition may be diluted with buffer, water (e.g., purified water), a non-aqueous solvent, or any combination thereof. The diluent may be stored in the automated apparatus prior to dispensation into a substrate partition. The automated apparatus may store a plurality of diluents differing in pH, salinity, osmolarity, viscosity, dielectric constant, or any combination thereof. The diluents may be used to adjust the chemical properties of a sample or sample partition. The automated apparatus may dilute a sample or sample partition by 2-fold, 3-fold, 4-fold, 5-fold, 6-fold, 8-fold, 10-fold, 15-fold, 20-fold, 30-fold, 40-fold, 50-fold, 75-fold, 100-fold, 150-fold, 200-fold, 300-fold, 400-fold, 500-fold or greater. The automated apparatus may perform different dilutions on two samples or sample partitions. The system may perform different dilutions on each partition from among a plurality of partitions. For example, the system may perform different dilutions on each of the 96 sample partitions in a 96 well plate. In some cases, the different dilutions comprise different degrees of dilution (e.g., 2-fold vs. 4-fold). In some cases, the different dilutions comprise dilution with different solutions (e.g., different buffers). In some cases, two sample partitions may be made to differ in one or more chemical properties, such as pH, salinity, or viscosity.

In some cases, the system may modify the chemical composition of a sample or sample partition. The system may modify or adjust the pH, salinity, osmolarity, dielectric constant, viscosity, buffer types, salt types, sugar types, detergent types, or any combination thereof for a sample or sample partition. Such modification or adjustments may comprise mixing a reagent from the fourth unit with a sample or sample partition. The system may differently modify the chemical composition of two samples or sample partitions.

A system or automated apparatus of the present disclosure may also comprise an incubation element. The incubation element may contact, support, or hold another component of the automated apparatus (e.g., the substrate or a unit). The incubation unit may contact, support, or hold multiple components of the automated apparatus. The incubation element may contact the substrate to facilitate heat transfer between the incubation element and the substrate. The incubation unit may be configured to control the temperature of the one or more components of the automated apparatus, such as by heating or cooling. The incubation element may be capable of cooling a component of the apparatus to from 20° C. to 1° C. The incubation element may be capable of heating a component of the apparatus to from 25° C. to 100° C. The incubation element may be capable of setting the temperature a component of the apparatus to from 4° C. to 37° C. The incubation element may be configured to heat or cool different portions of a component of the automated apparatus to different temperatures. For example, the incubation element may hold a first partition in the substrate at 30° C. and a second partition in the substrate at 35° C. The incubation element may control the temperature of a sample or partition. The incubation element may comprise a temperature sensor (e.g., a thermocouple) for detecting the temperature within a partition or container. The incubation element may calibrate its heating or cooling to the readout from the temperature sensor.

The incubation element may be configured to physically agitate a component of the automated apparatus. The agitation may be in the form of shaking or spinning, vibrating, rocking, sonicating, or any combination thereof. The incubation element may be capable of providing multiple agitation intensities and/or frequencies. For example, the incubation element may comprise multiple settings for shaking at different frequencies and amplitudes. The incubation element may also be capable of stirring and or mixing a volume (e.g., a portion of the biological sample).

The automated apparatus may comprise a unit comprising a resuspension solution. The loading unit may be capable of transferring a volume of the resuspension solution to a partition from among the plurality of partitions of the substrate. In some cases, this results in the dilution of a sample present within the partition and can further result in the desorption of a plurality of biomolecules from a biomolecule corona disposed on a sensor element within the partition. The quantity of biomolecules desorbed from a biomolecule corona can depend on the volume of the resuspension solution added to the partition, the temperature of the partition, the composition of the resuspension solution (e.g., the salinity, osmolarity, viscosity, dielectric constant, or pH), the volume of the biological sample within the partition, and the sensor element type and the composition of biomolecules in the biomolecule corona. The transfer of a volume of the resuspension solution into a partition may result in the desorption of less than 5% of the biomolecules from a biomolecule corona. The transfer of a volume of the resuspension solution into a partition may result in the desorption of 10% to 20% of the biomolecules from a biomolecule corona. The transfer of a volume of the resuspension solution into a partition may result in the desorption of 20% to 30% of the biomolecules from a biomolecule corona. The transfer of a volume of the resuspension solution into a partition may result in the desorption of 30% to 40% of the biomolecules from a biomolecule corona. The transfer of a volume of the resuspension solution into a partition may result in the desorption of 40% to 50% of the biomolecules from a biomolecule corona. The transfer of a volume of the resuspension solution into a partition may result in the desorption of 50% to 60% of the biomolecules from a biomolecule corona. The transfer of a volume of the resuspension solution into a partition may result in the desorption of 60% to 70% of the biomolecules from a biomolecule corona. The transfer of a volume of the resuspension solution into a partition may result in the desorption of 70% to 80% of the biomolecules from a biomolecule corona. The transfer of a volume of the resuspension solution into a partition may result in the desorption of 80% to 90% of the biomolecules from a biomolecule corona. The transfer of a volume of the resuspension solution into a partition may result in the desorption of more than 90% of the biomolecules from a biomolecule corona.

In some cases, multiple rounds of desorption are performed. In each round, the supernatant comprising the desorbed biomolecules may be collected, analyzed, or discarded. The types and abundances of biomolecules in the supernatant may differ between desorption rounds. The automated apparatus may perform one or more desorption and discard cycles (i.e., washes), followed by one or more desorption cycles comprising sample collection and/or analysis.

The resuspension solution may be tailored to optimize enrichment of particular biomarkers. The resuspension solution may comprise a buffer, such as Tris-EDTA (TE), CHAPS, PBS, citrate, HEPES, MES, CHES, or another bio buffer. The resuspension solution may comprise Tris EDTA (TE) 150 mM KCl 0.05% CHAPS buffer. The resuspension solution may comprise 10 mM TrisHCl pH 7.4, 1 mM EDTA. The resuspension solution may also contain or be highly purified water (e.g., distilled or deionized water). Biomolecule desorption may be augmented by heating or agitation by an incubation element. The supernatant may be transferred to a new partition following desorption. A resuspension solution may be used to dilute a sample.

The automated apparatus may comprise a unit comprising a denaturing solution. The denaturing solution may comprise a protease. The denaturing solution may comprise a chemical capable of performing peptide cleavage (e.g., cyanogen bromide, formic acid, or hydroxylamine, 2-nitro-5-thiocyanatobenzoic acid). The denaturing solution may comprise a chemical denaturant such as guanidine, urea, sodium deoxycholate, acetonitrile, trichloroacetic acid, acetic acid, sulfosalicylic acid, sodium bicarbonate, ethanol, perchlorate, dodecyl sulfate, or any combination thereof. The denaturing solution may comprise a reductant, such as 2-mercaptoethanol, dithiothreitol, or tris(2-carboxyethyl)phosphine. The protease may be trypsin and/or LysC. The denaturing solution may be added to a partition following desorption. The denaturing solution may be added to a partition comprising biomolecule coronas.

The automated apparatus may comprise a magnet or array of magnets. The automated apparatus may capable of moving the substrate onto and off of the magnet or array of magnets. The array of magnets may be structured so that a plurality of magnets from the array of magnets can rest directly underneath a plurality of partitions from the substrate. The magnet may be capable of immobilizing magnetic sensor elements (e.g., magnetic particles such as coated or uncoated super paramagnetic iron oxide nanoparticles) within a partition on the substrate. For example, the magnet may prevent magnetic nanoparticles from being removed from a partition during a wash step. The magnet may also create a pellet from a collection of magnetic particles. The magnet may create a particle pellet in less than 10 minutes. The magnet may create a particle pellet in less than 5 minutes. The particle pellet may comprise a particle with a biomolecule corona.

The automated apparatus may comprise a purification unit. The purification unit may comprise a plurality of partitions comprising an adsorbent or resin. The purification unit may comprise a solid-phase extraction array or plate. The solid-phase extraction array or plate may comprise a polar stationary phase material. The solid-phase extraction array or plate may comprise a non-polar stationary phase material. The solid-phase extraction array or plate may comprise a C18 stationary phase material (e.g., octadecyl group silica gel). The automated apparatus may comprises a unit with a conditioning solution for the purification unit (e.g., a conditioning solution for a solid-phase extraction material). The automated apparatus may comprise a unit with an elution solution for removing biomolecules from the purification unit.

In some embodiments, components are removed from a partition, except for the plurality of sensor elements and a population of analytes (e.g., proteins, metabolites, lipids, and the like) interacting with the plurality of sensor elements (i.e., a wash step). In some instances, the automated apparatus may perform a series of wash steps. A wash step may remove biomolecules that are not bound to the sensor elements within the partition. A wash step may desorb a subset of biomolecules bound to sensor elements within a partition. For example, a wash step may result in the desorption and removal of a subset of soft corona analytes, while leaving the majority of hard corona analytes bound to the sensor element.

In some aspects, the present disclosure provides an automated apparatus to identify proteins in a biological sample, the automated apparatus comprising: a sample preparation unit; a substrate comprising a plurality of channels; a plurality of pipettes; a plurality of solutions, a plurality of surfaces, and wherein the automated apparatus is configured to form a protein corona and digest the protein corona.

In some aspects, the present disclosure provides an automated apparatus to identify proteins in a biological sample, the automated apparatus comprising: a sample preparation unit; a substrate comprising a plurality of channels; a plurality of pipettes; a plurality of solutions, a plurality of nanoparticles, wherein the automated apparatus is configured to form a protein corona and digest the protein corona, and wherein at least one of the solutions is TE 150 mM KCl 0.05% CHAPS buffer.

In some embodiments, the sample preparation unit is configured to add the plurality of nanoparticles to the substrate with the plurality of pipettes. In some embodiments, wherein the sample preparation unit is configured to add the biological sample to the substrate with the plurality of pipettes. In some embodiments, the sample preparation unit is configured to incubate the plurality of nanoparticles and the biological sample to form the protein corona.

In some embodiments, the sample preparation unit is configured to separate the protein corona from the supernatant to form a protein corona pellet. In some embodiments, the sample preparation unit is configured to reconstitute the protein corona pellet with TE 150 mM KCl 0.05% CHAPS buffer.

In some embodiments, the automated apparatus further comprises a magnetic source. In some embodiments, the automated apparatus is configured for BCA, gel, or trypsin digestion of the protein corona.

In some embodiments, the automated apparatus is enclosed. In some embodiments, the automated apparatus is sterilized before use. In some embodiments, the automated apparatus is configured to a mass spectrometry. In some embodiments, the automated apparatus is temperature controlled.

### Kits

In one aspect, described herein is a kit for identifying biomolecules in a biological sample, wherein the kit may comprise one or more surfaces of the disclosure as described elsewhere herein. The kit may be used, in some embodiments, to perform the method of identifying proteins in a sample as disclosed herein.

A kit of the present disclosure may comprise one or more surfaces (e.g., particles) to interrogate a sample. In some embodiments, the kit comprises one or more surfaces (e.g., particles) as provided elsewhere herein. In some instances, the surfaces are capable of binding a plurality of proteins in a biological fluid (e.g. biofluid) to produce protein coronas.

In some embodiments, the protein coronas comprise a first protein from the biofluid. In some embodiments, the protein coronas comprise a second protein from the biofluid. In some embodiments, the second protein is present at a concentration greater than 6 magnitudes (e.g., 2, 3, 4, 5, 7, 8, 10 magnitudes) than the first protein in the biofluid.

The kit may be pre-packaged in discrete aliquots. In another example, the kit can comprise a plurality of different surface types (e.g., particles with different surface chemistries) that can be used to interrogate a sample. The plurality of particle types can be pre-packaged where each particle type of the plurality is packaged separately. Alternately, the plurality of particle types can be packaged together to contain combination of particle types in a single package. In some embodiments, the kit comprises two or more packages containing different particles, wherein at least one of the packages contains two or more different particles. The particles may, in some embodiments, be freeze dried and stored in sealed containers. The particles may, in some embodiments, be stored in a fluid, such as water with an appropriate preservative (e.g., sodium azide).

In some embodiments, the kit further comprises a denaturing agent. In some embodiments, the denaturing agent comprises at least one of sodium dodecyl sulfate, acetic acid, trichloroacetic acid, sulfosalicylic acid, sodium bicarbonate, ethanol, formaldehyde, glutaraldehyde, urea, guanidium chloride, lithium perchlorate, 2-mercaptoethanol, dithiothreitol, tris(2-carboxyethyl)phosphine (TCEP), or any combination thereof.

In some embodiments, the kit further comprises a reducing agent. In some embodiments, the reducing agent comprises TCEP, dithiothreitol, beta-mercaptoethanol, glutathione, cysteine, or any combination thereof.

In some embodiments, the kit further comprises an alkylating agent. In some instances, the alkylating agent is configured to alkylate proteins. In some embodiments, the alkylating agent comprises iodoacetamide, iodoacetic acid, acrylamide, chloroacetamide, or any combination thereof.

In some embodiments, the kit further comprises a digesting agent. In some instances the digesting agent is an enzymatic agent. In some instances, the digesting agent is configured to digest proteins. In some embodiments, the digesting agent comprises trypsin, lysin, serine protease, or any combination thereof. In some embodiments, the digesting agent comprises trypsin. In some embodiments, the digesting agent comprises trypsin and LysC.

In some embodiments, the kit further comprises a halting agent, configured to halt the digesting (e.g., enzymatic) agent.

In some embodiments, the kit further comprises a buffer. In some embodiments, the buffer comprises triethylammonium bicarbonate, tris(hydroxymethyl)aminomethane, citrate, Tris, phosphate, ethylenediaminetetraacetic acid, or any combination thereof. In some embodiments, the buffer may be configured to maintain a pH about 9 to about 10 when combined with a biofluid, such as plasma or serum.

In some embodiments, the kit further comprises a wash buffer. In some instances the wash buffer is a buffer described elsewhere herein. In some embodiments, the wash buffer is configured to remove unbound and loosely attached proteins from the protein corona.

In some embodiments, the kit further comprises an organic solvent.

In some embodiments, the kit further comprises a cysteine blocking reagent. In some embodiments, the cysteine blocking reagent comprises methyl methanethiosulfonate, iodoacetamide, N-ethylmaleimide, methylsulfonyl benzothiazole, or any combination thereof.

In some embodiments, the kit further comprises an adsorbent material. In some instances, the adsorbent material is configured for solid phase extraction of digested proteins obtained from the protein corona. In some embodiments, the kit further comprises an eluant for eluting biomolecules from the adsorbent material.

In some embodiments, a biological sample is processed using the commercially-available PROTEOGRAPH Assay Kit (v 1.2) or PROTEOGRAPH XT Assay Kit.

In some embodiments, the kit further comprises instructions to carry out any of the methods provided herein. In some embodiments, the kit further comprises instructions to incubate the biofluid with the particles at a temperature between 10° C. and 40° C. (e.g., between 0° C. and 80° C., 5° C. and 70° C., 10° C. and 50° C.). In some embodiments, the kit further comprises instructions to incubate the biofluid with the particles for about 15 to about 60 minutes (e.g., about 1 minute to about 90 minutes, about 5 minutes to about 60 minutes, about 10 minutes to about 60 minutes, about 15 minutes to about 45 minutes). In some embodiments, the kit further comprises instructions to obtain the protein coronas on the first particles and the second particle sin the same sample at the same time.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Performance Data

Provided herein Table 6 is performance data for nanoparticles containing the surfaces disclosed in Table 4 and Table 5. Briefly, the nanoparticles were incubated with plasma samples at 37° C. for 1 hour to form protein coronas and then washed three times. The proteins were then lysed, alkylated, and digested before analysis using liquid chromatography tandem mass spectrometry

TABLE 6

| | PG | PG | Peptide | Peptide | | | | | | JI: NP-1 \| |
|---|---|---|---|---|---|---|---|---|---|---|
| | Counts | Counts | Counts | Counts | JI: | JI: | JI: | JI: | JI: | NP-2 \| NP-3 \| |
| NP | Median | StdDev | Median | StdDev | NP-1 | NP-2 | NP-3 | NP-4 | NP-5 | NP-4 \| NP-5 |
| NP-1 | 718 | 46 | 4252 | 218 | 0.76 | 0.43 | 0.59 | 0.58 | 0.47 | 0.45 |
| NP-2 | 1066 | 44 | 5772 | 259 | 0.43 | 0.8 | 0.43 | 0.43 | 0.61 | 0.64 |
| NP-3 | 754 | 5 | 4261 | 42 | 0.59 | 0.43 | 0.78 | 0.58 | 0.44 | 0.48 |
| NP-4 | 791 | 53 | 4548 | 276 | 0.58 | 0.43 | 0.58 | 0.76 | 0.47 | 0.5 |
| NP-5 | 968 | 34 | 5489 | 55 | 0.47 | 0.61 | 0.44 | 0.47 | 0.79 | 0.6 |
| 400 | 839 | 30 | 4190 | 205 | 0.39 | 0.52 | 0.37 | 0.35 | 0.48 | 0.44 |
| 401 | 810 | 17 | 4201 | 69 | 0.39 | 0.56 | 0.37 | 0.36 | 0.5 | 0.44 |
| 402 | 851 | 8 | 4164 | 62 | 0.41 | 0.48 | 0.4 | 0.38 | 0.45 | 0.42 |
| 403 | 734 | 13 | 3792 | 26 | 0.45 | 0.45 | 0.44 | 0.42 | 0.42 | 0.39 |
| 404 | 948 | 26 | 4567 | 138 | 0.41 | 0.5 | 0.38 | 0.38 | 0.47 | 0.46 |
| 405 | 889 | 7 | 4414 | 23 | 0.4 | 0.5 | 0.38 | 0.37 | 0.46 | 0.44 |
| 413 | 1007 | 20 | 5774 | 119 | 0.54 | 0.45 | 0.55 | 0.56 | 0.44 | 0.52 |
| 414 | 928 | 33 | 5032 | 93 | 0.54 | 0.44 | 0.54 | 0.52 | 0.42 | 0.49 |
| 415 | 980 | 17 | 5510 | 72 | 0.56 | 0.44 | 0.55 | 0.55 | 0.44 | 0.51 |
| 416 | 939 | 43 | 5103 | 149 | 0.52 | 0.44 | 0.53 | 0.5 | 0.42 | 0.47 |
| 500 | 774 | 14 | 3784 | 72 | 0.42 | 0.47 | 0.4 | 0.38 | 0.44 | 0.4 |
| 407 | 816 | 9 | 3937 | 63 | 0.41 | 0.46 | 0.4 | 0.38 | 0.44 | 0.41 |
| 408 | 789 | 41 | 3936 | 123 | 0.4 | 0.49 | 0.38 | 0.36 | 0.45 | 0.41 |
| 409 | 806 | 10 | 3989 | 66 | 0.39 | 0.53 | 0.37 | 0.36 | 0.49 | 0.43 |
| 410 | 682 | 27 | 3586 | 66 | 0.45 | 0.44 | 0.44 | 0.42 | 0.42 | 0.37 |
| 411 | 760 | 13 | 4006 | 23 | 0.39 | 0.53 | 0.38 | 0.36 | 0.48 | 0.42 |
| 412 | 797 | 33 | 4105 | 125 | 0.4 | 0.51 | 0.39 | 0.37 | 0.47 | 0.42 |
| 501 | 429 | 17 | 2784 | 65 | 0.38 | 0.3 | 0.42 | 0.42 | 0.34 | 0.27 |
| 502 | 938 | 11 | 4892 | 32 | 0.43 | 0.44 | 0.41 | 0.39 | 0.44 | 0.44 |
| 503 | 948 | 18 | 5289 | 37 | 0.37 | 0.57 | 0.35 | 0.35 | 0.55 | 0.49 |
| 504 | 856 | 23 | 4765 | 63 | 0.59 | 0.44 | 0.57 | 0.66 | 0.45 | 0.5 |
| 433 | 845 | 8 | 4247 | 82 | 0.41 | 0.42 | 0.39 | 0.37 | 0.42 | 0.4 |
| 505 | 671 | 10 | 4104 | 47 | 0.52 | 0.4 | 0.53 | 0.62 | 0.42 | 0.41 |
| 506 | 863 | 36 | 4756 | 168 | 0.56 | 0.43 | 0.55 | 0.52 | 0.42 | 0.47 |
| 434 | 749 | 19 | 4123 | 60 | 0.57 | 0.43 | 0.55 | 0.63 | 0.46 | 0.45 |
| 424 | 1282 | 37 | 7485 | 215 | 0.41 | 0.46 | 0.41 | 0.42 | 0.47 | 0.53 |
| 507 | 946 | 13 | 4898 | 23 | 0.53 | 0.5 | 0.45 | 0.5 | 0.54 | 0.51 |
| 508 | 1060 | 36 | 5789 | 195 | 0.55 | 0.48 | 0.48 | 0.52 | 0.49 | 0.54 |
| 422 | 917 | 2 | 4778 | 50 | 0.43 | 0.46 | 0.4 | 0.39 | 0.44 | 0.44 |
| 435 | 906 | 15 | 4472 | 79 | 0.44 | 0.44 | 0.42 | 0.41 | 0.44 | 0.43 |
| 440 | 1166 | 73 | 6398 | 369 | 0.41 | 0.47 | 0.38 | 0.4 | 0.48 | 0.5 |
| 425 | 929 | 15 | 5349 | 11 | 0.57 | 0.45 | 0.55 | 0.65 | 0.46 | 0.52 |
| 426 | 904 | 14 | 5000 | 138 | 0.56 | 0.42 | 0.58 | 0.56 | 0.42 | 0.49 |
| 419 | 890 | 17 | 4520 | 132 | 0.41 | 0.46 | 0.38 | 0.37 | 0.45 | 0.43 |
| 420 | 1200 | 9 | 6785 | 55 | 0.4 | 0.53 | 0.36 | 0.4 | 0.53 | 0.54 |
| 421 | 1086 | 28 | 5775 | 80 | 0.37 | 0.53 | 0.34 | 0.35 | 0.53 | 0.51 |
| 443 | 846 | 10 | 4292 | 56 | 0.43 | 0.48 | 0.42 | 0.41 | 0.44 | 0.42 |
| 417 | 914 | 52 | 4759 | 188 | 0.51 | 0.47 | 0.48 | 0.47 | 0.44 | 0.46 |
| 444 | 807 | 23 | 4121 | 117 | 0.43 | 0.46 | 0.42 | 0.41 | 0.43 | 0.4 |
| 445 | 848 | 19 | 4260 | 55 | 0.42 | 0.49 | 0.41 | 0.39 | 0.44 | 0.42 |
| 446 | 809 | 17 | 4138 | 90 | 0.44 | 0.47 | 0.43 | 0.42 | 0.43 | 0.42 |
| 447 | 748 | 6 | 3867 | 32 | 0.43 | 0.44 | 0.42 | 0.4 | 0.41 | 0.38 |
| 418 | 1065 | 2 | 5671 | 26 | 0.52 | 0.48 | 0.5 | 0.53 | 0.46 | 0.53 |
| 448 | 790 | 23 | 4124 | 26 | 0.45 | 0.46 | 0.43 | 0.43 | 0.46 | 0.41 |
| 423 | 1251 | 61 | 7037 | 288 | 0.39 | 0.5 | 0.36 | 0.39 | 0.5 | 0.53 |
| 436 | 771 | 28 | 3988 | 78 | 0.48 | 0.47 | 0.45 | 0.45 | 0.43 | 0.42 |
| 437 | 790 | 51 | 4228 | 213 | 0.51 | 0.46 | 0.5 | 0.48 | 0.44 | 0.43 |
| 438 | 1063 | 38 | 5720 | 303 | 0.42 | 0.46 | 0.39 | 0.42 | 0.46 | 0.47 |
| 427 | 1059 | 21 | 5327 | 29 | 0.4 | 0.48 | 0.37 | 0.38 | 0.48 | 0.48 |
| 428 | 968 | 11 | 5379 | 20 | 0.54 | 0.45 | 0.55 | 0.55 | 0.45 | 0.51 |
| 439 | 906 | 29 | 5078 | 118 | 0.4 | 0.6 | 0.38 | 0.38 | 0.56 | 0.5 |
| 449 | 1124 | 8 | 5974 | 39 | 0.42 | 0.47 | 0.39 | 0.4 | 0.47 | 0.49 |
| 451 | 780 | 12 | 4001 | 65 | 0.41 | 0.49 | 0.39 | 0.36 | 0.45 | 0.41 |
| 429 | 820 | 32 | 4277 | 110 | 0.4 | 0.5 | 0.37 | 0.35 | 0.48 | 0.43 |
| 430 | 793 | 23 | 4197 | 95 | 0.39 | 0.51 | 0.37 | 0.35 | 0.48 | 0.43 |
| 431 | 959 | 36 | 5240 | 218 | 0.56 | 0.43 | 0.55 | 0.57 | 0.43 | 0.5 |
| 432 | 910 | 14 | 5001 | 97 | 0.56 | 0.42 | 0.54 | 0.56 | 0.42 | 0.49 |
| 450 | 1023 | 20 | 5583 | 137 | 0.55 | 0.46 | 0.52 | 0.57 | 0.46 | 0.53 |
| 452 | 1131 | 11 | 6594 | 70 | 0.37 | 0.57 | 0.35 | 0.37 | 0.56 | 0.54 |

NP-1 through NP-5 are nanoparticles found in commercially available Proteograph™ V1.2 kit. The Jaccard Index (JI) is a measure of similarity between in the protein groups (PG) identified between different nanoparticles.

### Example 2: Synthesis of P(GMA-Co-DVB)-Coated Particle

In a 4-neck round bottom flask, vinyl-containing silica coated nanoparticles (2 g) are added and dispersed in acetonitrile (400 mL). The mixture of the round bottom flask is mixed or sonicated at about 200 rpm for 20 minutes. In a separate vessel, purified glycidyl methacrylate (GMA) (3.000 g) divinyl benzene (DVB) (3.000 g), and acetonitrile (5 mL) are combined. The round bottom flask is cooled to 10 degrees Celsius while stirring. Using a plastic syringe, the mixture of GMA and DVB is added to the round bottom flask under an inert atmosphere of nitrogen. In a separate vial, azobisisobutyronitrile (AIBN) (450 mg) is dissolved in acetonitrile (4 mL). The round bottom flask is heated to 80 degrees Celsius, and once the flask reaches temperature, the AIBN solution is injected into the flask. The reaction suspension reacts at 80 degrees Celsius for 60 minutes. The reaction is monitored every 30 minutes until the particles have formed a size of about 350 nm and PDI of <0.2. The reaction is quenched by adding benzoquinone (0.108 g) in acetonitrile (5 mL). Once the reaction is complete, the flask is removed from the heat and the particles are purified using THF.

### Example 3: Synthesis of Surface 400 (P(GMA-Co-DVB)-EDA Particle

In a 4-neck round bottom flask, P(GMA-co-DVB)-coated particle (see Example 2) (1.10 g) and DMF (220 mL) are added. The mixture is mixed or sonicated at about 200 rpm for 20 minutes. The round bottom flask is cooled to 10 degrees Celsius while stirring under inert atmosphere, after which the flask is heated to 80 degrees Celsius. When the flask has reached 80 degrees Celsius, the ethylene diamine (6.86 g) is injected into the flask using a syringe. The mixture is reacted for 16 hours. Once the reaction is complete, the flask is removed from the heat and the particles are purified using DMF

### Example 4: Synthesis of Surface 414 (P(GMA-Co-DVB)-EDA-PA Particle

In a 4-neck round bottom flask, P(GMA-co-DVB)-EDA-PA particles (1.0 g) (see Example 3), N,N-dimethylformamide (DMF) (190 mL), and triethylamine (TEA) (1.44 g) are added. The mixture is mixed or sonicated at about 200 rpm for 20 minutes. In a separate vessel, phthalic anhydride (1.50 g) in DMF (10 mL) are combined The round bottom flask is cooled to 10 degrees Celsius while stirring under inert atmosphere, after which the flask is heated to 80 degrees Celsius. When the flask has reached 60 degrees Celsius, the pthalic anhydride solution is injected into the flask using a syringe. The mixture is reacted for 16 hours. Once the reaction is complete, the flask is removed from the heat and the particles are purified using ethanol and DMF

### Example 5: Synthesis of Peptide Modified Surface 428-MaganinII

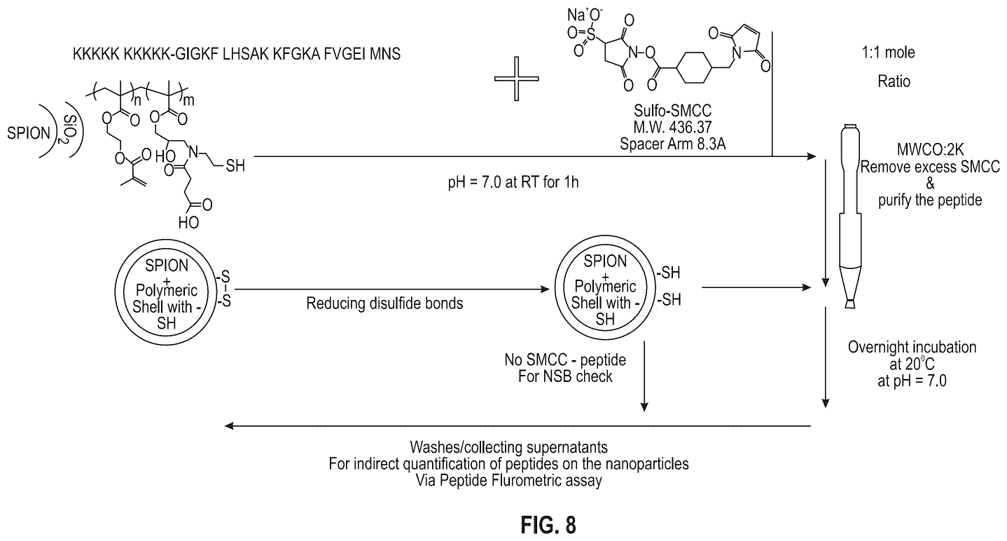

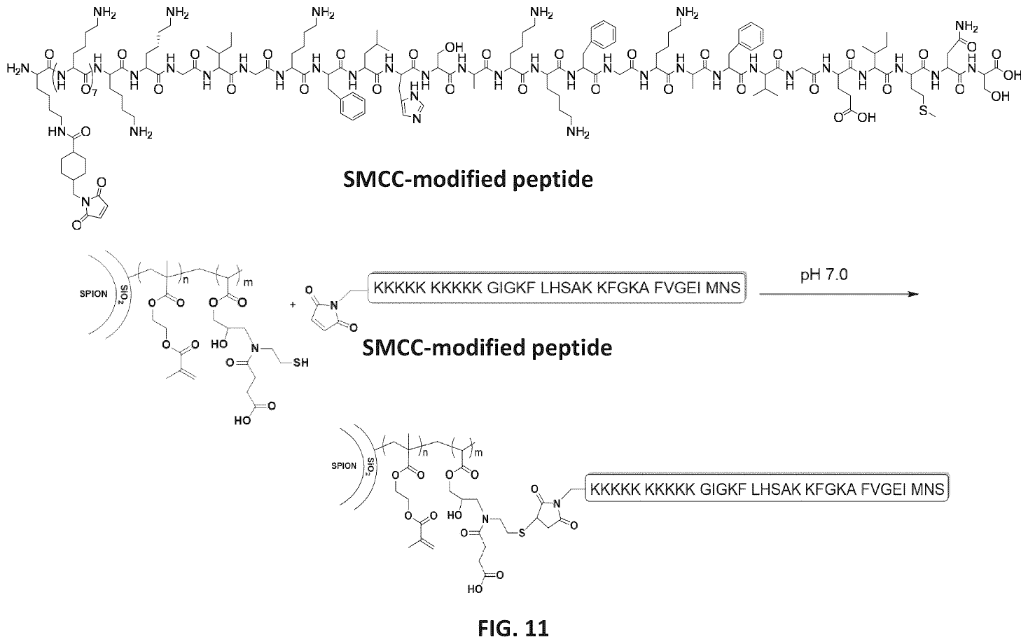

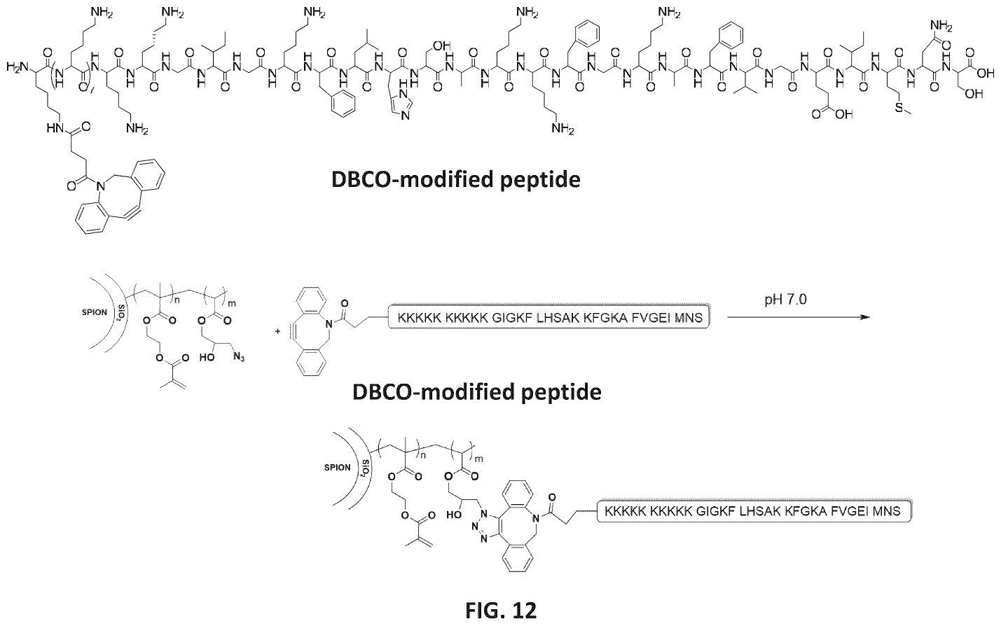

Surface 428 can be modified with peptides based on the scheme shown in FIG. 8 and FIG. 11. FIG. 12 shows a similar reaction scheme using DBCO as a functionalizing agent. Succinimidyl-4-(N-maleimidomethyl)cyclohexane-1-carboxylate (SMCC) and the 10K peptide-MaganinII (KKKKK KKKKK-GIGKF LHSAK KFGKA FVGEI MNS (SEQ ID NO: 3), MW-3748.62 Da), are thawed prior to use. 1 mM 10K-peptide (180 μL, prepared in 25 mM PB buffer at pH 7.0) is incubated with Sulfo-SMCC (5 mM, 36 μL, prepared in 25 mM PB buffer at pH 7.0) at 20° C. for 1 hour in a shaker at 1500 RPM. During incubation, 1 mL of a 1 nM solution of Surface 428 is added with tris(2-carboxyethyl)phosphine (TCEP) (18 μL, 50 nM) at room temperature for 30 minutes in DI water. The surfaces are washed with 1 mL DI water twice after 20 minute magnetic separation each time and then redistributed in 900 μL of pH 7 PB buffer. The peptide solution is filtered with 2K cutoff filters (Sartorius 2, 2K MWCO) by adding 284 μL PB buffer (pH 7). This is done twice by spinning down at 3354 rcf for 22 minutes at 4° C. resulting in the elution of about 400 μL of solution with about 100 μLL remaining in the filter. The concentration solution is collected by spinning the columns upside down for 4 minutes at the same speed and temperature in addition to the eluted peptide portion for quantification with a peptide fluorometric assay if anything was lot from the initial peptide count. Immediately, the concentrated peptide is added to the surface solution and the peptide-maleimide and surface-thiol solution is incubated overnight at 20° C. in this PB buffer (pH=7) overnight while shaking in a shaker at 1500 RPM. After incubation, the supernatant is collected after magnetic separation with a magnetic stand for 30 minutes. This supernatant can be used for peptide quantification. Then, the beads are washed with 1 mL of PB buffer (pH=7) three times. The second and third supernatants are collected after 30 minutes of magnetic separation for the peptide quantification for the second and third washes. The beads are washed 1 time with water and dispersed in DI water (1 mL) or are lyophilized for characterization via TGA, DLS, and zeta-potential

### Example 6: Characterization of Peptide Modified Surfaces

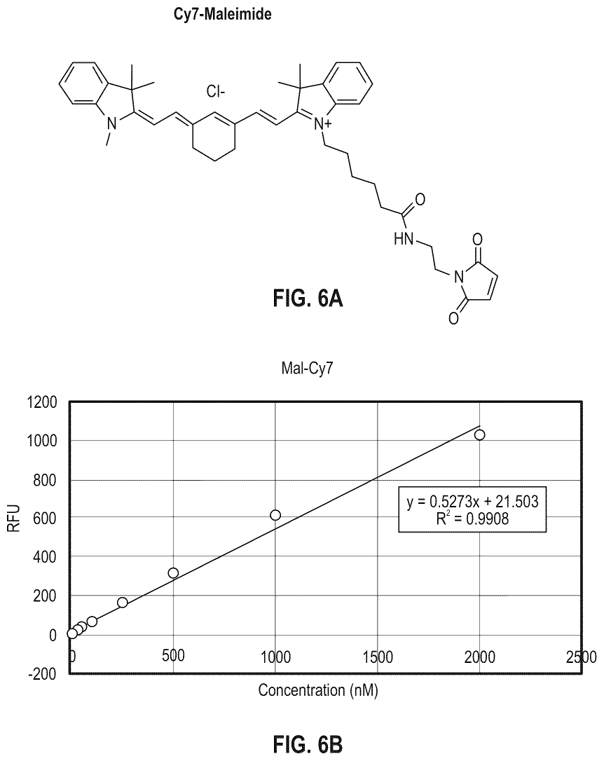

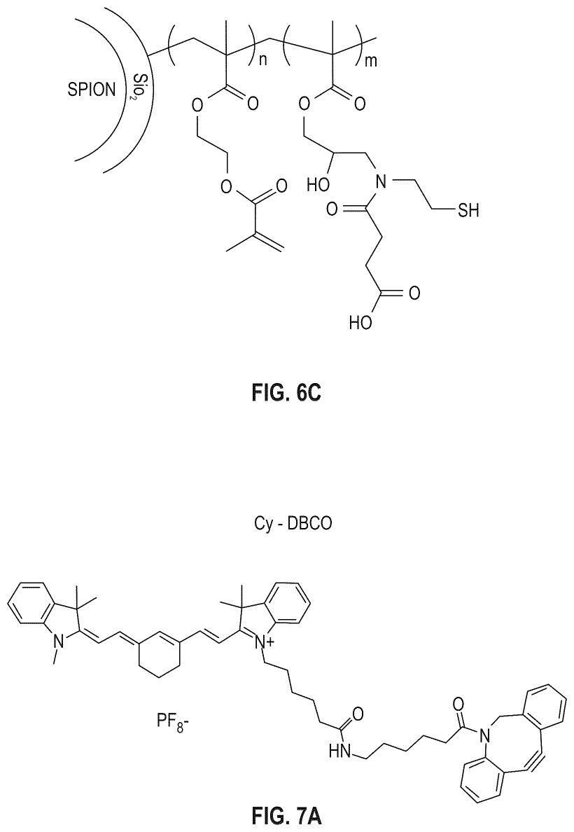

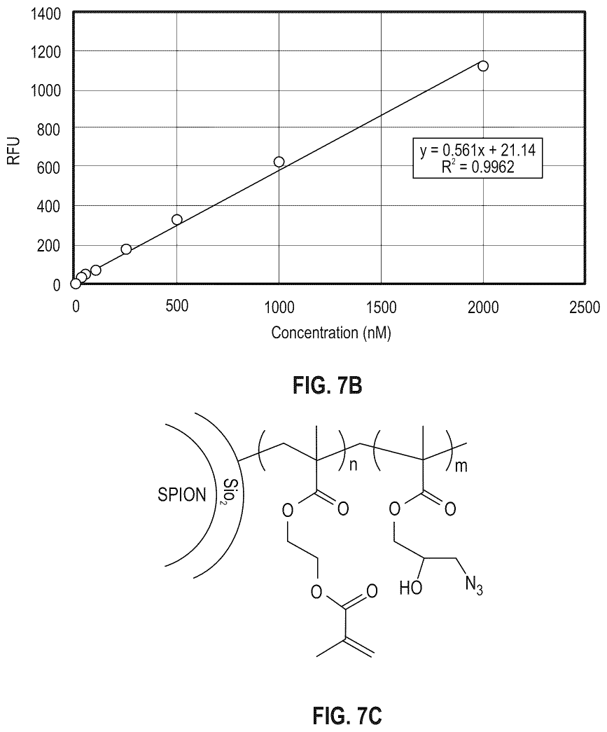

Provided herein Table 7 is thiol and azide quantification data for Surface 428 (FIG. 6C) and Surface 422 (FIG. 7C). Briefly, Surface 428 is coupled with Cy7-maleimide (FIG. 6A), and Surface 422 is coupled with Cy7-DBCO (FIG. 7A), wherein Cy7 are fluorescent handles allowing for analytical determination of reactive thiol and azide groups on the respective surfaces. The surfaces were incubated with the respective Cy7 handles fluorescence spectroscopy was used to quantify the thiol groups (FIG. 6B) and azide groups (FIG. 7B).

TABLE 7

| | Average | conc. Excess Cy7 | #Functional Groups |
|---|---|---|---|
| Reaction | RFU | (nM) | per NP |
| Surface 428-Cy7 | 476 | 862 | 93751 Thiols |
| Surface 422-Cy7 | 876 | 1524 | 27593 Azides |

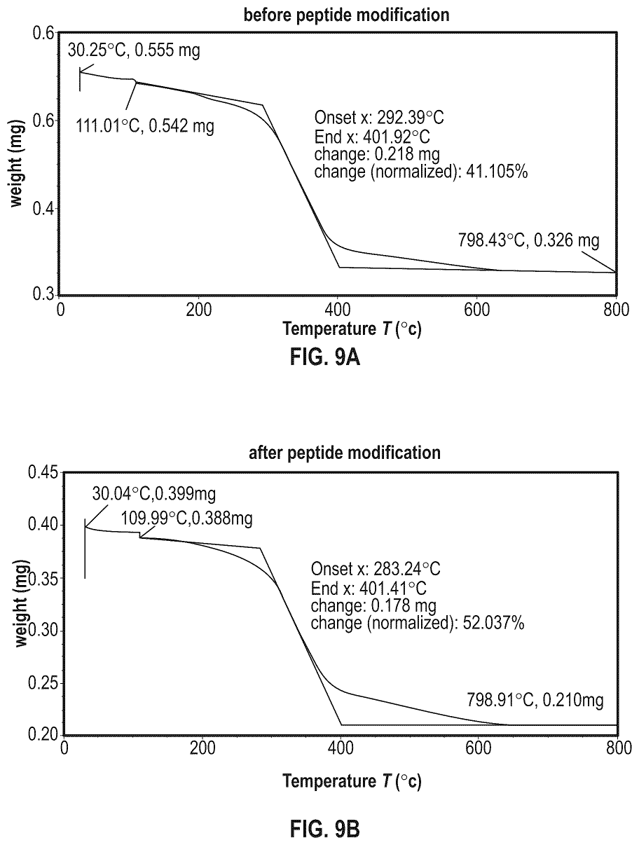

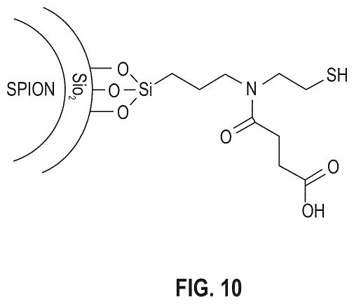

Surface 428 is modified as described in Example 5 with 10K peptide-MaganinII and characterized as seen in Table 8. Thermogravimetric analysis of the surfaces before modification (FIG. 9A) and after modification (FIG. 9B) indicate a 10% mass gain after covalent conjugation.

TABLE 8

| | DLS | Zeta | |
|---|---|---|---|
| Reaction | (nm) | Potential | Modification |
| Surface 428 | 412 ± 4 | −38 ± 1.5 | Thiol + COOH |
| Surface 428 + peptide | 438 ± 4 | 11 ± 0.5 | SMCC-10K- |
| | | | GIGKFLHSAKKFGKAFVGEIMNS |
| | | | (SEQ ID NO: 4) |

### Example 7: Protein Capture by Peptide Modified Surfaces

Provided herein are proteins that are captured by surfaces modified with peptides described herein. Briefly, surfaces are modified with the 10K-peptide-MaganinII. Pooled plasma samples containing proteins and the modified surfaces are combined and the surfaces are isolated and captured proteins are analyzed by liquid chromatography tandem mass spectrometry. The nanoparticle prepared in Example 5 was used and selected proteins that include those in Table 9. These proteins were captured by the peptide-modified surface, but not surfaces without peptide modification.

TABLE 9

| Uniprot | Median |
|---|---|
| ID | Intensity |
| O75369-9 | 4.52 |
| P10451-5 | 4.17 |
| O00533; O00533-2 | 4.07 |
| P39687 | 4.07 |
| P31431; P31431-2 | 3.84 |
| P09603 | 3.84 |
| A0A075B6K0 | 3.81 |
| Q9P281 | 4.04 |
| P19397 | 3.76 |
| P16112; P16112-2 | 3.51 |
| O14775-2; 014775-3 | 3.78 |
| O14594 | 3.63 |
| Q93008; Q93008-1 | 3.78 |
| P18827 | 3.37 |
| P19012-2 | 3.63 |
| P04229; 913760; P13761; P79483; | 3.64 |
| Q29974; Q30134; Q9TQE0 | |
| A0A0C4DGL1 | 3.62 |
| P50897; P50897-2 | 3.86 |
| P08779 | 3.60 |
| Q8TB24 | 3.53 |
| Q96RD9; Q96RD9-3 | 3.58 |
| P63162-2 | 3.41 |
| P02538; P48668 | 3.57 |
| A0A0C4DH33 | 3.89 |
| Q01105; Q01105-2; Q01105-3; Q01105-4 | 3.55 |
| F8W7Q4 | 3.55 |
| P31151 | 3.62 |
| E7EQ47 | 3.50 |
| P13591-3 | 3.51 |
| P54105 | 3.61 |
| Q8NBM8 | 3.45 |

### EMBODIMENTS

The following are exemplary embodiments of the disclosure herein:
1. A macromolecule comprising a recurring unit of Formula (II):

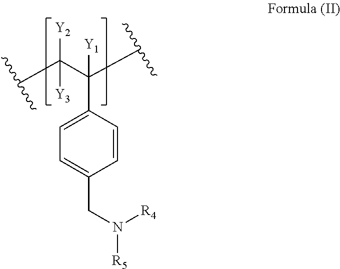

wherein each of Y₁, Y₂, and Y₃ is independently selected from hydrogen or C₁-C₆ alkyl;
   R₄ is hydrogen or C₁-C₆ thiol; and
   R₅ is succinate or optionally substituted —C₁-C₆ disulfide.
2. The macromolecule of embodiment 1, wherein Y₁ is C₁-C₃ alkyl
3. The macromolecule of embodiment 1 or 2, wherein Y₁ is C₁ alkyl
4. The macromolecule of any one of embodiments 1-3, wherein each of Y₂ and Y₃ is hydrogen
5. The macromolecule of any one of embodiments 1-4, wherein R₄ is hydrogen and R₅ is optionally substituted —C₁-C₆ disulfide.
6. The macromolecule of any one of embodiments 1-5, wherein R₅ is optionally substituted di-C₁-C₆ alkyl disulfide.
7. The macromolecule of any one of embodiments 1-6, wherein R₅ is —CH₂CH₂—S—S—CH₂CH₂NH₂.
8. The macromolecule of any one of embodiments 1-7, wherein R₄ is C₁-C₆ thiol and R₅ is succinate.
9. The macromolecule of any one of embodiments 1-8, wherein R₄ is C₁-C₃ thiol.
10. The macromolecule of any one of embodiments 1-9, wherein R₄ is —(CH₂)₂SH.
11. A macromolecule comprising a recurring unit of Formula (III):

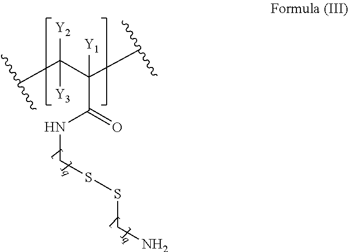

wherein each of Y₁, Y₂, and Y₃ is independently selected from hydrogen or C₁-C₆ alkyl; and
   q is an integer between 1 and 6.
12. The macromolecule of embodiment 11, wherein Y₁ is C₁-C₃ alkyl
13. The macromolecule of embodiment 11 or 12, wherein Y₁ is C₁ alkyl.
14. The macromolecule of any one of embodiments 11-13, wherein each of Y₂ and Y₃ is hydrogen
15. The macromolecule of any one of embodiments 11-14, wherein q is 2 or 3.
16. The macromolecule of any one of embodiments 11-15, wherein q is 2.
17. The macromolecule of any one of embodiments 1 to 16, wherein the recurring unit of Formula (I), (II), and (III) is selected from Table 1.
18. A macromolecule comprising recurring units of a first component and a second component,
   wherein the first component comprises a structure of Component (A) and the second component comprises a structure of Component (B):

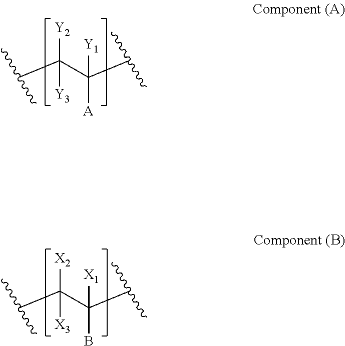

wherein each of Y₁, Y₂, and Y₃ is independently selected from hydrogen or C₁-C₆ alkyl;
   each of X₁, X₂, and X₃ is independently selected from hydrogen or C₁-C₆ alkyl;
      A is

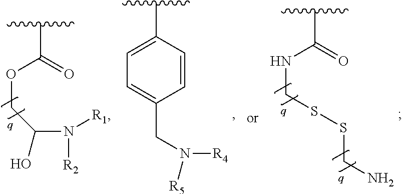

R₁ is hydrogen, nitrogen, optionally substituted succinate, C₁-C₆ alkyl sulfone, phthalate,
   R₂ is nitrogen, C₁-C₁₂ amine, C₁-C₆ hydroxy, optionally substituted aryl, optionally substituted heteroaryl, optionally substituted C₃-C₆ heterocycloalkyl, two or more fused 3-6 member rings; optionally substituted C₃-C₆ dicyloalkyl methane, C₁-C₆ alkyl guanidine, C₁-C₆ ether, optionally substituted C₁-C₆ disulfide, C₁-C₆ thiol, optionally substituted succinate, optionally substituted C₁-C₆ alkylamine, C₁-C₆ alkyl acetamide, C₁-C₆ aminophthalate, a boronic acid, or a monosaccharide;
      R₄ is hydrogen or C₁-C₆ thiol;
      R₅ is succinate or optionally substituted —C₁-C₆ disulfide;
      B is

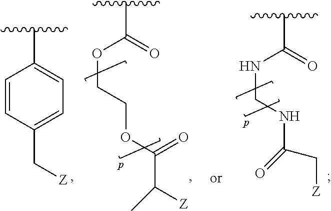

Z is a unit of Monomer (A) or Monomer (B);
   q is an integer between 1 and 6; and
      p is an integer between 1 and 20.
19. The macromolecule of embodiment 18, wherein B is

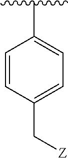

### and A is

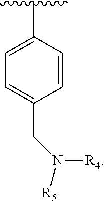

20. The macromolecule of embodiment 18 or 19, wherein R₄ is hydrogen and R₅ is optionally substituted —C₁-C₆ disulfide.
21. The macromolecule of embodiment 18 or 19, wherein R₅ is optionally substituted di-C₁-C₆ alkyl disulfide.
22. The macromolecule of embodiment 21, wherein R₅ is —CH₂CH₂—S—S—CH₂CH₂NH₂.

23. The macromolecule of embodiment 18, wherein B is

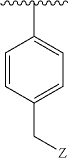

### and A is

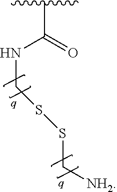

24. The macromolecule of embodiment 18, wherein B is

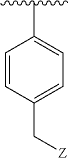

### and A is

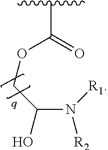

25. The macromolecule of embodiment 24, wherein R₁ is C₂-C₁₂ alkenyl succinate and R₂ is substituted succinate.
26. The macromolecule of embodiment 25, wherein R₁ is C₈ alkenyl succinate and R₂ is C₈ alkenyl ethylaminosuccinate.
27. The macromolecule of embodiment 25, wherein R₁ is C₁-C₆ alkyl sulfone and R₂ is optionally substituted C₁-C₆ alkylamine.
28. The macromolecule of embodiment 27, wherein R₁ is —(CH₂)₃SOOOH and R₂ is —(CH₂)₁₋₆N(CH₃)₂(CH₂CH₂CH₂SOOOH) or —(CH₂)₁₋₆N(CH₂CH₂CH₂SOOOH)₂.
29. The macromolecule of embodiment 28, wherein R₂ is —(CH₂)₂N(CH₃)₂(CH₂CH₂CH₂SOOOH).
30. The macromolecule of embodiment 18, wherein R₁ is phthalate and R₂ is C₁-C₆ aminophthalate.
31. The macromolecule of embodiment 30, wherein R₂ is C₂-C₆ aminophthalate.
32. The macromolecule of embodiment 31, wherein R₂ is C₂ aminophthalate.
33. The macromolecule of embodiment 31, wherein R₂ is C₆ aminophthalate.
34. The macromolecule of embodiment 18, wherein R₁ is succinate and R₂ is substituted succinate or optionally substituted aryl.
35. The macromolecule of embodiment 34, wherein R₂ is —(CH₂)₁₋₁₂NH(C═O)CH₂CH₂COOH.
36. The macromolecule of embodiment 35, wherein R₂ is —(CH₂)₁₋₃NH(C═O)CH₂CH₂COOH.
37. The macromolecule of embodiment 36, wherein R₂ is —(CH₂)₂NH(C═O)CH₂CH₂COOH.
38. The macromolecule of embodiment 34, wherein R₂ is optionally substituted aryl.
39. The macromolecule of embodiment 38, wherein R₂ is 2-flourotoluene.
40. The macromolecule of embodiment 18, wherein R₁ is hydrogen and R₂ is C₁-C₁₂ amine, optionally substituted C₁-C₆ alkylamine, C₁-C₆ acetamide, optionally substituted heterocyclalkyl, optionally substituted heteroaryl, a monosaccharide, two or more fused 3-6 member rings, optionally substituted aryl, or C₁-C₆ hydroxy.
41. The macromolecule of embodiment 40, wherein R₂ is C₁₋₁₂ amine.
42. The macromolecule of embodiment 41, wherein R₂ is C₂ amine.
43. The macromolecule of embodiment 42, wherein R₂ is C₆ amine.
44. The macromolecule of embodiment 43, wherein R₂ is optionally substituted C₁-C₆ alkylamine.
45. The macromolecule of embodiment 44, wherein R₂ is —(CH₂)₁₋₃ dimethylamine.
46. The macromolecule of embodiment 45, wherein R₂ is —(CH₂)₂ dimethylamine.
47. The macromolecule of embodiment 40, wherein R₂ is C₁-C₆ acetamide.
48. The macromolecule of embodiment 47, wherein R₂ is —(CH₂)₂ acetamide.
49. The macromolecule of embodiment 40, wherein R₂ is optionally substituted heterocyclalkyl.
50. The macromolecule of embodiment 49, wherein R₂ is —(CH₂)₁₋₆ pyrrolidine.
51. The macromolecule of embodiment 50, wherein R₂ is —(CH₂)₂ pyrrolidine.
52. The macromolecule of embodiment 40, wherein R₂ is optionally substituted heteroaryl.
53. The macromolecule of embodiment 52, wherein R₂ is —(CH₂)₁₋₆ imidazole.
54. The macromolecule of embodiment 53, wherein R₂ is —(CH₂)₃ imidazole.
55. The macromolecule of embodiment 52, wherein R₂ is —(CH₂)₁₋₆ pyridine.
56. The macromolecule of embodiment 55, wherein R₂—(CH₂)pyridine.
57. The macromolecule of embodiment 40, wherein R₂ is a monosaccharaide.
58. The macromolecule of embodiment 57, wherein R₂ is glucose.
59. The macromolecule of embodiment 58, wherein R₂ is D-glucose.
60. The macromolecule of embodiment 40, wherein R₂ is two or more fused 3-6 member rings.
61. The macromolecule of embodiment 60, wherein R₂ is three 6 member rings.
62. The macromolecule of embodiment 40, wherein R₂ is optionally substituted aryl.
63. The macromolecule of embodiment 62, wherein R₂ is halogenated toluene
64. The macromolecule of embodiment 63, wherein R₂ is 2-flourotoluene.
65. The macromolecule of embodiment 40, wherein R₂ is C₁-C₆ hydroxy.
66. The macromolecule of embodiment 65, wherein R₂ is C₂-C₆ hydroxy.
67. The macromolecule of embodiment 66, wherein R₂ is —(CH₂)₂OH.
68. The macromolecule of embodiment 67, wherein R₂ is —(CH₂)₆OH.
69. The macromolecule of any one of embodiments 18-68, wherein the macromolecule further comprises an second structure of Component (A), wherein a first structure of Component (A) and the second structure of Component (A) are different.
70. The macromolecule of embodiment 69, wherein in the first structure of Component (A), R₁ is hydrogen and R₂ is C₂ alkylamine and wherein the second structure of Component (A), R₁ is hydrogen and R₂ is C₁-C₆ hydroxy, optionally substituted aryl, optionally substituted heteroaryl, two or more fused 5 to 6 member rings, or a monosaccharide.
71. The macromolecule of embodiment 70, wherein in the second structure of Component (A), R₂ is C₁-C₆ hydroxy
72. The macromolecule of embodiment 71, wherein R₂ is C₂ alkylhydroxy.
73. The macromolecule of embodiment 70, wherein in the second structure of Component (A), R₂ is optionally substituted aryl
74. The macromolecule of embodiment 73, wherein R₂ is 2-fluorotoluene.
75. The macromolecule of embodiment 70, wherein in the second structure of Component (A), R₂ is optionally substituted heteroaryl
76. The macromolecule of embodiment 75, Wherein R₂ is 1-propylimidazole.
77. The macromolecule of embodiment 70, wherein in the second structure of Component (A), R₂ is two or more fused 5 to 6 member rings.
78. The macromolecule of embodiment 77, wherein R₂ is three fused 6-member rings.
79. The macromolecule of embodiment 70, wherein in the second structure of Component (A), R₂ is a monosaccharide.
80. The macromolecule of embodiment 79, wherein R₂ is d-glucose.
81. The macromolecule of embodiment 18, wherein B is

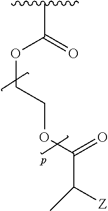

### and A is

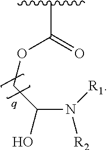

82. The macromolecule of embodiment 81, wherein R₁ is hydrogen and R₂ is selected from C₁-C₁₂ amine, optionally substituted C₃-C₆ dicyloalkyl methane, optionally substituted aryl, optionally substituted heteroaryl, optionally substituted C₃-C₆ heterocycloalkyl, C₁-C₆ hydroxy, C₁-C₆ ether, optionally substituted —C₁-C₆ disulfide, optionally substituted succinate, optionally substituted C₁-C₆ alkylamine, C₁-C₆ alkyl acetamide, a boronic acid, or C₁-C₆ alkyl guanidine.
83. The macromolecule of embodiment 82, wherein R₂ is optionally substituted dicyclohexyl methane.
84. The macromolecule of embodiment 83, wherein R₂ is amino dicyclohexylmethane.
85. The macromolecule of embodiment 82, wherein R₂ is optionally substituted aryl.
86. The macromolecule of embodiment 85, wherein R₂ is halogenated toluene
87. The macromolecule of embodiment 86, wherein R₂ is 2-flourotoluene.
88. The macromolecule of embodiment 82, wherein R₂ is a boronic acid.
89. The macromolecule of embodiment 88, wherein R₂ is phenylboronic acid.
90. The macromolecule of embodiment 82, wherein R₂ is C₁-C₆ hydroxy.
91. The macromolecule of embodiment 90, wherein R₂ is —(CH₂)₂OH.
92. The macromolecule of embodiment 82, wherein R₂ is C₁-C₆ ether.
93. The macromolecule of embodiment 92, wherein R₂ is —CH₂OCH₃, —CH₂CH₂OCH₃, —CH₂CH₂OCH₂CH₃, or —CH₂OCH₂CH₃.
94. The macromolecule of embodiment 93, wherein R₂ is —CH₂CH₂OCH₃.
95. The macromolecule of embodiment 82, wherein R₂ is optionally substituted di-C₁-C₆ alkyl disulfide.
96. The macromolecule of embodiment 95, wherein R₂ is —CH₂CH₂—S—S—CH₂CH₂NH₂.
97. The macromolecule of embodiment 82, wherein R₂ is C₁-C₆ thiol.
98. The macromolecule of embodiment 97, wherein R₂ is —(CH₂)₂SH.
99. The macromolecule of embodiment 82, wherein R₂ is optionally substituted succinate.
100. The macromolecule of embodiment 99, wherein R₂ is —(CH₂)₁₋₆NH(C═O)CH₂CH₂COOH.
101. The macromolecule of embodiment 100, wherein R₂ is —(CH₂)₆NH(C═O)CH₂CH₂COOH.
102. The macromolecule of embodiment 82, wherein R₂ is C₁-C₁₂ amine.
103. The macromolecule of embodiment 102, wherein R₂ is C₆-C₁₂ amine.
104. The macromolecule of embodiment 103, wherein R₂ is C₁₂ amine.
105. The macromolecule of embodiment 82, wherein R₂ is C₁-C₆ guanidine.
106. The macromolecule of embodiment 105, wherein R₂ is —(CH₂)₂ guanidine.
107. The macromolecule of embodiment 82, wherein R₂ is optionally substituted heteroaryl.
108. The macromolecule of embodiment 107, wherein R₂ is —(CH₂)₁₋₆ imidazole.
109. The macromolecule of embodiment 108, wherein R₂ is —(CH₂)₃ imidazole.
110. The macromolecule of embodiment 82, wherein R₁ is succinate and R₂ is optionally substituted C₃-C₆ dicyloalkyl methane, optionally substituted aryl, C₁-C₆ thiol, or optionally substituted succinate.
111. The macromolecule of embodiment 110, wherein R₂ is dicyclohexylmethane succinate.
112. The macromolecule of embodiment 111, wherein R₂ is —(CH₂)₁₋₁₂NH(C═O)CH₂CH₂COOH.
113. The macromolecule of embodiment 112, wherein R₂ is —(CH₂)₁₋₃NH(C═O)CH₂CH₂COOH.
114. The macromolecule of embodiment 113, wherein R₂ is —(CH₂)₂NH(C═O)CH₂CH₂COOH.
115. The macromolecule of embodiment 111, wherein R₂ is —(CH₂)₁₀₋₁₂NH(C═O)CH₂CH₂COOH.
116. The macromolecule of embodiment 110, wherein R₂ is Wherein R₂ is —(CH₂)₁₂NH(C═O)CH₂CH₂COOH.
117. The macromolecule of embodiment 81, wherein R₁ is C₂-C₁₂ alkenyl succinate and R₂ is substituted succinate.
118. The macromolecule of embodiment 117, wherein R₂ is C₈ alkenyl succinate and R₂ is C₈ alkenyl ethylaminosuccinate.
119. The macromolecule of embodiment 81, wherein each of R₁ and R₂ is nitrogen.
120. The macromolecule of embodiment 119, wherein R₁ and R₂ are taken together to form an optionally substituted heterocycle.
121. The macromolecule of embodiment 120, wherein the optionally substituted heterocycle is a triazole.
122. The macromolecule of embodiment 121, wherein the optionally substituted triazole comprises benzylamide.
123. The macromolecule of embodiment 122, wherein the benzylamide is halogenated.
124. The macromolecule of embodiment 81, wherein R₁ is C₁-C₆ alkyl sulfone and R₂ is optionally substituted C₁-C₆ alkylamine.
125. The macromolecule of embodiment 124, wherein R₁ is —(CH₂)₃SOOOH and R₂ is —(CH₂)₁₋₆N(CH₂CH₂CH₂SOOOH)₂.
126. The macromolecule of embodiment 125, wherein R₂ is —(CH₂)₂N(CH₂CH₂CH₂SOOOH)₂.
127. The macromolecule of embodiment 81, wherein R₁ is phthalate and R₂ is C₁-C₆ aminophthalate.
128. The macromolecule of embodiment 127, wherein R₂ is C₂-C₆ aminophthalate.
129. The macromolecule of embodiment 128, wherein R₂ is C₂ aminophthalate.
130. The macromolecule of embodiment 18, wherein B is

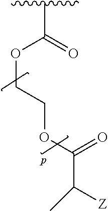

### and A is

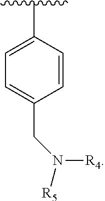

131. The macromolecule of embodiment 130, wherein R₄ is C₁-C₆ thiol and R₅ is succinate.
132. The macromolecule of embodiment 131, wherein R₄ is C₁-C₃ thiol.
133. The macromolecule of embodiment 132, wherein R₄ is —(CH₂)₂SH.
134. The macromolecule of any one of embodiments 18-133, wherein p is 1.
135. The macromolecule of embodiment 18, wherein B is

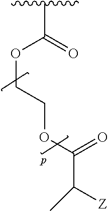

### and A is

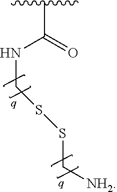

136. The macromolecule of embodiment 135, wherein p is 1.
137. The macromolecule of embodiment 135 or 136, wherein q is 2 or 3.
138. The macromolecule of embodiment 137, wherein q is 2.
139. The macromolecule of embodiment 18, wherein B is

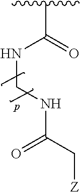

### and A is

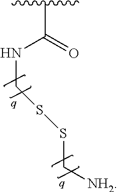

140. The macromolecule of embodiment 139, wherein p is 1.
141. The macromolecule of embodiment 139 or 140, wherein q is 2 or 3.
142. The macromolecule of embodiment 141, wherein q is 2.
143. The macromolecule of embodiment 18, wherein B is

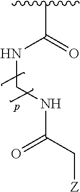

### and A is

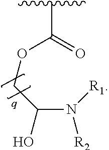

144. The macromolecule of embodiment 143, wherein p is 1.
145. The macromolecule of embodiment 143 or 144, wherein R₁ is hydrogen and R₂ is optionally substituted C₁-C₆ alkylamine.
146. The macromolecule of embodiment 145, wherein R₂ is —(CH₂)₁₋₃ dimethylamine.
147. The macromolecule of embodiment 146, wherein R₂ is —(CH₂)₂ dimethylamine.
148. The macromolecule of any one of embodiments 18-147, wherein the Component (A) comprises about 10 weight percent (wt %) to about 90 wt % of the macromolecule.
149. The macromolecule of embodiment 148, wherein Component (A) comprises about 20 wt % to about 80 wt % of the macromolecule.
150. The macromolecule of embodiment 149, wherein Component (A) comprises about 40 wt % to about 60 wt % of the macromolecule.
151. The macromolecule of embodiment 150, Wherein component (A) comprises about 50 wt % of the macromolecule.
152. The macromolecule of any one of embodiments 18-151, wherein the Component (B) comprises about 10 weight percent (wt %) to about 90 wt % of the macromolecule.
153. The macromolecule of embodiment 152, wherein Component (B) comprises about 20 wt % to about 80 wt % of the macromolecule.
154. The macromolecule of embodiment 153, wherein Component (B) comprises about 40 wt % to about 60 wt % of the macromolecule.
155. The macromolecule of embodiment 154, wherein component (B) comprises about 50 wt % of the macromolecule.
156. The macromolecule of any one of embodiments 1-155, wherein the macromolecule further comprises a peptide.
157. The macromolecule of embodiment 155, wherein the peptide is bound to the macromolecule through non-specific adsorption.
158. The macromolecule of any one of embodiments 18-157, wherein the structures of Component (A) and Component (B) is selected from Table 2.
159. A macromolecule comprising recurring units of a first component and a cross-linking recurring unit, wherein the first component comprises a structure of Component (A′):

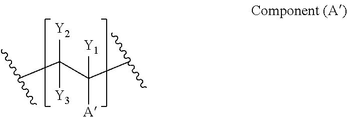

wherein
   each of Y₁, Y₂, and Y₃ is independently selected from hydrogen or C₁-C₆ alkyl;
      A′ is

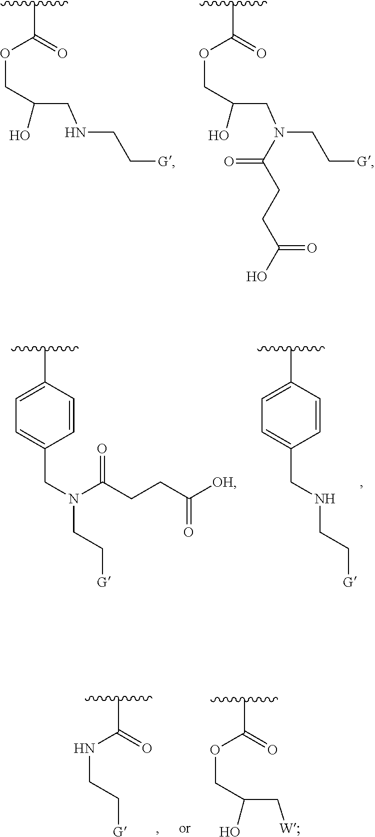

G′ or W′ comprise Q′;
   Q′ is a peptide.
      160. A macromolecule comprising recurring units of a first component and a second component, wherein the first component comprises a structure of Component (A′) and the second component comprises a structure of (B)

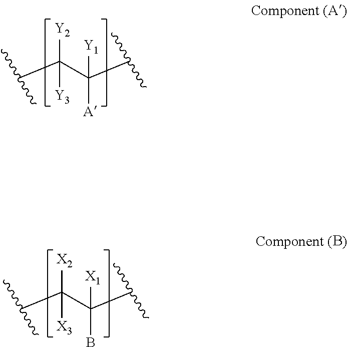

wherein
   each of Y₁, Y₂, and Y₃ is independently selected from hydrogen or C₁-C₆ alkyl;
      each of X₁, X₂, and X₃ is independently selected from hydrogen or C₁-C₆ alkyl;
      A′ is

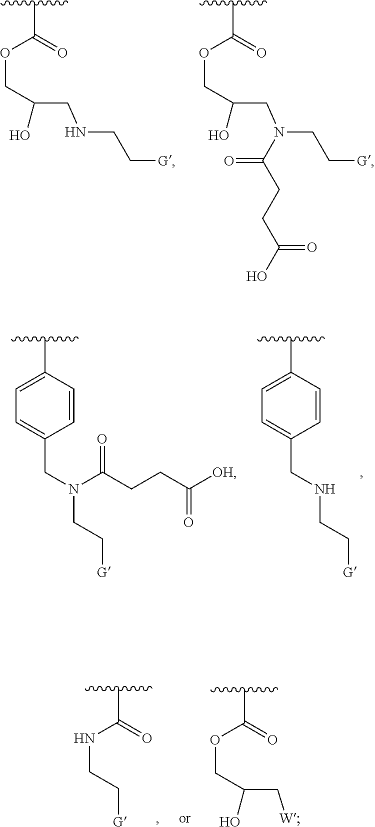

G′ or W′ comprise Q′;
   Q′ is a peptide;
      B is

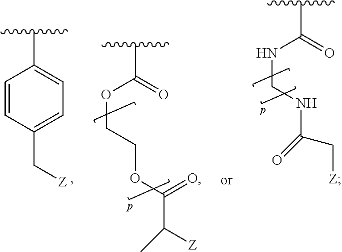

Z is a unit of Monomer (A′) or Monomer (B);
   q is an integer between 1 and 6; and
      p is an integer between 1 and 20.
161. The macromolecule of embodiment 159 or 160, wherein W′ is

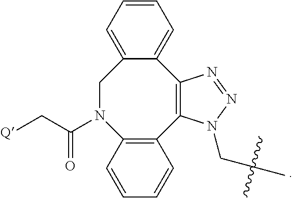

162. The macromolecule of embodiment 159 or 160, wherein G′ is

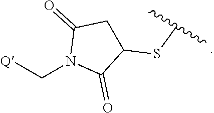

163. The macromolecule of any one of embodiments 159-162, wherein the peptide comprises at most about 40 amino acids.
164. The macromolecule of any one of embodiments 159-163, wherein the peptide comprises at least about 20 amino acids.
165. The macromolecule of any one of embodiments 159-164, wherein the peptide comprises a synthetic sequence.
166. The macromolecule of any one of embodiments 159-165, wherein the peptide comprises non-natural amino acids.
167. A system comprising:
   a. a surface;
   b. the macromolecule of any one of embodiments 1-166 coupled to the surface, wherein the peptide comprises a binding site;
   c. a protein interacting with the peptide at the binding site;
168. The system of embodiment 167, wherein the surface is a particle
169. The system of embodiment 168, wherein the particle is a nanoparticle or microparticle.
170. The system of embodiment 168, wherein the particle comprises a diameter of about 200 nanometers (nm) to about 500 nm.
171. The system of embodiment 168, wherein the particle is a superparamagnetic iron oxide particle.
172. The system of embodiment 168, wherein the particle comprises an iron oxide material.
173. The system of embodiment 168, wherein the particle has an iron oxide core.
174. The system of embodiment 168, wherein the particle has iron oxide crystals embedded in a polystyrene core.
175. The system of embodiment 168, wherein the particle comprises an iron oxide core with a silica shell coating.
176. The system of any one of embodiments 167-175, wherein the peptide is coupled to the surface at a density of at least 1 peptide per 5 nanometers squared, 1 peptide per 50 nanometers squared, or 1 peptide per 500 nanometers squared.
177. The system of any one of embodiments 167-176, wherein the surface further comprises a plurality of peptides coupled thereto, wherein each peptide in the plurality of peptides is configured to bind to at least three different proteins.
178. The system of any one of embodiments 167-177, wherein at least two different protein are specifically bound to the peptide.
179. The system of any one of embodiments 167-178, wherein the system further comprises a plurality of biomolecules adsorbed on the surface.
180. The system of embodiment 179, wherein the plurality of biomolecules comprises at least about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 proteins not specifically bound to the peptide.
181. The system of embodiment 179, wherein the plurality of biomolecules comprises at a dynamic range of at least about 5, 6, 7, 8, 9, 10, 11, 12, 13, 14, 15, 16, 17, 18, 19, or 20.
182. The system of any one of embodiments 167-181, wherein the surface is provided in a solution with at least about 0.05, 0.1, 0.2, 0.3, 0.4, or 0.5 cm² in surface area of the surface per μL of the solution.
183. The system of any one of embodiments 179-182, wherein a plurality of biomolecules are captured on the surface such that a ratio of abundance between at least two biomolecules in the plurality of biomolecules in solution is changed for the at least two biomolecules in the plurality of biomolecules captured on the surface.
184. The system of any one of embodiments 179-183, wherein a plurality of biomolecules is increased in visibility in a downstream assay.
185. The system of embodiment 184, wherein the visibility of a biomolecule in the plurality of biomolecules is measurable by an intensity as measured by mass spectrometry.
186. The system of any one of embodiments 167-185, wherein the protein comprises a targeting protein.
187. The system of any one of embodiments 167-186, wherein the protein comprises a vacuolar lumen, lysosomal lumen, spliceosomal tri-snRNP complex, U4/U6 xU5 tri-snRNP complex, secretory granule lumen, intracellular organelle lumen, membrane raft, spliceosomal snRNP, complex, spermatoproteasome complex, or Golgi lumen protein.
188. A surface comprising a moiety of Formula (IV′):

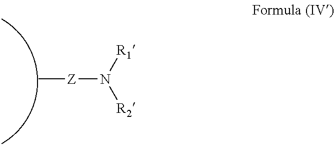

wherein
   Z is a linking moiety comprising a linear chain with 2 to 20 atoms selected from the group consisting of carbon, oxygen, and nitrogen and optional substituents on the carbon and any nitrogen atoms in the linear chain;
      R₁′ is hydrogen or succinate; and
      R₂′ is C₁-C₆ alkyl-G′;
      G′ comprises Q′;
      Q′ is peptide;
wherein peptide does not comprise cysteine.
189. The surface of embodiment 188, wherein G′ is

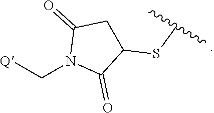

190. The surface of any one of embodiments 188 or 189, wherein the peptide comprises at most about 40 amino acids.
191. The surface of any one of embodiments 188-190, wherein the peptide comprises at least about 20 amino acids.
192. The surface of any one of embodiments 188-191, wherein the peptide comprises a synthetic sequence.
193. The surface of any one of embodiments 188-192, wherein the peptide comprises non-natural amino acids.
194. A method of preparing a surface comprising recurring units of a first component and a second component, said method comprising:
   a. providing a mixture of monomers in a solvent comprising a first monomer and a second monomer, wherein the first monomer comprises a vinyl group and wherein the second monomer comprises an epoxide group;
   b. contacting a surface and the mixture of monomers, thereby producing a reaction mixture;
   c. initiating free radical polymerization to produce a macromolecule immobilized to the surface;
   d. contacting the macromolecule immobilized to the surface and an amine, thereby producing an aminated macromolecule; and
   e. optionally contacting the aminated macromolecule with a compound comprising succinate, phthalate, or propanesulfone.
195. The method of embodiment 194, wherein the surface is a particle or bead.
196. The method of embodiment 195, wherein the particle is a superparamagnetic iron oxide particle.
197. The method of embodiment 195, wherein the particle comprises an iron oxide material.
198. The method of embodiment 195, wherein the particle has an iron oxide core.
199. The method of embodiment 198, wherein the particle has iron oxide crystals embedded in a polystyrene core.
200. The method of embodiment 198, wherein the particle comprises an iron oxide core with a silica shell coating.
201. The method of any one of embodiments 194-200, wherein the solvent is a polar solvent
202. The method of embodiment 201, wherein the solvent is acetonitrile, THF, or DMF
203. The method of any one of embodiments 194-202, wherein free radical polymerization is initiated with a radical initiator.
204. The method of embodiment 203, wherein the radical initiator is AIBN.
205. The method of any one of embodiments 194 to 204, wherein the method comprises, subsequent to (c) and prior to (d), contacting the macromolecule immobilized to the surface with a quenching agent.
206. The method of embodiment 205, wherein the quenching agent is introduced to the reaction mixture when the macromolecule immobilized to the surface comprises a diameter of about 300 nm to about 500 nm.
207. The method of embodiment 206, wherein the diameter is about 325 nm to about 375 nm
208. The method of embodiment 205, wherein the quenching agent comprises benzoquinone.
209. The method of any one of embodiments 194-208, wherein the method further comprises purifying the macromolecule immobilized to the surface
210. The method of embodiment 209, wherein purifying comprises washing the macromolecule immobilized to the surface with THF or ethanol
211. The method of any one of embodiments 194-210, wherein the first monomer is divinylbenzene (DVB), ethyleneglycol dimethacrylate (EGDMA), or N,N′-methylenebisacrylamide (MBA).
212. The method of any one of embodiments 194-211, wherein the second monomer comprises glycidyl methacrylate.
213. The method of any one of embodiments 194-212, wherein the amine is C₁-C₁₂ alkylamine, C₁-C₆hydroxyamine, C₁-C₆ alkoxyethylamine.
214. The method of embodiment 213, wherein the C₁-C₁₂ alkylamine is diethylamine.
215. The method of embodiment 213, wherein the C₁-C₆ hydroxyamine is ethanolamine or hexanolamine.
216. The method of embodiment 213, wherein the C₁-C₆ alkoxyethylamine is methoxyethylamine.
217. The method of any one of embodiments 194 to 216, wherein the method comprises (e) contacting the aminated macromolecule with a compound comprising succinate, phthalate, thiol, or propylsulfone.
218. The method of embodiment 217, wherein the compound comprises a succinate.
219. The method of embodiment 218, wherein the succinate is C₈ alkenyl succinate or C₈ alkenyl ethylaminosuccinate.
220. The method of embodiment 217, wherein the compound comprises a thiol.
221. The method of embodiment 220, wherein the thiol is C₂ alkyl thiol.
222. The method of embodiment 217, wherein the compound comprises a phthalate.
223. The method of embodiment 222, wherein the phthalate is C₁-C₆ aminophthalate.
224. The method of embodiment 217, wherein the compound comprises a propylsulfone.
225. The method of embodiment 224, wherein the propylsulfone is dipropylsulfone ethylamine.
226. The method of any one of embodiments 194-225, wherein the first monomer comprises about 10 weight percent (wt %) to about 90 wt % of the mixture of monomers.
227. The method of embodiment 226, wherein the first monomer comprises about 20 wt % to about 80 wt % of the mixture of monomers.
228. The method of embodiment 227, wherein the first monomer comprises about 40 wt % to about 60 wt % of the mixture of monomers.
229. The method of embodiment 228, wherein the first monomer comprises about 50 wt % of the mixture of monomers.
230. The method of any one of embodiments 194 to 229, wherein the second monomer comprises about 10 weight percent (wt %) to about 90 wt % of the mixture of monomers.
231. The method of embodiment 230, wherein the second monomer comprises about 20 wt % to about 80 wt % of the mixture of monomers.
232. The method of embodiment 231, wherein the second monomer comprises about 40 wt % to about 60 wt % of the mixture of monomers.
233. The method of embodiment 232, wherein the second monomer comprises about 50 wt % of the mixture of monomers.
234. A method of preparing a surface comprising recurring units of a first component and second component, the method comprising:
   (a) providing a mixture of monomers in a solvent comprising a first monomer and a second monomer, wherein the first monomer comprises a vinyl group and the second monomer comprises an epoxide group;
   (b) contacting a surface and the mixture of monomers, thereby producing a reaction mixture;
   (c) initiating a free radical polymerization to produce a macromolecule immobilized to a surface;
   (d) contacting the macromolecule immobilized to the surface with an azide salt, thereby producing an azide containing macromolecule; and
   (e) optionally contacting the azide-containing macromolecule with an alkyne-containing molecule to form a triazole-containing macromolecule.
235. The method of embodiment 234, wherein the surface is a particle or a bead.
236. The method of embodiment 235, wherein the particle is a superparamagnetic iron oxide particle.
237. The method of embodiment 235, wherein the particle comprises an iron oxide material.
238. The method of embodiment 235, wherein the particle has an iron oxide core.
239. The method of embodiment 238, wherein the particle has iron oxide crystals embedded in a polystyrene core.
240. The method of embodiment 238, wherein the particle comprises an iron oxide core with a silica shell coating.
241. The method of any one of embodiments 234-240, wherein the solvent is a polar solvent.
242. The method of embodiment 241, wherein the solvent is acetonitrile, THF, or DMF.
243. The method of any one of embodiments 234-242, wherein free radical polymerization is initiated with a radical initiator.
244. The method of embodiment 243, wherein the radical initiator is AIBN.
245. The method of any one of embodiments 234-244, wherein the method comprises, subsequent to (c) and prior to (d), contacting the macromolecule immobilized to the surface with a quenching agent.
246. A composition for identifying biomolecules in a biological sample, the composition comprising one or more surfaces of embodiments 188-193 and a biological sample in contact with the surfaces.
247. The composition of embodiment 246, wherein the biological sample is plasma, serum or blood.
248. The composition of embodiment 246 or 247, wherein the one or more surfaces comprises at least 2 distinct surfaces, at least 3 distinct surfaces, at least 4 distinct surfaces, at least 5 distinct surfaces, at least 6 distinct surfaces, at least 7 distinct surfaces, at least 8 distinct surfaces, at least 9 distinct surfaces, at least 10 distinct surfaces, at least 11 distinct surfaces, at least 12 distinct surfaces, at least 13 distinct surfaces, at least 14 distinct surfaces, at least 15 distinct surfaces, at least 20 distinct surfaces, at least 25 surfaces, or at least 30 distinct surfaces.
249. The composition of any one of embodiments 246-248, wherein the one or more surfaces comprises a first distinct surface and a second distinct surface, wherein the first distinct surface and the second distinct surface share at least one physicochemical property and differ by at least one physicochemical property, such that the first distinct surface and the second distinct surface are different.
250. The composition of any one of embodiments 246-249, wherein the one or more surfaces comprises a first distinct surface and a second distinct surface, wherein the first distinct surface and the second distinct surface share at least two physicochemical properties and differ by at least two physicochemical properties, such that the first distinct surface and the second distinct surface are different.
251. The composition of any one of embodiments 246-250, wherein the one or more surfaces comprises a first distinct surface and a second distinct surface, wherein the first distinct surface and the second distinct surface share at least one physicochemical property and differ by at least two physicochemical properties, such that the first distinct surface and the second distinct surface are different.
252. The composition of any one of embodiments 246-251, wherein the one or more surfaces comprises a first distinct surface and a second distinct surface, wherein the first distinct surface and the second distinct surface share at least two physicochemical properties and differ by at least one physicochemical property, such that the first distinct surface and the second distinct surface are different.
253. The composition of any one of embodiments 246-252, wherein the physicochemical property comprises size, charge, core material, shell material, porosity, or surface hydrophobicity.
254. The composition of any one of embodiments 246-253, wherein the size is diameter or radius, as measured by dynamic light scattering, SEM, TEM, or any combination thereof.
255. The composition of any one of embodiments 246-254, wherein the one or more surfaces comprises a first distinct surface and a second distinct surface, wherein the first distinct surface and the second distinct surface comprise a carboxylate material, wherein the first distinct particle is a microparticle, and wherein the second distinct surface is a nanoparticle.
256. The composition of any one of embodiments 246-255, wherein the one or more surfaces comprises a first distinct surface and a second distinct surface, wherein the first distinct surface and the second distinct surface comprise a surface charge of from 0 mV and −50 mV, wherein the first distinct surface has a diameter of less than 200 nm, and wherein the second distinct surface has a diameter of greater than 200 nm.
257. The composition of any one of embodiments 246-256, wherein the one or more surfaces comprises a first distinct surface and a second distinct surface, wherein the first distinct surface and the second distinct surface comprise a diameter of 100 to 400 nm, wherein the first distinct surface has a positive surface change, and wherein the second distinct surface has a neutral surface charge.
258. The composition of any one of embodiments 246-257, wherein the one or more surfaces comprises a first distinct surface and a second distinct surface, wherein the first distinct surface and the second distinct surface are nanoparticles, wherein the first distinct surface has a surface change less than −20 mV and the second distinct surface has a surface charge greater than −20 mV.
259. The composition of any one of embodiments 246-258, wherein the one or more surfaces comprises a first distinct surface and a second distinct surface, wherein the first distinct surface and the second distinct surface are microparticles, wherein the first distinct surface has a negative surface charge, and wherein the second distinct surface has a positive surface charge.
260. The composition of any one of embodiments 246-259, wherein the one or more surfaces comprises a subset of negatively charged nanoparticles, wherein each particle of the subset differ by at least one surface chemical group.
261. The composition of any one of embodiments 246-260, the one or more surfaces comprises a first distinct surface, a second particle, and a third distinct surface, wherein the first distinct surface, the second distinct surface, and the third distinct surface comprise iron oxide cores, polymer shells, and are less than about 500 nm in diameter and wherein the first distinct surface comprises a negative charge, the second distinct surface comprises a positive charge, and the third distinct surface comprises a neutral charge, wherein the diameter is a mean diameter as measured by dynamic light scattering.
262. The composition of any one of embodiments 246-261, wherein at least one distinct surface of the one or more surfaces is a superparamagnetic iron oxide particle.
263. The composition of any one of embodiments 246-262, wherein each surface of the one or more surfaces comprise an iron oxide material.
264. The composition of any one of embodiments 246-263, wherein at least one distinct surface of the one or more surfaces has an iron oxide core.
265. The composition of any one of embodiments 246-264, wherein at least one distinct surface of the one or more surfaces has iron oxide crystals embedded in a polystyrene core.
266. The composition of any one of embodiments 246-265, wherein each distinct surface of the one or more surfaces is a superparamagnetic iron oxide particle.
267. The composition of any one of embodiments 246-266, wherein each distinct surface of the one or more surfaces comprises an iron oxide core.
268. The composition of any one of embodiments 246-267, wherein each one distinct surface of the one or more surfaces has iron oxide crystals embedded in a polystyrene core.
269. The composition of any one of embodiments 246-268, wherein at least one surface of the one or more surfaces comprises an iron oxide core with a silica shell coating.
270. A system for identifying biomolecules in a biological sample, the composition comprising:
   i. the surface of embodiments 188-193;
   ii. a suspension solution;
   iii. a biological sample comprising a concentration of proteins; and
   iv. an automated system comprising a network of units with differentiated functions in distinguishing states of a complex biological sample using a plurality of surfaces with different physiochemical properties, and wherein the automated system is programed to perform a series of steps.
271. The system of embodiment 270, wherein the network of units comprises:
   i. a first unit comprises a multichannel fluid transfer instrument for transferring fluids between units within the system;
   ii. a second unit comprises a support for storing a plurality of biological samples;
   iii. a third unit comprises a support for a sensor array plate possessing partitions that comprise the plurality of particles having surfaces with different physiochemical properties for detecting a binding interaction between a population of analytes within the complex biological sample and the plurality of particles;
   iv. a fourth unit comprises supports for storing a plurality of reagents;
   v. a fifth unit comprises supports for storing a reagent to be disposed of; and
   vi. a sixth unit comprises supports for storing consumables used by the multichannel fluid transfer instrument
272. The system of embodiment 270 or 271, wherein the series of steps comprises:
   i. contacting the biological sample with a specified partition of the sensor array;
   ii. incubating the biological sample with the plurality of particles contained within the partition of the sensor array plate;
   iii. removing all components from a partition except the plurality of particles and a population of analytes interacting with a particle; and
   iv. preparing a sample for mass spectrometry.
273. The system of any one of embodiments 270-272, wherein i.-iii. are incubated at a temperature of about 20 degrees Celsius to about 100 degrees Celsius.
274. The system of any one of embodiments 270-273, wherein the suspension solution comprises Tris EDTA 150 mM KCl, 0.05% CHAPS buffer.
275. The system of any one of embodiments 270-274, wherein the suspension solution comprises 10 mM Tris HCl pH 7.4, 1 mM EDTA.
276. A method of identifying proteins in a sample, the method comprising:
   a. incubating one or more surfaces of embodiments 188-193 with a biological sample comprising biomolecules to form a distinct biomolecule corona;
   b. isolating at least a portion of the biomolecules in the distinct biomolecule corona; and
   c. assaying the distinct biomolecule corona.
277. The method of embodiment 276, wherein the assaying is capable of identifying from 1 to 20,000 protein groups.
278. The method of any one of embodiments 276 or 277, wherein the assaying is capable of identifying from 1000 to 10,000 protein groups.
279. The method of any one of embodiments 276-278, wherein the assaying is capable of identifying from 1,000 to 5,000 protein groups.
280. The method of any one of embodiments 276-279, wherein the assaying is capable of identifying from 1,200 to 2,200 protein groups.
281. The method of any one of embodiments 276-280, wherein the protein group comprises a peptide sequence having a minimum length of 7 amino acid residues.
282. The method of any one of embodiments 276-281, wherein the assaying is capable of identifying from 1,000 to 10,000 proteins.
283. The method of any one of embodiments 276-282, wherein the assaying is capable of identifying from 1,800 to 5,000 proteins.
284. The method of any one of embodiments 276-283, wherein the sample comprises a plurality of samples.
285. The method of any one of embodiments 276-284, wherein the plurality of samples comprises at least two or more spatially isolated samples.
286. The method of any one of embodiments 276-285, wherein the incubating comprises contacting the at least two or more spatially isolated samples with the one or more surfaces at the same time.
287. The method of any one of embodiments 276-286, wherein isolating comprises magnetically isolating the one or more surfaces from unbound protein in the at least two or more spatially isolated samples of the plurality of samples at the same time.
288. The method of any one of embodiments 276-287, wherein the assaying comprises assaying a plurality of distinct biomolecule coronas to identify proteins in the at least two or more spatially isolated samples at the same time.
289. The method of any one of embodiments 276-288, further comprising repeating the wherein, when repeated, the incubating, isolating, and assaying yields a percent quantile normalized coefficient (QNCV) of variation of 20% or less, as determined by comparing a peptide mass spectrometry feature from at least three full-assay replicates for each surface in the one or more surfaces.
290. The method of any one of embodiments 276-289, wherein, when repeated, the incubating, isolating, and assaying yields a percent quantile normalized coefficient (QNCV) of variation of 10% or less, as determined by comparing a peptide mass spectrometry feature from at least three full-assay replicates for each surface in the one or more surfaces.
291. The method of any one of embodiments 276-290, wherein the assaying is capable of identifying proteins over a dynamic range of at least 7, at least 8, at least 9, or at least 10.
292. The method of any one of embodiments 276-291, further comprising washing the one or more surfaces at least one time or at least two times after magnetically isolating the one or more surfaces from the unbound protein.
293. The method of any one of embodiments 276-292, wherein after the assaying the method further comprises lysing the proteins in the plurality of distinct biomolecule coronas.
294. The method of any one of embodiments 276-293, further comprising digesting the proteins in the plurality of distinct biomolecule coronas to generate digested peptides.
295. The method of any one of embodiments 276-294, further comprising purifying the digested peptides.
296. The method of any one of embodiments 276-295, wherein the assaying comprises using mass spectrometry to identify proteins in the sample.
297. The method of any one of embodiments 276-296, wherein the assaying is performed in about 2 to about 4 hours.
298. The method of any one of embodiments 276-297, wherein the method is performed in about 1 to about 20 hours.
299. The method of any one of embodiments 276-298, wherein the method is performed in about 2 to about 10 hours.
300. The method of any one of embodiments 276-299, wherein the method is performed in about 4 to about 6 hours.
301. The method of any one of embodiments 276-300, wherein the isolating takes no more than about 30 minutes, no more than about 15 minutes, no more than about 10 minutes, no more than about 5 minutes, or no more than about 2 minutes.
302. The method of any one of embodiments 276-301, wherein the plurality of samples comprises at least 10 spatially isolated samples, at least 50 spatially isolated samples, at least 100 spatially isolated samples, at least 150 spatially isolated samples, at least 200 spatially isolated samples, at least 250 spatially isolated samples, or at least 300 spatially isolated samples.
303. The method of any one of embodiments 276-302, wherein the plurality of samples comprises at least 96 samples.
304. The method of any one of embodiments 276-303, wherein the one or more surfaces comprises at least 2 distinct surfaces, at least 3 distinct surfaces, at least 4 distinct surfaces, at least 5 distinct surfaces, at least 6 distinct surfaces, at least 7 distinct surfaces, at least 8 distinct surfaces, at least 9 distinct surfaces, at least 10 distinct surfaces, at least 11 distinct surfaces, at least 12 distinct surfaces, at least 13 distinct surfaces, at least 14 distinct surfaces, at least 15 distinct surfaces, at least 20 distinct surfaces, at least 25 surfaces, or at least 30 distinct surfaces.
305. The method of any one of embodiments 276-304, wherein the one or more surfaces comprises at least 10 distinct surfaces.
306. The method of any one of embodiments 276-305, wherein the at least two spatially isolated samples differ by at least one physicochemical property.
307. The method of any one of embodiments 276-306, wherein the one or more surfaces comprises a first distinct surface and a second distinct surface, wherein the first distinct surface and the second distinct surface share at least one physicochemical property and differ by at least one physicochemical property, such that the first distinct surface and the second distinct surface are different.
308. The method of any one of embodiments 276-307, wherein the one or more surfaces comprises a first distinct surface and a second distinct surface, wherein the first distinct surface and the second distinct surface share at least two physicochemical properties and differ by at least two physicochemical properties, such that the first distinct surface and the second distinct surface are different.
309. The method of any one of embodiments 276-308, wherein the one or more surfaces comprises a first distinct surface and a second distinct surface, wherein the first distinct surface and the second distinct surface share at least one physicochemical property and differ by at least two physicochemical properties, such that the first distinct surface and the second distinct surface are different.
310. The method of any one of embodiments 276-309, wherein the one or more surfaces comprises a first distinct surface and a second distinct surface, wherein the first distinct surface and the second distinct surface share at least two physicochemical properties and differ by at least one physicochemical property, such that the first distinct surface and the second distinct surface are different.
311. The method of any one of embodiments 276-310, wherein the physicochemical property comprises size, charge, core material, shell material, porosity, or surface hydrophobicity.
312. The method of any one of embodiments 276-311, wherein the size is diameter or radius, as measured by dynamic light scattering, SEM, TEM, or any combination thereof.
313. The method of any one of embodiments 276-312, wherein the one or more surfaces comprises a first distinct surface and a second distinct surface, wherein the first distinct surface and the second distinct surface comprise a carboxylate material, wherein the first distinct particle is a microparticle, and wherein the second distinct surface is a nanoparticle.
314. The method of any one of embodiments 276-313, wherein the one or more surfaces comprises a first distinct surface and a second distinct surface, wherein the first distinct surface and the second distinct surface comprise a surface charge of from 0 mV and −50 mV, wherein the first distinct surface has a diameter of less than 200 nm, and wherein the second distinct surface has a diameter of greater than 200 nm.
315. The method of any one of embodiments 276-314, wherein the one or more surfaces comprises a first distinct surface and a second distinct surface, wherein the first distinct surface and the second distinct surface comprise a diameter of 100 to 400 nm, wherein the first distinct surface has a positive surface change, and wherein the second distinct surface has a neutral surface charge.
316. The method of any one of embodiments 276-315, wherein the one or more surfaces comprises a first distinct surface and a second distinct surface, wherein the first distinct surface and the second distinct surface are nanoparticles, wherein the first distinct surface has a surface change less than −20 mV and the second distinct surface has a surface charge greater than −20 mV.
317. The method of any one of embodiments 276-316, wherein the one or more surfaces comprises a first distinct surface and a second distinct surface, wherein the first distinct surface and the second distinct surface are microparticles, wherein the first distinct surface has a negative surface charge, and wherein the second distinct surface has a positive surface charge.
318. The method of any one of embodiments 276-317, wherein the one or more surfaces comprises a subset of negatively charged nanoparticles, wherein each particle of the subset differ by at least one surface chemical group.
319. The method of any one of embodiments 276-318, wherein the one or more surfaces comprises a first distinct surface, a second particle, and a third distinct surface, wherein the first distinct surface, the second distinct surface, and the third distinct surface comprise iron oxide cores, polymer shells, and are less than about 500 nm in diameter and wherein the first distinct surface comprises a negative charge, the second distinct surface comprises a positive charge, and the third distinct surface comprises a neutral charge, wherein the diameter is a mean diameter as measured by dynamic light scattering.
320. The method of any one of embodiments 276-319, wherein at least one distinct surface of the one or more surfaces is a nanoparticle.
321. The method of any one of embodiments 276-320, wherein at least one distinct surface of the one or more surfaces is a microparticle.
322. The method of any one of embodiments 276-321, wherein at least one distinct surface of the one or more surfaces is a superparamagnetic iron oxide particle.
323. The method of any one of embodiments 276-322, wherein each particle of the one or more surfaces comprise an iron oxide material.
324. The method of any one of embodiments 276-323, wherein at least one distinct surface of the one or more surfaces has an iron oxide core.
325. The method of any one of embodiments 276-324, wherein at least one distinct surface of the one or more surfaces has iron oxide crystals embedded in a polystyrene core.
326. The method of any one of embodiments 276-325, wherein each distinct surface of the one or more surfaces is a superparamagnetic iron oxide particle.
327. The method of any one of embodiments 276-326, wherein each distinct surface of the one or more surfaces comprises an iron oxide core.
328. The method of any one of embodiments 276-327, wherein each one distinct surface of the one or more surfaces has iron oxide crystals embedded in a polystyrene core.
329. The method of any one of embodiments 276-328, wherein at least one distinct surface of one or more surfaces comprises a carboxylated polymer, an aminated polymer, a zwitterionic polymer, or any combination thereof.
330. The method of any one of embodiments 276-329, wherein at least one surface of the one or more surfaces comprises an iron oxide core with a silica shell coating.
331. The method of any one of embodiments 276-330, wherein at least one distinct surface of the one or more surfaces comprises a negative surface charge.
332. The method of any one of embodiments 276-331, wherein at least one distinct surface of the one or more surfaces comprises a positive surface charge.
333. The method of any one of embodiments 276-332, wherein at least one distinct surface of the one or more surfaces comprises a neutral surface charge.
334. Use of the macromolecule of any one of embodiments 1-166 in a method for identifying proteins in a biological sample.
335. Use of the macromolecule of any one of embodiments 1-166 to adsorb proteins in a biological sample.
336. Use of the surface of any one of embodiments 188-193 in a method for identifying proteins in a biological sample.
337. Use of the surface of any one of embodiments 188-193 to adsorb proteins in a biological sample.
338. A macromolecule comprising a recurring unit of Formula (I-A):

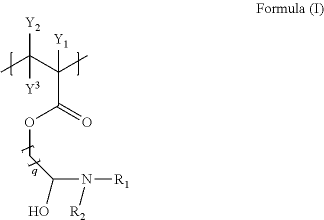

wherein each of Y₁, Y₂, and Y₃ is independently selected from hydrogen or C₁-C₆ alkyl;
   R¹ is hydrogen, nitrogen, optionally substituted succinate, C₁-C₆ alkyl sulfone, phthalate;
   R₂ is nitrogen, C₁-C₆ hydroxy, optionally substituted aryl, optionally substituted heteroaryl, optionally substituted C₃-C₆ heterocycloalkyl, optionally substituted C₃-C₆ dicyloalkyl methane, C₁-C₆ alkyl guanidine, C₁-C₆ ether, optionally substituted —C₁-C₆ disulfide, C₁-C₆ thiol, optionally substituted succinate, optionally substituted C₁-C₆ alkylamine, C₁-C₆ alkyl acetamide, or C₁-C₆ aminophthalate; and
   q is an integer between 1 and 6,
wherein if R₁ and R₂ are each nitrogen, then R₁ and R₂ are optionally taken together with the atom to which they are attached to form an optionally substituted heterocycle.
339. A surface for adsorbing biomolecules from a biological sample, wherein the surface is functionalized with a carboxylic acid and a thiol, and wherein the functionalization facilitates the adsorption of biomolecules when contacted with the biological sample.
340. The surface of embodiment 339, wherein the surface comprises a plurality of particles.
341. The surface of embodiment 340, wherein the particles are magnetic.
342. The surface of any one of embodiments 339 to 341, wherein the carboxylic acid and the thiol are covalently coupled to the substrate.
343. The surface of any one of embodiments 339 to 341, wherein the carboxylic acid and the thiol are covalently coupled to the substrate via a secondary or tertiary amine.
344. The surface of any one of embodiments 339 to 341, wherein the surface is functionalized with a tertiary amine that is covalently coupled to both the carboxylic acid and the thiol.
345. The surface of any one of embodiments 339 to 344, wherein the surface is functionalized with a moiety having the structure

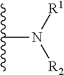

wherein,
   R¹ is optionally substituted succinate, optionally substituted glutarate, optionally substituted adipate, optionally substituted pimelate, optionally substituted suberate, optionally substituted azelate, or optionally substituted sebacate; and
   R₂ is optionally substituted C₁-C₆ thiol.
346. The surface of any one of embodiments 339 to 344, wherein the surface is identified as Compound 508, Compound 328, or Compound 509.
347. The surface of any one of embodiments 339 to 346, wherein the surface comprises silica.
348. The surface of any one of embodiments 339 to 348, wherein the surface comprises a polymer.
349. The surface of embodiment 348, wherein the polymer is an acrylamide or acrylate.
350. The surface of embodiment 348 or 349, wherein the polymer is functionalized with the carboxylic acid and the thiol.
351. The surface of embodiment 350, wherein the polymer comprises a recurring unit having both the carboxylic acid and the thiol.
352. The surface of any one of embodiments 339 to 351, wherein the surface has a zeta potential of less than about −25 mV.
353. The surface of any one of embodiments 339 to 352, wherein the surface is a microparticle or nanoparticle.
354. The surface of any one of embodiments 339 to 353, wherein the surface has a largest dimension of about 100 nm to about 600 nm.
355. The surface of any one of embodiments 339 to 354, wherein a molar ratio of the thiol and the carboxylic acid is about 3:1 to about 1:3 (e.g., about 2:1 to about 1:2, or about 3:2 to about 2:3).
356. A method comprising:
   (a) contacting a biological sample with the surface of any one of embodiments 339 to 355 such that biomolecules adsorb onto the surface, wherein the surface is functionalized with a carboxylic acid and a thiol;
   (b) separating the biological sample from the surface; and
   (c) analyzing the biomolecules adsorbed to the surface.
357. The method of embodiment 356, wherein the analyzing identifies a range of biomolecules including at least 500 unique biomolecules.
358. The method of embodiment 356, wherein the analyzing identifies a range of biomolecules including at least 1000 unique biomolecules.
359. The method of embodiment 356, wherein the analyzing identifies a range of biomolecules including at least 500 unique proteins.
360. The method of embodiment 356, wherein the analyzing identifies a range of biomolecules including at least 1000 unique proteins.
361. The method of any one of embodiments 356 to 360, wherein the separating comprises magnetically isolating the surface from unbound biomolecules.
362. The method of any one of embodiments 356 to 361, wherein the surface is functionalized with a carboxylic acid and a thiol in a manner that, when repeated, yields a reproducible biomolecular adsorption profile.
363. The method of any one of embodiments 356 to 362, wherein the analyzing is capable of identifying biomolecules over a dynamic range of at least 7, at least 8, at least 9, or at least 10.
364. The method of any one of embodiments 356 to 363, further comprising washing the surface at least one time or at least two times after separating the surface from the biological sample.
365. The method of any one of embodiments 356 to 364, wherein the analyzing comprises using mass spectroscopy to identify biomolecules in the sample.
366. The method of any one of embodiments 356 to 365, wherein the separating takes no more than about 30 minutes, no more than about 15 minutes, no more than about 10 minutes, no more than about 5 minutes, or no more than about 2 minutes.
367. The method of any one of embodiments 356 to 366, wherein the biomolecules comprise proteins.
368. The method of embodiment 367, further comprising digesting the proteins before analyzing the proteins.
369. The method of any one of embodiments 356 to 368, wherein the biological sample is a biofluid.
370. The method of any one of embodiments 356 to 369, wherein the biological sample is a plasma or serum.
371. The method of any one of embodiments 356 to 370, wherein the biological sample is cell culture media.
372. The method of any one of embodiments 356 to 371, wherein the contacting comprises incubating the particles with the biological sample for at a temperature about 5° C. to 90° C. for at least about 15 minutes.
373. The method of any one of embodiments 356 to 372, wherein the surface is not functionalized with a biomolecule, such as an amino acid, peptide or protein.
374. The method of any one of embodiments 356 to 373, wherein the surface is not functionalized with cysteine.
375. A composition comprising the surface of any one of embodiments 339 to 355 and a biological sample in contact with the surface.
376. A kit comprising the surface of any one of embodiments 339 to 355.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby. The section headings used herein are for organizational purposes only and are not to be construed as limiting the subject matter described.

1-187. (canceled)

188. A macromolecule comprising recurring units of a first component and a second component, wherein the first component comprises a structure of Component (A) and the second component comprises a structure of Component (B′): 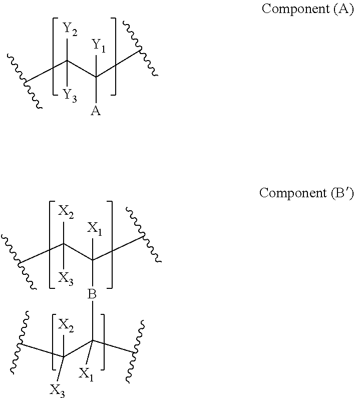
wherein each of Y₁, Y₂, and Y₃ is independently selected from hydrogen or C₁-C₆ alkyl;
each of X₁, X₂, and X₃ is independently selected from hydrogen or C₁-C₆ alkyl;
A is 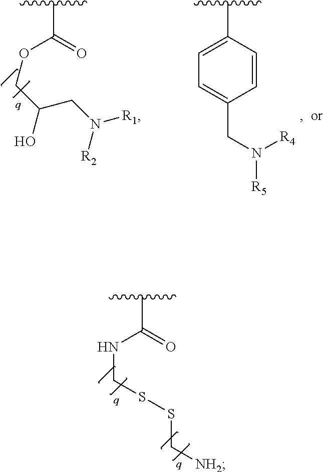
R₁ is hydrogen, optionally substituted succinate, C₁-C₆ alkyl sulfone, or phthalate;
R₂ is C₁-C₁₂ amine, C₁-C₆ hydroxy, optionally substituted aryl, optionally substituted heteroaryl, optionally substituted C₃-C₆ heterocycloalkyl, two or more fused
3-6 member rings; optionally substituted C₃-C₆ dicyloalkyl methane, C₁-C₆ alkyl guanidine, C₁-C₆ ether, optionally substituted C₁-C₆ disulfide, C₁-C₆ thiol, optionally substituted succinate, optionally substituted C₁-C₆ alkylamine, C₁-C₆ alkyl acetamide, C₁-C₆ aminophthalate, a boronic acid, C₁-C₆ thiol, C₅-C₁₁ optionally substituted cycloalkyl, or a monosaccharide; or
R₁ and R₂ are taken together with the nitrogen to which they are attached to form an optionally substituted heterocycle; or
R₁ and R₂ are taken together with the nitrogen to which they are attached to form an azide group;
R₄ is hydrogen or C₁-C₆ thiol;
R₅ is succinate, optionally substituted aryl, or optionally substituted C₁-C₆ disulfide;
B is 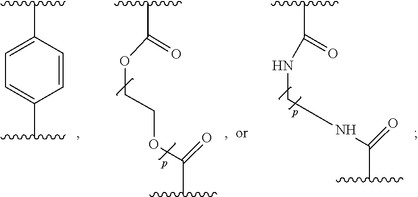
each q is independently an integer between 1 and 6; and
p is an integer between 1 and 20.

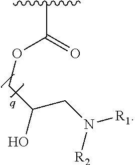

189. The macromolecule of claim 188, wherein A is R₂₁.

190. The macromolecule of claim 189, wherein q is 1.

191. The macromolecule of claim 190, wherein R₁ is hydrogen,

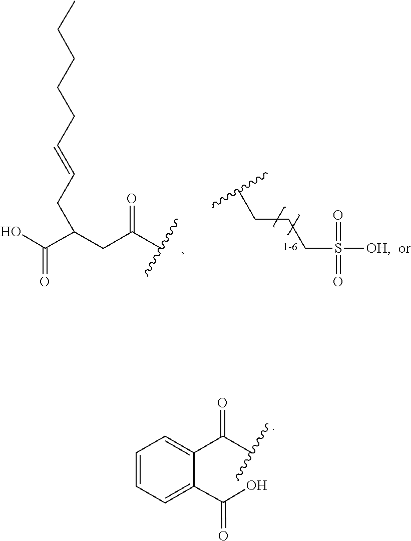

192. The macromolecule of claim 191, wherein R₂ is

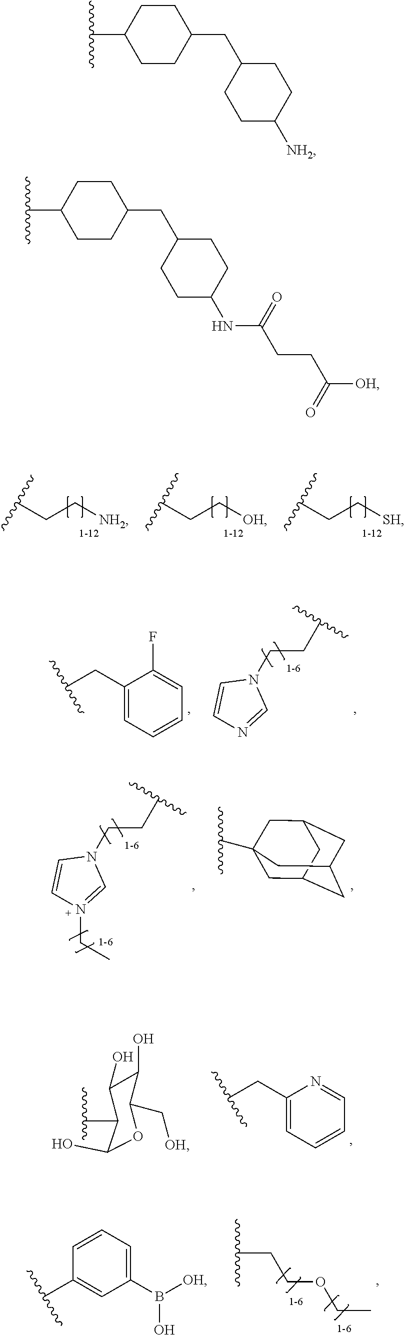

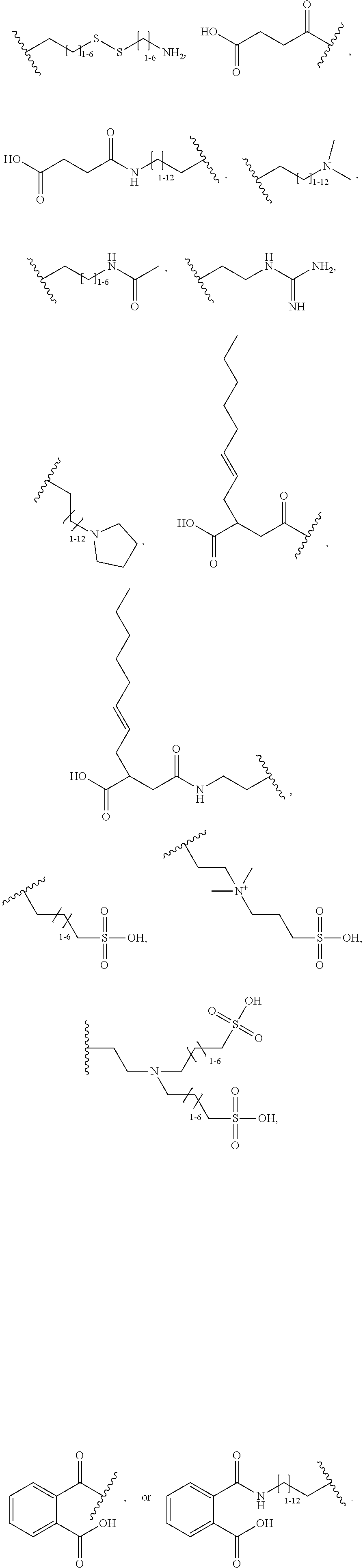

193. The macromolecule of claim 188, wherein the macromolecule comprises a structure selected from: 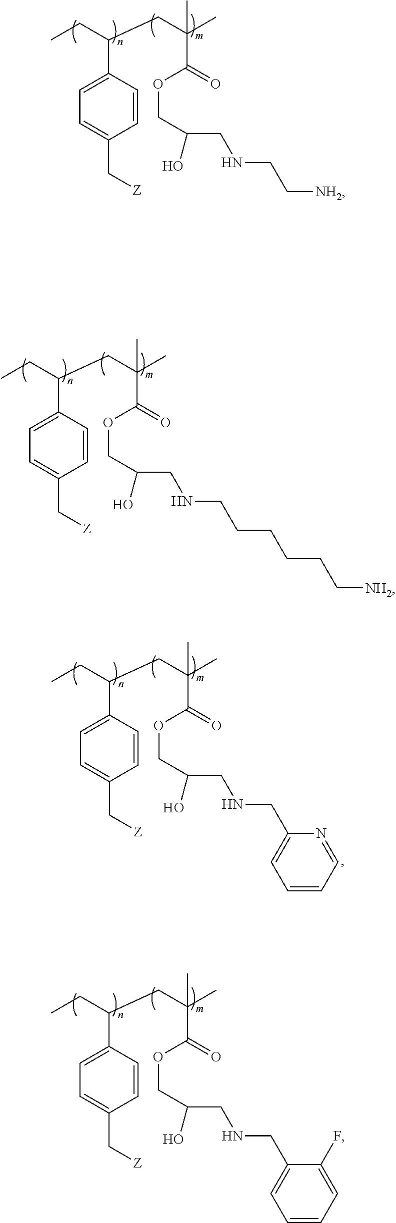 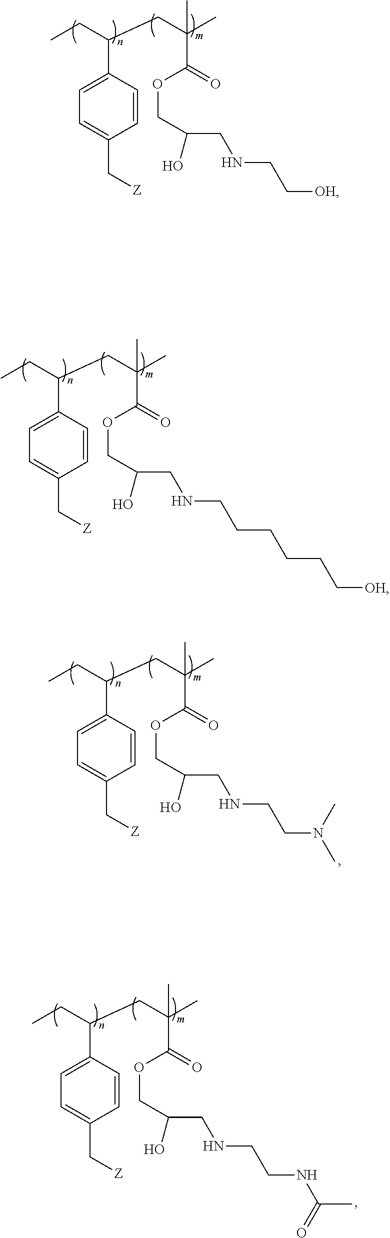 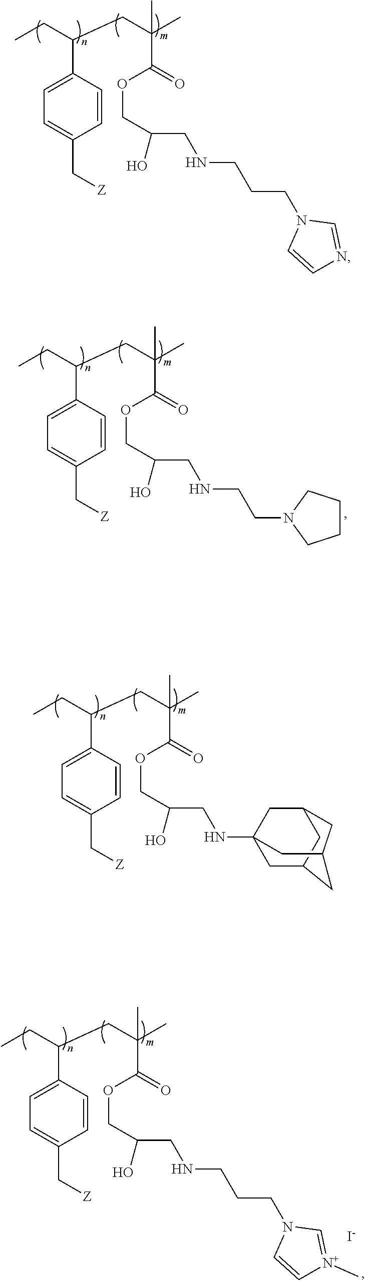 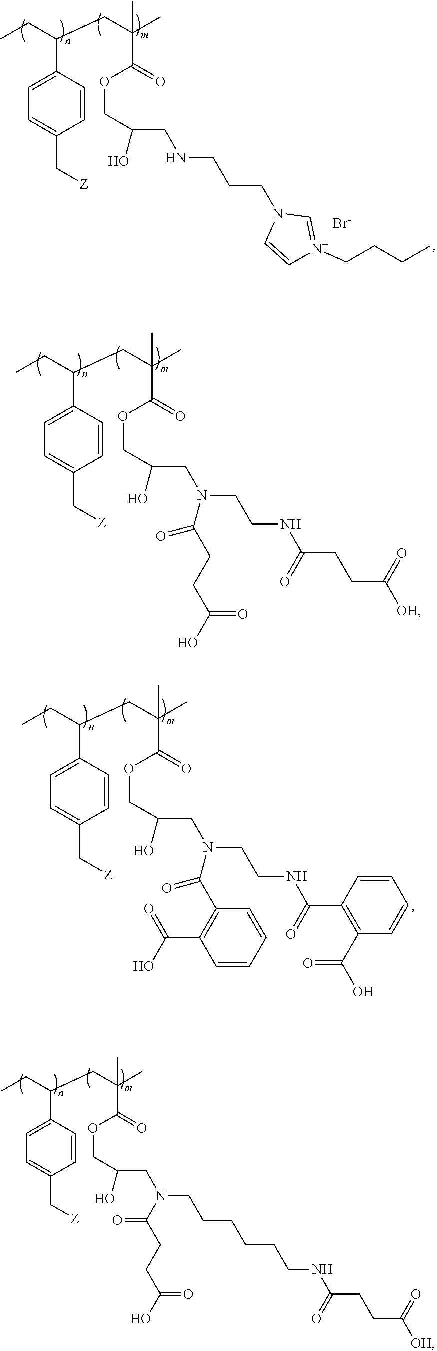 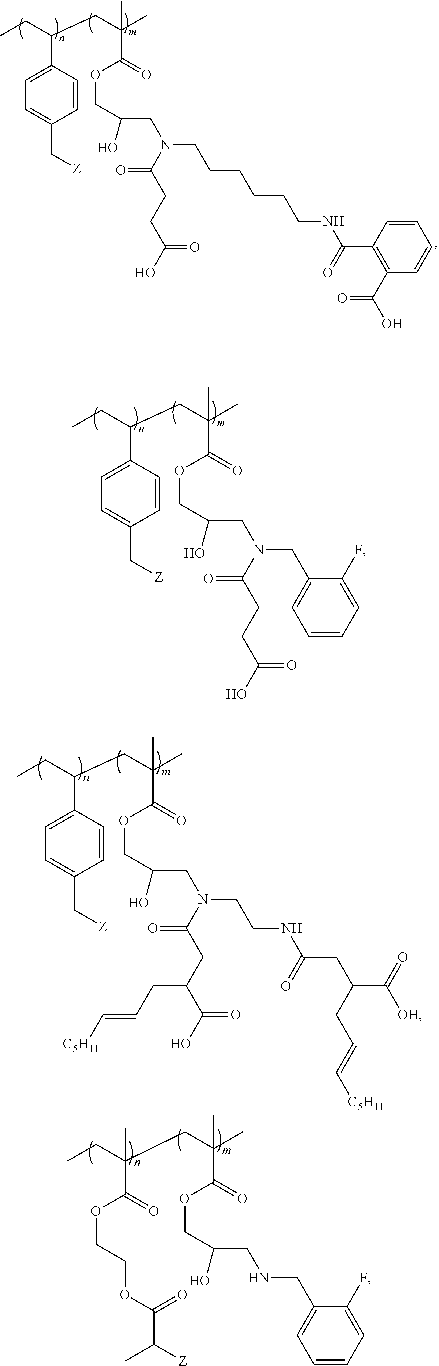 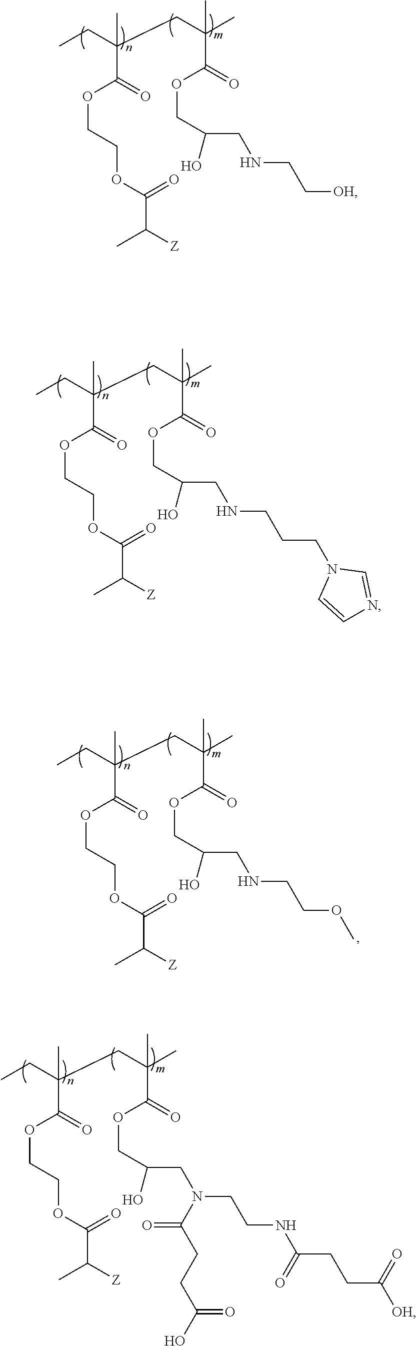 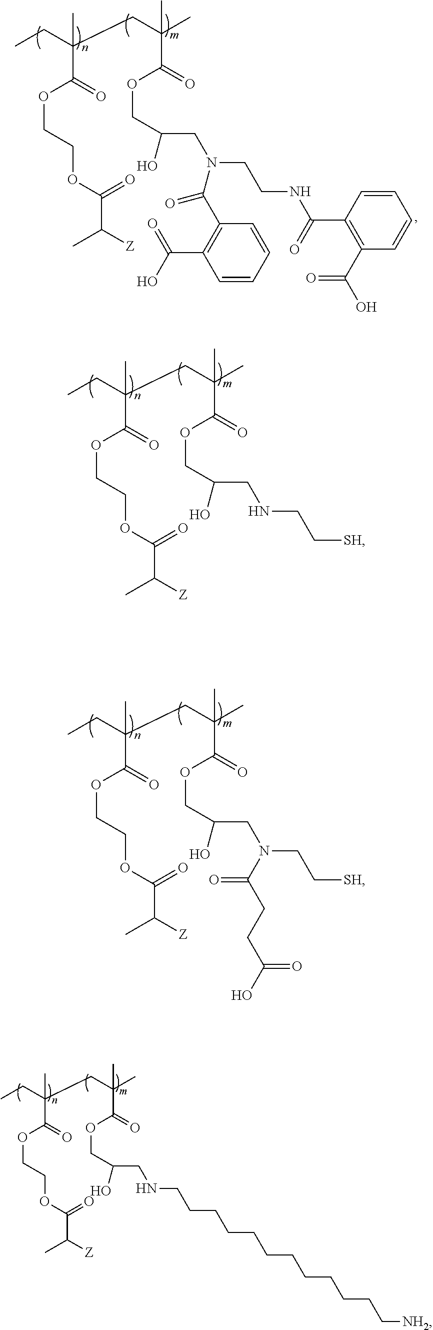 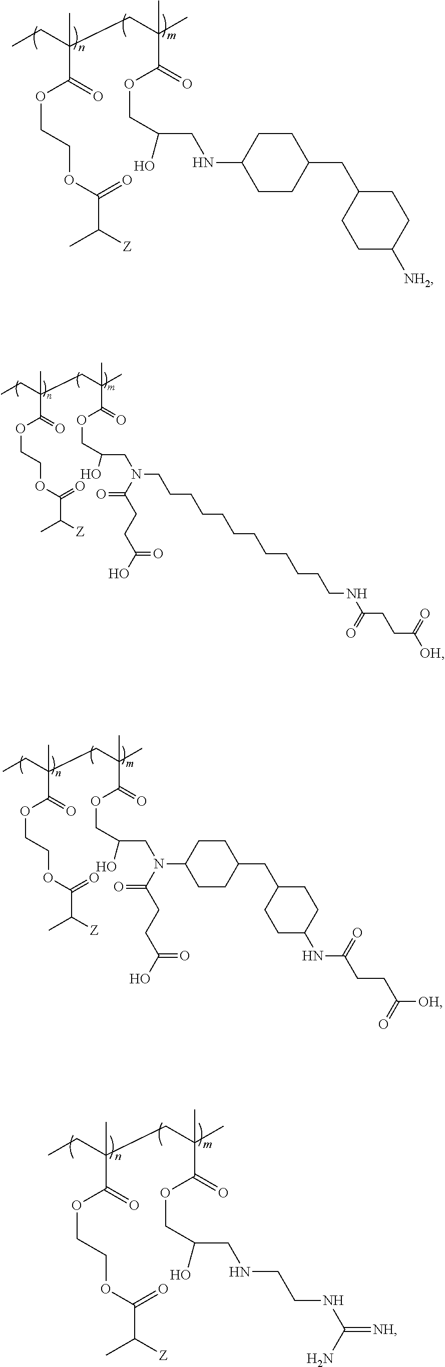 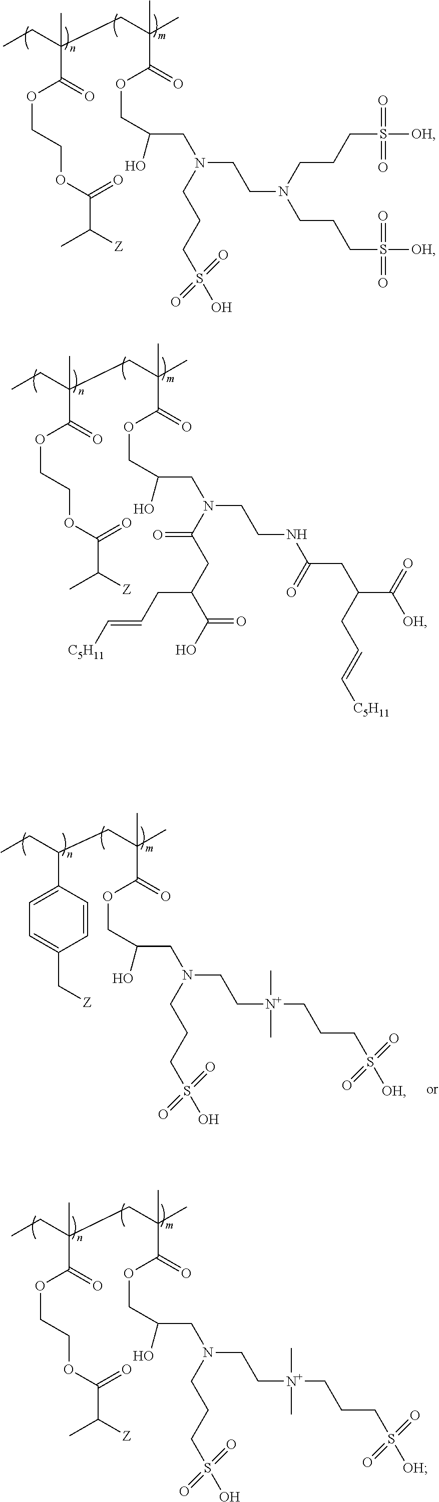
wherein 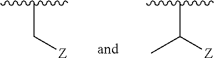
independently represent an attachment point for a unit of Component (A) or Component (B′);
n is an integer greater than 2; and
m is an integer greater than 2.

194. The macromolecule of claim 188, wherein B is

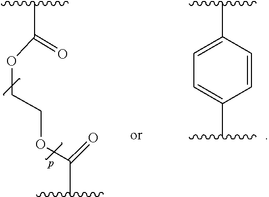

195. The macromolecule of claim 193, wherein B is

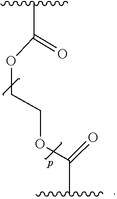

196. The macromolecule of claim 194, wherein p is 1.

197. The macromolecule of claim 188, wherein the macromolecule comprises a structure selected from: 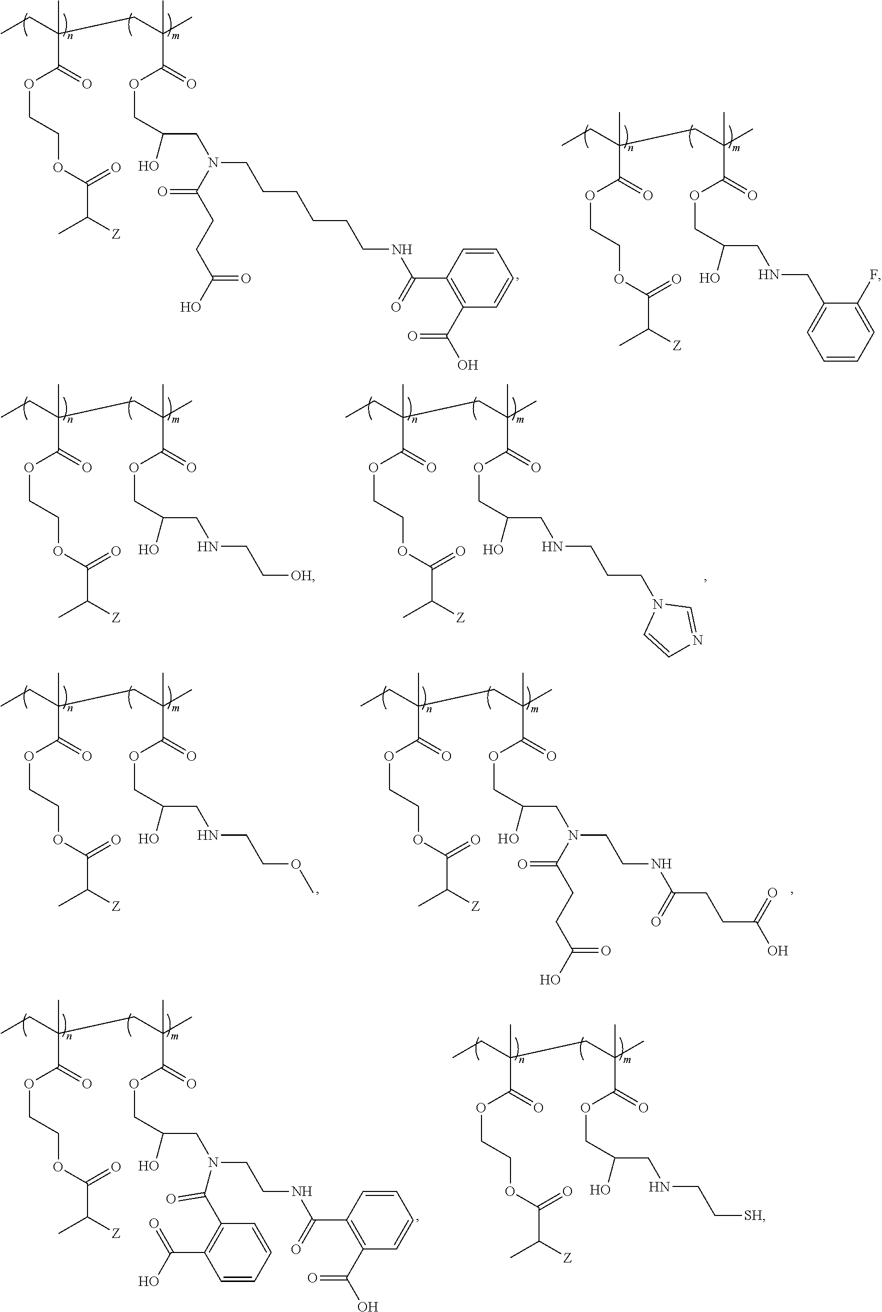 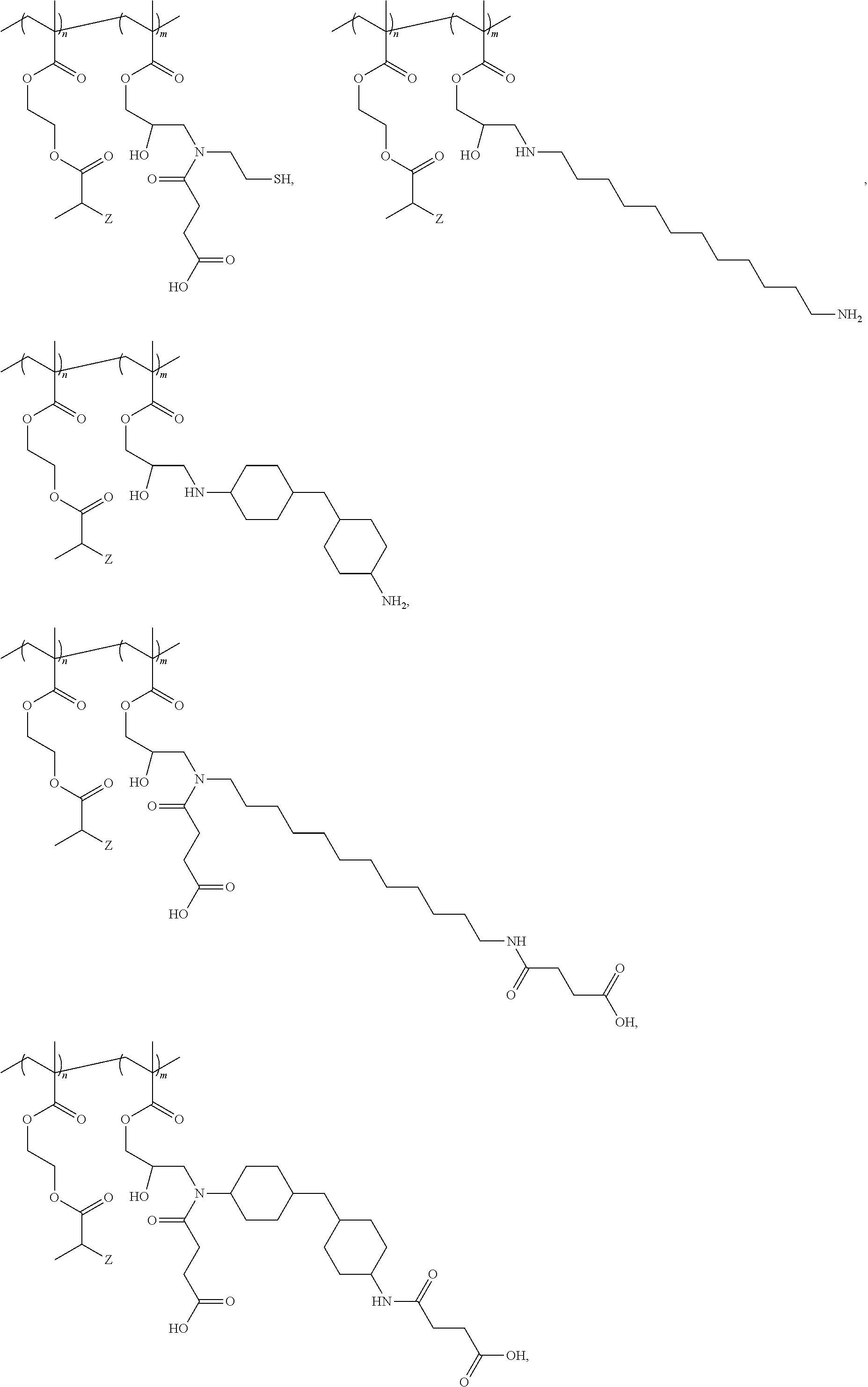 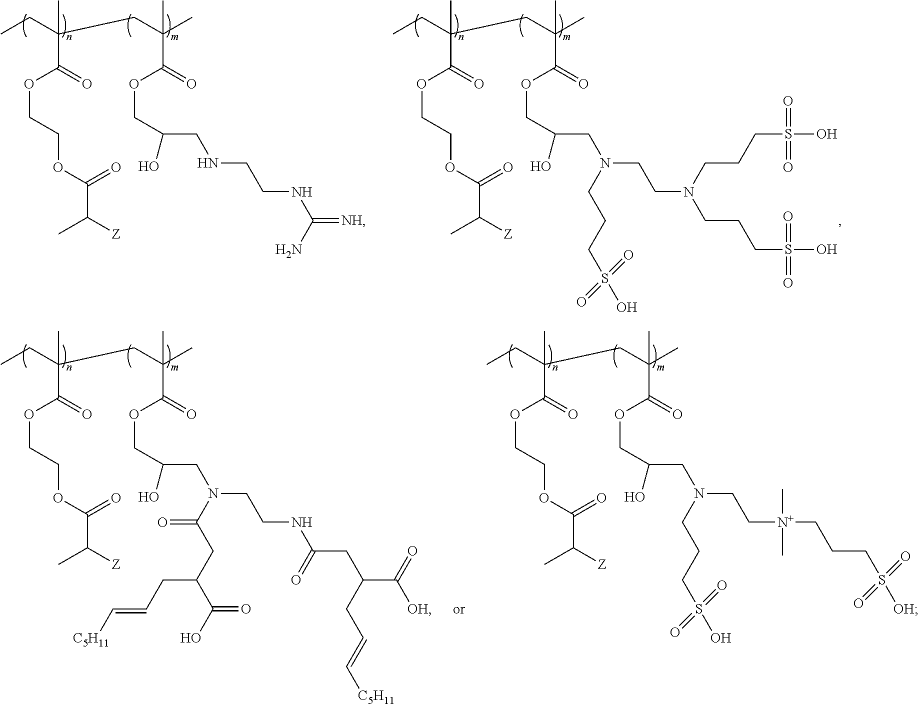
wherein 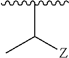
represents an attachment point for a unit of Component (A) or Component (B′);
n is an integer greater than 2; and
m is an integer greater than 2.

198. The macromolecule of claim 188, wherein the first component and the second component are randomly distributed throughout the macromolecule.

199. A particle comprising the macromolecule of claim 188, wherein the macromolecule is immobilized to the particle.

200. The particle of claim 199, wherein the macromolecule is covalently coupled to the particle.

201. The particle of claim 199, wherein the particle is a nanoparticle or microparticle.

202. The particle of claim 201, wherein the particle is a nanoparticle.

203. The particle of claim 202, wherein the particle has a diameter of about 200 nanometers (nm) to about 400 nm.

204. The particle of claim 203, wherein the particle comprises iron oxide.

205. The particle of claim 199, wherein the particle comprises an iron oxide core and a silica shell coating.

206. The particle of claim 199, wherein the particle comprises a structure selected from: 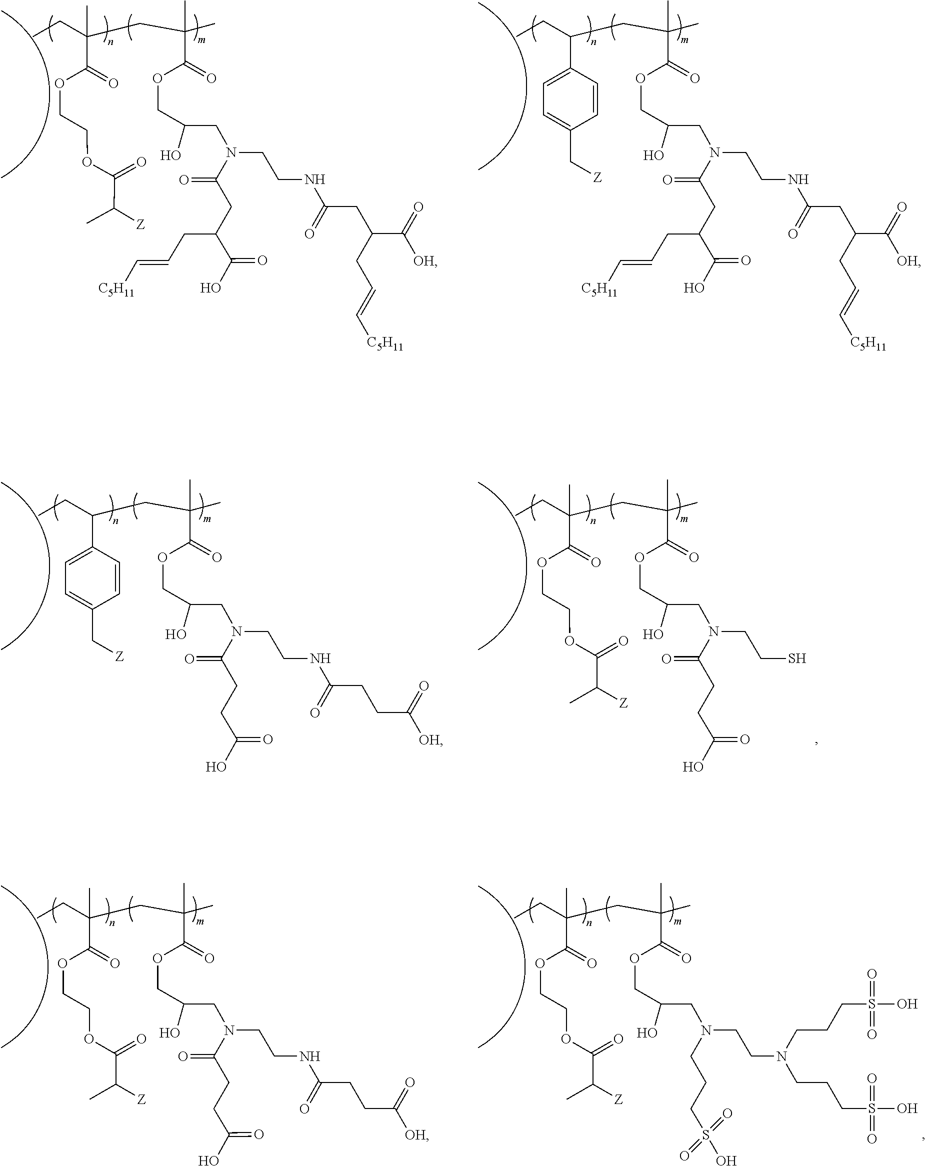 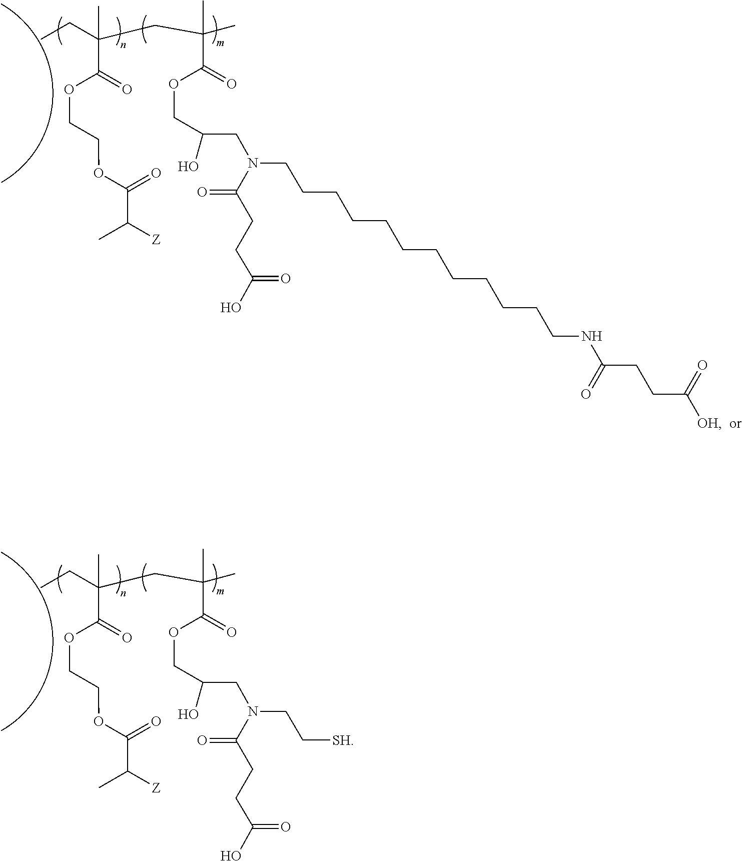
wherein 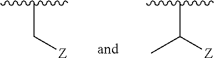
independently, represent an attachment point for a unit of Component (A) or Component (B′);
n is an integer greater than 2; and
m is an integer greater than 2.

207. A method comprising:
a. incubating at least two unique particles with a biological sample comprising a plurality of biomolecules to form a biomolecule corona, wherein at a one of the particles is a particle of claim 199;
b. isolating at least a portion of the biomolecules in the biomolecule corona; and
c. assaying the biomolecules or a portion thereof.

208. The method of claim 207, wherein the at least two unique particles comprise a first particle and a second particle.

209. The method of claim 208, wherein the first particle or the second particle comprise the structure: 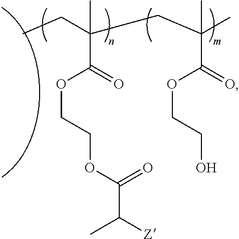
wherein 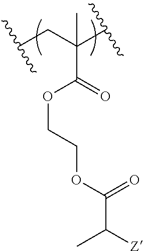
represents ethylene glycol dimethacrylate or a crosslinked version thereof,
n is an integer greater than 2; and
m is an integer greater than 2.

210. The method of claim 208, wherein the first particle and the second particle independently comprise a structure selected from: 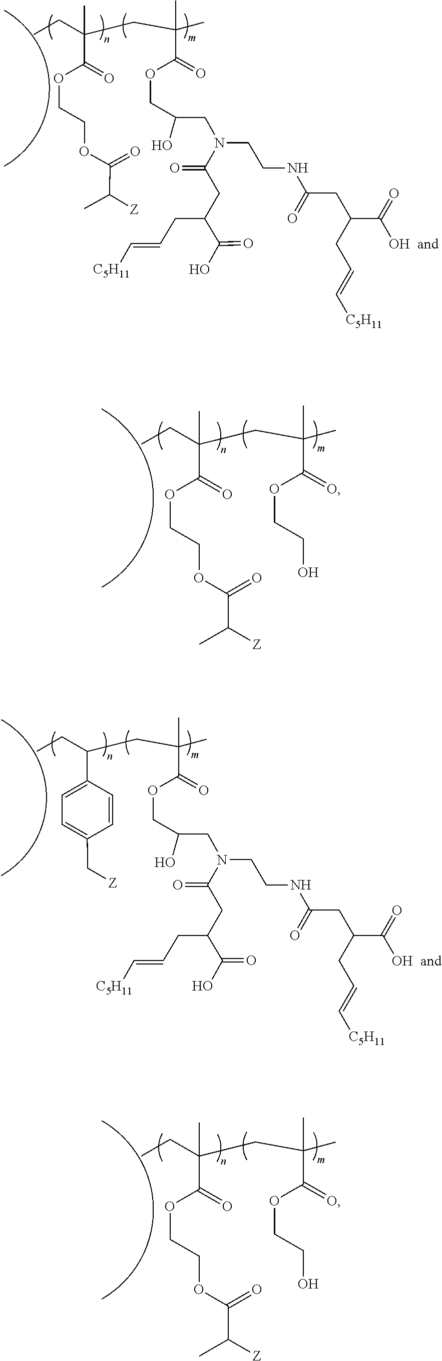 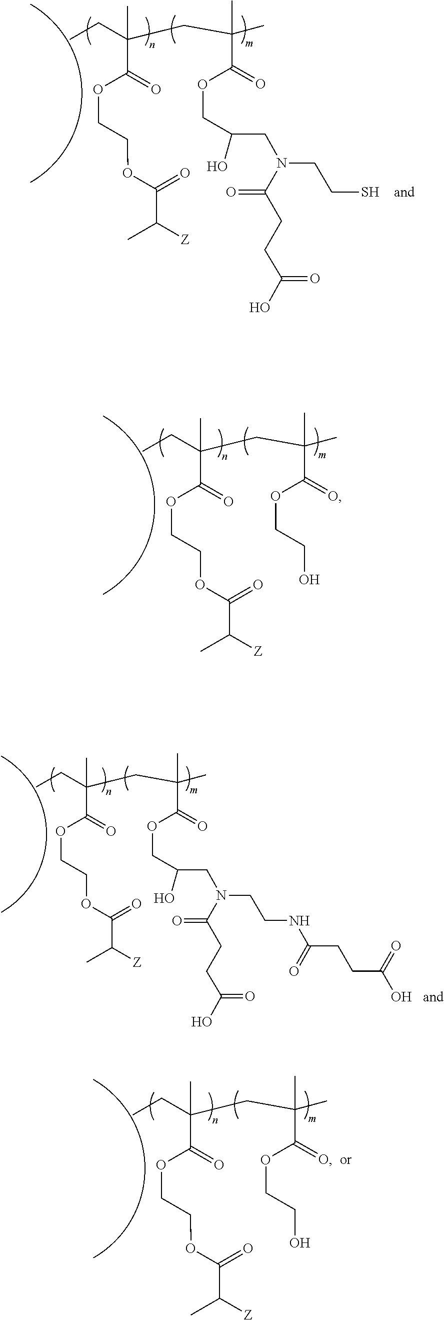 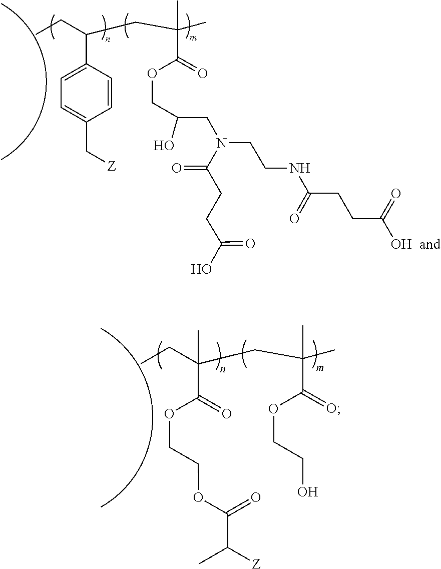
wherein 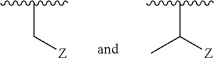
independently represent attachment points for a unit of Component (A) or Component (B′);
n is an integer greater than 2; and
m is an integer greater than 2.

211. The method of claim 207, wherein the biomolecule comprises a protein.

212. The method of claim 207, wherein the assaying is capable of identifying from 1,000 to 10,000 protein groups.

213. The method of claim 212, wherein the protein group comprises a peptide sequence having a minimum length of 7 amino acid residues.

214. The method of claim 213, wherein the assaying comprises using mass spectrometry to identify biomolecules in the sample.
